SEQUENCE LISTING (42 sequences)

SEQ ID NO: 1 - DNA, 21 bp, synthetic construct
  misc_feature 1..21: note = "A class" CpG oligonucleotide
  source 1..21: mol_type = other DNA
ggggacgacgtcgtggggggg

SEQ ID NO: 2 - DNA, 21 bp, synthetic construct
  misc_feature 1..21: note = "A class" CpG oligonucleotide
  source 1..21: mol_type = other DNA
ggggacgacgtcgtggggggg

SEQ ID NO: 3 - DNA, 21 bp, synthetic construct
  misc_feature 1..21: note = "B class" CpG oligonucleotide
  source 1..21: mol_type = other DNA
tcgtcgtttttcggtgctttt

SEQ ID NO: 4 - DNA, 21 bp, synthetic construct
  misc_feature 1..21: note = "B class" CpG oligonucleotide
  source 1..21: mol_type = other DNA
tcgtcgtttttcggtcgtttt

SEQ ID NO: 5 - DNA, 24 bp, synthetic construct
  misc_feature 1..24: note = "B class" CpG oligonucleotide
  source 1..24: mol_type = other DNA
tcgtcgttttgtcgttttgtcgtt

SEQ ID NO: 6 - DNA, 24 bp, synthetic construct
  misc_feature 1..24: note = "B class" CpG oligonucleotide
  source 1..24: mol_type = other DNA
tcgtcgtttcgtcgttttgtcgtt

SEQ ID NO: 7 - DNA, 24 bp, synthetic construct
  misc_feature 1..24: note = "B class" CpG oligonucleotide
  source 1..24: mol_type = other DNA
tcgtcgttttgtcgtttttttcga

SEQ ID NO: 8 - DNA, 21 bp, synthetic construct
  misc_feature 1..21: note = B-Class oligonucleotide
  source 1..21: mol_type = other DNA
tcgtcgtttttcggtgctttt

SEQ ID NO: 9 - DNA, 21 bp, synthetic construct
  misc_feature 1..21: note = B-Class oligonucleotide
  source 1..21: mol_type = other DNA
tcgtcgtttttcggtcgtttt

SEQ ID NO: 10 - DNA, 24 bp, synthetic construct
  misc_feature 1..24: note = B-Class oligonucleotide
  source 1..24: mol_type = other DNA
tcgtcgttttgtcgttttgtcgtt

SEQ ID NO: 11 - DNA, 24 bp, synthetic construct
  misc_feature 1..24: note = B-Class oligonucleotide
  source 1..24: mol_type = other DNA
tcgtcgtttcgtcgttttgtcgtt

SEQ ID NO: 12 - DNA, 24 bp, synthetic construct
  misc_feature 1..24: note = B-Class oligonucleotide
  source 1..24: mol_type = other DNA
tcgtcgttttgtcgtttttttcga

SEQ ID NO: 13 - DNA, 22 bp, synthetic construct
  misc_feature 1..22: note = C class CpG Oligonucleotide
  source 1..22: mol_type = other DNA
tcgcgtcgttcggcgcgcgccg

SEQ ID NO: 14 - DNA, 23 bp, synthetic construct
  misc_feature 1..23: note = C class CpG Oligonucleotide
  source 1..23: mol_type = other DNA
tcgtcgacgttcggcgcgcgccg

SEQ ID NO: 15 - DNA, 21 bp, synthetic construct
  misc_feature 1..21: note = C class CpG Oligonucleotide
  source 1..21: mol_type = other DNA
tcggacgttcggcgcgcgccg

SEQ ID NO: 16 - DNA, 19 bp, synthetic construct
  misc_feature 1..19: note = C class CpG Oligonucleotide
  source 1..19: mol_type = other DNA
tcggacgttcggcgcgccg

SEQ ID NO: 17 - DNA, 20 bp, synthetic construct
  misc_feature 1..20: note = C class CpG Oligonucleotide
  source 1..20: mol_type = other DNA
tcgcgtcgttcggcgcgccg

SEQ ID NO: 18 - DNA, 20 bp, synthetic construct
  misc_feature 1..20: note = C class CpG Oligonucleotide
  source 1..20: mol_type = other DNA
tcgacgttcggcgcgcgccg

SEQ ID NO: 19 - DNA, 18 bp, synthetic construct
  misc_feature 1..18: note = C class CpG Oligonucleotide
  source 1..18: mol_type = other DNA
tcgacgttcggcgcgccg

SEQ ID NO: 20 - DNA, 18 bp, synthetic construct
  misc_feature 1..18: note = C class CpG Oligonucleotide
  source 1..18: mol_type = other DNA
tcgcgtcgttcggcgccg

SEQ ID NO: 21 - DNA, 22 bp, synthetic construct
  misc_feature 1..22: note = C class CpG Oligonucleotide
  source 1..22: mol_type = other DNA
tcgcgacgttcggcgcgcgccg

SEQ ID NO: 22 - DNA, 22 bp, synthetic construct
  misc_feature 1..22: note = C class CpG Oligonucleotide
  source 1..22: mol_type = other DNA
tcgtcgttttcggcgcgcgccg

SEQ ID NO: 23 - DNA, 22 bp, synthetic construct
  misc_feature 1..22: note = C class CpG Oligonucleotide
  source 1..22: mol_type = other DNA
tcgtcgttttcggcggccgccg

SEQ ID NO: 24 - DNA, 24 bp, synthetic construct
  misc_feature 1..24: note = C class CpG Oligonucleotide
  source 1..24: mol_type = other DNA
tcgtcgttttacggcgccgtgccg

SEQ ID NO: 25 - DNA, 23 bp, synthetic construct
  misc_feature 1..23: note = C class CpG Oligonucleotide
  source 1..23: mol_type = other DNA
tcgtcgttttcggcgcgcgccgt

SEQ ID NO: 26 - DNA, 22 bp, synthetic construct
  misc_feature 1..22: note = C-Class oligonucleotides
  source 1..22: mol_type = other DNA
tcgcgtcgttcggcgcgcgccg

SEQ ID NO: 27 - DNA, 23 bp, synthetic construct
  misc_feature 1..23: note = C-Class oligonucleotide
  source 1..23: mol_type = other DNA
tcgtcgacgttcggcgcgcgccg

SEQ ID NO: 28 - DNA, 21 bp, synthetic construct
  misc_feature 1..21: note = C-Class oligonucleotide
  source 1..21: mol_type = other DNA
tcggacgttcggcgcgcgccg

SEQ ID NO: 29 - DNA, 19 bp, synthetic construct
  misc_feature 1..19: note = C-Class oligonucleotide
  source 1..19: mol_type = other DNA
tcggacgttcggcgcgccg

SEQ ID NO: 30 - DNA, 20 bp, synthetic construct
  misc_feature 1..20: note = C-Class oligonucleotide
  source 1..20: mol_type = other DNA
tcgcgtcgttcggcgcgccg

SEQ ID NO: 31 - DNA, 20 bp, synthetic construct
  misc_feature 1..20: note = C-Class oligonucleotide
  source 1..20: mol_type = other DNA
tcgacgttcggcgcgcgccg

SEQ ID NO: 32 - DNA, 18 bp, synthetic construct
  misc_feature 1..18: note = C-Class oligonucleotide
  source 1..18: mol_type = other DNA
tcgacgttcggcgcgccg

SEQ ID NO: 33 - DNA, 18 bp, synthetic construct
  misc_feature 1..18: note = C-Class oligonucleotide
  source 1..18: mol_type = other DNA
tcgcgtcgttcggcgccg

SEQ ID NO: 34 - DNA, 22 bp, synthetic construct
  misc_feature 1..22: note = C-Class oligonucleotide
  source 1..22: mol_type = other DNA
tcgcgacgttcggcgcgcgccg

SEQ ID NO: 35 - DNA, 22 bp, synthetic construct
  misc_feature 1..22: note = C-Class oligonucleotide
  source 1..22: mol_type = other DNA
tcgtcgttttcggcgcgcgccg

SEQ ID NO: 36 - DNA, 22 bp, synthetic construct
  misc_feature 1..22: note = C-Class oligonucleotide
  source 1..22: mol_type = other DNA
tcgtcgttttcggcggccgccg

SEQ ID NO: 37 - DNA, 24 bp, synthetic construct
  misc_feature 1..24: note = C-Class oligonucleotide
  source 1..24: mol_type = other DNA
tcgtcgttttacggcgccgtgccg

SEQ ID NO: 38 - DNA, 23 bp, synthetic construct
  misc_feature 1..23: note = C-Class oligonucleotide
  source 1..23: mol_type = other DNA
tcgtcgttttcggcgcgcgccgt

SEQ ID NO: 39 - DNA, 23 bp, synthetic construct
  misc_feature 1..23: note = P class CpG oligonucleotide
  source 1..23: mol_type = other DNA
tcgtcgacgatcggcgcgcgccg

SEQ ID NO: 40 - DNA, 23 bp, synthetic construct
  misc_feature 1..23: note = P class CpG oligonucleotide
  source 1..23: mol_type = other DNA
tcgtcgacgatcggcgcgcgccg

SEQ ID NO: 41 - protein, 950 aa, synthetic construct
  REGION 1..950: note = Enzymatically inactive fragment of SCP
MAKTADTPATSKATIRDLNDPSQVKTLQEKAGKGAGTVVAVIAAGFDKNHEAWRLTDKAKARYQSKEDLEKAKKEHGITYGEWVNDKVAYYHDYSKDGKTAVDQEHGTHVSGILSGNAPSETKEPYRLEGAMPEAQLLLMRVEIVNGLADYARNYAQAIRDAINLGAKVINMSFGNAALAYANLPDETKKAFDYAKSKGVSIVTSAGNDSSFGGKTRLPLADHPDYGVVGTPAAADSTLTVASYSPDKQLTETVTVKTADQQDKEMPVLSTNRFEPNKAYDYAYANRGTKEDDFKDVKGKIALIERGDIDFKDKIAKAKKAGAVGVLIYDNQDKGFPIELPNVDQMPAAFISRKDGLLLKDNPQKTITFNATPKVLPTASGTKLSRFSSWGLTADGNIKPDIAAPGQDILSSVANNKYAKLSGTAMSAPLVAGIMGLLQEQYETQYPDMTPSERLDLAKKVLMSSATALYDEDEKAYFSPRQQGAGAVDAKKASAATMYVTDKDNTSSKVHLNNVSDKFEVTVTVHNKSDKPQELYYQATVQTDKVDGKHFALAPKALYETSWQKITIPANSSKQVTVPIDASRFSKDLLAQMKNGYFLEGFVRFKQDPKKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQVVKNYNNDLASTLGSTRFEKTRWDGKDKDGKVVANGTYTYRVRYTPISSGAKEQHTDFDVIVDNTTPEVATSATFSTEDRRLTLASKPKTSQPVYRERIAYTYMDEDLPTTEYISPNEDGTFTLPEEAETMEGATVPLKMSDFTYVVEDMAGNITYTPVTKLLEGHSNKPEQ

SEQ ID NO: 42 - protein, 949 aa, synthetic construct
  REGION 1..949: note = Enzymatically inactive fragment of SCP
AKTADTPATSKATIRDLNDPSQVKTLQEKAGKGAGTVVAVIAAGFDKNHEAWRLTDKAKARYQSKEDLEKAKKEHGITYGEWVNDKVAYYHDYSKDGKTAVDQEHGTHVSGILSGNAPSETKEPYRLEGAMPEAQLLLMRVEIVNGLADYARNYAQAIRDAINLGAKVINMSFGNAALAYANLPDETKKAFDYAKSKGVSIVTSAGNDSSFGGKTRLPLADHPDYGVVGTPAAADSTLTVASYSPDKQLTETVTVKTADQQDKEMPVLSTNRFEPNKAYDYAYANRGTKEDDFKDVKGKIALIERGDIDFKDKIAKAKKAGAVGVLIYDNQDKGFPIELPNVDQMPAAFISRKDGLLLKDNPQKTITFNATPKVLPTASGTKLSRFSSWGLTADGNIKPDIAAPGQDILSSVANNKYAKLSGTAMSAPLVAGIMGLLQEQYETQYPDMTPSERLDLAKKVLMSSATALYDEDEKAYFSPRQQGAGAVDAKKASAATMYVTDKDNTSSKVHLNNVSDKFEVTVTVHNKSDKPQELYYQATVQTDKVDGKHFALAPKALYETSWQKITIPANSSKQVTVPIDASRFSKDLLAQMKNGYFLEGFVRFKQDPKKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQVVKNYNNDLASTLGSTRFEKTRWDGKDKDGKVVANGTYTYRVRYTPISSGAKEQHTDFDVIVDNTTPEVATSATFSTEDRRLTLASKPKTSQPVYRERIAYTYMDEDLPTTEYISPNEDGTFTLPEEAETMEGATVPLKMSDFTYVVEDMAGNITYTPVTKLLEGHSNKPEQ

(19) United States
(12) Patent Application Publication
(10) Pub. No.: US 2026/0014241 A1
(43) Pub. Date: Jan. 15, 2026

(54) Immunogenic compositions comprising conjugated capsular saccharide antigens and uses thereof

(71) Applicant: PFIZER INC., New York, NY (US)

(72) Inventors: Caitlyn GALLAGHER, Ringwood, NJ (US); Jianxin GU, Paramus, NJ (US); Isis KANEVSKY, New York, NY (US); Suddham SINGH, Monroe, NY (US); Sandeep THANNA, Montvale, NJ (US); Justin Keith MORAN, West Nyack, NY (US)

(21) Appl. No.: 19/071,620

(22) Filed: Mar. 5, 2025

Related U.S. Application Data
(60) Provisional application No. 63/561,954, filed on Mar. 6, 2024.
(60) Provisional application No. 63/754,947, filed on Feb. 6, 2025.

(51) Int. Cl. A61K 39/09; A61K 39/00; A61K 47/64

(52) U.S. Cl. CPC A61K 39/092; A61K 47/6415; A61K 47/646; A61K 2039/6037

(57) ABSTRACT

The present invention relates to new conjugated capsular saccharide antigens (glycoconjugates), immunogenic compositions comprising said glycoconjugates and uses thereof.

### CROSS-REFERENCE TO RELATED APPLICATIONS

This application claims the benefit under 35 U.S.C. § 119(e) of U.S. Provisional Application No. 63/561,954, filed Mar. 6, 2024, and U.S. Provisional Application No. 63/754,947, filed Feb. 6, 2025, the disclosure of each of which is incorporated by reference herein in its entirety.

Reference to Sequence Listing This application is being filed electronically via Patent Center and includes an electronically submitted Sequence Listing XML file named “PC073064A.xml” created on Mar. 3, 2025 and having a size of 56,178 bytes. The material in the Sequence Listing XML file PC073064.xml is part of the specification and is incorporated by reference herein in its entirety.

### FIELD OF THE INVENTION

The present invention relates to new conjugated capsular saccharide antigens (glycoconjugates), immunogenic compositions comprising said glycoconjugates and uses thereof. Immunogenic compositions of the present invention will typically comprise glycoconjugates, wherein the saccharides are derived from serotypes of Streptococcus pneumoniae. The invention also relates to vaccination of human subjects, in particular infants and elderly, against pneumoccocal infections using said glycoconjugates.

More particularly, the invention relates to Streptococcus pneumoniae serotype 9N glycoconjugates and immunogenic composition comprising a Streptococcus pneumoniae serotype 9N glycoconjugate.

The Streptococcus pneumoniae serotype 9N glycoconjugates of the invention can be used as a vaccine.

### BACKGROUND OF THE INVENTION

Infections caused by pneumococci are a major cause of morbidity and mortality all over the world. Pneumonia, febrile bacteraemia and meningitis are the most common manifestations of invasive pneumococcal disease, whereas bacterial spread within the respiratory tract may result in middle-ear infection, sinusitis or recurrent bronchitis. Compared with invasive disease, the non-invasive manifestations are usually less severe, but considerably more common.

The etiological agent of pneumococcal diseases, Streptococcus pneumoniae (pneumococcus), is a Gram-positive encapsulated coccus, surrounded by a polysaccharide capsule. Differences in the composition of this capsule permit serological differentiation between about 91 capsular types, some of which are frequently associated with pneumococcal disease, others rarely. Invasive pneumococcal infections include pneumonia, meningitis and febrile bacteraemia; among the common non-invasive manifestations are otitis media, sinusitis and bronchitis.

Pneumococcal polysaccharides, in particular capsular polysaccharides, are important immunogens found on the surface of the bacteria. This has led to them being an important component in the design of pneumococcal vaccines. They have proved useful in eliciting immune responses especially when linked to carrier proteins.

Some serotypes, in particular Streptococcus pneumoniae serotype 9N, produce large and viscous polysaccharide chains. Its viscosity has made it difficult to handle.

Thus, there is a need for antigens which are able to generate a more robust immune response to Streptococcus pneumoniae serotype 9N.

The present invention provides in particular Streptococcus pneumoniae serotype 9N glycoconjugates which show improved immunogenicity. The present invention also provides processes which generate Streptococcus pneumoniae serotype 9N glycoconjugates with fewer operational steps, and better conjugation yields.

### FIGURES

FIG. 1 shows a repeating polysaccharide structure of the S. pneumoniae serotype 9N capsular polysaccharide.

FIG. 2 shows opsonophagocytic activity (OPA) titers for S. pneumoniae serotype 9N conjugates in mice. Different chemistries (Reductive Amination in DMSO or click chemistry (Click)) and polysaccharides of different size have been used.

FIG. 3 shows opsonophagocytic activity (OPA) titers for S. pneumoniae serotype 9N conjugates in mice. Conjugates with different attributes have been used.

The present invention is directed in part to conjugated capsular saccharide antigens (also named glycoconjugates). For the purpose of the invention the term ‘glycoconjugate’ indicates a capsular saccharide conjugated to a carrier protein via covalent or non-covalent bonds. In an embodiment, the capsular saccharide is conjugated to a carrier protein via non-covalent bonds. Preferably, the capsular saccharide is conjugated via covalent bonds. In one embodiment the capsular saccharide is conjugated directly to a carrier protein. In a second embodiment the capsular saccharide is conjugated to a carrier protein through a spacer/linker.

1. Capsular Streptococcus pneumoniae Serotype 9N Saccharide of the Invention

The structure of Streptococcus pneumoniae serotype 9N polysaccharide is known in the art. The polysaccharide repeating unit of serotype 9N consists of a pentasaccharide unit (see e.g. Geno K et al. (2015) Clin Microbiol Rev Vol 28:3, p 871-899):

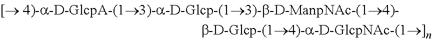

The term “saccharide” throughout this specification may indicate polysaccharide or oligosaccharide and includes both. In frequent embodiments, the saccharide is a polysaccharide, in particular a S. pneumoniae capsular polysaccharide.

In an embodiment, the capsular S. pneumoniae serotype 9N saccharide used in the present invention is an oligosaccharide. Oligosaccharides have a low number of repeat units (typically 5-15 repeat units) and are typically derived synthetically or by hydrolysis of polysaccharides.

In an embodiment, the capsular S. pneumoniae serotype 9N saccharide used in the present invention is a synthetic carbohydrate.

In a preferred embodiment though, the source of bacterial capsular saccharide according to this invention can be Streptococcus pneumoniae serotype 9N bacterial cells. Bacterial strains which can be used as source of S. pneumoniae serotype 9N polysaccharides may be obtained from established culture collections (such as for example from the Streptococcal Reference Laboratory (Centers for Disease Control and Prevention, Atlanta, GA USA)) or clinical specimens.

Capsular S. pneumoniae serotype 9N saccharide can be obtained directly from bacteria using isolation procedures known to one of ordinary skill in the art (see for example methods disclosed in US2006/0228380, US2006/0228381, US2007/0184071, US2007/0184072, US2007/0231340, and US2008/0102498 and WO2008/118752). They can also be purchased (such as for example from the American Type Culture Collection (ATCC, Manassas, VA USA) (e.g., reference No. ATCC 192-X or ATCC 21-X)).

In case the capsular S. pneumoniae serotype 9N saccharide is obtained directly from bacteria, the bacterial cells can be grown in a medium, preferably in a soy based medium. Following fermentation of bacterial cells that produce S. pneumoniae serotype 9N capsular saccharides, the bacterial cells can be lysed to produce a cell lysate. The capsular S. pneumoniae serotype 9N saccharide may then be isolated from the cell lysate using purification techniques known in the art, including the use of centrifugation, depth filtration, precipitation, ultra-filtration, treatment with activate carbon, diafiltration and/or column chromatography (see, for example, US2006/0228380, US2006/0228381 and WO2008/118752). The isolated capsular S. pneumoniae serotype 9N saccharide can then be used for the preparation of immunogenic conjugates.

As used herein, the term “isolated” in connection with a saccharide refers to isolation of S. pneumoniae serotype specific capsular saccharide from purified saccharide using purification techniques known in the art, including the use of centrifugation, depth filtration, precipitation, ultrafiltration, treatment with activate carbon, diafiltration and/or column chromatography. Generally, an isolated saccharide refers to partial removal of proteins, nucleic acids and non-specific endogenous polysaccharide (C-polysaccharide). The isolated saccharide contains less than 10%, 8%, 6%, 4%, or 2% protein impurities and/or nucleic acids. The isolated saccharide contains less than 20% of C-polysaccharide with respect to type specific saccharides.

The isolated capsular S. pneumoniae serotype 9N saccharide obtained by purification from the S. pneumoniae lysate can be characterized by different parameters including, for example the weight average molecular weight (Mw).

The molecular weight of the saccharide can be measured by Size Exclusion Chromatography (SEC) combined with Multiangle Laser Light Scattering detector (MALLS).

In an embodiment, the isolated capsular S. pneumoniae serotype 9N saccharide (i.e. purified before further treatment) has a weight average molecular weight between 5 kDa and 5,000 kDa. In an embodiment, the isolated capsular S. pneumoniae serotype 9N polysaccharide has a weight average molecular weight between 100 kDa and 4,000 kDa. In a preferred embodiment, the isolated capsular polysaccharide has a weight average molecular weight between 500 kDa and 2,000 kDa. In an even preferred embodiment, the isolated capsular polysaccharide has a weight average molecular weight between 500 kDa and 1,000 kDa.

The capsular Streptococcus pneumoniae serotype 9N saccharides of the invention may be sized and/or activated (e.g., chemically activated) to make them capable of reacting (e.g., either directly to the carrier protein of via a linker) and then incorporated into glycoconjugates of the invention, as further described herein.

2. S. pneumoniae Serotype 9N Glycoconjugates of the Invention

For the purpose of the invention the term ‘glycoconjugate’ indicates a capsular saccharide conjugated to a carrier protein via covalent or non-covalent bonds. In an embodiment, the capsular saccharide is conjugated to a carrier protein via non-covalent bonds (such as the rhizavidin/biotin system, see e.g. WO2012155007, WO2020056202). Preferably, the capsular saccharide is conjugated via covalent bonds. In one embodiment the capsular saccharide is conjugated directly to a carrier protein. In a second embodiment the capsular saccharide is conjugated to a carrier protein through a spacer/linker.

The present invention provides glycoconjugates in which saccharides as provided for above are conjugated to a carrier protein. Therefore, in an embodiment, the invention provides a glycoconjugate comprising a saccharide having the above disclosed repeating unit conjugated to a carrier protein.

In an embodiment, the invention provides a glycoconjugate consisting of a saccharide having the above disclosed repeating unit conjugated to a carrier protein.

2.1 Attributes of the S. pneumoniae Serotype 9N Glycoconjugates of the Invention

The serotype 9N saccharide described above may be activated (e.g., chemically activated) to make them capable of reacting (e.g. with a linker or directly with the carrier protein) and then incorporated into glycoconjugates, as further described herein.

In order to generate serotype 9N conjugates with advantageous filterability characteristics and/or yields, sizing of polysaccharide to a target molecular weight range may be performed prior to the conjugation to a carrier protein. Advantageously, the size of isolated serotype 9N polysaccharide is reduced while preserving critical features of the structure of the polysaccharide. mechanical or chemical sizing maybe employed.

In an embodiment, the size of isolated serotype 9N polysaccharide is reduced by chemical hydrolysis. Chemical hydrolysis maybe conducted using a mild acid (e.g acetic acid, formic acid, propanoic acid). In an embodiment, chemical hydrolysis is conducted using formic acid. In an embodiment, chemical hydrolysis is conducted using propanoic acid. In a preferred embodiment, chemical hydrolysis is conducted using acetic acid. In an embodiment, chemical hydrolysis is conducted in acetic acid at 0.15-0.25 M. In a preferred embodiment, chemical hydrolysis is conducted in acetic acid at about 0.2 M. In an embodiment, chemical hydrolysis is conducted in acetic acid at 0.15-0.25 M, at a temperature of 80 to 90° C. In a preferred embodiment, chemical hydrolysis is conducted in acetic acid at about 0.2 M at a temperature of about 85° C. for 3 to 7 h. In a preferred embodiment, chemical hydrolysis is conducted in acetic acid at about 0.2 M at a temperature of about 85° C. for about 5 h.

Chemical hydrolysis may also be conducted using a diluted strong acid (such as diluted hydrochloric acid, diluted sulfuric acid, diluted phosphoric acid, diluted nitric acid or diluted perchloric acid). In an embodiment, chemical hydrolysis is conducted using diluted hydrochloric acid. In an embodiment, chemical hydrolysis is conducted using diluted sulfuric acid. In an embodiment, chemical hydrolysis is conducted using diluted phosphoric acid. In an embodiment, chemical hydrolysis is conducted using diluted nitric acid. In an embodiment, chemical hydrolysis is conducted using diluted perchloric acid.

The size of isolated serotype 9N polysaccharide can also be reduced by mechanical homogenization. In an embodiment, the size of isolated serotype 9N polysaccharide is reduced by high pressure homogenization. High pressure homogenization achieves high shear rates by pumping the process stream through a flow path with sufficiently small dimensions. The shear rate is increased by using a larger applied homogenization pressure, and exposure time can be increased by recirculating the feed stream through the homogenizer. The high-pressure homogenization process can be appropriate for reducing the size of isolated serotype 9N polysaccharide while preserving the structural features of the polysaccharide.

In an embodiment, the isolated serotype 9N capsular polysaccharide is sized to a weight average molecular weight between 5 kDa and 500 kDa. In an embodiment, the isolated serotype 9N capsular polysaccharide is sized to a weight average molecular weight between 50 kDa and 450 kDa. In a preferred embodiment, the isolated serotype 9N capsular polysaccharide is sized to a weight average molecular weight between 100 kDa and 400 kDa.

In an embodiment, the isolated serotype 9N capsular polysaccharide is sized to a weight average molecular weight of between about 200 kDa and about 300 kDa.

In an embodiment, the isolated serotype 9N capsular polysaccharide is sized to a weight average molecular weight of between about 100 kDa and about 200 kDa.

In an embodiment, the isolated serotype 9N capsular polysaccharide is not sized.

In a preferred embodiment, the capsular S. pneumoniae serotype 9N saccharide used in the present invention is a polysaccharide. High molecular weight capsular polysaccharides are able to induce certain antibody immune responses due to the epitopes present on the antigenic surface. The isolation and purification of high molecular weight capsular polysaccharides is preferably contemplated for use in the conjugates, compositions and methods of the present invention.

In an embodiment, the serotype 9N glycoconjugate of the present invention comprises a serotype 9N capsular polysaccharide wherein the weight average molecular weight (Mw) of said polysaccharide before conjugation is between 50 kDa and 1,000 kDa. Preferably, the weight average molecular weight (Mw) is between 100 kDa and 800 kDa.

The weight average molecular weight (Mw) of the serotype 9N saccharide before conjugation refers to the Mw before the activation of the serotype 9N polysaccharide (i.e. after an eventual sizing step but before reacting the polysaccharide with an activating agent). In the context of the present invention the Mw of the serotype 9N polysaccharide is not substantially modified by the activation step and the Mw of the serotype 9N polysaccharide incorporated in the conjugate is similar to the Mw of the polysaccharide as measured before activation.

In an embodiment, the serotype 9N glycoconjugate of the present invention comprises a serotype 9N capsular polysaccharide wherein the weight average molecular weight (Mw) of said polysaccharide before conjugation is between 150 kDa and 650 kDa.

In an embodiment, the serotype 9N glycoconjugate of the present invention comprises a serotype 9N capsular polysaccharide wherein the weight average molecular weight (Mw) of said polysaccharide before conjugation is between 200 kDa and 500 kDa.

In an embodiment, the serotype 9N glycoconjugate of the present invention comprises a serotype 9N capsular polysaccharide wherein the weight average molecular weight (Mw) of said polysaccharide before conjugation is between 200 kDa and 400 kDa.

In some embodiments, the serotype 9N glycoconjugate of the invention has a weight average molecular weight (Mw) of between 250 kDa and 20,000 kDa. In other embodiments, the serotype 9N glycoconjugate has a weight average molecular weight (Mw) of between 500 kDa and 15,000 kDa. In yet other embodiments, the serotype 9N glycoconjugate has a weight average molecular weight (Mw) of between 500 kDa and 10,000 kDa. Preferably, the serotype 9N glycoconjugate has a weight average molecular weight (Mw) of between 500 kDa and 5,000 kDa.

In an embodiment, the serotype 9N glycoconjugate has a weight average molecular weight (Mw) of between 800 kDa and 8,000 kDa.

In an embodiment, the serotype 9N glycoconjugate has a weight average molecular weight (Mw) of between 800 kDa and 6,000 kDa.

In an embodiment, the serotype 9N glycoconjugate has a weight average molecular weight (Mw) of between 1000 kDa and 5,000 kDa.

In an embodiment, the serotype 9N glycoconjugate has a weight average molecular weight (Mw) of between 1,500 kDa and 4,500 kDa.

The molecular weight of the glycoconjugate can be measured by Size Exclusion Chromatography (SEC) combined with Multiangle Laser Light Scattering detector (MALLS).

Another way to characterize the serotype 9N glycoconjugates of the invention is by the number of lysine residues in the carrier protein (e.g., CRM₁₉₇, SCP, DT or TT) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered compared to an embodiment, the degree of conjugation of the serotype 9N glycoconjugate of the invention is between 2 and 15. In a preferred embodiment, the degree of conjugation of the serotype 9N glycoconjugate of the invention is between 4 and 12.

In a very preferred embodiment, the degree of conjugation of the serotype 9N glycoconjugate of the invention is between 4 and 7. In some such embodiments, the carrier protein is CRM₁₉₇. In other such embodiments, the carrier protein is SCP.

The serotype 9N glycoconjugates of the invention may also be characterized by the ratio (weight/weight) of saccharide to carrier protein. In some embodiments, the ratio of serotype 9N polysaccharide to carrier protein in the glycoconjugate (w/w) is between 0.5 and 3.0. In other embodiments, the saccharide to carrier protein ratio (w/w) is between 0.5 and 1.5. In a preferred embodiment, the ratio of serotype 9N capsular polysaccharide to carrier protein in the conjugate is between 0.8 and 1.2.

The serotype 9N glycoconjugates of the invention may also be characterized by the number of covalent linkages between the carrier protein and the saccharide as a function of repeat units of the saccharide. In one embodiment, the serotype 9N glycoconjugate of the invention comprises at least one covalent linkage between the carrier protein and the polysaccharide for every 4 saccharide repeat units of the polysaccharide. In another embodiment, the covalent linkage between the carrier protein and the polysaccharide occurs at least once in every 10 saccharide repeat units of the polysaccharide. In another embodiment, the covalent linkage between the carrier protein and the polysaccharide occurs at least once in every 15 saccharide repeat units of the polysaccharide. In a further embodiment, the covalent linkage between the carrier protein and the polysaccharide occurs at least once in every 25 saccharide repeat units of the polysaccharide. In a further embodiment, the covalent linkage between the carrier protein and the polysaccharide occurs at least once in every 50 saccharide repeat units of the polysaccharide. In yet a further embodiment, the covalent linkage between the carrier protein and the polysaccharide occurs at least once in every 100 saccharide repeat units of the polysaccharide.

In other embodiments, the serotype 9N glycoconjugate of the invention comprises at least one covalent linkage between the carrier protein and the polysaccharide for every 5 to 30 saccharide repeat units of the polysaccharide.

In other embodiments, the serotype 9N glycoconjugate of the invention comprises at least one covalent linkage between the carrier protein and the polysaccharide for every 10 to 20 saccharide repeat units of the polysaccharide.

In some embodiments, the carrier protein is CRM₁₉₇ and the covalent linkage between the CRM₁₉₇ and the polysaccharide occurs at least once in every 4, 10, 15 or 25 saccharide repeat units of the polysaccharide. In frequent embodiments, the carrier protein is SCP and the covalent linkage between the SCP and the polysaccharide occurs at least once in every 4, 10, 15 or 25 saccharide repeat units of the polysaccharide.

The serotype 9N glycoconjugates and immunogenic compositions of the invention may contain free saccharide that is not covalently conjugated to the carrier protein but is nevertheless present in the glycoconjugate composition. The free saccharide may be noncovalently associated with (i.e., noncovalently bound to, adsorbed to, or entrapped in or with) the glycoconjugate.

In a preferred embodiment, the serotype 9N glycoconjugate comprises less than about 50% of free serotype 9N polysaccharide compared to the total amount of serotype 9N polysaccharide. In a preferred embodiment the serotype 9N glycoconjugate comprises less than about 40% of free serotype 9N polysaccharide compared to the total amount of serotype 9N polysaccharide. In a yet preferred embodiment, the serotype 9N glycoconjugate comprises less than about 25% of free serotype 9N polysaccharide compared to the total amount of serotype 9N polysaccharide. In an even preferred embodiment, the serotype 9N glycoconjugate comprises less than about 20% of free serotype 9N polysaccharide compared to the total amount of serotype 9N polysaccharide. In a yet preferred embodiment, the serotype 9N glycoconjugate comprises less than about 15% of free serotype 9N polysaccharide compared to the total amount of serotype 9N polysaccharide.

The serotype 9N glycoconjugates may also be characterized by their molecular size distribution (K_{d}). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of K_{d}, columns are calibrated to establish the fraction at which molecules are fully excluded (V₀), (K_{d}=0), and the fraction representing the maximum retention (Vᵢ), (K_{d}=1). The fraction at which a specified sample attribute is reached (Vₑ), is related to K_{d} by the expression, K_{d}=(Vₑ−V₀)/(Vᵢ−V₀).

In a preferred embodiment, at least 30% of the serotype 9N glycoconjugate has a K_{d} below or equal to 0.3 in a CL-4B column. In a preferred embodiment, at least 40% of the glycoconjugate has a K_{d} below or equal to 0.3 in a CL-4B column. In a preferred embodiment, at least 45%, 50%, 55%, 60%, 65%, 70%, 75%, 80%, or 85% of the serotype 9N glycoconjugate has a K_{d} below or equal to 0.3 in a CL-4B column. In a preferred embodiment, at least 60% of the serotype 9N glycoconjugate has a K_{d} below or equal to 0.3 in a CL-4B column. In a preferred embodiment, between 50% and 80% of the serotype 9N glycoconjugate has a K_{d} below or equal to 0.3 in a CL-4B column. In a preferred embodiment, between 65% and 80% of the serotype 9N glycoconjugate has a K_{d} below or equal to 0.3 in a CL-4B column.

2.2 Mode of Preparation of the Streptococcus pneumoniae Serotype 9N Glycoconjugates of the Invention

The serotype 9N glycoconjugate of the present invention can be prepared by any coupling technique known to those of ordinary skill in the art.

In an embodiment, the serotype 9N saccharide is coupled to the carrier protein via non-covalent bonds (see e.g. WO2012155007, WO2020056202).

In a preferred embodiment, the serotype 9N saccharide is conjugated via covalent bonds. In one embodiment the capsular saccharide is conjugated directly to a carrier protein. In a second embodiment the capsular saccharide is conjugated to a carrier protein through a spacer/linker.

In an embodiment, the serotype 9N glycoconjugate of the present invention is conjugated to the carrier protein via a linker, for instance a bifunctional linker. The linker is optionally heterobifunctional or homobifunctional, having for example a reactive amino group and a reactive carboxylic acid group, two reactive amino groups or two reactive carboxylic acid groups. The linker has for example between 4 and 20, 4 and 12, 5 and 10 carbon atoms.

A possible linker is adipic acid dihydrazide (ADH). Other linkers include B-propionamido (WO 00/10599), nitrophenyl-ethylamine (Gever et al (1979) Med. Microbiol. Immunol. 165; 171-288), haloalkyl halides (U.S. Pat. No. 4,057,685), glycosidic linkages (U.S. Pat. Nos. 4,673,574, 4,808,700), hexane diamine and 6-aminocaproic acid (U.S. Pat. No. 4,459,286).

In an embodiment, the serotype 9N glycoconjugate of the present invention is conjugated directly to the carrier protein (without a linker).

In general the following types of chemical groups on a protein carrier can be used for coupling/conjugation:
1) Amino group (for instance via lysine). In one embodiment this group is linked to carboxyl groups on saccharides directly or to a carboxyl group on a linker with carbodiimide chemistry e.g. with EDAC (1-Ethyl-3-(3-dimethylaminopropyl)carbodiimide). In another embodiment this group is linked to hydroxyl groups activated with CDAP or CNBr on saccharides directly or to such groups on a linker; to saccharides or linkers having an aldehyde group; to saccharides or linkers having a succinimide ester group.
2) Carboxyl (for instance via aspartic acid or glutamic acid). In one embodiment this group is linked to amino groups on saccharides directly or to an amino group on a linker with carbodiimide chemistry e.g. with EDAC.
3) Sulphydryl (for instance via cysteine). In one embodiment this group is linked to a bromo or chloro acetylated saccharide or linker with maleimide chemistry. In one embodiment this group is activated/modified with bis diazobenzidine.
4) Hydroxyl group (for instance via tyrosine). In one embodiment this group is activated/modified with bis diazobenzidine.
5) Imidazolyl group (for instance via histidine). In one embodiment this group is activated/modified with bis diazobenzidine.
6) Guanidyl group (for instance via arginine).
7) Indolyl group (for instance via tryptophan).

On the serotype 9N saccharide, in general the following groups can be used for a coupling: OH, COOH or NH2. Aldehyde groups can be generated after different treatments known in the art such as: periodate, acid hydrolysis, hydrogen peroxide, etc.

In an embodiment, the serotype 9N glycoconjugate of the present invention is prepared using CDAP chemistry. In said embodiment, the serotype 9N saccharide is activated with 1-cyano-4-dimethylamino pyridinium tetrafluoroborate (CDAP) to form a cyanate ester. The activated saccharide can then be coupled directly or via a spacer (linker) group to an amino group on the carrier protein. For example, the spacer could be cystamine or cysteamine to give a thiolated polysaccharide which can be coupled to the carrier via a thioether linkage obtained after reaction with a maleimide-activated carrier protein (for example using N-[γ-maleimidobutyryloxy]succinimide ester (GMBS)) or a haloacetylated carrier protein (for example using iodoacetimide, N-succinimidyl bromoacetate (SBA; SIB), N-succinimidyl(4-iodoacetyl)aminobenzoate (SIAB), sulfosuccinimidyl(4-iodoacetyl)aminobenzoate (sulfo-SIAB), N-succinimidyl iodoacetate (SIA), or succinimidyl 3-[bromoacetamido]proprionate (SBAP)). In a preferred embodiment, the cyanate ester of the activated saccharide is coupled with hexane diamine or adipic acid dihydrazide (ADH) and the amino-derivatised saccharide is conjugated to the carrier protein using carbodiimide (e.g., EDAC or EDC) chemistry via a carboxyl group on the protein carrier. Such conjugates are described for example in WO 93/15760, WO 95/08348 and WO 96/129094.

In an embodiment, the serotype 9N glycoconjugate of the present invention is prepared using carbodiimides, hydrazides, active esters, norborane, p-nitrobenzoic acid, N-hydroxysuccinimide, S-NHS, EDC, TSTU. Many are described in International Patent Application Publication No. WO 98/42721. Conjugation may involve a carbonyl linker which may be formed by reaction of a free hydroxyl group of the saccharide with CDI (see Bethell et al. (1979) 1. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

CDI and/or CDT Chemistry

In an embodiment, the serotype 9N glycoconjugate of the present invention is prepared by CDI and/or CDT chemistry as disclosed in WO2022249107.

CDI and/or CDT chemistry involves two steps, (1) reacting the serotype 9N saccharide with CDI and/or CDT in an aprotic solvent to produce an activated saccharide (activation), (2) reacting the activated saccharide with a carrier protein (e.g. CRM₁₉₇, TT or SCP) to form a glycoconjugate.

In an embodiment, the activating agent of step (1) is 1,1′-carbonyldiimidazole (CDI). In an embodiment, the activating agent of step (1) is 1,1′-Carbonyl-di-(1,2,4-triazole) (CDT).

As mentioned above, before activation with CDI and/or CDT, sizing of the serotype 9N saccharide to a target molecular weight (MW) range can be performed.

Therefore, in an embodiment, the serotype 9N saccharide is sized before activation with CDI. In an embodiment, the isolated polysaccharide is sized before activation with CDT. In an embodiment, the serotype 9N saccharide is sized to any of the target molecular weight (MW) range defined above.

Therefore, in an embodiment, the serotype 9N saccharide is conjugated to a carrier protein by a process comprising the step of:
(a) reacting said isolated polysaccharide with CDI and/or CDT in an aprotic solvent;
(b) reacting the activated polysaccharide of step (a) with a carrier protein in an aprotic solvent to form a glycoconjugate.

Following step (a) the polysaccharide is said to be activated and is referred to as “activated polysaccharide”.

In one embodiment step a) comprises reacting the serotype 9N saccharide with CDI.

In one embodiment step a) comprises reacting the serotype 9N saccharide with an amount of CDI that is between 0.5-10 molar equivalent to the amount of serotype 9N saccharide present in the reaction mixture.

In one embodiment step a) comprises reacting the serotype 9N saccharide with CDT.

In one embodiment step a) comprises reacting the serotype 9N saccharide with an amount of CDT hat is between 0.5-10 molar equivalent to the amount of serotype 9N saccharide present in the reaction mixture.

In an embodiment, the activating reaction a) is carried out in the presence of dimethylsulphoxide (DMSO), dimethylformamide (DMF), dimethylacetamide, N-methyl-2-pyrrolidone or hexamethylphosphoramide (HMPA). In an embodiment, the activating reaction a) is carried out in the presence of dimethylsulphoxide (DMSO).

In one embodiment the activating reaction a) is carried out in a solution consisting essentially of dimethylsulphoxide (DMSO) or dimethylformamide (DMF). In one embodiment the activating reaction a) is carried out in a solution consisting essentially of dimethylsulphoxide (DMSO).

In an embodiment, the conjugation reaction b) is carried out in the presence of dimethylsulphoxide (DMSO), dimethylformamide (DMF), dimethylacetamide, N-methyl-2-pyrrolidone or hexamethylphosphoramide (HMPA).

In an embodiment, the conjugation reaction b) is carried out in the presence of dimethylsulphoxide (DMSO).

In one embodiment the conjugation reaction b) is carried out in a solution consisting essentially of dimethylsulphoxide (DMSO) or dimethylformamide (DMF). In one embodiment the conjugation reaction b) is carried out in a solution consisting essentially of dimethylsulphoxide (DMSO).

In one embodiment, weak organic base can be added to the reaction mixture after the activating reaction a) but before the conjugation reaction b). The weak organic base can be added before or after the carrier protein is introduced the reaction mixture. Therefore, in one embodiment, the weak organic base is added to the reaction mixture before the carrier protein is introduced. In another embodiment, the weak organic base is added to the reaction mixture after the carrier protein is introduced. Weak organic base can be selected from alkanamines, imidazole, triazole, pyridine, histidine and guanidine. Alkanamines include alkyl primary amines such as methyl amine, ethylamine, propylamine, isopropylamine; alkyl secondary amines such as dimethyl amine, diethylamine, dipropylamine, diisopropylamine; alkyl tertially amines such as trimethyl amine, triethylamine, tri-isopropylamine, di-N,N′-isopropylethylamine, et al. In an embodiment, the weak organic base is an alkanamine. In an embodiment, the weak organic base is an imidazole. In an embodiment, the weak organic base is a triazole. In an embodiment, the weak organic base is pyridine. In an embodiment, the weak organic base is histidine. In an embodiment, the weak organic base is guanidine.

In one embodiment following the conjugation reaction b) unconjugated reactive sites of the activated polysaccharide are hydrolyzed. In one embodiment unconjugated reactive sites are hydrolyzed by addition to the conjugation solution of an aqueous solution. In one embodiment unconjugated reactive sites are hydrolyzed by addition to the conjugation solution of an aqueous buffered solution. In one embodiment unconjugated reactive sites are hydrolyzed by addition to the conjugation solution of an aqueous buffered solution and adjustment of the pH to between about 3.0 to about 10.0. In one embodiment unconjugated reactive sites are hydrolyzed by addition to the conjugation solution of an aqueous buffered solution and adjustment of the pH to between about 7.0 to about 10.0. In one embodiment unconjugated reactive sites are hydrolyzed by addition to the conjugation solution of an aqueous buffered solution and adjustment of the pH to between about 3.0 to about 7.0. In one embodiment unconjugated reactive sites are hydrolyzed by addition to the conjugation solution of an aqueous buffered solution and adjustment of the pH to about 4.0. In one embodiment unconjugated reactive sites are hydrolyzed by addition to the conjugation solution of an aqueous buffered solution and adjustment of the pH to about 9.0.

Following conjugation to the carrier protein, the serotype 9N glycoconjugate of the invention can be purified (enriched with respect to the amount of saccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration.

Therefore, in one embodiment the process for producing the glycoconjugate of the present invention comprises the step of purifying the glycoconjugate after it is produced.

eTEC Chemistry

In an embodiment, the serotype 9N glycoconjugate of the present invention is prepared by eTEC chemistry as disclosed WO2014027302

The eTEC spacer includes seven linear atoms (i.e., —C(O)NH(CH2)2SCH2C(O)—) and provides stable thioether and amide bonds between the saccharide and carrier protein. Synthesis of the eTEC linked glycoconjugate involves reaction of an activated hydroxyl group of the saccharide with the amino group of a thioalkylamine reagent, e.g., cystamine or cysteinamine or a salt thereof, forming a carbamate linkage to the saccharide to provide a thiolated saccharide. Generation of one or more free sulfhydryl groups is accomplished by reaction with a reducing agent to provide an activated thiolated saccharide. Reaction of the free sulfhydryl groups of the activated thiolated saccharide with an activated carrier protein having one or more α-haloacetamide groups on amine containing residues generates a thioether bond to form the conjugate, wherein the carrier protein is attached to the eTEC spacer through an amide bond.

Therefore, in an embodiment, the serotype 9N glycoconjugate of the present invention comprises a serotype 9N saccharide covalently conjugated to a carrier protein through a (2-((2-oxoethyl)thio)ethyl)carbamate (eTEC) spacer.

In an embodiment, the serotype 9N glycoconjugate of the present invention comprises a serotype 9N saccharide conjugated to a carrier protein through a (2-((2-oxoethyl)thio)ethyl)carbamate (eTEC) spacer, wherein the saccharide is covalently linked to the eTEC spacer through a carbamate linkage, and wherein the carrier protein is covalently linked to the eTEC spacer through an amide linkage.

The eTEC linked glycoconjugates of the invention may be represented by the general formula (III):

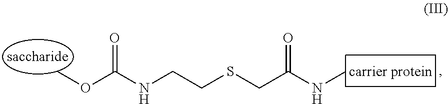

represents the serotype 9N saccharide.

Formula (III) is a schematic representation of glycoconjugates of the invention. It should not be understood that only one linkage is present between the saccharide and the carrier protein. Rather, an individual carrier protein (CP) molecule may be linked to more than one serotype 9N saccharide molecule and an individual saccharide molecule can be linked to more than one individual carrier protein (CP) molecule. Additionally, a majority of the saccharide repeating unit remains unmodified and covalent linkages between the carrier protein and the saccharide is for a minority of the saccharide repeat units.

### Direct Reductive Amination

In a preferred embodiment, the serotype 9N glycoconjugate of the present invention is prepared by direct reductive amination (see e.g. U.S. Pat. Nos. 4,365,170, 4,673,574, WO2006/110381, WO2008/079653, WO2008/143709, WO2008/079732, WO2011/110531, WO2012/119972, WO2015110941, WO2015110940, WO2018/144439, WO2018/156491).

According to the present invention, reductive amination involves two steps, (1) oxidation (activation) of the serotype 9N purified saccharide, (2) reduction of the activated saccharide and the carrier protein (e.g., CRM₁₉₇ or SCP) to form a glycoconjugate.

As mentioned above, before oxidation, sizing of the serotype 9N saccharide to a target molecular weight (MW) range can be performed. Therefore, in an embodiment, the isolated polysaccharide is sized before oxidation.

In an embodiment, the serotype 9N saccharide of the invention is conjugated to a carrier protein by a process comprising the step of:
(a) reacting said serotype 9N saccharide with an oxidizing agent;
(b) compounding the activated saccharide of step (a) with a carrier protein; and
(c) reacting the compounded activated saccharide and carrier protein with a reducing agent to form a glycoconjugate.

Following the oxidation step (a) the saccharide is said to be activated and is referred to as “activated saccharide”.

In an embodiment, the serotype 9N saccharide of the invention is conjugated to a carrier protein by a process comprising the step of:
(a) reacting said serotype 9N saccharide with an oxidizing agent;
(a′) quenching the oxidation reaction by addition of a quenching agent;
(b) compounding the activated saccharide of step (a′) with a carrier protein; and
(c) reacting the compounded activated saccharide and carrier protein with a reducing agent to form a glycoconjugate.

Following the oxidation step (a) the saccharide is said to be activated and is referred to as “activated saccharide”.

In an embodiment, the oxidizing agent is any oxidizing agent which oxidizes a terminal hydroxyl group to an aldehyde. In an embodiment, the oxidizing agent is periodate. For the purpose of the present invention, the term “periodate” includes both periodate and periodic acid, the term also includes both metaperiodate (IO₄_{^{−}}) and orthoperiodate (IO₆⁵⁻) and the various salts of periodate (e.g., sodium periodate and potassium periodate).

In an embodiment, the oxidizing agent is periodate in the presence of bivalent cations (see WO2008/143709).

In an embodiment, the oxidizing agent is periodic acid. In an embodiment, the oxidizing agent is periodic acid in the presence of bivalent cations. In an embodiment, the oxidizing agent is periodic acid in the presence of Mg²⁺. In an embodiment, the oxidizing agent is periodic acid in the presence of Ca²⁺. In an embodiment, the oxidizing agent is orthoperiodate.

In a preferred embodiment, the oxidizing agent is sodium periodate. In an embodiment, the periodate used for the oxidation is metaperiodate. In an embodiment the periodate used for the oxidation is sodium metaperiodate.

When a polysaccharide reacts with periodate, periodate oxidises vicinal hydroxyl groups to form carbonyl or aldehyde groups and causes cleavage of a C—C bond. For this reason, the term “reacting a polysaccharide with periodate” includes oxidation of vicinal hydroxyl groups by periodate.

In one embodiment step a) comprises reacting the polysaccharide with 0.01-2 molar equivalents of periodate. In one embodiment step a) comprises reacting the polysaccharide with 0.1-1.0 molar equivalents of periodate. In one embodiment step a) comprises reacting the polysaccharide with 0.1-0.5 molar equivalents of periodate.

In an embodiment, the oxidizing agent is a mixture of a stable nitroxyl radical compound with an oxidant (see WO2014097099).

In an aspect, said stable nitroxyl radical compound is a molecule bearing a TEMPO or a PROXYL (2,2,5,5-tetramethyl-1-pyrrolidinyloxy) moiety. Preferably said molecule has the ability to selectively oxidize primary alcohol in the presence of an oxidant, to generate aldehyde groups, without affecting secondary hydroxyl groups. More preferably said molecule has the ability to selectively oxidize primary alcohol in the presence of an oxidant, to generate aldehyde groups, without over oxidation to carboxyl groups. In an aspect, said stable nitroxyl radical compound is TEMPO, 2,2,6,6-Tetramethyl-4-(methylsulfonyloxy)-1-piperidinooxy, 4-Phosphonooxy-TEMPO, 4-Oxo-TEMPO, 4-Methoxy-TEMPO, 4-Isothiocyanato-TEMPO, 4-(2-Iodoacetamido)-TEMPO free radical, 4-Hydroxy-TEMPO, 4-Cyano-TEMPO, 4-Carboxy-TEMPO, 4-(2-Bromoacetamido)-TEMPO or 4-Amino-TEMPO, 4-Acetamido-2,2,6,6-tetramethylpiperidine 1-oxyl. Preferably said stable nitroxyl radical compound is TEMPO. In an aspect, said stable nitroxyl radical compound is selected from the groups consisting of TEMPO, 2,2,6,6-Tetramethyl-4-(methylsulfonyloxy)-1-piperidinooxy, 4-Phosphonooxy-TEMPO, 4-Oxo-TEMPO, 4-Methoxy-TEMPO, 4-Isothiocyanato-TEMPO, 4-(2-Iodoacetamido)-TEMPO free radical, 4-Hydroxy-TEMPO, 4-Cyano-TEMPO, 4-Carboxy-TEMPO, 4-(2-Bromoacetamido)-TEMPO, 4-Amino-TEMPO, 4-Acetamido-2,2,6,6-tetramethylpiperidine 1-oxyl. Preferably said stable nitroxyl radical compound is TEMPO. In a further aspect, said stable nitroxyl radical compound is 3β-DOXYL-5α-cholestane, 5-DOXYL-stearic acid, 16-DOXYL-stearic acid, Methyl 5-DOXYL-stearate, 3-(Aminomethyl)-PROXYL, 3-Carbamoyl-PROXYL, 3-Carbamoyl-2,2,5,5-tetramethyl-3-pyrrolin-1-oxyl, 3-Carboxy-PROXYL or 3-Cyano-PROXYL. In a further aspect, said stable nitroxyl radical compound is selected from the groups consisting of 3β-DOXYL-5α-cholestane, 5-DOXYL-stearic acid, 16-DOXYL-stearic acid, Methyl 5-DOXYL-stearate, 3-(Aminomethyl)-PROXYL, 3-Carbamoyl-PROXYL, 3-Carbamoyl-2,2,5,5-tetramethyl-3-pyrrolin-1-oxyl, 3-Carboxy-PROXYL, 3-Cyano-PROXYL.

In an aspect, the oxidant is a molecule bearing a N-halo moiety. Preferably said molecule has the ability to selectively oxidize primary alcohol in the presence of a nitroxyl radical compound. In an aspect, said oxidant is N-Chlorosuccinimide, N-Bromosuccinimide, N-Iodosuccinimide, Dichloroisocyanuric acid, 1,3,5-trichloro-1,3,5-triazinane-2,4,6-trione, Dibromoisocyanuric acid, 1,3,5-tribromo-1,3,5-triazinane-2,4,6-trione, Diiodoisocyanuric acid or 1,3,5-triiodo-1,3,5-triazinane-2,4,6-trione. In an aspect, said oxidant is selected from the group consisting of N-Chlorosuccinimide, N-Bromosuccinimide, N-Iodosuccinimide, Dichloroisocyanuric acid, 1,3,5-trichloro-1,3,5-triazinane-2,4,6-trione, Dibromoisocyanuric acid, 1,3,5-tribromo-1,3,5-triazinane-2,4,6-trione, Diiodoisocyanuric acid and 1,3,5-triiodo-1,3,5-triazinane-2,4,6-trione. Preferably said oxidant is N-Chlorosuccinimide.

In an aspect, said stable nitroxyl radical compound is 2,2,6,6-Tetramethyl-1-piperidinyloxy free radical (TEMPO) and said oxidant is N-Chlorosuccinimide (NCS).

In one embodiment, the quenching agent of step a′) is selected from vicinal diols, 1,2-aminoalcohols, amino acids, glutathione, sulfite, bisulfate, dithionite, metabisulfite, thiosulfate, phosphites, hypophosphites or phosphorous acid.

In one embodiment, the quenching agent is a 1,2-aminoalcohols of formula (I):

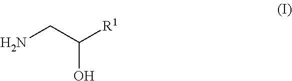

wherein R¹ is selected from H, methyl, ethyl, propyl or isopropyl.

In one embodiment, the quenching agent is selected from sodium and potassium salts of sulfite, bisulfate, dithionite, metabisulfite, thiosulfate, phosphites, hypophosphites or phosphorous acid.

In one embodiment, the quenching agent is an amino acid. In such embodiments, said amino acid may be selected from serine, threonine, cysteine, cystine, methionine, proline, hydroxyproline, tryptophan, tyrosine, and histidine.

In one embodiment, the quenching agent is a sulfite such as bisulfate, dithionite, metabisulfite, thiosulfate.

In one embodiment, the quenching agent is a compound comprising two vicinal hydroxyl groups (vicinal diols), i.e., two hydroxyl groups covalently linked to two adjacent carbon atoms. Preferably, the quenching agent is a compound of formula (II):

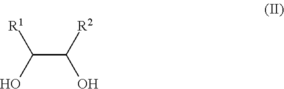

wherein R¹ and R² are each independently selected from H, methyl, ethyl, propyl or isopropyl.

In a preferred embodiment, the quenching agent is glycerol, ethylene glycol, propan-1,2-diol, butan-1,2-diol or butan-2,3-diol, or ascorbic acid. In an even preferred embodiment, the quenching agent is butan-2,3-diol.

In a preferred embodiment the degree of oxidation (also named “degree of activation” in the present document) of the activated serotype 9N saccharide is between 2 and 30. In an embodiment the degree of oxidation (DO) of the activated serotype 9N saccharide is between 5 and 15.

In one embodiment the activated saccharide and the carrier protein are lyophilised before step b).

In an embodiment the initial input ratio (weight by weight) of activated serotype 9N saccharide to carrier protein at step b) is between 4:1 and 0.1:1. In an embodiment the initial input ratio (weight by weight) of activated serotype 9N saccharide to carrier protein at step b) is between 1.5:1 and 0.5:1.

In an embodiment, the reduction reaction (c) is carried out in aqueous solvent.

In a preferred embodiment, the reduction reaction (c) is carried out in aprotic solvent. In some such embodiment, the reduction reaction (c) is carried out in the presence of dimethylsulphoxide (DMSO) or dimethylformamide (DMF). Preferably, the reduction reaction (c) is carried out in aprotic solvent in the presence of dimethylsulphoxide (DMSO).

In a preferred embodiment the reduction reaction (c) is carried out in a solution consisting essentially of dimethylsulphoxide (DMSO) or dimethylformamide (DMF). Even more preferably, the reduction reaction (c) is carried out in a solution consisting essentially of dimethylsulphoxide (DMSO).

In an embodiment, the reduction reaction (c) is carried out in DMSO (dimethylsulfoxide) solvent.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMe′PrN-BH₃, benzylamine-BH₃ or 5-ethyl-2-methylpyridine borane (PEMB). In an embodiment, the reducing agent is sodium triacetoxyborohydride. In a preferred embodiment, the reducing agent is sodium cyanoborohydride. In an embodiment, the reducing agent is sodium cyanoborohydride in the present of nickel (see WO2018144439).

In one embodiment between 0.2 and 20 molar equivalents of reducing agent is used at step c). In one embodiment between 0.5 and 10 molar equivalents of reducing agent is used at step c). In a preferred embodiment between 0.5 and 2.5 molar equivalents of reducing agent is used at step c).

At the end of the reduction reaction, there may be unreacted aldehyde groups remaining in the conjugates, these may be capped using a suitable capping agent. In one embodiment this capping agent is sodium borohydride (NaBH₄). In an embodiment capping is achieved by mixing the product of step c) with 1 to 20 molar equivalents of sodium borohydride. In an embodiment capping is achieved by mixing the product of step c) with 1 to 10 molar equivalents of sodium borohydride. In an embodiment capping is achieved by mixing the product of step c) with 1 to 5 molar equivalents of sodium borohydride.

Following conjugation to the carrier protein, the serotype 9N glycoconjugate of the invention can be purified (enriched with respect to the amount of saccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration. Therefore, in one embodiment the process for producing the glycoconjugate of the present invention comprises the step of purifying the glycoconjugate after it is produced.

### Click Chemistry

In a very preferred embodiment of the present invention, the serotype 9N glycoconjugate of the present invention is prepared by click chemistry (see e.g. WO2023/135515).

Therefore, in an embodiment, the serotype 9N glycoconjugate of the present invention comprises a serotype 9N saccharide covalently conjugated to a carrier protein (CP) through a spacer and having the general formula (IV):

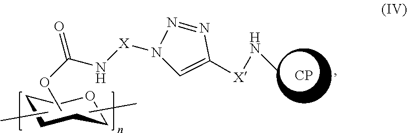

wherein X is selected from the group consisting of CH₂(CH₂)_{n′}, (CH₂CH₂O)ₘCH₂CH₂, NHCO(CH₂)_{n′}, NHCO(CH₂CH₂O)ₘCH₂CH₂, OCH₂(CH₂)_{n′} and O(CH₂CH₂O)ₘCH₂CH₂; where n′ is selected from 1 to 10 and m is selected from 1 to 4,
wherein X′ is selected from the group consisting of CH₂O(CH₂)_{n″}CH₂C═O, CH₂O(CH₂CH₂O)_{m′}(CH₂)_{n″}CH₂C═O, where n″ is selected from 0 to 10 and m′ is selected from 0 to 4,
wherein the structure in square backet represents a repeat unit of the serotype 9N saccharide and wherein n represents the number of repeating units.

In a preferred embodiment, the invention is directed to a serotype 9N glycoconjugate comprising a serotype 9N saccharide covalently conjugated to a carrier protein (CP) through a spacer and having the general formula (IV), wherein X is CH₂(CH₂)_{n′}, where n′ is 2 and wherein X′ is CH₂O(CH₂)_{n″}CH₂C═O where n″ is 1.

Therefore, in a preferred embodiment, the invention pertains to a serotype 9N glycoconjugate comprising a serotype 9N saccharide covalently conjugated to a carrier protein (CP) through a spacer and having the general formula (V),

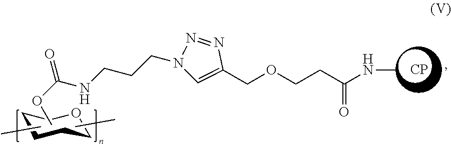

wherein the structure in square backet represents a repeat unit of the serotype 9N saccharide and wherein n represents the number of repeating units.

Formulas (IV) and (V) are schematic representations of the preferred glycoconjugates of the invention. It should not be understood that a linkage is present at every repeating unit of the saccharide (the structure in square brackets). Rather, a majority of the saccharide repeating unit remains unmodified and covalent linkages between the carrier protein and the saccharide is for a minority of the saccharide repeat units. Additionally, an individual carrier protein (CP) molecule may be linked to more than one saccharide molecule and an individual saccharide molecule can be linked to more than one individual carrier protein (CP) molecule. The structure in square brackets represents a repeat unit of the serotype 9N saccharide.

In an embodiment, the invention provides a serotype 9N glycoconjugate comprising a serotype 9N saccharide covalently conjugated to a carrier protein (CP) through a spacer and having the general formula (IV), wherein X is CH₂(CH₂)_{n′}, where n′ is selected from 1 to 10 and wherein X′ is CH₂O(CH₂)_{n″}CH₂C═O where n″ is selected from 0 to 10. In an embodiment, n′ is selected from 1 to 5 and n″ is selected from 0 to 10. In an embodiment, n′ is selected from 1 to 5 and n″ is selected from 0 to 5. In an embodiment, n′ is selected from 1 to 3 and n″ is selected from 0 to 3. In an embodiment, n′ is selected from 1 to 2 and n″ is selected from 0 to 2. In a particular embodiment, n′ is 1 and n″ is 0. In another embodiment, n′ is 2 and n″ is 0. In yet another embodiment, n′ is 3 and n″ is 0. In yet a further embodiment, n′ is 4 and n″ is 0. In yet a further embodiment, n′ is 5 and n″ is 0. In yet a further embodiment, n′ is 6 and n″ is 0. In a particular embodiment, n′ is 1 and n″ is 1. In another embodiment, n′ is 2 and n″ is 1. In yet another embodiment, n′ is 3 and n″ is 1. In yet a further embodiment, n′ is 4 and n″ is 1. In yet a further embodiment, n′ is 5 and n″ is 1. In yet a further embodiment, n′ is 6 and n″ is 1. In a particular embodiment, n′ is 1 and n″ is 2. In another embodiment, n′ is 2 and n″ is 2. In yet another embodiment, n′ is 3 and n″ is 2. In yet a further embodiment, n′ is 4 and n″ is 2. In yet a further embodiment, n′ is 5 and n″ is 2. In yet a further embodiment, n′ is 6 and n″ is 2. In a particular embodiment, n′ is 1 and n″ is 3. In another embodiment, n′ is 2 and n″ is 3. In yet another embodiment, n′ is 3 and n″ is 3. In yet a further embodiment, n′ is 4 and n″ is 3. In yet a further embodiment, n′ is 5 and n″ is 3. In yet a further embodiment, n′ is 6 and n″ is 3. In a particular embodiment, n′ is 1 and n″ is 4. In another embodiment, n′ is 2 and n″ is 4. In yet another embodiment, n′ is 3 and n″ is 4. In yet a further embodiment, n′ is 4 and n″ is 4. In yet a further embodiment, n′ is 5 and n″ is 4. In yet a further embodiment, n′ is 6 and n″ is 4. In a particular embodiment, n′ is 1 and n″ is 5. In another embodiment, n′ is 2 and n″ is 5. In yet another embodiment, n′ is 3 and n″ is 5. In yet a further embodiment, n′ is 4 and n″ is 5. In yet a further embodiment, n′ is 5 and n″ is 5. In yet a further embodiment, n′ is 6 and n″ is 5. In a particular embodiment, n′ is 1 and n″ is 6. In another embodiment, n′ is 2 and n″ is 6. In yet another embodiment, n′ is 3 and n″ is 6. In yet a further embodiment, n′ is 4 and n″ is 6. In yet a further embodiment, n′ is 5 and n″ is 6. In yet a further embodiment, n′ is 6 and n″ is 6.

In an embodiment, the invention provides a serotype 9N glycoconjugate comprising a serotype 9N saccharide covalently conjugated to a carrier protein (CP) through a spacer and having the general formula (IV), wherein X is CH₂(CH₂)_{n′}, where n′ is selected from 1 to 10 and wherein CH₂O(CH₂CH₂O)_{m′}(CH₂)_{n″}CH₂C═O, where n″ is selected from 0 to 10 and m′ is selected from 0 to 4.

In an embodiment, n′ is selected from 1 to 5, m′ is selected from 0 to 4 and n″ is selected from 0 to 10. In an embodiment, n′ is selected from 1 to 5, m′ is selected from 0 to 4 and n″ is selected from 0 to 5. In an embodiment, n′ is selected from 1 to 3, m′ is selected from 0 to 2 and n″ is selected from 0 to 3. In an embodiment, n′ is selected from 1 to 2, m′ is selected from 0 to 2 and n″ is selected from 0 to 1.

In a particular embodiment, n′ is 1, m′ is 0 and n″ is 0. In another embodiment, n′ is 1, m′ is 1 and n″ is 0. In another embodiment, n′ is 1, m′ is 2 and n″ is 0. In another embodiment, n′ is 1, m′ is 3 and n″ is 0.

In another embodiment, n′ is 2, m′ is 0 and n″ is 0. In another embodiment, n′ is 2, m′ is 1 and n″ is 0. In another embodiment, n′ is 2, m′ is 2 and n″ is 0. In another embodiment, n′ is 2, m′ is 3 and n″ is 0.

In yet another embodiment, n′ is 3, m′ is 0 and n″ is 0. In yet another embodiment, n′ is 3, m′ is 1 and n″ is 0. In yet another embodiment, n′ is 3, m′ is 2 and n″ is 0. In yet another embodiment, n′ is 3, m′ is 3 and n″ is 0.

In yet a further embodiment, n′ is 4, m′ is 0 and n″ is 0. In yet a further embodiment, n′ is 4, m′ is 1 and n″ is 0. In yet a further embodiment, n′ is 4, m′ is 2 and n″ is 0. In yet a further embodiment, n′ is 4, m′ is 3 and n″ is 0.

In yet a further embodiment, n′ is 5, m′ is 0 and n″ is 0. In yet a further embodiment, n′ is 5, m′ is 1 and n″ is 0. In yet a further embodiment, n′ is 5, m′ is 2 and n″ is 0. In yet a further embodiment, n′ is 5, m′ is 3 and n″ is 0.

In a particular embodiment, n′ is 1, m′ is 0 and n″ is 1. In a particular embodiment, n′ is 1, m′ is 1 and n″ is 1. In a particular embodiment, n′ is 1, m′ is 2 and n″ is 1. In a particular embodiment, n′ is 1, m′ is 3 and n″ is 1.

In another embodiment, n′ is 2, m′ is 0 and n″ is 1. In another embodiment, n′ is 2, m′ is 1 and n″ is 1. In another embodiment, n′ is 2, m′ is 2 and n″ is 1. In another embodiment, n′ is 2, m′ is 3 and n″ is 1.

In yet another embodiment, n′ is 3, m′ is 0 and n″ is 1. In yet another embodiment, n′ is 3, m′ is 1 and n″ is 1. In yet another embodiment, n′ is 3, m′ is 2 and n″ is 1. In yet another embodiment, n′ is 3, m′ is 3 and n″ is 1.

In yet a further embodiment, n′ is 4, m′ is 0 and n″ is 1. In yet a further embodiment, n′ is 4, m′ is 1 and n″ is 1. In yet a further embodiment, n′ is 4, m′ is 2 and n″ is 1. In yet a further embodiment, n′ is 4, m′ is 3 and n″ is 1.

In yet a further embodiment, n′ is 5, m′ is 0 and n″ is 1. In yet a further embodiment, n′ is 5, m′ is 1 and n″ is 1. In yet a further embodiment, n′ is 5, m′ is 2 and n″ is 1. In yet a further embodiment, n′ is 5, m′ is 3 and n″ is 1.

In a particular embodiment, n′ is 1, m′ is 0 and n″ is 2. In a particular embodiment, n′ is 1, m′ is 1 and n″ is 2. In a particular embodiment, n′ is 1, m′ is 2 and n″ is 2. In a particular embodiment, n′ is 1, m′ is 3 and n″ is 2.

In another embodiment, n′ is 2, m′ is 0 and n″ is 2. In another embodiment, n′ is 2, m′ is 1 and n″ is 2. In another embodiment, n′ is 2, m′ is 2 and n″ is 2. In another embodiment, n′ is 2, m′ is 3 and n″ is 2.

In yet another embodiment, n′ is 3, m′ is 0 and n″ is 2. In yet another embodiment, n′ is 3, m′ is 1 and n″ is 2. In yet another embodiment, n′ is 3, m′ is 2 and n″ is 2. In yet another embodiment, n′ is 3, m′ is 3 and n″ is 2.

In yet a further embodiment, n′ is 4, m′ is 0 and n″ is 2. In yet a further embodiment, n′ is 4, m′ is 1 and n″ is 2. In yet a further embodiment, n′ is 4, m′ is 2 and n″ is 2. In yet a further embodiment, n′ is 4, m′ is 3 and n″ is 2.

In yet a further embodiment, n′ is 5, m′ is 0 and n″ is 2. In yet a further embodiment, n′ is 5, m′ is 1 and n″ is 2. In yet a further embodiment, n′ is 5, m′ is 2 and n″ is 2. In yet a further embodiment, n′ is 5, m′ is 3 and n″ is 2.

In a particular embodiment, n′ is 1, m′ is 0 and n″ is 3. In a particular embodiment, n′ is 1, m′ is 1 and n″ is 3. In a particular embodiment, n′ is 1, m′ is 2 and n″ is 3. In a particular embodiment, n′ is 1, m′ is 3 and n″ is 3.

In another embodiment, n′ is 2, m′ is 0 and n″ is 3. In another embodiment, n′ is 2, m′ is 1 and n″ is 3. In another embodiment, n′ is 2, m′ is 2 and n″ is 3. In another embodiment, n′ is 2, m′ is 3 and n″ is 3.

In yet another embodiment, n′ is 3, m′ is 0 and n″ is 3. In yet another embodiment, n′ is 3, m′ is 1 and n″ is 3. In yet another embodiment, n′ is 3, m′ is 2 and n″ is 3. In yet another embodiment, n′ is 3, m′ is 3 and n″ is 3.

In yet a further embodiment, n′ is 4, m′ is 0 and n″ is 3. In yet a further embodiment, n′ is 4, m′ is 1 and n″ is 3. In yet a further embodiment, n′ is 4, m′ is 2 and n″ is 3. In yet a further embodiment, n′ is 4, m′ is 3 and n″ is 3.

In yet a further embodiment, n′ is 5, m′ is 0 and n″ is 3. In yet a further embodiment, n′ is 5, m′ is 1 and n″ is 3. In yet a further embodiment, n′ is 5, m′ is 2 and n″ is 3. In yet a further embodiment, n′ is 5, m′ is 3 and n″ is 3.

In a particular embodiment, n′ is 1, m′ is 0 and n″ is 4. In a particular embodiment, n′ is 1, m′ is 1 and n″ is 4. In a particular embodiment, n′ is 1, m′ is 2 and n″ is 4. In a particular embodiment, n′ is 1, m′ is 3 and n″ is 4.

In another embodiment, n′ is 2, m′ is 0 and n″ is 4. In another embodiment, n′ is 2, m′ is 1 and n″ is 4. In another embodiment, n′ is 2, m′ is 2 and n″ is 4. In another embodiment, n′ is 2, m′ is 3 and n″ is 4.

In yet another embodiment, n′ is 3, m′ is 0 and n″ is 4. In yet another embodiment, n′ is 3, m′ is 1 and n″ is 4. In yet another embodiment, n′ is 3, m′ is 2 and n″ is 4. In yet another embodiment, n′ is 3, m′ is 3 and n″ is 4.

In yet a further embodiment, n′ is 4, m′ is 0 and n″ is 4. In yet a further embodiment, n′ is 4, m′ is 1 and n″ is 4. In yet a further embodiment, n′ is 4, m′ is 2 and n″ is 4. In yet a further embodiment, n′ is 4, m′ is 3 and n″ is 4.

In yet a further embodiment, n′ is 5, m′ is 0 and n″ is 4. In yet a further embodiment, n′ is 5, m′ is 1 and n″ is 4. In yet a further embodiment, n′ is 5, m′ is 2 and n″ is 4. In yet a further embodiment, n′ is 5, m′ is 3 and n″ is 4.

In a particular embodiment, n′ is 1, m′ is 0 and n″ is 5. In a particular embodiment, n′ is 1, m′ is 1 and n″ is 5. In a particular embodiment, n′ is 1, m′ is 2 and n″ is 5. In a particular embodiment, n′ is 1, m′ is 3 and n″ is 5.

In another embodiment, n′ is 2, m′ is 0 and n″ is 5. In another embodiment, n′ is 2, m′ is 1 and n″ is 5. In another embodiment, n′ is 2, m′ is 2 and n″ is 5. In another embodiment, n′ is 2, m′ is 3 and n″ is 5.

In yet another embodiment, n′ is 3, m′ is 0 and n″ is 5. In yet another embodiment, n′ is 3, m′ is 1 and n″ is 5. In yet another embodiment, n′ is 3, m′ is 2 and n″ is 5. In yet another embodiment, n′ is 3, m′ is 3 and n″ is 5.

In yet a further embodiment, n′ is 4, m′ is 0 and n″ is 5. In yet a further embodiment, n′ is 4, m′ is 1 and n″ is 5. In yet a further embodiment, n′ is 4, m′ is 2 and n″ is 5. In yet a further embodiment, n′ is 4, m′ is 3 and n″ is 5.

In yet a further embodiment, n′ is 5, m′ is 0 and n″ is 5. In yet a further embodiment, n′ is 5, m′ is 1 and n″ is 5. In yet a further embodiment, n′ is 5, m′ is 2 and n″ is 5. In yet a further embodiment, n′ is 5, m′ is 3 and n″ is 5.

In an embodiment, the invention provides a serotype 9N glycoconjugate comprising a serotype 9N saccharide covalently conjugated to a carrier protein (CP) through a spacer and having the general formula (IV), wherein X is (CH₂CH₂O)ₘCH₂CH₂, where m is selected from 1 to 4 and wherein X′ is CH₂O(CH₂)_{n″}CH₂C═O, where n″ is selected from 0 to 10. In an embodiment, m is selected from 1 to 3 and n″ is selected from 0 to 10. In an embodiment, m is selected from 1 to 3 and n″ is selected from 0 to 5. In an embodiment, m is selected from 1 to 2 and n″ is selected from 0 to 3. In an embodiment, m is selected from 1 to 2 and n″ is selected from 0 to 2. In a particular embodiment, m is 1 and n″ is 0. In another embodiment, m is 2 and n″ is 0. In yet another embodiment, m is 3 and n″ is 0. In yet a further embodiment, m is 4 and n″ is 0. In a particular embodiment, m is 1 and n″ is 1. In another embodiment, m is 2 and n″ is 1. In yet another embodiment, m is 3 and n″ is 1. In yet a further embodiment, m is 4 and n″ is 1. In a particular embodiment, m is 1 and n″ is 2. In another embodiment, m is 2 and n″ is 2. In yet another embodiment, m is 3 and n″ is 2. In yet a further embodiment, m is 4 and n″ is 2. In a particular embodiment, m is 1 and n″ is 3. In another embodiment, m is 2 and n″ is 3. In yet another embodiment, m is 3 and n″ is 3. In yet a further embodiment, m is 4 and n″ is 3. In a particular embodiment, m is 1 and n″ is 4. In another embodiment, m is 2 and n″ is 4. In yet another embodiment, m is 3 and n″ is 4. In yet a further embodiment, m is 4 and n″ is 4. In a particular embodiment, m is 1 and n″ is 5. In another embodiment, m is 2 and n″ is 5. In yet another embodiment, m is 3 and n″ is 5. In yet a further embodiment, m is 4 and n″ is 5. In a particular embodiment, m is 1 and n″ is 6. In another embodiment, m is 2 and n″ is 6. In yet another embodiment, m is 3 and n″ is 6. In yet a further embodiment, m is 4 and n″ is 6.

In an embodiment, the invention provides a serotype 9N glycoconjugate comprising a serotype 9N saccharide covalently conjugated to a carrier protein (CP) through a spacer and having the general formula (IV), wherein X is (CH₂CH₂O)ₘCH₂CH₂, where m is selected from 1 to 4 and wherein X′ is CH₂O(CH₂CH₂O)_{m′}(CH₂)_{n″}CH₂C═O, where n″ is selected from 0 to 10 and m′ is selected from 0 to 4.

In an embodiment, m is selected from 1 to 3, m′ is selected from 0 to 4 and n″ is selected from 0 to 10. In an embodiment, m is selected from 1 to 2, m′ is selected from 0 to 4 and n″ is selected from 0 to 5. In an embodiment, m is selected from 1 to 2, m′ is selected from 0 to 2 and n″ is selected from 0 to 3. In an embodiment, m is selected from 1 to 2, m′ is selected from 0 to 2 and n″ is selected from 0 to 1.

In a particular embodiment, m is 1, m′ is 0 and n″ is 0. In another embodiment, m is 1, m′ is 1 and n″ is 0. In another embodiment, m is 1, m′ is 2 and n″ is 0. In another embodiment, m is 1, m′ is 3 and n″ is 0.

In another embodiment, m is 2, m′ is 0 and n″ is 0. In another embodiment, m is 2, m′ is 1 and n″ is 0. In another embodiment, m is 2, m′ is 2 and n″ is 0. In another embodiment, m is 2, m′ is 3 and n″ is 0.

In yet another embodiment, m is 3, m′ is 0 and n″ is 0. In yet another embodiment, m is 3, m′ is 1 and n″ is 0. In yet another embodiment, m is 3, m′ is 2 and n″ is 0. In yet another embodiment, m is 3, m′ is 3 and n″ is 0.

In yet a further embodiment, m is 4, m′ is 0 and n″ is 0. In yet a further embodiment, m is 4, m′ is 1 and n″ is 0. In yet a further embodiment, m is 4, m′ is 2 and n″ is 0. In yet a further embodiment, m is 4, m′ is 3 and n″ is 0.

In a particular embodiment, m is 1, m′ is 0 and n″ is 1. In a particular embodiment, m is 1, m′ is 1 and n″ is 1. In a particular embodiment, m is 1, m′ is 2 and n″ is 1. In a particular embodiment, m is 1, m′ is 3 and n″ is 1.

In another embodiment, m is 2, m′ is 0 and n″ is 1. In another embodiment, m is 2, m′ is 1 and n″ is 1. In another embodiment, m is 2, m′ is 2 and n″ is 1. In another embodiment, m is 2, m′ is 3 and n″ is 1.

In yet another embodiment, m is 3, m′ is 0 and n″ is 1. In yet another embodiment, m is 3, m′ is 1 and n″ is 1. In yet another embodiment, m is 3, m′ is 2 and n″ is 1. In yet another embodiment, m is 3, m′ is 3 and n″ is 1.

In yet a further embodiment, m is 4, m′ is 0 and n″ is 1. In yet a further embodiment, m is 4, m′ is 1 and n″ is 1. In yet a further embodiment, m is 4, m′ is 2 and n″ is 1. In yet a further embodiment, m is 4, m′ is 3 and n″ is 1.

In a particular embodiment, m is 1, m′ is 0 and n″ is 2. In a particular embodiment, m is 1, m′ is 1 and n″ is 2. In a particular embodiment, m is 1, m′ is 2 and n″ is 2. In a particular embodiment, m is 1, m′ is 3 and n″ is 2.

In another embodiment, m is 2, m′ is 0 and n″ is 2. In another embodiment, m is 2, m′ is 1 and n″ is 2. In another embodiment, m is 2, m′ is 2 and n″ is 2. In another embodiment, m is 2, m′ is 3 and n″ is 2.

In yet another embodiment, m is 3, m′ is 0 and n″ is 2. In yet another embodiment, m is 3, m′ is 1 and n″ is 2. In yet another embodiment, m is 3, m′ is 2 and n″ is 2. In yet another embodiment, m is 3, m′ is 3 and n″ is 2.

In yet a further embodiment, m is 4, m′ is 0 and n″ is 2. In yet a further embodiment, m is 4, m′ is 1 and n″ is 2. In yet a further embodiment, m is 4, m′ is 2 and n″ is 2. In yet a further embodiment, m is 4, m′ is 3 and n″ is 2.

In a particular embodiment, m is 1, m′ is 0 and n″ is 3. In a particular embodiment, m is 1, m′ is 1 and n″ is 3. In a particular embodiment, m is 1, m′ is 2 and n″ is 3. In a particular embodiment, m is 1, m′ is 3 and n″ is 3.

In another embodiment, m is 2, m′ is 0 and n″ is 3. In another embodiment, m is 2, m′ is 1 and n″ is 3. In another embodiment, m is 2, m′ is 2 and n″ is 3. In another embodiment, m is 2, m′ is 3 and n″ is 3.

In yet another embodiment, m is 3, m′ is 0 and n″ is 3. In yet another embodiment, m is 3, m′ is 1 and n″ is 3. In yet another embodiment, m is 3, m′ is 2 and n″ is 3. In yet another embodiment, m is 3, m′ is 3 and n″ is 3.

In yet a further embodiment, m is 4, m′ is 0 and n″ is 3. In yet a further embodiment, m is 4, m′ is 1 and n″ is 3. In yet a further embodiment, m is 4, m′ is 2 and n″ is 3. In yet a further embodiment, m is 4, m′ is 3 and n″ is 3.

In a particular embodiment, m is 1, m′ is 0 and n″ is 4. In a particular embodiment, m is 1, m′ is 1 and n″ is 4. In a particular embodiment, m is 1, m′ is 2 and n″ is 4. In a particular embodiment, m is 1, m′ is 3 and n″ is 4.

In another embodiment, m is 2, m′ is 0 and n″ is 4. In another embodiment, m is 2, m′ is 1 and n″ is 4. In another embodiment, m is 2, m′ is 2 and n″ is 4. In another embodiment, m is 2, m′ is 3 and n″ is 4.

In yet another embodiment, m is 3, m′ is 0 and n″ is 4. In yet another embodiment, m is 3, m′ is 1 and n″ is 4. In yet another embodiment, m is 3, m′ is 2 and n″ is 4. In yet another embodiment, m is 3, m′ is 3 and n″ is 4.

In yet a further embodiment, m is 4, m′ is 0 and n″ is 4. In yet a further embodiment, m is 4, m′ is 1 and n″ is 4. In yet a further embodiment, m is 4, m′ is 2 and n″ is 4. In yet a further embodiment, m is 4, m′ is 3 and n″ is 4.

In a particular embodiment, m is 1, m′ is 0 and n″ is 5. In a particular embodiment, m is 1, m′ is 1 and n″ is 5. In a particular embodiment, m is 1, m′ is 2 and n″ is 5. In a particular embodiment, m is 1, m′ is 3 and n″ is 5.

In another embodiment, m is 2, m′ is 0 and n″ is 5. In another embodiment, m is 2, m′ is 1 and n″ is 5. In another embodiment, m is 2, m′ is 2 and n″ is 5. In another embodiment, m is 2, m′ is 3 and n″ is 5.

In yet another embodiment, m is 3, m′ is 0 and n″ is 5. In yet another embodiment, m is 3, m′ is 1 and n″ is 5. In yet another embodiment, m is 3, m′ is 2 and n″ is 5. In yet another embodiment, m is 3, m′ is 3 and n″ is 5.

In yet a further embodiment, m is 4, m′ is 0 and n″ is 5. In yet a further embodiment, m is 4, m′ is 1 and n″ is 5. In yet a further embodiment, m is 4, m′ is 2 and n″ is 5. In yet a further embodiment, m is 4, m′ is 3 and n″ is 5.

In an embodiment, the invention provides a serotype 9N glycoconjugate comprising a serotype 9N saccharide covalently conjugated to a carrier protein (CP) through a spacer and having the general formula (IV), wherein X is NHCO(CH₂)_{n′}, where n′ is selected from 1 to 10 and wherein X′ is CH₂O(CH₂)_{n″}CH₂C═O, where n″ is selected from 0 to 10. In an embodiment, n′ is selected from 1 to 5 and n″ is selected from 0 to 10. In an embodiment, n′ is selected from 1 to 5 and n″ is selected from 0 to 5. In an embodiment, n′ is selected from 1 to 3 and n″ is selected from 0 to 3. In an embodiment, n′ is selected from 1 to 2 and n″ is selected from 0 to 2. In a particular embodiment, n′ is 1 and n″ is 0. In another embodiment, n′ is 2 and n″ is 0. In yet another embodiment, n′ is 3 and n″ is 0. In yet a further embodiment, n′ is 4 and n″ is 0. In yet a further embodiment, n′ is 5 and n″ is 0. In yet a further embodiment, n′ is 6 and n″ is 0. In a particular embodiment, n′ is 1 and n″ is 1. In another embodiment, n′ is 2 and n″ is 1. In yet another embodiment, n′ is 3 and n″ is 1. In yet a further embodiment, n′ is 4 and n″ is 1. In yet a further embodiment, n′ is 5 and n″ is 1. In yet a further embodiment, n′ is 6 and n″ is 1. In a particular embodiment, n′ is 1 and n″ is 2. In another embodiment, n′ is 2 and n″ is 2. In yet another embodiment, n′ is 3 and n″ is 2. In yet a further embodiment, n′ is 4 and n″ is 2. In yet a further embodiment, n′ is 5 and n″ is 2. In yet a further embodiment, n′ is 6 and n″ is 2. In a particular embodiment, n′ is 1 and n″ is 3. In another embodiment, n′ is 2 and n″ is 3. In yet another embodiment, n′ is 3 and n″ is 3. In yet a further embodiment, n′ is 4 and n″ is 3. In yet a further embodiment, n′ is 5 and n″ is 3. In yet a further embodiment, n′ is 6 and n″ is 3. In a particular embodiment, n′ is 1 and n″ is 4. In another embodiment, n′ is 2 and n″ is 4. In yet another embodiment, n′ is 3 and n″ is 4. In yet a further embodiment, n′ is 4 and n″ is 4. In yet a further embodiment, n′ is 5 and n″ is 4. In yet a further embodiment, n′ is 6 and n″ is 4. In a particular embodiment, n′ is 1 and n″ is 5. In another embodiment, n′ is 2 and n″ is 5. In yet another embodiment, n′ is 3 and n″ is 5. In yet a further embodiment, n′ is 4 and n″ is 5. In yet a further embodiment, n′ is 5 and n″ is 5. In yet a further embodiment, n′ is 6 and n″ is 5. In a particular embodiment, n′ is 1 and n″ is 6. In another embodiment, n′ is 2 and n″ is 6. In yet another embodiment, n′ is 3 and n″ is 6. In yet a further embodiment, n′ is 4 and n″ is 6. In yet a further embodiment, n′ is 5 and n″ is 6. In yet a further embodiment, n′ is 6 and n″ is 6.

In an embodiment, the invention provides a serotype 9N glycoconjugate comprising a serotype 9N saccharide covalently conjugated to a carrier protein (CP) through a spacer and having the general formula (IV), wherein X is NHCO(CH₂)_{n′}, where n′ is selected from 1 to 10 and wherein X′ is CH₂O(CH₂CH₂O)_{m′}(CH₂)_{n″}CH₂C═O, where n″ is selected from 0 to 10 and m′ is selected from 0 to 4.

In an embodiment, n′ is selected from 1 to 5, m′ is selected from 0 to 4 and n″ is selected from 0 to 10. In an embodiment, n′ is selected from 1 to 5, m′ is selected from 0 to 4 and n″ is selected from 0 to 5. In an embodiment, n′ is selected from 1 to 3, m′ is selected from 0 to 2 and n″ is selected from 0 to 3. In an embodiment, n′ is selected from 1 to 2, m′ is selected from 0 to 2 and n″ is selected from 0 to 1.

In a particular embodiment, n′ is 1, m′ is 0 and n″ is 0. In another embodiment, n′ is 1, m′ is 1 and n″ is 0. In another embodiment, n′ is 1, m′ is 2 and n″ is 0. In another embodiment, n′ is 1, m′ is 3 and n″ is 0.

In another embodiment, n′ is 2, m′ is 0 and n″ is 0. In another embodiment, n′ is 2, m′ is 1 and n″ is 0. In another embodiment, n′ is 2, m′ is 2 and n″ is 0. In another embodiment, n′ is 2, m′ is 3 and n″ is 0.

In yet another embodiment, n′ is 3, m′ is 0 and n″ is 0. In yet another embodiment, n′ is 3, m′ is 1 and n″ is 0. In yet another embodiment, n′ is 3, m′ is 2 and n″ is 0. In yet another embodiment, n′ is 3, m′ is 3 and n″ is 0.

In yet a further embodiment, n′ is 4, m′ is 0 and n″ is 0. In yet a further embodiment, n′ is 4, m′ is 1 and n″ is 0. In yet a further embodiment, n′ is 4, m′ is 2 and n″ is 0. In yet a further embodiment, n′ is 4, m′ is 3 and n″ is 0.

In yet a further embodiment, n′ is 5, m′ is 0 and n″ is 0. In yet a further embodiment, n′ is 5, m′ is 1 and n″ is 0. In yet a further embodiment, n′ is 5, m′ is 2 and n″ is 0. In yet a further embodiment, n′ is 5, m′ is 3 and n″ is 0.

In a particular embodiment, n′ is 1, m′ is 0 and n″ is 1. In a particular embodiment, n′ is 1, m′ is 1 and n″ is 1. In a particular embodiment, n′ is 1, m′ is 2 and n″ is 1. In a particular embodiment, n′ is 1, m′ is 3 and n″ is 1.

In another embodiment, n′ is 2, m′ is 0 and n″ is 1. In another embodiment, n′ is 2, m′ is 1 and n″ is 1. In another embodiment, n′ is 2, m′ is 2 and n″ is 1. In another embodiment, n′ is 2, m′ is 3 and n″ is 1.

In yet another embodiment, n′ is 3, m′ is 0 and n″ is 1. In yet another embodiment, n′ is 3, m′ is 1 and n″ is 1. In yet another embodiment, n′ is 3, m′ is 2 and n″ is 1. In yet another embodiment, n′ is 3, m′ is 3 and n″ is 1.

In yet a further embodiment, n′ is 4, m′ is 0 and n″ is 1. In yet a further embodiment, n′ is 4, m′ is 1 and n″ is 1. In yet a further embodiment, n′ is 4, m′ is 2 and n″ is 1. In yet a further embodiment, n′ is 4, m′ is 3 and n″ is 1.

In yet a further embodiment, n′ is 5, m′ is 0 and n″ is 1. In yet a further embodiment, n′ is 5, m′ is 1 and n″ is 1. In yet a further embodiment, n′ is 5, m′ is 2 and n″ is 1. In yet a further embodiment, n′ is 5, m′ is 3 and n″ is 1.

In a particular embodiment, n′ is 1, m′ is 0 and n″ is 2. In a particular embodiment, n′ is 1, m′ is 1 and n″ is 2. In a particular embodiment, n′ is 1, m′ is 2 and n″ is 2. In a particular embodiment, n′ is 1, m′ is 3 and n″ is 2.

In another embodiment, n′ is 2, m′ is 0 and n″ is 2. In another embodiment, n′ is 2, m′ is 1 and n″ is 2. In another embodiment, n′ is 2, m′ is 2 and n″ is 2. In another embodiment, n′ is 2, m′ is 3 and n″ is 2.

In yet another embodiment, n′ is 3, m′ is 0 and n″ is 2. In yet another embodiment, n′ is 3, m′ is 1 and n″ is 2. In yet another embodiment, n′ is 3, m′ is 2 and n″ is 2. In yet another embodiment, n′ is 3, m′ is 3 and n″ is 2.

In yet a further embodiment, n′ is 4, m′ is 0 and n″ is 2. In yet a further embodiment, n′ is 4, m′ is 1 and n″ is 2. In yet a further embodiment, n′ is 4, m′ is 2 and n″ is 2. In yet a further embodiment, n′ is 4, m′ is 3 and n″ is 2.

In yet a further embodiment, n′ is 5, m′ is 0 and n″ is 2. In yet a further embodiment, n′ is 5, m′ is 1 and n″ is 2. In yet a further embodiment, n′ is 5, m′ is 2 and n″ is 2. In yet a further embodiment, n′ is 5, m′ is 3 and n″ is 2.

In a particular embodiment, n′ is 1, m′ is 0 and n″ is 3. In a particular embodiment, n′ is 1, m′ is 1 and n″ is 3. In a particular embodiment, n′ is 1, m′ is 2 and n″ is 3. In a particular embodiment, n′ is 1, m′ is 3 and n″ is 3.

In another embodiment, n′ is 2, m′ is 0 and n″ is 3. In another embodiment, n′ is 2, m′ is 1 and n″ is 3. In another embodiment, n′ is 2, m′ is 2 and n″ is 3. In another embodiment, n′ is 2, m′ is 3 and n″ is 3.

In yet another embodiment, n′ is 3, m′ is 0 and n″ is 3. In yet another embodiment, n′ is 3, m′ is 1 and n″ is 3. In yet another embodiment, n′ is 3, m′ is 2 and n″ is 3. In yet another embodiment, n′ is 3, m′ is 3 and n″ is 3.

In yet a further embodiment, n′ is 4, m′ is 0 and n″ is 3. In yet a further embodiment, n′ is 4, m′ is 1 and n″ is 3. In yet a further embodiment, n′ is 4, m′ is 2 and n″ is 3. In yet a further embodiment, n′ is 4, m′ is 3 and n″ is 3.

In yet a further embodiment, n′ is 5, m′ is 0 and n″ is 3. In yet a further embodiment, n′ is 5, m′ is 1 and n″ is 3. In yet a further embodiment, n′ is 5, m′ is 2 and n″ is 3. In yet a further embodiment, n′ is 5, m′ is 3 and n″ is 3.

In a particular embodiment, n′ is 1, m′ is 0 and n″ is 4. In a particular embodiment, n′ is 1, m′ is 1 and n″ is 4. In a particular embodiment, n′ is 1, m′ is 2 and n″ is 4. In a particular embodiment, n′ is 1, m′ is 3 and n″ is 4.

In another embodiment, n′ is 2, m′ is 0 and n″ is 4. In another embodiment, n′ is 2, m′ is 1 and n″ is 4. In another embodiment, n′ is 2, m′ is 2 and n″ is 4. In another embodiment, n′ is 2, m′ is 3 and n″ is 4.

In yet another embodiment, n′ is 3, m′ is 0 and n″ is 4. In yet another embodiment, n′ is 3, m′ is 1 and n″ is 4. In yet another embodiment, n′ is 3, m′ is 2 and n″ is 4. In yet another embodiment, n′ is 3, m′ is 3 and n″ is 4.

In yet a further embodiment, n′ is 4, m′ is 0 and n″ is 4. In yet a further embodiment, n′ is 4, m′ is 1 and n″ is 4. In yet a further embodiment, n′ is 4, m′ is 2 and n″ is 4. In yet a further embodiment, n′ is 4, m′ is 3 and n″ is 4.

In yet a further embodiment, n′ is 5, m′ is 0 and n″ is 4. In yet a further embodiment, n′ is 5, m′ is 1 and n″ is 4. In yet a further embodiment, n′ is 5, m′ is 2 and n″ is 4. In yet a further embodiment, n′ is 5, m′ is 3 and n″ is 4.

In a particular embodiment, n′ is 1, m′ is 0 and n″ is 5. In a particular embodiment, n′ is 1, m′ is 1 and n″ is 5. In a particular embodiment, n′ is 1, m′ is 2 and n″ is 5. In a particular embodiment, n′ is 1, m′ is 3 and n″ is 5.

In another embodiment, n′ is 2, m′ is 0 and n″ is 5. In another embodiment, n′ is 2, m′ is 1 and n″ is 5. In another embodiment, n′ is 2, m′ is 2 and n″ is 5. In another embodiment, n′ is 2, m′ is 3 and n″ is 5.

In yet another embodiment, n′ is 3, m′ is 0 and n″ is 5. In yet another embodiment, n′ is 3, m′ is 1 and n″ is 5. In yet another embodiment, n′ is 3, m′ is 2 and n″ is 5. In yet another embodiment, n′ is 3, m′ is 3 and n″ is 5.

In yet a further embodiment, n′ is 4, m′ is 0 and n″ is 5. In yet a further embodiment, n′ is 4, m′ is 1 and n″ is 5. In yet a further embodiment, n′ is 4, m′ is 2 and n″ is 5. In yet a further embodiment, n′ is 4, m′ is 3 and n″ is 5.

In yet a further embodiment, n′ is 5, m′ is 0 and n″ is 5. In yet a further embodiment, n′ is 5, m′ is 1 and n″ is 5. In yet a further embodiment, n′ is 5, m′ is 2 and n″ is 5. In yet a further embodiment, n′ is 5, m′ is 3 and n″ is 5.

In an embodiment, the invention provides a serotype 9N glycoconjugate comprising a serotype 9N saccharide covalently conjugated to a carrier protein (CP) through a spacer and having the general formula (IV), wherein X is NHCO(CH₂CH₂O)ₘCH₂CH₂, where m is selected from 1 to 4 and wherein X′ is CH₂O(CH₂)_{n″}CH₂C═O, where n″ is selected from 0 to 10. In an embodiment, m is selected from 1 to 3 and n″ is selected from 0 to 10. In an embodiment, m is selected from 1 to 3 and n″ is selected from 0 to 5. In an embodiment, m is selected from 1 to 2 and n″ is selected from 0 to 3. In an embodiment, m is selected from 1 to 2 and n″ is selected from 0 to 2. In a particular embodiment, m is 1 and n″ is 0. In another embodiment, m is 2 and n″ is 0. In yet another embodiment, m is 3 and n″ is 0. In yet a further embodiment, m is 4 and n″ is 0. In a particular embodiment, m is 1 and n″ is 1. In another embodiment, m is 2 and n″ is 1. In yet another embodiment, m is 3 and n″ is 1. In yet a further embodiment, m is 4 and n″ is 1. In a particular embodiment, m is 1 and n″ is 2. In another embodiment, m is 2 and n″ is 2. In yet another embodiment, m is 3 and n″ is 2. In yet a further embodiment, m is 4 and n″ is 2. In a particular embodiment, m is 1 and n″ is 3. In another embodiment, m is 2 and n″ is 3. In yet another embodiment, m is 3 and n″ is 3. In yet a further embodiment, m is 4 and n″ is 3. In a particular embodiment, m is 1 and n″ is 4. In another embodiment, m is 2 and n″ is 4. In yet another embodiment, m is 3 and n″ is 4. In yet a further embodiment, m is 4 and n″ is 4. In a particular embodiment, m is 1 and n″ is 5. In another embodiment, m is 2 and n″ is 5. In yet another embodiment, m is 3 and n″ is 5. In yet a further embodiment, m is 4 and n″ is 5. In a particular embodiment, m is 1 and n″ is 6. In another embodiment, m is 2 and n″ is 6. In yet another embodiment, m is 3 and n″ is 6. In yet a further embodiment, m is 4 and n″ is 6.

In an embodiment, the invention provides a serotype 9N glycoconjugate comprising a serotype 9N saccharide covalently conjugated to a carrier protein (CP) through a spacer and having the general formula (IV), wherein X is NHCO(CH₂CH₂O)ₘCH₂CH₂, where m is selected from 1 to 4 and wherein X′ is CH₂O(CH₂CH₂O)_{m′}(CH₂)_{n″}CH₂C═O, where n″ is selected from 0 to 10 and m′ is selected from 0 to 4.

In an embodiment, m is selected from 1 to 3, m′ is selected from 0 to 4 and n″ is selected from 0 to 10. In an embodiment, m is selected from 1 to 2, m′ is selected from 0 to 4 and n″ is selected from 0 to 5. In an embodiment, m is selected from 1 to 2, m′ is selected from 0 to 2 and n″ is selected from 0 to 3. In an embodiment, m is selected from 1 to 2, m′ is selected from 0 to 2 and n″ is selected from 0 to 1.

In a particular embodiment, m is 1, m′ is 0 and n″ is 0. In another embodiment, m is 1, m′ is 1 and n″ is 0. In another embodiment, m is 1, m′ is 2 and n″ is 0. In another embodiment, m is 1, m′ is 3 and n″ is 0.

In another embodiment, m is 2, m′ is 0 and n″ is 0. In another embodiment, m is 2, m′ is 1 and n″ is 0. In another embodiment, m is 2, m′ is 2 and n″ is 0. In another embodiment, m is 2, m′ is 3 and n″ is 0.

In yet another embodiment, m is 3, m′ is 0 and n″ is 0. In yet another embodiment, m is 3, m′ is 1 and n″ is 0. In yet another embodiment, m is 3, m′ is 2 and n″ is 0. In yet another embodiment, m is 3, m′ is 3 and n″ is 0.

In yet a further embodiment, m is 4, m′ is 0 and n″ is 0. In yet a further embodiment, m is 4, m′ is 1 and n″ is 0. In yet a further embodiment, m is 4, m′ is 2 and n″ is 0. In yet a further embodiment, m is 4, m′ is 3 and n″ is 0.

In a particular embodiment, m is 1, m′ is 0 and n″ is 1. In a particular embodiment, m is 1, m′ is 1 and n″ is 1. In a particular embodiment, m is 1, m′ is 2 and n″ is 1. In a particular embodiment, m is 1, m′ is 3 and n″ is 1.

In another embodiment, m is 2, m′ is 0 and n″ is 1. In another embodiment, m is 2, m′ is 1 and n″ is 1. In another embodiment, m is 2, m′ is 2 and n″ is 1. In another embodiment, m is 2, m′ is 3 and n″ is 1.

In yet another embodiment, m is 3, m′ is 0 and n″ is 1. In yet another embodiment, m is 3, m′ is 1 and n″ is 1. In yet another embodiment, m is 3, m′ is 2 and n″ is 1. In yet another embodiment, m is 3, m′ is 3 and n″ is 1.

In yet a further embodiment, m is 4, m′ is 0 and n″ is 1. In yet a further embodiment, m is 4, m′ is 1 and n″ is 1. In yet a further embodiment, m is 4, m′ is 2 and n″ is 1. In yet a further embodiment, m is 4, m′ is 3 and n″ is 1.

In a particular embodiment, m is 1, m′ is 0 and n″ is 2. In a particular embodiment, m is 1, m′ is 1 and n″ is 2. In a particular embodiment, m is 1, m′ is 2 and n″ is 2. In a particular embodiment, m is 1, m′ is 3 and n″ is 2.

In another embodiment, m is 2, m′ is 0 and n″ is 2. In another embodiment, m is 2, m′ is 1 and n″ is 2. In another embodiment, m is 2, m′ is 2 and n″ is 2. In another embodiment, m is 2, m′ is 3 and n″ is 2.

In yet another embodiment, m is 3, m′ is 0 and n″ is 2. In yet another embodiment, m is 3, m′ is 1 and n″ is 2. In yet another embodiment, m is 3, m′ is 2 and n″ is 2. In yet another embodiment, m is 3, m′ is 3 and n″ is 2.

In yet a further embodiment, m is 4, m′ is 0 and n″ is 2. In yet a further embodiment, m is 4, m′ is 1 and n″ is 2. In yet a further embodiment, m is 4, m′ is 2 and n″ is 2. In yet a further embodiment, m is 4, m′ is 3 and n″ is 2.

In a particular embodiment, m is 1, m′ is 0 and n″ is 3. In a particular embodiment, m is 1, m′ is 1 and n″ is 3. In a particular embodiment, m is 1, m′ is 2 and n″ is 3. In a particular embodiment, m is 1, m′ is 3 and n″ is 3.

In another embodiment, m is 2, m′ is 0 and n″ is 3. In another embodiment, m is 2, m′ is 1 and n″ is 3. In another embodiment, m is 2, m′ is 2 and n″ is 3. In another embodiment, m is 2, m′ is 3 and n″ is 3.

In yet another embodiment, m is 3, m′ is 0 and n″ is 3. In yet another embodiment, m is 3, m′ is 1 and n″ is 3. In yet another embodiment, m is 3, m′ is 2 and n″ is 3. In yet another embodiment, m is 3, m′ is 3 and n″ is 3.

In yet a further embodiment, m is 4, m′ is 0 and n″ is 3. In yet a further embodiment, m is 4, m′ is 1 and n″ is 3. In yet a further embodiment, m is 4, m′ is 2 and n″ is 3. In yet a further embodiment, m is 4, m′ is 3 and n″ is 3.

In a particular embodiment, m is 1, m′ is 0 and n″ is 4. In a particular embodiment, m is 1, m′ is 1 and n″ is 4. In a particular embodiment, m is 1, m′ is 2 and n″ is 4. In a particular embodiment, m is 1, m′ is 3 and n″ is 4.

In another embodiment, m is 2, m′ is 0 and n″ is 4. In another embodiment, m is 2, m′ is 1 and n″ is 4. In another embodiment, m is 2, m′ is 2 and n″ is 4. In another embodiment, m is 2, m′ is 3 and n″ is 4.

In yet another embodiment, m is 3, m′ is 0 and n″ is 4. In yet another embodiment, m is 3, m′ is 1 and n″ is 4. In yet another embodiment, m is 3, m′ is 2 and n″ is 4. In yet another embodiment, m is 3, m′ is 3 and n″ is 4.

In yet a further embodiment, m is 4, m′ is 0 and n″ is 4. In yet a further embodiment, m is 4, m′ is 1 and n″ is 4. In yet a further embodiment, m is 4, m′ is 2 and n″ is 4. In yet a further embodiment, m is 4, m′ is 3 and n″ is 4.

In a particular embodiment, m is 1, m′ is 0 and n″ is 5. In a particular embodiment, m is 1, m′ is 1 and n″ is 5. In a particular embodiment, m is 1, m′ is 2 and n″ is 5. In a particular embodiment, m is 1, m′ is 3 and n″ is 5.

In another embodiment, m is 2, m′ is 0 and n″ is 5. In another embodiment, m is 2, m′ is 1 and n″ is 5. In another embodiment, m is 2, m′ is 2 and n″ is 5. In another embodiment, m is 2, m′ is 3 and n″ is 5.

In yet another embodiment, m is 3, m′ is 0 and n″ is 5. In yet another embodiment, m is 3, m′ is 1 and n″ is 5. In yet another embodiment, m is 3, m′ is 2 and n″ is 5. In yet another embodiment, m is 3, m′ is 3 and n″ is 5.

In yet a further embodiment, m is 4, m′ is 0 and n″ is 5. In yet a further embodiment, m is 4, m′ is 1 and n″ is 5. In yet a further embodiment, m is 4, m′ is 2 and n″ is 5. In yet a further embodiment, m is 4, m′ is 3 and n″ is 5.

In an embodiment, the invention provides a serotype 9N glycoconjugate comprising a serotype 9N saccharide covalently conjugated to a carrier protein (CP) through a spacer and having the general formula (IV), wherein X is OCH₂(CH₂)_{n′}, where n′ is selected from 1 to 10 and wherein X′ is CH₂O(CH₂)_{n″}CH₂C═O, where n″ is selected from 0 to 10.

In an embodiment, n′ is selected from 1 to 5 and n″ is selected from 0 to 10. In an embodiment, n′ is selected from 1 to 5 and n″ is selected from 0 to 5. In an embodiment, n′ is selected from 1 to 3 and n″ is selected from 0 to 3. In an embodiment, n′ is selected from 1 to 2 and n″ is selected from 0 to 2. In a particular embodiment, n′ is 1 and n″ is 0. In another embodiment, n′ is 2 and n″ is 0. In yet another embodiment, n′ is 3 and n″ is 0. In yet a further embodiment, n′ is 4 and n″ is 0. In yet a further embodiment, n′ is 5 and n″ is 0. In yet a further embodiment, n′ is 6 and n″ is 0. In a particular embodiment, n′ is 1 and n″ is 1. In another embodiment, n′ is 2 and n″ is 1. In yet another embodiment, n′ is 3 and n″ is 1. In yet a further embodiment, n′ is 4 and n″ is 1. In yet a further embodiment, n′ is 5 and n″ is 1. In yet a further embodiment, n′ is 6 and n″ is 1. In a particular embodiment, n′ is 1 and n″ is 2. In another embodiment, n′ is 2 and n″ is 2. In yet another embodiment, n′ is 3 and n″ is 2. In yet a further embodiment, n′ is 4 and n″ is 2. In yet a further embodiment, n′ is 5 and n″ is 2. In yet a further embodiment, n′ is 6 and n″ is 2. In a particular embodiment, n′ is 1 and n″ is 3. In another embodiment, n′ is 2 and n″ is 3. In yet another embodiment, n′ is 3 and n″ is 3. In yet a further embodiment, n′ is 4 and n″ is 3. In yet a further embodiment, n′ is 5 and n″ is 3. In yet a further embodiment, n′ is 6 and n″ is 3. In a particular embodiment, n′ is 1 and n″ is 4. In another embodiment, n′ is 2 and n″ is 4. In yet another embodiment, n′ is 3 and n″ is 4. In yet a further embodiment, n′ is 4 and n″ is 4. In yet a further embodiment, n′ is 5 and n″ is 4. In yet a further embodiment, n′ is 6 and n″ is 4. In a particular embodiment, n′ is 1 and n″ is 5. In another embodiment, n′ is 2 and n″ is 5. In yet another embodiment, n′ is 3 and n″ is 5. In yet a further embodiment, n′ is 4 and n″ is 5. In yet a further embodiment, n′ is 5 and n″ is 5. In yet a further embodiment, n′ is 6 and n″ is 5. In a particular embodiment, n′ is 1 and n″ is 6. In another embodiment, n′ is 2 and n″ is 6. In yet another embodiment, n′ is 3 and n″ is 6. In yet a further embodiment, n′ is 4 and n″ is 6. In yet a further embodiment, n′ is 5 and n″ is 6. In yet a further embodiment, n′ is 6 and n″ is 6.

In an embodiment, the invention provides a serotype 9N glycoconjugate comprising a serotype 9N saccharide covalently conjugated to a carrier protein (CP) through a spacer and having the general formula (IV), wherein X is OCH₂(CH₂)_{n′}, where n′ is selected from 1 to 10 and wherein X′ is CH₂O(CH₂CH₂O)_{m′}(CH₂)_{n″}CH₂C═O, where n″ is selected from 0 to 10 and m′ is selected from 0 to 4.

In an embodiment, n′ is selected from 1 to 5, m′ is selected from 0 to 4 and n″ is selected from 0 to 10. In an embodiment, n′ is selected from 1 to 5, m′ is selected from 0 to 4 and n″ is selected from 0 to 5. In an embodiment, n′ is selected from 1 to 3, m′ is selected from 0 to 2 and n″ is selected from 0 to 3. In an embodiment, n′ is selected from 1 to 2, m′ is selected from 0 to 2 and n″ is selected from 0 to 1.

In a particular embodiment, n′ is 1, m′ is 0 and n″ is 0. In another embodiment, n′ is 1, m′ is 1 and n″ is 0. In another embodiment, n′ is 1, m′ is 2 and n″ is 0. In another embodiment, n′ is 1, m′ is 3 and n″ is 0.

In another embodiment, n′ is 2, m′ is 0 and n″ is 0. In another embodiment, n′ is 2, m′ is 1 and n″ is 0. In another embodiment, n′ is 2, m′ is 2 and n″ is 0. In another embodiment, n′ is 2, m′ is 3 and n″ is 0.

In yet another embodiment, n′ is 3, m′ is 0 and n″ is 0. In yet another embodiment, n′ is 3, m′ is 1 and n″ is 0. In yet another embodiment, n′ is 3, m′ is 2 and n″ is 0. In yet another embodiment, n′ is 3, m′ is 3 and n″ is 0.

In yet a further embodiment, n′ is 4, m′ is 0 and n″ is 0. In yet a further embodiment, n′ is 4, m′ is 1 and n″ is 0. In yet a further embodiment, n′ is 4, m′ is 2 and n″ is 0. In yet a further embodiment, n′ is 4, m′ is 3 and n″ is 0.

In yet a further embodiment, n′ is 5, m′ is 0 and n″ is 0. In yet a further embodiment, n′ is 5, m′ is 1 and n″ is 0. In yet a further embodiment, n′ is 5, m′ is 2 and n″ is 0. In yet a further embodiment, n′ is 5, m′ is 3 and n″ is 0.

In a particular embodiment, n′ is 1, m′ is 0 and n″ is 1. In a particular embodiment, n′ is 1, m′ is 1 and n″ is 1. In a particular embodiment, n′ is 1, m′ is 2 and n″ is 1. In a particular embodiment, n′ is 1, m′ is 3 and n″ is 1.

In another embodiment, n′ is 2, m′ is 0 and n″ is 1. In another embodiment, n′ is 2, m′ is 1 and n″ is 1. In another embodiment, n′ is 2, m′ is 2 and n″ is 1. In another embodiment, n′ is 2, m′ is 3 and n″ is 1.

In yet another embodiment, n′ is 3, m′ is 0 and n″ is 1. In yet another embodiment, n′ is 3, m′ is 1 and n″ is 1. In yet another embodiment, n′ is 3, m′ is 2 and n″ is 1. In yet another embodiment, n′ is 3, m′ is 3 and n″ is 1.

In yet a further embodiment, n′ is 4, m′ is 0 and n″ is 1. In yet a further embodiment, n′ is 4, m′ is 1 and n″ is 1. In yet a further embodiment, n′ is 4, m′ is 2 and n″ is 1. In yet a further embodiment, n′ is 4, m′ is 3 and n″ is 1.

In yet a further embodiment, n′ is 5, m′ is 0 and n″ is 1. In yet a further embodiment, n′ is 5, m′ is 1 and n″ is 1. In yet a further embodiment, n′ is 5, m′ is 2 and n″ is 1. In yet a further embodiment, n′ is 5, m′ is 3 and n″ is 1.

In a particular embodiment, n′ is 1, m′ is 0 and n″ is 2. In a particular embodiment, n′ is 1, m′ is 1 and n″ is 2. In a particular embodiment, n′ is 1, m′ is 2 and n″ is 2. In a particular embodiment, n′ is 1, m′ is 3 and n″ is 2.

In another embodiment, n′ is 2, m′ is 0 and n″ is 2. In another embodiment, n′ is 2, m′ is 1 and n″ is 2. In another embodiment, n′ is 2, m′ is 2 and n″ is 2. In another embodiment, n′ is 2, m′ is 3 and n″ is 2.

In yet another embodiment, n′ is 3, m′ is 0 and n″ is 2. In yet another embodiment, n′ is 3, m′ is 1 and n″ is 2. In yet another embodiment, n′ is 3, m′ is 2 and n″ is 2. In yet another embodiment, n′ is 3, m′ is 3 and n″ is 2.

In yet a further embodiment, n′ is 4, m′ is 0 and n″ is 2. In yet a further embodiment, n′ is 4, m′ is 1 and n″ is 2. In yet a further embodiment, n′ is 4, m′ is 2 and n″ is 2. In yet a further embodiment, n′ is 4, m′ is 3 and n″ is 2.

In yet a further embodiment, n′ is 5, m′ is 0 and n″ is 2. In yet a further embodiment, n′ is 5, m′ is 1 and n″ is 2. In yet a further embodiment, n′ is 5, m′ is 2 and n″ is 2. In yet a further embodiment, n′ is 5, m′ is 3 and n″ is 2.

In a particular embodiment, n′ is 1, m′ is 0 and n″ is 3. In a particular embodiment, n′ is 1, m′ is 1 and n″ is 3. In a particular embodiment, n′ is 1, m′ is 2 and n″ is 3. In a particular embodiment, n′ is 1, m′ is 3 and n″ is 3.

In another embodiment, n′ is 2, m′ is 0 and n″ is 3. In another embodiment, n′ is 2, m′ is 1 and n″ is 3. In another embodiment, n′ is 2, m′ is 2 and n″ is 3. In another embodiment, n′ is 2, m′ is 3 and n″ is 3.

In yet another embodiment, n′ is 3, m′ is 0 and n″ is 3. In yet another embodiment, n′ is 3, m′ is 1 and n″ is 3. In yet another embodiment, n′ is 3, m′ is 2 and n″ is 3. In yet another embodiment, n′ is 3, m′ is 3 and n″ is 3.

In yet a further embodiment, n′ is 4, m′ is 0 and n″ is 3. In yet a further embodiment, n′ is 4, m′ is 1 and n″ is 3. In yet a further embodiment, n′ is 4, m′ is 2 and n″ is 3. In yet a further embodiment, n′ is 4, m′ is 3 and n″ is 3.

In yet a further embodiment, n′ is 5, m′ is 0 and n″ is 3. In yet a further embodiment, n′ is 5, m′ is 1 and n″ is 3. In yet a further embodiment, n′ is 5, m′ is 2 and n″ is 3. In yet a further embodiment, n′ is 5, m′ is 3 and n″ is 3.

In a particular embodiment, n′ is 1, m′ is 0 and n″ is 4. In a particular embodiment, n′ is 1, m′ is 1 and n″ is 4. In a particular embodiment, n′ is 1, m′ is 2 and n″ is 4. In a particular embodiment, n′ is 1, m′ is 3 and n″ is 4.

In another embodiment, n′ is 2, m′ is 0 and n″ is 4. In another embodiment, n′ is 2, m′ is 1 and n″ is 4. In another embodiment, n′ is 2, m′ is 2 and n″ is 4. In another embodiment, n′ is 2, m′ is 3 and n″ is 4.

In yet another embodiment, n′ is 3, m′ is 0 and n″ is 4. In yet another embodiment, n′ is 3, m′ is 1 and n″ is 4. In yet another embodiment, n′ is 3, m′ is 2 and n″ is 4. In yet another embodiment, n′ is 3, m′ is 3 and n″ is 4.

In yet a further embodiment, n′ is 4, m′ is 0 and n″ is 4. In yet a further embodiment, n′ is 4, m′ is 1 and n″ is 4. In yet a further embodiment, n′ is 4, m′ is 2 and n″ is 4. In yet a further embodiment, n′ is 4, m′ is 3 and n″ is 4.

In yet a further embodiment, n′ is 5, m′ is 0 and n″ is 4. In yet a further embodiment, n′ is 5, m′ is 1 and n″ is 4. In yet a further embodiment, n′ is 5, m′ is 2 and n″ is 4. In yet a further embodiment, n′ is 5, m′ is 3 and n″ is 4.

In a particular embodiment, n′ is 1, m′ is 0 and n″ is 5. In a particular embodiment, n′ is 1, m′ is 1 and n″ is 5. In a particular embodiment, n′ is 1, m′ is 2 and n″ is 5. In a particular embodiment, n′ is 1, m′ is 3 and n″ is 5.

In another embodiment, n′ is 2, m′ is 0 and n″ is 5. In another embodiment, n′ is 2, m′ is 1 and n″ is 5. In another embodiment, n′ is 2, m′ is 2 and n″ is 5. In another embodiment, n′ is 2, m′ is 3 and n″ is 5.

In yet another embodiment, n′ is 3, m′ is 0 and n″ is 5. In yet another embodiment, n′ is 3, m′ is 1 and n″ is 5. In yet another embodiment, n′ is 3, m′ is 2 and n″ is 5. In yet another embodiment, n′ is 3, m′ is 3 and n″ is 5.

In yet a further embodiment, n′ is 4, m′ is 0 and n″ is 5. In yet a further embodiment, n′ is 4, m′ is 1 and n″ is 5. In yet a further embodiment, n′ is 4, m′ is 2 and n″ is 5. In yet a further embodiment, n′ is 4, m′ is 3 and n″ is 5.

In yet a further embodiment, n′ is 5, m′ is 0 and n″ is 5. In yet a further embodiment, n′ is 5, m′ is 1 and n″ is 5. In yet a further embodiment, n′ is 5, m′ is 2 and n″ is 5. In yet a further embodiment, n′ is 5, m′ is 3 and n″ is 5.

In an embodiment, the invention provides a serotype 9N glycoconjugate comprising a serotype 9N saccharide covalently conjugated to a carrier protein (CP) through a spacer and having the general formula (IV), wherein X is O(CH₂CH₂O)ₘCH₂CH₂, where m is selected from 1 to 4 and wherein X′ is CH₂O(CH₂)_{n″}CH₂C═O, where n″ is selected from 0 to 10.

In an embodiment, m is selected from 1 to 3 and n″ is selected from 0 to 10. In an embodiment, m is selected from 1 to 3 and n″ is selected from 0 to 5. In an embodiment, m is selected from 1 to 2 and n″ is selected from 0 to 3. In an embodiment, m is selected from 1 to 2 and n″ is selected from 0 to 2. In a particular embodiment, m is 1 and n″ is 0. In another embodiment, m is 2 and n″ is 0. In yet another embodiment, m is 3 and n″ is 0. In yet a further embodiment, m is 4 and n″ is 0. In a particular embodiment, m is 1 and n″ is 1. In another embodiment, m is 2 and n″ is 1. In yet another embodiment, m is 3 and n″ is 1. In yet a further embodiment, m is 4 and n″ is 1. In a particular embodiment, m is 1 and n″ is 2. In another embodiment, m is 2 and n″ is 2. In yet another embodiment, m is 3 and n″ is 2. In yet a further embodiment, m is 4 and n″ is 2. In a particular embodiment, m is 1 and n″ is 3. In another embodiment, m is 2 and n″ is 3. In yet another embodiment, m is 3 and n″ is 3. In yet a further embodiment, m is 4 and n″ is 3. In a particular embodiment, m is 1 and n″ is 4. In another embodiment, m is 2 and n″ is 4. In yet another embodiment, m is 3 and n″ is 4. In yet a further embodiment, m is 4 and n″ is 4. In a particular embodiment, m is 1 and n″ is 5. In another embodiment, m is 2 and n″ is 5. In yet another embodiment, m is 3 and n″ is 5. In yet a further embodiment, m is 4 and n″ is 5. In a particular embodiment, m is 1 and n″ is 6. In another embodiment, m is 2 and n″ is 6. In yet another embodiment, m is 3 and n″ is 6. In yet a further embodiment, m is 4 and n″ is 6.

In an embodiment, the invention provides a serotype 9N glycoconjugate comprising a serotype 9N saccharide covalently conjugated to a carrier protein (CP) through a spacer and having the general formula (IV), wherein X is O(CH₂CH₂O)ₘCH₂CH₂, where m is selected from 1 to 4 and wherein X′ is CH₂O(CH₂CH₂O)_{m′}(CH₂)_{n″}CH₂C═O, where n″ is selected from 0 to 10 and m′ is selected from 0 to 4.

In an embodiment, m is selected from 1 to 3, m′ is selected from 0 to 4 and n″ is selected from 0 to 10. In an embodiment, m is selected from 1 to 2, m′ is selected from 0 to 4 and n″ is selected from 0 to 5. In an embodiment, m is selected from 1 to 2, m′ is selected from 0 to 2 and n″ is selected from 0 to 3. In an embodiment, m is selected from 1 to 2, m′ is selected from 0 to 2 and n″ is selected from 0 to 1.

In a particular embodiment, m is 1, m′ is 0 and n″ is 0. In another embodiment, m is 1, m′ is 1 and n″ is 0. In another embodiment, m is 1, m′ is 2 and n″ is 0. In another embodiment, m is 1, m′ is 3 and n″ is 0.

In another embodiment, m is 2, m′ is 0 and n″ is 0. In another embodiment, m is 2, m′ is 1 and n″ is 0. In another embodiment, m is 2, m′ is 2 and n″ is 0. In another embodiment, m is 2, m′ is 3 and n″ is 0.

In yet another embodiment, m is 3, m′ is 0 and n″ is 0. In yet another embodiment, m is 3, m′ is 1 and n″ is 0. In yet another embodiment, m is 3, m′ is 2 and n″ is 0. In yet another embodiment, m is 3, m′ is 3 and n″ is 0.

In yet a further embodiment, m is 4, m′ is 0 and n″ is 0. In yet a further embodiment, m is 4, m′ is 1 and n″ is 0. In yet a further embodiment, m is 4, m′ is 2 and n″ is 0. In yet a further embodiment, m is 4, m′ is 3 and n″ is 0.

In a particular embodiment, m is 1, m′ is 0 and n″ is 1. In a particular embodiment, m is 1, m′ is 1 and n″ is 1. In a particular embodiment, m is 1, m′ is 2 and n″ is 1. In a particular embodiment, m is 1, m′ is 3 and n″ is 1.

In another embodiment, m is 2, m′ is 0 and n″ is 1. In another embodiment, m is 2, m′ is 1 and n″ is 1. In another embodiment, m is 2, m′ is 2 and n″ is 1. In another embodiment, m is 2, m′ is 3 and n″ is 1.

In yet another embodiment, m is 3, m′ is 0 and n″ is 1. In yet another embodiment, m is 3, m′ is 1 and n″ is 1. In yet another embodiment, m is 3, m′ is 2 and n″ is 1. In yet another embodiment, m is 3, m′ is 3 and n″ is 1.

In yet a further embodiment, m is 4, m′ is 0 and n″ is 1. In yet a further embodiment, m is 4, m′ is 1 and n″ is 1. In yet a further embodiment, m is 4, m′ is 2 and n″ is 1. In yet a further embodiment, m is 4, m′ is 3 and n″ is 1.

In a particular embodiment, m is 1, m′ is 0 and n″ is 2. In a particular embodiment, m is 1, m′ is 1 and n″ is 2. In a particular embodiment, m is 1, m′ is 2 and n″ is 2. In a particular embodiment, m is 1, m′ is 3 and n″ is 2.

In another embodiment, m is 2, m′ is 0 and n″ is 2. In another embodiment, m is 2, m′ is 1 and n″ is 2. In another embodiment, m is 2, m′ is 2 and n″ is 2. In another embodiment, m is 2, m′ is 3 and n″ is 2.

In yet another embodiment, m is 3, m′ is 0 and n″ is 2. In yet another embodiment, m is 3, m′ is 1 and n″ is 2. In yet another embodiment, m is 3, m′ is 2 and n″ is 2. In yet another embodiment, m is 3, m′ is 3 and n″ is 2.

In yet a further embodiment, m is 4, m′ is 0 and n″ is 2. In yet a further embodiment, m is 4, m′ is 1 and n″ is 2. In yet a further embodiment, m is 4, m′ is 2 and n″ is 2. In yet a further embodiment, m is 4, m′ is 3 and n″ is 2.

In a particular embodiment, m is 1, m′ is 0 and n″ is 3. In a particular embodiment, m is 1, m′ is 1 and n″ is 3. In a particular embodiment, m is 1, m′ is 2 and n″ is 3. In a particular embodiment, m is 1, m′ is 3 and n″ is 3.

In another embodiment, m is 2, m′ is 0 and n″ is 3. In another embodiment, m is 2, m′ is 1 and n″ is 3. In another embodiment, m is 2, m′ is 2 and n″ is 3. In another embodiment, m is 2, m′ is 3 and n″ is 3.

In yet another embodiment, m is 3, m′ is 0 and n″ is 3. In yet another embodiment, m is 3, m′ is 1 and n″ is 3. In yet another embodiment, m is 3, m′ is 2 and n″ is 3. In yet another embodiment, m is 3, m′ is 3 and n″ is 3.

In yet a further embodiment, m is 4, m′ is 0 and n″ is 3. In yet a further embodiment, m is 4, m′ is 1 and n″ is 3. In yet a further embodiment, m is 4, m′ is 2 and n″ is 3. In yet a further embodiment, m is 4, m′ is 3 and n″ is 3.

In a particular embodiment, m is 1, m′ is 0 and n″ is 4. In a particular embodiment, m is 1, m′ is 1 and n″ is 4. In a particular embodiment, m is 1, m′ is 2 and n″ is 4. In a particular embodiment, m is 1, m′ is 3 and n″ is 4.

In another embodiment, m is 2, m′ is 0 and n″ is 4. In another embodiment, m is 2, m′ is 1 and n″ is 4. In another embodiment, m is 2, m′ is 2 and n″ is 4. In another embodiment, m is 2, m′ is 3 and n″ is 4.

In yet another embodiment, m is 3, m′ is 0 and n″ is 4. In yet another embodiment, m is 3, m′ is 1 and n″ is 4. In yet another embodiment, m is 3, m′ is 2 and n″ is 4. In yet another embodiment, m is 3, m′ is 3 and n″ is 4.

In yet a further embodiment, m is 4, m′ is 0 and n″ is 4. In yet a further embodiment, m is 4, m′ is 1 and n″ is 4. In yet a further embodiment, m is 4, m′ is 2 and n″ is 4. In yet a further embodiment, m is 4, m′ is 3 and n″ is 4.

In a particular embodiment, m is 1, m′ is 0 and n″ is 5. In a particular embodiment, m is 1, m′ is 1 and n″ is 5. In a particular embodiment, m is 1, m′ is 2 and n″ is 5. In a particular embodiment, m is 1, m′ is 3 and n″ is 5.

In another embodiment, m is 2, m′ is 0 and n″ is 5. In another embodiment, m is 2, m′ is 1 and n″ is 5. In another embodiment, m is 2, m′ is 2 and n″ is 5. In another embodiment, m is 2, m′ is 3 and n″ is 5.

In yet another embodiment, m is 3, m′ is 0 and n″ is 5. In yet another embodiment, m is 3, m′ is 1 and n″ is 5. In yet another embodiment, m is 3, m′ is 2 and n″ is 5. In yet another embodiment, m is 3, m′ is 3 and n″ is 5.

In yet a further embodiment, m is 4, m′ is 0 and n″ is 5. In yet a further embodiment, m is 4, m′ is 1 and n″ is 5. In yet a further embodiment, m is 4, m′ is 2 and n″ is 5. In yet a further embodiment, m is 4, m′ is 3 and n″ is 5.

In a very preferred embodiment of the present invention, the serotype 9N glycoconjugate of the present invention are prepared using click chemistry. The invention also relates to a method of making serotype 9N glycoconjugate, as disclosed herein above.

In an embodiment, click chemistry may comprise three steps, (a) reacting an isolated serotype 9N saccharide with a carbonic acid derivative and an azido linker in an aprotic solvent to produce an activated azido saccharide (activation of the saccharide), (b) reacting a carrier protein with an agent bearing an N-Hydroxysuccinimide (NHS) moiety and an alkyne group where the NHS moiety reacts with the amino groups to form an amide linkage thereby obtaining an alkyne functionalized carrier protein (activation of the carrier protein), (c) reacting the activated azido saccharide of step (a) with the activated alkyne-carrier protein of step (b) by Cu⁺¹ mediated azide-alkyne cycloaddition reaction to form a glycoconjugate.

Following step (a) the saccharide is said to be activated and is referred to herein as “activated saccharide” or “activated azido saccharide”.

Following step (b) the carrier is said to be activated and is referred to as “activated carrier”.

As mentioned above, before the activation (a), sizing of the saccharide to a target molecular weight (MW) range may be performed. Therefore, in an embodiment, the isolated serotype 9N saccharide is sized before activation with a carbonic acid derivative and an azido linker. In an embodiment, the isolated serotype 9N saccharide is sized to any of the target molecular weight (MW) range defined above. In an embodiment, the isolated serotype 9N saccharide is not sized before activation with a carbonic acid derivative and an azido linker.

In an embodiment, said carbonic acid derivative is selected from the group consisting of 1,1′-carbonyldiimidazole (CDI), 1,1′-carbonyl-di-(1,2,4-triazole) (CDT), N,N′-Disuccinimidyl carbonate (DSC) and N-hydroxysuccinimidyl chloroformate.

In an embodiment, said carbonic acid derivative is 1,1′-carbonyldiimidazole (CDI). In another embodiment, said carbonic acid derivative is 1,1′-Carbonyl-di-(1,2,4-triazole) (CDT). In another embodiment, said carbonic acid derivative is N,N′-Disuccinimidyl carbonate (DSC). In yet a further embodiment, said carbonic acid derivative is N-hydroxysuccinimidyl chloroformate.

In an embodiment, said carbonic acid derivative is 1,1′-carbonyldiimidazole (CDI) or 1,1′-Carbonyl-di-(1,2,4-triazole) (CDT). In an embodiment, said carbonic acid derivative is 1,1′-carbonyldiimidazole (CDI). Preferably, said carbonic acid derivative N,N′-Disuccinimidyl carbonate (DSC).

In an embodiment, said azido linker is a compound of formula (VI),

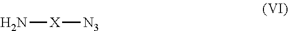

wherein X is selected from the group consisting of CH₂(CH₂)ₙ, (CH₂CH₂O)ₘCH₂CH₂, NHCO(CH₂)ₙ, NHCO(CH₂CH₂O)ₘCH₂CH₂, OCH₂(CH₂)ₙ and O(CH₂CH₂O)ₘCH₂CH₂; where n is selected from 1 to 10 and m is selected from 1 to 4.

In an embodiment, said azido linker is a compound of formula (VI), wherein X is CH₂(CH₂)ₙ, and n is selected from 1 to 10. In an embodiment, n is selected from 1 to 5. In an embodiment, n is selected from 1 to 4. In an embodiment, n is selected from 1 to 3. In an embodiment, n is selected from 1 to 2. In a particular embodiment, n is 1. In another embodiment, n is 2. In yet another embodiment, n is 3. In yet a further embodiment, n is 4. In yet a further embodiment, n is 5. In yet a further embodiment, n is 6. In yet a further embodiment, n is 7. In yet a further embodiment, n is 8. In yet a further embodiment, n is 9. In yet a further embodiment, n is 10.

In an embodiment, said azido linker is a compound of formula (VI), wherein X is (CH₂CH₂O)ₘCH₂CH₂, wherein m is selected from 1 to 4. In an embodiment, m is selected from 1 to 3. In an embodiment, m is selected from 1 to 2. In a particular embodiment, m is 1. In another embodiment, m is 2. In yet another embodiment, m is 3. In yet a further embodiment, m is 4.

In an embodiment, said azido linker is a compound of formula (VI), wherein X is NHCO(CH₂)ₙ, and n is selected from 1 to 10. In an embodiment, n is selected from 1 to 5. In an embodiment, n is selected from 1 to 4. In an embodiment, n is selected from 1 to 3. In an embodiment, n is selected from 1 to 2. In a particular embodiment, n is 1. In another embodiment, n is 2. In yet another embodiment, n is 3. In yet a further embodiment, n is 4. In yet a further embodiment, n is 5. In yet a further embodiment, n is 6. In yet a further embodiment, n is 7. In yet a further embodiment, n is 8. In yet a further embodiment, n is 9. In yet a further embodiment, n is 10.

In an embodiment, said azido linker is a compound of formula (VI), wherein X is NHCO(CH₂CH₂O)ₘCH₂CH₂, where m is selected from 1 to 4. In an embodiment, m is selected from 1 to 3. In an embodiment, m is selected from 1 to 2. In a particular embodiment, m is 1. In another embodiment, m is 2. In yet another embodiment, m is 3. In yet a further embodiment, m is 4.

In an embodiment, said azido linker is a compound of formula (VI), wherein X is OCH₂(CH₂)ₙ, and n is selected from 1 to 10. In an embodiment, n is selected from 1 to 5. In an embodiment, n is selected from 1 to 4. In an embodiment, n is selected from 1 to 3. In an embodiment, n is selected from 1 to 2. In a particular embodiment, n is 1. In another embodiment, n is 2. In yet another embodiment, n is 3. In yet a further embodiment, n is 4. In yet a further embodiment, n is 5. In yet a further embodiment, n is 6. In yet a further embodiment, n is 7. In yet a further embodiment, n is 8. In yet a further embodiment, n is 9. In yet a further embodiment, n is 10.

In an embodiment, said azido linker is a compound of formula (VI), wherein X is O(CH₂CH₂O)ₘCH₂CH₂, where m is selected from 1 to 4. In an embodiment, m is selected from 1 to 3. In an embodiment, m is selected from 1 to 2. In a particular embodiment, m is 1. In another embodiment, m is 2. In yet another embodiment, m is 3. In yet a further embodiment, m is 4.

In an embodiment, said azido linker is a compound of formula (VII),

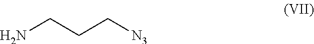

In a preferred embodiment, said azido linker is 3-azido-propylamine.

In an embodiment, said agent bearing an N-Hydroxysuccinimide (NHS) moiety and an alkyne group is an agent bearing an N-Hydroxysuccinimide (NHS) moiety and a terminal alkyne.

In an embodiment, said agent bearing an N-Hydroxysuccinimide (NHS) moiety and an alkyne group is an agent bearing an N-Hydroxysuccinimide (NHS) moiety and a cycloalkyne.

In an embodiment, said agent bearing an N-Hydroxysuccinimide (NHS) moiety and an alkyne group is a compound of formula (VIII),

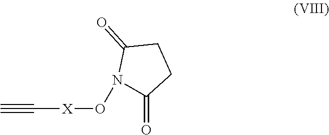

where X is selected from the group consisting of CH₂O(CH₂)ₙCH₂C═O and CH₂O(CH₂CH₂O)ₘ(CH₂)ₙCH₂C═O, where n is selected from 0 to 10 and m is selected from 0 to 4.

In an embodiment, said agent bearing an N-Hydroxysuccinimide (NHS) moiety and an alkyne group is a compound of formula (VIII), wherein X is CH₂O(CH₂)ₙCH₂C═O, where n is selected from 0 to 10. In an embodiment, n is selected from 0 to 5. In an embodiment, n is selected from 0 to 4. In an embodiment, n is selected from 0 to 3. In an embodiment, n is selected from 0 to 2. In a particular embodiment, n is 0. In a particular embodiment, n is 1. In another embodiment, n is 2. In yet another embodiment, n is 3. In yet a further embodiment, n is 4. In yet a further embodiment, n is 5. In yet a further embodiment, n is 6. In yet a further embodiment, n is 7. In yet a further embodiment, n is 8. In yet a further embodiment, n is 9. In yet a further embodiment, n is 10.

In an embodiment, said agent bearing an N-Hydroxysuccinimide (NHS) moiety and an alkyne group is a compound of formula (VIII), wherein X is CH₂O(CH₂CH₂O)ₘ(CH₂)ₙCH₂C═O, where n is selected from 0 to 10 and m is selected from 0 to 4. In an embodiment, n is selected from 0 to 5. In an embodiment, n is selected from 0 to 4. In an embodiment, n is selected from 0 to 3. In an embodiment, n is selected from 0 to 2. In a particular embodiment, n is 0. In a particular embodiment, n is 1. In another embodiment, n is 2. In yet another embodiment, n is 3. In yet a further embodiment, n is 4. In yet a further embodiment, n is 5. In yet a further embodiment, n is 6. In yet a further embodiment, n is 7. In yet a further embodiment, n is 8. In yet a further embodiment, n is 9. In yet a further embodiment, n is 10. In an embodiment, m is selected from 0 to 3. In an embodiment, m is selected from 0 to 2. In a particular embodiment, m is 1. In a particular embodiment, m is 1. In another embodiment, m is 2. In yet another embodiment, m is 3. In yet a further embodiment, m is 4.

In an embodiment, n is selected from 0 to 5 and m is selected from 0 to 3. In an embodiment, n is selected from 0 to 5 and m is selected from 0 to 2.

In an embodiment, n is selected from 0 to 4 and m is selected from 0 to 3. In an embodiment, n is selected from 0 to 4 and m is selected from 0 to 2.

In an embodiment, n is selected from 0 to 3 and m is selected from 0 to 3. In an embodiment, n is selected from 0 to 3 and m is selected from 0 to 2.

In an embodiment, n is selected from 0 to 2 and m is selected from 0 to 3. In an embodiment, n is selected from 0 to 2 and m is selected from 0 to 2.

In an embodiment, n is selected from 0 to 1 and m is selected from 0 to 3. In an embodiment, n is selected from 0 to 1 and m is selected from 0 to 2.

In an embodiment, n is 0 and m is 0. In an embodiment, n is 1 and m is 0. In an embodiment, n is 2 and m is 0. In an embodiment, n is 3 and m is 0. In an embodiment, n is 4 and m is 0. In an embodiment, n is 5 and m is 0. In an embodiment, n is 6 and m is 0. In an embodiment, n is 7 and m is 0. In an embodiment, n is 8 and m is 0. In an embodiment, n is 9 and m is 0. In an embodiment, n is 10 and m is 0.

In an embodiment, n is 0 and m is 1. In an embodiment, n is 1 and m is 1. In an embodiment, n is 2 and m is 1. In an embodiment, n is 3 and m is 1. In an embodiment, n is 4 and m is 1. In an embodiment, n is 5 and m is 1. In an embodiment, n is 6 and m is 1. In an embodiment, n is 7 and m is 1. In an embodiment, n is 8 and m is 1. In an embodiment, n is 9 and m is 1. In an embodiment, n is 10 and m is 1.

In an embodiment, n is 0 and m is 2. In an embodiment, n is 1 and m is 2. In an embodiment, n is 2 and m is 2. In an embodiment, n is 3 and m is 2. In an embodiment, n is 4 and m is 2. In an embodiment, n is 5 and m is 2. In an embodiment, n is 6 and m is 2. In an embodiment, n is 7 and m is 2. In an embodiment, n is 8 and m is 2. In an embodiment, n is 9 and m is 2. In an embodiment, n is 10 and m is 2.

In an embodiment, n is 0 and m is 3. In an embodiment, n is 1 and m is 3. In an embodiment, n is 2 and m is 3. In an embodiment, n is 3 and m is 3. In an embodiment, n is 4 and m is 3. In an embodiment, n is 5 and m is 3. In an embodiment, n is 6 and m is 3. In an embodiment, n is 7 and m is 3. In an embodiment, n is 8 and m is 3. In an embodiment, n is 9 and m is 3. In an embodiment, n is 10 and m is 3.

In an embodiment, n is 0 and m is 4. In an embodiment, n is 1 and m is 4. In an embodiment, n is 2 and m is 4. In an embodiment, n is 3 and m is 4. In an embodiment, n is 4 and m is 4. In an embodiment, n is 5 and m is 4. In an embodiment, n is 6 and m is 4. In an embodiment, n is 7 and m is 4. In an embodiment, n is 8 and m is 4. In an embodiment, n is 9 and m is 4. In an embodiment, n is 10 and m is 4.

In an embodiment, said agent bearing an N-Hydroxysuccinimide (NHS) moiety and an alkyne group is a compound of formula (IX):

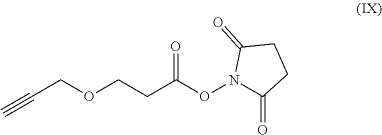 [SMILES: C#CCOCCC(=O)ON1C(=O)CCC1=O]

In an embodiment, step a) comprises reacting the saccharide with a carbonic acid derivative followed by reacting the carbonic acid derivative-activated saccharide with an azido linker in an aprotic solvent to produce an activated azido saccharide.

In one embodiment step a) comprises reacting the saccharide with an amount of carbonic acid derivative that is between 0.01-10 molar equivalent to the amount of saccharide present in the reaction mixture.

In one embodiment step a) comprises reacting the saccharide with an amount of carbonic acid derivative that is between 0.1-10 molar equivalent to the amount of saccharide present in the reaction mixture.

In one embodiment step a) comprises reacting the saccharide with an amount of carbonic acid derivative that is between 0.5-5 molar equivalent to the amount of saccharide present in the reaction mixture.

In one embodiment step a) comprises reacting the saccharide with an amount of carbonic acid derivative that is between 1-5 molar equivalent to the amount of saccharide present in the reaction mixture.

In one embodiment step a) comprises reacting the saccharide with an amount of carbonic acid derivative that is between 2-5 molar equivalent to the amount of saccharide present in the reaction mixture.

In one embodiment step a) comprises reacting the saccharide with an amount of carbonic acid derivative that is between 5-10 molar equivalent to the amount of saccharide present in the reaction mixture.

In one embodiment step a) comprises reacting the saccharide with an amount of carbonic acid derivative that is between 0.1-5 molar equivalent to the amount of saccharide present in the reaction mixture.

In one embodiment step a) comprises reacting the saccharide with an amount of carbonic acid derivative that is between 0.5-2 molar equivalent to the amount of saccharide present in the reaction mixture.

In one embodiment step a) comprises reacting the saccharide with an amount of carbonic acid derivative of about 0.01 molar equivalent to the amount of saccharide present in the reaction mixture.

In one embodiment step a) comprises reacting the saccharide with an amount of carbonic acid derivative of about 0.1 molar equivalent to the amount of saccharide present in the reaction mixture.

In one embodiment step a) comprises reacting the saccharide with an amount of carbonic acid derivative of about 0.2 molar equivalent to the amount of saccharide present in the reaction mixture.

In one embodiment step a) comprises reacting the saccharide with an amount of carbonic acid derivative of about 0.5 molar equivalent to the amount of saccharide present in the reaction mixture.

In one embodiment step a) comprises reacting the saccharide with an amount of carbonic acid derivative of about 1 molar equivalent to the amount of saccharide present in the reaction mixture.

In one embodiment step a) comprises reacting the saccharide with an amount of carbonic acid derivative of about 2 molar equivalent to the amount of saccharide present in the reaction mixture.

In one embodiment step a) comprises reacting the saccharide with an amount of carbonic acid derivative of about 5 molar equivalent to the amount of saccharide present in the reaction mixture.

In one embodiment step a) comprises reacting the saccharide with an amount of carbonic acid derivative of about 10 molar equivalent to the amount of saccharide present in the reaction mixture.

In an embodiment, at step a) the isolated saccharide is reacted with a carbonic acid derivative in an aprotic solvent.

In one embodiment the isolated saccharide is reacted with a carbonic acid derivative in a solution consisting essentially of dimethylsulphoxide (DMSO) or dimethylformamide (DMF). In one embodiment the isolated saccharide is reacted with a carbonic acid derivative in a solution consisting essentially of dimethylformamide (DMF). In one embodiment the isolated saccharide is reacted with a carbonic acid derivative in a solution consisting essentially of dimethylsulphoxide (DMSO).

In an embodiment, the isolated saccharide is reacted with a carbonic acid derivative in a solution consisting essentially of dimethylacetamide. In an embodiment, the isolated saccharide is reacted with a carbonic acid derivative in a solution consisting essentially of N-methyl-2-pyrrolidone. In an embodiment, the isolated saccharide is reacted with a carbonic acid derivative in a solution consisting essentially of hexamethylphosphoramide (HMPA).

In a preferred embodiment the isolated saccharide is reacted with a carbonic acid derivative in a solution consisting essentially of dimethylsulphoxide (DMSO).

In one embodiment the isolated saccharide is reacted with a carbonic acid derivative in dimethylsulphoxide (DMSO) or dimethylformamide (DMF). In one embodiment the isolated saccharide is reacted with a carbonic acid derivative in dimethylformamide (DMF). In one embodiment the isolated saccharide is reacted with a carbonic acid derivative in dimethylsulphoxide (DMSO).

In an embodiment, the isolated saccharide is reacted with a carbonic acid derivative in dimethylacetamide. In an embodiment, the isolated saccharide is reacted with a carbonic acid derivative in N-methyl-2-pyrrolidone. In an embodiment, the isolated saccharide is reacted with a carbonic acid derivative in hexamethylphosphoramide (HMPA).

In a preferred embodiment the isolated saccharide is reacted with CDI in dimethylsulphoxide (DMSO). In an embodiment the isolated saccharide is reacted with CDI in anhydrous DMSO.

It has been surprisingly found that reacting the isolated saccharide with CDI in an environment with a moisture level of about 0.1% to 1% (v/v) allows to avoid side reactions.

Therefore, in one embodiment the isolated saccharide is reacted with CDI in an aprotic solvent comprising 0.1% to 1% (v/v) water. In one embodiment the isolated saccharide is reacted with CDI in an aprotic solvent comprising 0.1% to 0.5% (v/v) water. In one embodiment the isolated saccharide is reacted with CDI in an aprotic solvent comprising 0.1% to 0.2% (v/v) water.

In one embodiment the isolated saccharide is reacted with CDI in an aprotic solvent comprising about 0.1% (v/v) water. In one embodiment the isolated saccharide is reacted with CDI in an aprotic solvent comprising about 0.2% (v/v) water. In one embodiment the isolated saccharide is reacted with CDI in an aprotic solvent comprising about 0.3% (v/v) water. In one embodiment the isolated saccharide is reacted with CDI in an aprotic solvent comprising about 0.4% (v/v) water. In one embodiment the isolated saccharide is reacted with CDI in an aprotic solvent comprising about 0.5% (v/v) water. In one embodiment the isolated saccharide is reacted with CDI in an aprotic solvent comprising about 0.6% (v/v) water. In one embodiment the isolated saccharide is reacted with CDI in an aprotic solvent comprising about 0.7% (v/v) water. In one embodiment the isolated saccharide is reacted with CDI in an aprotic solvent comprising about 0.8% (v/v) water. In one embodiment the isolated saccharide is reacted with CDI in an aprotic solvent comprising about 0.9% (v/v) water.

In one embodiment the isolated saccharide is reacted with CDI in DMSO comprising 0.1% to 1% (v/v) water. In one embodiment the isolated saccharide is reacted with CDI in DMSO comprising 0.1% to 0.5% (v/v) water. In one embodiment the isolated saccharide is reacted with CDI in DMSO comprising 0.1% to 0.2% (v/v) water.

In one embodiment the isolated saccharide is reacted with CDI in DMSO comprising about 0.1% (v/v) water. In one embodiment the isolated saccharide is reacted with CDI in DMSO comprising about 0.2% (v/v) water. In one embodiment the isolated saccharide is reacted with CDI in DMSO comprising about 0.3% (v/v) water. In one embodiment the isolated saccharide is reacted with CDI in DMSO comprising about 0.4% (v/v) water. In one embodiment the isolated saccharide is reacted with CDI in DMSO comprising about 0.5% (v/v) water. In one embodiment the isolated saccharide is reacted with CDI in DMSO comprising about 0.6% (v/v) water. In one embodiment the isolated saccharide is reacted with CDI in DMSO comprising about 0.7% (v/v) water. In one embodiment the isolated saccharide is reacted with CDI in DMSO comprising about 0.8% (v/v) water. In one embodiment the isolated saccharide is reacted with CDI in DMSO comprising about 0.9% (v/v) water.

In one embodiment the free carbonic acid derivative is then quenched by the addition of water before the addition of the azido linker. Water can inactivate free CDI.

Therefore, in an embodiment, carbonic acid derivative activation is followed by the addition of water. In an embodiment, water is added to bring the total water content in the mixture to between about 1% to about 10% (v/v). In an embodiment, water is added to bring the total water content in the mixture to between about 1% to about 5% (v/v). In an embodiment, water is added to bring the total water content in the mixture to about 1% (v/v). In an embodiment, water is added to bring the total water content in the mixture to about 2% (v/v). In an embodiment, water is added to bring the total water content in the mixture to about 5% (v/v).

Once the saccharide has been reacted with carbonic acid derivative and following an eventual quenching of carbonic acid derivative with water, the carbonic acid derivative-activated saccharide is reacted with an azido linker.

In one embodiment step a) further comprises reacting the carbonic acid derivative-activated saccharide with an amount of azido linker that is between 0.01-10 molar equivalent to the amount of polysaccharide Repeat Unit of the activated saccharide (molar equivalent of RU).

In one embodiment step a) further comprises reacting the carbonic acid derivative-activated saccharide with an amount of azido linker that is between 0.1-5 molar equivalent to the amount of polysaccharide repeat unit of the activated saccharide.

In one embodiment step a) further comprises reacting the carbonic acid derivative-activated saccharide with an amount of azido linker that is between 0.5-2 molar equivalent to the amount of polysaccharide repeat unit of the activated saccharide.

In one embodiment step a) further comprises reacting the carbonic acid derivative-activated saccharide with an amount of azido linker that is between 1-5 molar equivalent to the amount of polysaccharide repeat unit of the activated saccharide.

In the above embodiments, said carbonic acid derivative may be CDI. In another embodiment, said carbonic acid derivative is CDT. In a preferred embodiment, said carbonic acid derivative is DSC (N,N′-Disuccinimidyl carbonate).

In one embodiment the degree of activation of the activated saccharide following step a) is between 1.0 to 100%. The degree of activation of the azido saccharide being defined as the percentage of Repeating Unit linked to an azido linker.

In one embodiment the degree of activation of the activated saccharide following step a) is between 5 to 70%. In another embodiment the degree of activation of the activated saccharide following step a) is between 5 to 50%. In another embodiment the degree of activation of the activated saccharide following step a) is between 10 to 40%.

In another embodiment the degree of activation of the activated saccharide following step a) is between 5 to 15%.

In another embodiment the degree of activation of the activated saccharide following step a) is between 15 to 35%.

In another embodiment the degree of activation of the activated saccharide following step a) is between 15 to 25%.

In an embodiment the degree of activation of the activated saccharide following step a) is about 25%.

In one embodiment step b) comprises reacting the carrier protein with an amount of agent bearing an N-Hydroxysuccinimide (NHS) moiety and an alkyne group that is 0.1-10 molar equivalents to the lysines on the carrier.

In one embodiment step b) comprises reacting the carrier protein with an amount of agent bearing an N-Hydroxysuccinimide (NHS) moiety and an alkyne group that is 0.5-10 molar equivalents to the lysines on the carrier.

In one embodiment step b) comprises reacting the carrier protein with an amount of agent bearing an N-Hydroxysuccinimide (NHS) moiety and an alkyne group that is 1-5 molar equivalents to the lysines on the carrier.

In one embodiment step b) comprises reacting the carrier protein with an amount of agent bearing an N-Hydroxysuccinimide (NHS) moiety and an alkyne group that is 2-5 molar equivalents to the lysines on the carrier.

In one embodiment step b) comprises reacting the carrier protein with an amount of agent bearing an N-Hydroxysuccinimide (NHS) moiety and an alkyne group that is 5-10 molar equivalents to the lysines on the carrier.

In one embodiment step b) comprises reacting the carrier protein with an amount of agent bearing an N-Hydroxysuccinimide (NHS) moiety and an alkyne group that is 1-5 molar equivalents to the lysines on the carrier.

In one embodiment step b) comprises reacting the carrier protein with an amount of agent bearing an N-Hydroxysuccinimide (NHS) moiety and an alkyne group that is about 10 molar equivalents to the lysines on the carrier.

In one embodiment step b) comprises reacting the carrier protein with an amount of agent bearing an N-Hydroxysuccinimide (NHS) moiety and an alkyne group that is about 5 molar equivalents to the lysines on the carrier.

In one embodiment step b) comprises reacting the carrier protein with an amount of agent bearing an N-Hydroxysuccinimide (NHS) moiety and an alkyne group that is about 2 molar equivalents to the lysines on the carrier.

In one embodiment step b) comprises reacting the carrier protein with an amount of agent bearing an N-Hydroxysuccinimide (NHS) moiety and an alkyne group that is about 1 molar equivalent to the lysines on the carrier.

In one embodiment step b) comprises reacting the carrier protein with an amount of agent bearing an N-Hydroxysuccinimide (NHS) moiety and an alkyne group that is about 0.5 molar equivalents to the lysines on the carrier.

In one embodiment step b) comprises reacting the carrier protein with an amount of agent bearing an N-Hydroxysuccinimide (NHS) moiety and an alkyne group that is about 0.1 molar equivalents to the lysines on the carrier.

In one embodiment the degree of activation of the activated carrier following step b) is between 1 and 50. The degree of activation of the activated carrier being defined as the number of lysine residues in the carrier protein that become linked to the agent bearing an N-Hydroxysuccinimide (NHS) moiety and an alkyne group.

In an embodiment, the carrier protein is CRM₁₉₇, which contains 39 lysine residues. In said embodiment the degree of activation of the activated carrier following step b) may be between 1 to 30. In another embodiment the degree of activation of the activated carrier (CRM₁₉₇) following step b) is between 5 to 20. In another embodiment the degree of activation of the activated carrier (CRM₁₉₇) following step b) is between 9 to 18. In another embodiment the degree of activation of the activated carrier (CRM₁₉₇) following step b) is between 8 to 11. In another embodiment the degree of activation of the activated carrier (CRM₁₉₇) following step b) is between 15 to 20. In another embodiment the degree of activation of the activated carrier (CRM₁₉₇) following step b) is about 5. In another embodiment the degree of activation of the activated carrier (CRM₁₉₇) following step b) is about 6. In another embodiment the degree of activation of the activated carrier (CRM₁₉₇) following step b) is about 7. In another embodiment the degree of activation of the activated carrier (CRM₁₉₇) following step b) is about 8. In another embodiment the degree of activation of the activated carrier (CRM₁₉₇) following step b) is about 9. In another embodiment the degree of activation of the activated carrier (CRM₁₉₇) following step b) is about 10. In another embodiment the degree of activation of the activated carrier (CRM₁₉₇) following step b) is about 11. In another embodiment the degree of activation of the activated carrier (CRM₁₉₇) following step b) is about 12. In another embodiment the degree of activation of the activated carrier (CRM₁₉₇) following step b) is about 13. In another embodiment the degree of activation of the activated carrier (CRM₁₉₇) following step b) is about 14. In another embodiment the degree of activation of the activated carrier (CRM₁₉₇) following step b) is about 15. In another embodiment the degree of activation of the activated carrier (CRM₁₉₇) following step b) is about 16. In another embodiment the degree of activation of the activated carrier (CRM₁₉₇) following step b) is about 17. In another embodiment the degree of activation of the activated carrier (CRM₁₉₇) following step b) is about 18. In another embodiment the degree of activation of the activated carrier (CRM₁₉₇) following step b) is about 19. In another embodiment the degree of activation of the activated carrier (CRM₁₉₇) following step b) is about 20. In another embodiment the degree of activation of the activated carrier (CRM₁₉₇) following step b) is about 21. In another embodiment the degree of activation of the activated carrier (CRM₁₉₇) following step b) is about 22. In another embodiment the degree of activation of the activated carrier (CRM₁₉₇) following step b) is about 23. In another embodiment the degree of activation of the activated carrier (CRM₁₉₇) following step b) is about 24. In another embodiment the degree of activation of the activated carrier (CRM₁₉₇) following step b) is about 25.

In an embodiment, the carrier protein is SCP or a fragment thereof. In said embodiment the degree of activation of the activated carrier following step b) may be between 1 to 50. In another embodiment the degree of activation of the activated carrier (SCP) following step b) is between 5 to 50. In another embodiment the degree of activation of the activated carrier (SCP) following step b) is between 7 to 45. In another embodiment the degree of activation of the activated carrier (SCP) following step b) is between 5 to 25. In another embodiment the degree of activation of the activated carrier (SCP) following step b) is between 10 to 25. In another embodiment the degree of activation of the activated carrier (SCP) following step b) is between 17 to 22. In another embodiment the degree of activation of the activated carrier (SCP) following step b) is about 5. In another embodiment the degree of activation of the activated carrier (SCP) following step b) is about 7. In another embodiment the degree of activation of the activated carrier (SCP) following step b) is about 10. In another embodiment the degree of activation of the activated carrier (SCP) following step b) is about 13. In another embodiment the degree of activation of the activated carrier (SCP) following step b) is about 15. In another embodiment the degree of activation of the activated carrier (SCP) following step b) is about 20. In another embodiment the degree of activation of the activated carrier (SCP) following step b) is about 26.

In another embodiment the degree of activation of the activated carrier (SCP) following step b) is about 30. In another embodiment the degree of activation of the activated carrier (SCP) following step b) is about 35. In another embodiment the degree of activation of the activated carrier (SCP) following step b) is about 37. In another embodiment the degree of activation of the activated carrier (SCP) following step b) is about 40. In another embodiment the degree of activation of the activated carrier (SCP) following step b) is about 45. In another embodiment the degree of activation of the activated carrier (SCP) following step b) is about 50.

In an embodiment, the carrier protein is TT or a fragment thereof. In said embodiment the degree of activation of the activated carrier following step b) may be between 1 to 30. In another embodiment the degree of activation of the activated carrier (TT) following step b) is between 5 to 25. In another embodiment the degree of activation of the activated carrier (TT) following step b) is between 7 to 25. In another embodiment the degree of activation of the activated carrier (TT) following step b) is between 10 to 20. In another embodiment the degree of activation of the activated carrier (TT) following step b) is about 5. In another embodiment the degree of activation of the activated carrier (TT) following step b) is about 7. In another embodiment the degree of activation of the activated carrier (TT) following step b) is about 10. In another embodiment the degree of activation of the activated carrier (TT) following step b) is about 12. In another embodiment the degree of activation of the activated carrier (TT) following step b) is about 15. In another embodiment the degree of activation of the activated carrier (TT) following step b) is about 20. In another embodiment the degree of activation of the activated carrier (TT) following step b) is about 25. In another embodiment the degree of activation of the activated carrier (TT) following step b) is about 30.

In an embodiment, the conjugation reaction c) is carried out in aqueous buffer. In an embodiment, the conjugation reaction c) is carried out in aqueous buffer in the presence of copper (I) as catalyst. In an embodiment, the conjugation reaction c) is carried out in aqueous buffer in the presence an oxidant and of copper (I) as catalyst. In a preferred embodiment, the conjugation reaction c) is carried out in aqueous buffer in the presence of copper (I) as catalyst and ascorbate as oxidant. In an embodiment, THPTA (tris(3-hydroxypropyltriazolylmethyl)amine) and aminoguanidine may be further added to protect the protein from side reactions. Therefore, in a preferred embodiment, the conjugation reaction c) is carried out in aqueous buffer in the presence of copper (I) as catalyst and ascorbate as oxidant, wherein the reaction mixture further comprises THPTA (tris(3-hydroxypropyltriazolylmethyl)amine) and aminoguanidine.

In an embodiment the initial input ratio (weight by weight) of activated azido saccharide to activated alkyne-carrier at step c) is between 0.1 and 3. In an embodiment the initial input ratio (weight by weight) of activated azido saccharide to activated alkyne-carrier at step c) is between 0.5 and 2. In an embodiment the initial input ratio (weight by weight) of activated azido saccharide to activated alkyne-carrier at step c) is between 0.6 and 1.5. In a preferred embodiment the initial input ratio (weight by weight) of activated azido saccharide to activated alkyne-carrier at step c) is between 0.8 and 1. In an embodiment the initial input ratio (weight by weight) of activated azido saccharide to activated alkyne-carrier at step c) is about 0.5. In an embodiment the initial input ratio (weight by weight) of activated azido saccharide to activated alkyne-carrier at step c) is about 0.6. In an embodiment the initial input ratio (weight by weight) of activated azido saccharide to activated alkyne-carrier at step c) is about 0.7. In an embodiment the initial input ratio (weight by weight) of activated azido saccharide to activated alkyne-carrier at step c) is about 0.8. In an embodiment the initial input ratio (weight by weight) of activated azido saccharide to activated alkyne-carrier at step c) is about 0.9. In an embodiment the initial input ratio (weight by weight) of activated azido saccharide to activated alkyne-carrier at step c) is about 1. In an embodiment the initial input ratio (weight by weight) of activated azido saccharide to activated alkyne-carrier at step c) is about 1.1. In an embodiment the initial input ratio (weight by weight) of activated azido saccharide to activated alkyne-carrier at step c) is about 1.2. In an embodiment the initial input ratio (weight by weight) of activated azido saccharide to activated alkyne-carrier at step c) is about 1.3. In an embodiment the initial input ratio (weight by weight) of activated azido saccharide to activated alkyne-carrier at step c) is about 1.4. In an embodiment the initial input ratio (weight by weight) of activated azido saccharide to activated alkyne-carrier at step c) is about 1.5. In an embodiment the initial input ratio (weight by weight) of activated azido saccharide to activated alkyne-carrier at step c) is about 1.6. In an embodiment the initial input ratio (weight by weight) of activated azido saccharide to activated alkyne-carrier at step c) is about 1.7. In an embodiment the initial input ratio (weight by weight) of activated azido saccharide to activated alkyne-carrier at step c) is about 1.8. In an embodiment the initial input ratio (weight by weight) of activated azido saccharide to activated alkyne-carrier at step c) is about 1.9. In an embodiment the initial input ratio (weight by weight) of activated azido saccharide to activated alkyne-carrier at step c) is about 2.

Following the click conjugation reaction, there may remain unreacted azido groups in the conjugates, these may be capped using a suitable azido group capping agent. Therefore, in an embodiment, following step c), unreacted azido groups in the conjugates, are capped using a suitable azido group capping agent. In one embodiment this azido group capping agent is an agent bearing an alkyne group. In one embodiment this azido group capping agent is an agent bearing a terminal alkyne. In one embodiment this azido group capping agent is an agent bearing a cycloalkyne.

In an embodiment, said azido group capping agent is a compound of formula (X),

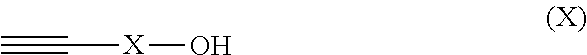

wherein X is (CH₂)ₙ wherein n is selected from 1 to 15.

In one embodiment this azido group capping agent is propargyl alcohol.

Therefore, in an embodiment, following step (c) the process further comprises a step of capping the unreacted azido groups remained in the conjugates with an azido group capping agent.

In an embodiment the capping of the unreacted azido groups is performed with an amount of capping agent that is between 0.05 to 20 molar equivalents to the amount of polysaccharide repeat unit of the activated saccharide.

In an embodiment the capping of the unreacted azido groups is performed with an amount of capping agent that is between 0.1 to 15 molar equivalents to the amount of polysaccharide repeat unit of the activated saccharide.

In an embodiment the capping of the unreacted azido groups is performed with an amount of capping agent that is between 0.5 to 10 molar equivalents to the amount of polysaccharide repeat unit of the activated saccharide.

In an embodiment the capping of the unreacted azido groups is performed with an amount of capping agent that is between 0.5 to 5 molar equivalents to the amount of polysaccharide repeat unit of the activated saccharide.

In an embodiment the capping of the unreacted azido groups is performed with an amount of capping agent that is between 0.5 to 2 molar equivalents to the amount of polysaccharide repeat unit of the activated saccharide.

In an embodiment the capping of the unreacted azido groups is performed with an amount of capping agent that is between 0.5 to 1 molar equivalents to the amount of polysaccharide repeat unit of the activated saccharide.

In an embodiment the capping of the unreacted azido groups is performed with an amount of capping agent that is between 1 to 2 molar equivalents to the amount of polysaccharide repeat unit of the activated saccharide.

In an embodiment the capping of the unreacted azido groups is performed with an amount of capping agent that is between 0.75 to 1.5 molar equivalents to the amount of polysaccharide repeat unit of the activated saccharide.

In an embodiment the capping of the unreacted azido groups is performed with an amount of capping agent that is about 1 molar equivalent to the amount of polysaccharide repeat unit of the activated saccharide.

In an embodiment the capping of the unreacted azido groups is performed with an amount of capping agent that is about 1.5 molar equivalent to the amount of polysaccharide repeat unit of the activated saccharide.

In an embodiment the capping of the unreacted azido groups is performed with an amount of capping agent that is about 0.5 molar equivalent to the amount of polysaccharide repeat unit of the activated saccharide.

In an embodiment the capping of the unreacted azido groups is performed with an amount of capping agent that is about 2 molar equivalents to the amount of polysaccharide repeat unit of the activated saccharide.

Following the click conjugation reaction, unreacted alkyne groups may remain present in the conjugates, these may be capped using a suitable alkyne group capping agent. In one embodiment this alkyne group capping agent is an agent bearing an azido group.

In an embodiment, said alkyne group capping agent is a compound of formula (XI),

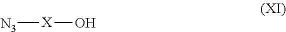

wherein is (CH₂)ₙ wherein n is selected from 1 to 15.

In one embodiment this alkyne group capping agent is 3-azido-1-propanol.

Therefore, in an embodiment, following step (c) the process further comprises a step of capping the unreacted alkyne groups remained in the conjugates with an alkyne group capping agent.

In an embodiment the capping of the unreacted alkyne groups is performed with an amount of capping agent that is between 0.05 to 20 molar equivalents to the amount of polysaccharide repeat unit of the activated saccharide.

In an embodiment the capping of the unreacted alkyne groups is performed with an amount of capping agent that is between 0.1 to 15 molar equivalents to the amount of polysaccharide repeat unit of the activated saccharide.

In an embodiment the capping of the unreacted alkyne groups is performed with an amount of capping agent that is between 0.5 to 10 molar equivalents to the amount of polysaccharide repeat unit of the activated saccharide.

In an embodiment the capping of the unreacted alkyne groups is performed with an amount of capping agent that is between 0.5 to 5 molar equivalents to the amount of polysaccharide repeat unit of the activated saccharide.

In an embodiment the capping of the unreacted alkyne groups is performed with an amount of capping agent that is between 0.5 to 2 molar equivalents to the amount of polysaccharide repeat unit of the activated saccharide.

In an embodiment the capping of the unreacted alkyne groups is performed with an amount of capping agent that is between 0.5 to 1 molar equivalents to the amount of polysaccharide repeat unit of the activated saccharide.

In an embodiment the capping of the unreacted alkyne groups is performed with an amount of capping agent that is between 1 to 5 molar equivalents to the amount of polysaccharide repeat unit of the activated saccharide.

In an embodiment the capping of the unreacted alkyne groups is performed with an amount of capping agent that is between 1 to 2 molar equivalents to the amount of polysaccharide repeat unit of the activated saccharide.

In an embodiment the capping of the unreacted alkyne groups is performed with an amount of capping agent that is between 1.5 to 2.5 molar equivalents to the amount of polysaccharide repeat unit of the activated saccharide.

In an embodiment the capping of the unreacted alkyne groups is performed with an amount of capping agent that about 0.5 molar equivalent to the amount of polysaccharide repeat unit of the activated saccharide.

In an embodiment the capping of the unreacted alkyne groups is performed with an amount of capping agent that about 1 molar equivalent to the amount of polysaccharide repeat unit of the activated saccharide.

In an embodiment the capping of the unreacted alkyne groups is performed with an amount of capping agent that about 1.5 molar equivalent to the amount of polysaccharide repeat unit of the activated saccharide.

In an embodiment the capping of the unreacted alkyne groups is performed with an amount of capping agent that about 2 molar equivalents to the amount of polysaccharide repeat unit of the activated saccharide.

In an embodiment the capping of the unreacted alkyne groups is performed with an amount of capping agent that about 2.5 molar equivalents to the amount of polysaccharide repeat unit of the activated saccharide.

In an embodiment the capping of the unreacted alkyne groups is performed with an amount of capping agent that about 5 molar equivalents to the amount of polysaccharide repeat unit of the activated saccharide.

Following conjugation to the carrier protein, the glycoconjugate can be purified (enriched with respect to the amount of saccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration. Therefore, in one embodiment the process for producing the glycoconjugate of the present invention comprises the step of purifying the glycoconjugate after it is produced.

In an aspect, the invention provides a serotype 9N glycoconjugate produced according to any of the methods disclosed herein.

### Alternative Click Chemistry

In an embodiment of the present invention, the serotype 9N glycoconjugate of the present invention is prepared by alternative click chemistry as disclosed e.g. U.S. Provisional App. No. 63/484,228 (filed on Feb. 10, 2023).

Therefore, in an embodiment, the serotype 9N glycoconjugate of the present invention comprises a serotype 9N saccharide covalently conjugated to a carrier protein (CP) through a spacer and having the general formula (XII):

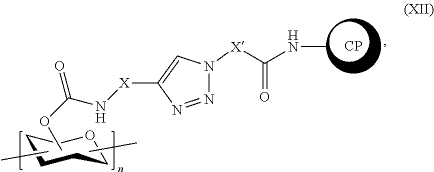

wherein X is selected from the group consisting of CH₂(CH₂)_{n′}, (CH₂CH₂O)ₘCH₂CH₂, NHCO(CH₂)_{n′}, NHCO(CH₂CH₂O)ₘCH₂CH₂, OCH₂(CH₂)_{n′}, and O(CH₂CH₂O)ₘCH₂CH₂; where n′ is selected from 0 to 10 and m is selected from 1 to 4,
and wherein X′ is selected from the group consisting of CH₂(CH₂)_{n″}, CH₂O(CH₂)_{n″}CH₂, CH₂O(CH₂CH₂O)_{m′}(CH₂)_{n″}CH₂, where n″ is selected from 0 to 10 and m′ is selected from 0 to 4, wherein the structure in square backet represents a repeat unit of the serotype 9N saccharide and wherein n represents the number of repeating units.

In a preferred embodiment, the invention is directed to a serotype 9N glycoconjugate comprising a serotype 9N saccharide covalently conjugated to a carrier protein (CP) through a spacer and having the general formula (XII), wherein X is CH₂(CH₂)_{n′}, where n′ is 0 and wherein X′ is CH₂(CH₂)_{n″}, where n″ is 0.

Therefore, in a preferred embodiment, the invention provides a serotype 9N glycoconjugate comprising a serotype 9N saccharide covalently conjugated to a carrier protein (CP) through a spacer and having the general formula (XIII),

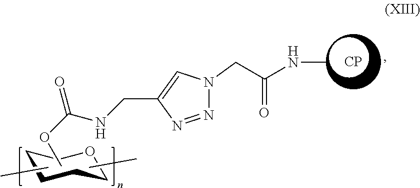

wherein the structure in square backet represents a repeat unit of the serotype 9N saccharide and wherein n represents the number of repeating units.

Formulas (XII) and (XIII) are a schematic representation of glycoconjugates of the invention. It should not be understood that a linkage is present at every repeat unit of the saccharide (the structure in square brackets). Rather, a majority of the saccharide repeating unit remains unmodified and covalent linkages between the carrier protein and the saccharide is for a minority of the saccharide repeat units. Additionally, an individual carrier protein (CP) molecule may be linked to more than one saccharide molecule and an individual saccharide molecule can be linked to more than one individual carrier protein (CP) molecule. The structure in square brackets is a schematic representation of a repeat unit of the serotype 9N saccharide.

In an embodiment, the invention provides a serotype 9N glycoconjugate comprising a serotype 9N saccharide covalently conjugated to a carrier protein (CP) through a spacer and having the general formula (XII), wherein X is CH₂(CH₂)_{n′}, where n′ is selected from 0 to 10 and wherein X′ is CH₂(CH₂)_{n″} where n″ is selected from 0 to 10. In an embodiment, n′ is selected from 0 to 5 and n″ is selected from 0 to 10. In an embodiment, n′ is selected from 0 to 5 and n″ is selected from 0 to 5. In an embodiment, n′ is selected from 0 to 3 and n″ is selected from 0 to 3. In an embodiment, n′ is selected from 0 to 2 and n″ is selected from 0 to 2. In a particular embodiment, n′ is 0 and n″ is 0. In a particular embodiment, n′ is 1 and n″ is 0. In another embodiment, n′ is 2 and n″ is 0. In yet another embodiment, n′ is 3 and n″ is 0. In yet a further embodiment, n′ is 4 and n″ is 0. In yet a further embodiment, n′ is 5 and n″ is 0. In yet a further embodiment, n′ is 6 and n″ is 0. In a particular embodiment, n′ is a and n″ is 1. In a particular embodiment, n′ is 1 and n″ is 1. In another embodiment, n′ is 2 and n″ is 1. In yet another embodiment, n′ is 3 and n″ is 1. In yet a further embodiment, n′ is 4 and n″ is 1. In yet a further embodiment, n′ is 5 and n″ is 1. In yet a further embodiment, n′ is 6 and n″ is 1. In a particular embodiment, n′ is 0 and n″ is 2. In a particular embodiment, n′ is 1 and n″ is 2. In another embodiment, n′ is 2 and n″ is 2. In yet another embodiment, n′ is 3 and n″ is 2. In yet a further embodiment, n′ is 4 and n″ is 2. In yet a further embodiment, n′ is 5 and n″ is 2. In yet a further embodiment, n′ is 6 and n″ is 2. In a particular embodiment, n′ is 0 and n″ is 3. In a particular embodiment, n′ is 1 and n″ is 3. In another embodiment, n′ is 2 and n″ is 3. In yet another embodiment, n′ is 3 and n″ is 3. In yet a further embodiment, n′ is 4 and n″ is 3. In yet a further embodiment, n′ is 5 and n″ is 3. In yet a further embodiment, n′ is 6 and n″ is 3. In a particular embodiment, n′ is 0 and n″ is 4. In a particular embodiment, n′ is 1 and n″ is 4. In another embodiment, n′ is 2 and n″ is 4. In yet another embodiment, n′ is 3 and n″ is 4. In yet a further embodiment, n′ is 4 and n″ is 4. In yet a further embodiment, n′ is 5 and n″ is 4. In yet a further embodiment, n′ is 6 and n″ is 4. In a particular embodiment, n′ is 0 and n″ is 5. In a particular embodiment, n′ is 1 and n″ is 5. In another embodiment, n′ is 2 and n″ is 5. In yet another embodiment, n′ is 3 and n″ is 5. In yet a further embodiment, n′ is 4 and n″ is 5. In yet a further embodiment, n′ is 5 and n″ is 5. In yet a further embodiment, n′ is 6 and n″ is 5. In a particular embodiment, n′ is 0 and n″ is 6. In a particular embodiment, n′ is 1 and n″ is 6. In another embodiment, n′ is 2 and n″ is 6. In yet another embodiment, n′ is 3 and n″ is 6. In yet a further embodiment, n′ is 4 and n″ is 6. In yet a further embodiment, n′ is 5 and n″ is 6. In yet a further embodiment, n′ is 6 and n″ is 6.

In an embodiment, the invention provides a serotype 9N glycoconjugate comprising a serotype 9N saccharide covalently conjugated to a carrier protein (CP) through a spacer and having the general formula (XII), wherein X is CH₂(CH₂)_{n′}, where n′ is selected from 0 to 10 and wherein X′ is CH₂O(CH₂)_{n″}CH₂ where n″ is selected from 0 to 10. In an embodiment, n′ is selected from 0 to 5 and n″ is selected from 0 to 10. In an embodiment, n′ is selected from 0 to 5 and n″ is selected from 0 to 5. In an embodiment, n′ is selected from 0 to 3 and n″ is selected from 0 to 3. In an embodiment, n′ is selected from 0 to 2 and n″ is selected from 0 to 2. In a particular embodiment, n′ is 0 and n″ is 0. In a particular embodiment, n′ is 1 and n″ is 0. In another embodiment, n′ is 2 and n″ is 0. In yet another embodiment, n′ is 3 and n″ is 0. In yet a further embodiment, n′ is 4 and n″ is 0. In yet a further embodiment, n′ is 5 and n″ is 0. In yet a further embodiment, n′ is 6 and n″ is 0. In a particular embodiment, n′ is a and n″ is 1. In a particular embodiment, n′ is 1 and n″ is 1. In another embodiment, n′ is 2 and n″ is 1. In yet another embodiment, n′ is 3 and n″ is 1. In yet a further embodiment, n′ is 4 and n″ is 1. In yet a further embodiment, n′ is 5 and n″ is 1. In yet a further embodiment, n′ is 6 and n″ is 1. In a particular embodiment, n′ is 0 and n″ is 2. In a particular embodiment, n′ is 1 and n″ is 2. In another embodiment, n′ is 2 and n″ is 2. In yet another embodiment, n′ is 3 and n″ is 2. In yet a further embodiment, n′ is 4 and n″ is 2. In yet a further embodiment, n′ is 5 and n″ is 2. In yet a further embodiment, n′ is 6 and n″ is 2. In a particular embodiment, n′ is 0 and n″ is 3. In a particular embodiment, n′ is 1 and n″ is 3. In another embodiment, n′ is 2 and n″ is 3. In yet another embodiment, n′ is 3 and n″ is 3. In yet a further embodiment, n′ is 4 and n″ is 3. In yet a further embodiment, n′ is 5 and n″ is 3. In yet a further embodiment, n′ is 6 and n″ is 3. In a particular embodiment, n′ is 0 and n″ is 4. In a particular embodiment, n′ is 1 and n″ is 4. In another embodiment, n′ is 2 and n″ is 4. In yet another embodiment, n′ is 3 and n″ is 4. In yet a further embodiment, n′ is 4 and n″ is 4. In yet a further embodiment, n′ is 5 and n″ is 4. In yet a further embodiment, n′ is 6 and n″ is 4. In a particular embodiment, n′ is 0 and n″ is 5. In a particular embodiment, n′ is 1 and n″ is 5. In another embodiment, n′ is 2 and n″ is 5. In yet another embodiment, n′ is 3 and n″ is 5. In yet a further embodiment, n′ is 4 and n″ is 5. In yet a further embodiment, n′ is 5 and n″ is 5. In yet a further embodiment, n′ is 6 and n″ is 5. In a particular embodiment, n′ is 0 and n″ is 6. In a particular embodiment, n′ is 1 and n″ is 6. In another embodiment, n′ is 2 and n″ is 6. In yet another embodiment, n′ is 3 and n″ is 6. In yet a further embodiment, n′ is 4 and n″ is 6. In yet a further embodiment, n′ is 5 and n″ is 6. In yet a further embodiment, n′ is 6 and n″ is 6.

In an embodiment, the invention provides a serotype 9N glycoconjugate comprising a serotype 9N saccharide covalently conjugated to a carrier protein (CP) through a spacer and having the general formula (XII), wherein X is CH₂(CH₂)_{n′}, where n′ is selected from 0 to 10 and wherein X′ is CH₂O(CH₂CH₂O)_{m′}(CH₂)_{n″}CH₂, where n″ is selected from 0 to 10 and m′ is selected from 0 to 4.

In an embodiment, n′ is selected from 0 to 5, m′ is selected from 0 to 4 and n″ is selected from 0 to 10. In an embodiment, n′ is selected from 0 to 5, m′ is selected from 0 to 4 and n″ is selected from 0 to 5. In an embodiment, n′ is selected from 0 to 3, m′ is selected from 0 to 2 and n″ is selected from 0 to 3. In an embodiment, n′ is selected from 0 to 2, m′ is selected from 0 to 2 and n″ is selected from 0 to 1. In an embodiment, n′ is selected from 0 to 1, m′ is selected from 0 to 1 and n″ is selected from 0 to 1.

In a particular embodiment, n′ is 0, m′ is 0 and n″ is 0. In another embodiment, n′ is 0, m′ is 1 and n″ is 0. In another embodiment, n′ is 0, m′ is 2 and n″ is 0. In another embodiment, n′ is 1, m′ is 3 and n″ is 0.

In a particular embodiment, n′ is 1, m′ is 0 and n″ is 0. In another embodiment, n′ is 1, m′ is 1 and n″ is 0. In another embodiment, n′ is 1, m′ is 2 and n″ is 0. In another embodiment, n′ is 1, m′ is 3 and n″ is 0.

In another embodiment, n′ is 2, m′ is 0 and n″ is 0. In another embodiment, n′ is 2, m′ is 1 and n″ is 0. In another embodiment, n′ is 2, m′ is 2 and n″ is 0. In another embodiment, n′ is 2, m′ is 3 and n″ is 0.

In yet another embodiment, n′ is 3, m′ is 0 and n″ is 0. In yet another embodiment, n′ is 3, m′ is 1 and n″ is 0. In yet another embodiment, n′ is 3, m′ is 2 and n″ is 0. In yet another embodiment, n′ is 3, m′ is 3 and n″ is 0.

In yet a further embodiment, n′ is 4, m′ is 0 and n″ is 0. In yet a further embodiment, n′ is 4, m′ is 1 and n″ is 0. In yet a further embodiment, n′ is 4, m′ is 2 and n″ is 0. In yet a further embodiment, n′ is 4, m′ is 3 and n″ is 0.

In yet a further embodiment, n′ is 5, m′ is 0 and n″ is 0. In yet a further embodiment, n′ is 5, m′ is 1 and n″ is 0. In yet a further embodiment, n′ is 5, m′ is 2 and n″ is 0. In yet a further embodiment, n′ is 5, m′ is 3 and n″ is 0.

In a particular embodiment, n′ is 0, m′ is 0 and n″ is 1. In a particular embodiment, n′ is 0, m′ is 1 and n″ is 1. In a particular embodiment, n′ is 0, m′ is 2 and n″ is 1. In a particular embodiment, n′ is 0, m′ is 3 and n″ is 1.

In a particular embodiment, n′ is 1, m′ is 0 and n″ is 1. In a particular embodiment, n′ is 1, m′ is 1 and n″ is 1. In a particular embodiment, n′ is 1, m′ is 2 and n″ is 1. In a particular embodiment, n′ is 1, m′ is 3 and n″ is 1.

In another embodiment, n′ is 2, m′ is 0 and n″ is 1. In another embodiment, n′ is 2, m′ is 1 and n″ is 1. In another embodiment, n′ is 2, m′ is 2 and n″ is 1. In another embodiment, n′ is 2, m′ is 3 and n″ is 1.

In yet another embodiment, n′ is 3, m′ is 0 and n″ is 1. In yet another embodiment, n′ is 3, m′ is 1 and n″ is 1. In yet another embodiment, n′ is 3, m′ is 2 and n″ is 1. In yet another embodiment, n′ is 3, m′ is 3 and n″ is 1.

In yet a further embodiment, n′ is 4, m′ is 0 and n″ is 1. In yet a further embodiment, n′ is 4, m′ is 1 and n″ is 1. In yet a further embodiment, n′ is 4, m′ is 2 and n″ is 1. In yet a further embodiment, n′ is 4, m′ is 3 and n″ is 1.

In yet a further embodiment, n′ is 5, m′ is 0 and n″ is 1. In yet a further embodiment, n′ is 5, m′ is 1 and n″ is 1. In yet a further embodiment, n′ is 5, m′ is 2 and n″ is 1. In yet a further embodiment, n′ is 5, m′ is 3 and n″ is 1.

In a particular embodiment, n′ is 0, m′ is 0 and n″ is 2. In a particular embodiment, n′ is 0, m′ is 1 and n″ is 2. In a particular embodiment, n′ is 0, m′ is 2 and n″ is 2. In a particular embodiment, n′ is 0, m′ is 3 and n″ is 2.

In a particular embodiment, n′ is 1, m′ is 0 and n″ is 2. In a particular embodiment, n′ is 1, m′ is 1 and n″ is 2. In a particular embodiment, n′ is 1, m′ is 2 and n″ is 2. In a particular embodiment, n′ is 1, m′ is 3 and n″ is 2.

In another embodiment, n′ is 2, m′ is 0 and n″ is 2. In another embodiment, n′ is 2, m′ is 1 and n″ is 2. In another embodiment, n′ is 2, m′ is 2 and n″ is 2. In another embodiment, n′ is 2, m′ is 3 and n″ is 2.

In yet another embodiment, n′ is 3, m′ is 0 and n″ is 2. In yet another embodiment, n′ is 3, m′ is 1 and n″ is 2. In yet another embodiment, n′ is 3, m′ is 2 and n″ is 2. In yet another embodiment, n′ is 3, m′ is 3 and n″ is 2.

In yet a further embodiment, n′ is 4, m′ is 0 and n″ is 2. In yet a further embodiment, n′ is 4, m′ is 1 and n″ is 2. In yet a further embodiment, n′ is 4, m′ is 2 and n″ is 2. In yet a further embodiment, n′ is 4, m′ is 3 and n″ is 2.

In yet a further embodiment, n′ is 5, m′ is 0 and n″ is 2. In yet a further embodiment, n′ is 5, m′ is 1 and n″ is 2. In yet a further embodiment, n′ is 5, m′ is 2 and n″ is 2. In yet a further embodiment, n′ is 5, m′ is 3 and n″ is 2.

In a particular embodiment, n′ is 0, m′ is 0 and n″ is 3. In a particular embodiment, n′ is 0, m′ is 1 and n″ is 3. In a particular embodiment, n′ is 0, m′ is 2 and n″ is 3. In a particular embodiment, n′ is 0, m′ is 3 and n″ is 3.

In a particular embodiment, n′ is 1, m′ is 0 and n″ is 3. In a particular embodiment, n′ is 1, m′ is 1 and n″ is 3. In a particular embodiment, n′ is 1, m′ is 2 and n″ is 3. In a particular embodiment, n′ is 1, m′ is 3 and n″ is 3.

In another embodiment, n′ is 2, m′ is 0 and n″ is 3. In another embodiment, n′ is 2, m′ is 1 and n″ is 3. In another embodiment, n′ is 2, m′ is 2 and n″ is 3. In another embodiment, n′ is 2, m′ is 3 and n″ is 3.

In yet another embodiment, n′ is 3, m′ is 0 and n″ is 3. In yet another embodiment, n′ is 3, m′ is 1 and n″ is 3. In yet another embodiment, n′ is 3, m′ is 2 and n″ is 3. In yet another embodiment, n′ is 3, m′ is 3 and n″ is 3.

In yet a further embodiment, n′ is 4, m′ is 0 and n″ is 3. In yet a further embodiment, n′ is 4, m′ is 1 and n″ is 3. In yet a further embodiment, n′ is 4, m′ is 2 and n″ is 3. In yet a further embodiment, n′ is 4, m′ is 3 and n″ is 3.

In yet a further embodiment, n′ is 5, m′ is 0 and n″ is 3. In yet a further embodiment, n′ is 5, m′ is 1 and n″ is 3. In yet a further embodiment, n′ is 5, m′ is 2 and n″ is 3. In yet a further embodiment, n′ is 5, m′ is 3 and n″ is 3.

In a particular embodiment, n′ is 0, m′ is 0 and n″ is 4. In a particular embodiment, n′ is 0, m′ is 1 and n″ is 4. In a particular embodiment, n′ is 0, m′ is 2 and n″ is 4. In a particular embodiment, n′ is 0, m′ is 3 and n″ is 4.

In a particular embodiment, n′ is 1, m′ is 0 and n″ is 4. In a particular embodiment, n′ is 1, m′ is 1 and n″ is 4. In a particular embodiment, n′ is 1, m′ is 2 and n″ is 4. In a particular embodiment, n′ is 1, m′ is 3 and n″ is 4.

In another embodiment, n′ is 2, m′ is 0 and n″ is 4. In another embodiment, n′ is 2, m′ is 1 and n″ is 4. In another embodiment, n′ is 2, m′ is 2 and n″ is 4. In another embodiment, n′ is 2, m′ is 3 and n″ is 4.

In yet another embodiment, n′ is 3, m′ is 0 and n″ is 4. In yet another embodiment, n′ is 3, m′ is 1 and n″ is 4. In yet another embodiment, n′ is 3, m′ is 2 and n″ is 4. In yet another embodiment, n′ is 3, m′ is 3 and n″ is 4.

In yet a further embodiment, n′ is 4, m′ is 0 and n″ is 4. In yet a further embodiment, n′ is 4, m′ is 1 and n″ is 4. In yet a further embodiment, n′ is 4, m′ is 2 and n″ is 4. In yet a further embodiment, n′ is 4, m′ is 3 and n″ is 4.

In yet a further embodiment, n′ is 5, m′ is 0 and n″ is 4. In yet a further embodiment, n′ is 5, m′ is 1 and n″ is 4. In yet a further embodiment, n′ is 5, m′ is 2 and n″ is 4. In yet a further embodiment, n′ is 5, m′ is 3 and n″ is 4.

In a particular embodiment, n′ is 0, m′ is 0 and n″ is 5. In a particular embodiment, n′ is 0, m′ is 1 and n″ is 5. In a particular embodiment, n′ is 0, m′ is 2 and n″ is 5. In a particular embodiment, n′ is 0, m′ is 3 and n″ is 5.

In a particular embodiment, n′ is 1, m′ is 0 and n″ is 5. In a particular embodiment, n′ is 1, m′ is 1 and n″ is 5. In a particular embodiment, n′ is 1, m′ is 2 and n″ is 5. In a particular embodiment, n′ is 1, m′ is 3 and n″ is 5.

In another embodiment, n′ is 2, m′ is 0 and n″ is 5. In another embodiment, n′ is 2, m′ is 1 and n″ is 5. In another embodiment, n′ is 2, m′ is 2 and n″ is 5. In another embodiment, n′ is 2, m′ is 3 and n″ is 5.

In yet another embodiment, n′ is 3, m′ is 0 and n″ is 5. In yet another embodiment, n′ is 3, m′ is 1 and n″ is 5. In yet another embodiment, n′ is 3, m′ is 2 and n″ is 5. In yet another embodiment, n′ is 3, m′ is 3 and n″ is 5.

In yet a further embodiment, n′ is 4, m′ is 0 and n″ is 5. In yet a further embodiment, n′ is 4, m′ is 1 and n″ is 5. In yet a further embodiment, n′ is 4, m′ is 2 and n″ is 5. In yet a further embodiment, n′ is 4, m′ is 3 and n″ is 5.

In yet a further embodiment, n′ is 5, m′ is 0 and n″ is 5. In yet a further embodiment, n′ is 5, m′ is 1 and n″ is 5. In yet a further embodiment, n′ is 5, m′ is 2 and n″ is 5. In yet a further embodiment, n′ is 5, m′ is 3 and n″ is 5.

In an embodiment, the invention provides a serotype 9N glycoconjugate comprising a serotype 9N saccharide covalently conjugated to a carrier protein (CP) through a spacer and having the general formula (XII), wherein X is (CH₂CH₂O)ₘCH₂CH₂, where m is selected from 1 to 4 and wherein X′ is CH₂(CH₂)_{n″}, where n″ is selected from 0 to 10. In an embodiment, m is selected from 1 to 3 and n″ is selected from 0 to 10. In an embodiment, m is selected from 1 to 3 and n″ is selected from 0 to 5. In an embodiment, m is selected from 1 to 2 and n″ is selected from 0 to 3. In an embodiment, m is selected from 1 to 2 and n″ is selected from 0 to 2. In a particular embodiment, m is 1 and n″ is 0. In another embodiment, m is 2 and n″ is 0. In yet another embodiment, m is 3 and n″ is 0. In yet a further embodiment, m is 4 and n″ is 0. In a particular embodiment, m is 1 and n″ is 1. In another embodiment, m is 2 and n″ is 1. In yet another embodiment, m is 3 and n″ is 1. In yet a further embodiment, m is 4 and n″ is 1. In a particular embodiment, m is 1 and n″ is 2. In another embodiment, m is 2 and n″ is 2. In yet another embodiment, m is 3 and n″ is 2. In yet a further embodiment, m is 4 and n″ is 2. In a particular embodiment, m is 1 and n″ is 3. In another embodiment, m is 2 and n″ is 3. In yet another embodiment, m is 3 and n″ is 3. In yet a further embodiment, m is 4 and n″ is 3. In a particular embodiment, m is 1 and n″ is 4. In another embodiment, m is 2 and n″ is 4. In yet another embodiment, m is 3 and n″ is 4. In yet a further embodiment, m is 4 and n″ is 4. In a particular embodiment, m is 1 and n″ is 5. In another embodiment, m is 2 and n″ is 5. In yet another embodiment, m is 3 and n″ is 5. In yet a further embodiment, m is 4 and n″ is 5. In a particular embodiment, m is 1 and n″ is 6. In another embodiment, m is 2 and n″ is 6. In yet another embodiment, m is 3 and n″ is 6. In yet a further embodiment, m is 4 and n″ is 6.

In an embodiment, the invention provides a serotype 9N glycoconjugate comprising a serotype 9N saccharide covalently conjugated to a carrier protein (CP) through a spacer and having the general formula (XII), wherein X is (CH₂CH₂O)ₘCH₂CH₂, where m is selected from 1 to 4 and wherein X′ is CH₂O(CH₂)_{n″}CH₂, where n″ is selected from 0 to 10. In an embodiment, m is selected from 1 to 3 and n″ is selected from 0 to 10. In an embodiment, m is selected from 1 to 3 and n″ is selected from 0 to 5. In an embodiment, m is selected from 1 to 2 and n″ is selected from 0 to 3. In an embodiment, m is selected from 1 to 2 and n″ is selected from 0 to 2. In a particular embodiment, m is 1 and n″ is 0. In another embodiment, m is 2 and n″ is 0. In yet another embodiment, m is 3 and n″ is 0. In yet a further embodiment, m is 4 and n″ is 0. In a particular embodiment, m is 1 and n″ is 1. In another embodiment, m is 2 and n″ is 1. In yet another embodiment, m is 3 and n″ is 1. In yet a further embodiment, m is 4 and n″ is 1. In a particular embodiment, m is 1 and n″ is 2. In another embodiment, m is 2 and n″ is 2. In yet another embodiment, m is 3 and n″ is 2. In yet a further embodiment, m is 4 and n″ is 2. In a particular embodiment, m is 1 and n″ is 3. In another embodiment, m is 2 and n″ is 3. In yet another embodiment, m is 3 and n″ is 3. In yet a further embodiment, m is 4 and n″ is 3. In a particular embodiment, m is 1 and n″ is 4. In another embodiment, m is 2 and n″ is 4. In yet another embodiment, m is 3 and n″ is 4. In yet a further embodiment, m is 4 and n″ is 4. In a particular embodiment, m is 1 and n″ is 5. In another embodiment, m is 2 and n″ is 5. In yet another embodiment, m is 3 and n″ is 5. In yet a further embodiment, m is 4 and n″ is 5. In a particular embodiment, m is 1 and n″ is 6. In another embodiment, m is 2 and n″ is 6. In yet another embodiment, m is 3 and n″ is 6. In yet a further embodiment, m is 4 and n″ is 6.

In an embodiment, the invention provides a serotype 9N glycoconjugate comprising a serotype 9N saccharide covalently conjugated to a carrier protein (CP) through a spacer and having the general formula (XII), wherein X is (CH₂CH₂O)ₘCH₂CH₂, where m is selected from 1 to 4 and wherein X′ is CH₂O(CH₂CH₂O)_{m′}(CH₂)_{n″}CH₂, where n″ is selected from 0 to 10 and m′ is selected from 0 to 4.

In an embodiment, m is selected from 1 to 3, m′ is selected from 0 to 4 and n″ is selected from 0 to 10. In an embodiment, m is selected from 1 to 2, m′ is selected from 0 to 4 and n″ is selected from 0 to 5. In an embodiment, m is selected from 1 to 2, m′ is selected from 0 to 2 and n″ is selected from 0 to 3. In an embodiment, m is selected from 1 to 2, m′ is selected from 0 to 2 and n″ is selected from 0 to 1.

In a particular embodiment, m is 1, m′ is 0 and n″ is 0. In another embodiment, m is 1, m′ is 1 and n″ is 0. In another embodiment, m is 1, m′ is 2 and n″ is 0. In another embodiment, m is 1, m′ is 3 and n″ is 0.

In another embodiment, m is 2, m′ is 0 and n″ is 0. In another embodiment, m is 2, m′ is 1 and n″ is 0. In another embodiment, m is 2, m′ is 2 and n″ is 0. In another embodiment, m is 2, m′ is 3 and n″ is 0.

In yet another embodiment, m is 3, m′ is 0 and n″ is 0. In yet another embodiment, m is 3, m′ is 1 and n″ is 0. In yet another embodiment, m is 3, m′ is 2 and n″ is 0. In yet another embodiment, m is 3, m′ is 3 and n″ is 0.

In yet a further embodiment, m is 4, m′ is 0 and n″ is 0. In yet a further embodiment, m is 4, m′ is 1 and n″ is 0. In yet a further embodiment, m is 4, m′ is 2 and n″ is 0. In yet a further embodiment, m is 4, m′ is 3 and n″ is 0.

In a particular embodiment, m is 1, m′ is 0 and n″ is 1. In a particular embodiment, m is 1, m′ is 1 and n″ is 1. In a particular embodiment, m is 1, m′ is 2 and n″ is 1. In a particular embodiment, m is 1, m′ is 3 and n″ is 1.

In another embodiment, m is 2, m′ is 0 and n″ is 1. In another embodiment, m is 2, m′ is 1 and n″ is 1. In another embodiment, m is 2, m′ is 2 and n″ is 1. In another embodiment, m is 2, m′ is 3 and n″ is 1.

In yet another embodiment, m is 3, m′ is 0 and n″ is 1. In yet another embodiment, m is 3, m′ is 1 and n″ is 1. In yet another embodiment, m is 3, m′ is 2 and n″ is 1. In yet another embodiment, m is 3, m′ is 3 and n″ is 1.

In yet a further embodiment, m is 4, m′ is 0 and n″ is 1. In yet a further embodiment, m is 4, m′ is 1 and n″ is 1. In yet a further embodiment, m is 4, m′ is 2 and n″ is 1. In yet a further embodiment, m is 4, m′ is 3 and n″ is 1.

In a particular embodiment, m is 1, m′ is 0 and n″ is 2. In a particular embodiment, m is 1, m′ is 1 and n″ is 2. In a particular embodiment, m is 1, m′ is 2 and n″ is 2. In a particular embodiment, m is 1, m′ is 3 and n″ is 2.

In another embodiment, m is 2, m′ is 0 and n″ is 2. In another embodiment, m is 2, m′ is 1 and n″ is 2. In another embodiment, m is 2, m′ is 2 and n″ is 2. In another embodiment, m is 2, m′ is 3 and n″ is 2.

In yet another embodiment, m is 3, m′ is 0 and n″ is 2. In yet another embodiment, m is 3, m′ is 1 and n″ is 2. In yet another embodiment, m is 3, m′ is 2 and n″ is 2. In yet another embodiment, m is 3, m′ is 3 and n″ is 2.

In yet a further embodiment, m is 4, m′ is 0 and n″ is 2. In yet a further embodiment, m is 4, m′ is 1 and n″ is 2. In yet a further embodiment, m is 4, m′ is 2 and n″ is 2. In yet a further embodiment, m is 4, m′ is 3 and n″ is 2.

In a particular embodiment, m is 1, m′ is 0 and n″ is 3. In a particular embodiment, m is 1, m′ is 1 and n″ is 3. In a particular embodiment, m is 1, m′ is 2 and n″ is 3. In a particular embodiment, m is 1, m′ is 3 and n″ is 3.

In another embodiment, m is 2, m′ is 0 and n″ is 3. In another embodiment, m is 2, m′ is 1 and n″ is 3. In another embodiment, m is 2, m′ is 2 and n″ is 3. In another embodiment, m is 2, m′ is 3 and n″ is 3.

In yet another embodiment, m is 3, m′ is 0 and n″ is 3. In yet another embodiment, m is 3, m′ is 1 and n″ is 3. In yet another embodiment, m is 3, m′ is 2 and n″ is 3. In yet another embodiment, m is 3, m′ is 3 and n″ is 3.

In yet a further embodiment, m is 4, m′ is 0 and n″ is 3. In yet a further embodiment, m is 4, m′ is 1 and n″ is 3. In yet a further embodiment, m is 4, m′ is 2 and n″ is 3. In yet a further embodiment, m is 4, m′ is 3 and n″ is 3.

In a particular embodiment, m is 1, m′ is 0 and n″ is 4. In a particular embodiment, m is 1, m′ is 1 and n″ is 4. In a particular embodiment, m is 1, m′ is 2 and n″ is 4. In a particular embodiment, m is 1, m′ is 3 and n″ is 4.

In another embodiment, m is 2, m′ is 0 and n″ is 4. In another embodiment, m is 2, m′ is 1 and n″ is 4. In another embodiment, m is 2, m′ is 2 and n″ is 4. In another embodiment, m is 2, m′ is 3 and n″ is 4.

In yet another embodiment, m is 3, m′ is 0 and n″ is 4. In yet another embodiment, m is 3, m′ is 1 and n″ is 4. In yet another embodiment, m is 3, m′ is 2 and n″ is 4. In yet another embodiment, m is 3, m′ is 3 and n″ is 4.

In yet a further embodiment, m is 4, m′ is 0 and n″ is 4. In yet a further embodiment, m is 4, m′ is 1 and n″ is 4. In yet a further embodiment, m is 4, m′ is 2 and n″ is 4. In yet a further embodiment, m is 4, m′ is 3 and n″ is 4.

In a particular embodiment, m is 1, m′ is 0 and n″ is 5. In a particular embodiment, m is 1, m′ is 1 and n″ is 5. In a particular embodiment, m is 1, m′ is 2 and n″ is 5. In a particular embodiment, m is 1, m′ is 3 and n″ is 5.

In another embodiment, m is 2, m′ is 0 and n″ is 5. In another embodiment, m is 2, m′ is 1 and n″ is 5. In another embodiment, m is 2, m′ is 2 and n″ is 5. In another embodiment, m is 2, m′ is 3 and n″ is 5.

In yet another embodiment, m is 3, m′ is 0 and n″ is 5. In yet another embodiment, m is 3, m′ is 1 and n″ is 5. In yet another embodiment, m is 3, m′ is 2 and n″ is 5. In yet another embodiment, m is 3, m′ is 3 and n″ is 5.

In yet a further embodiment, m is 4, m′ is 0 and n″ is 5. In yet a further embodiment, m is 4, m′ is 1 and n″ is 5. In yet a further embodiment, m is 4, m′ is 2 and n″ is 5. In yet a further embodiment, m is 4, m′ is 3 and n″ is 5.

In an embodiment, the invention provides a serotype 9N glycoconjugate comprising a serotype 9N saccharide covalently conjugated to a carrier protein (CP) through a spacer and having the general formula (XII), wherein X is NHCO(CH₂)_{n′}, where n′ is selected from 1 to 10 and wherein X is CH₂(CH₂)_{n″}, where n″ is selected from 0 to 10. In an embodiment, n′ is selected from 1 to 5 and n″ is selected from 0 to 10. In an embodiment, n′ is selected from 1 to 5 and n″ is selected from 0 to 5. In an embodiment, n′ is selected from 1 to 3 and n″ is selected from 0 to 3. In an embodiment, n′ is selected from 1 to 2 and n″ is selected from 0 to 2. In a particular embodiment, n′ is 1 and n″ is 0. In another embodiment, n′ is 2 and n″ is 0. In yet another embodiment, n′ is 3 and n″ is 0. In yet a further embodiment, n′ is 4 and n″ is 0. In yet a further embodiment, n′ is 5 and n″ is 0. In yet a further embodiment, n′ is 6 and n″ is 0. In a particular embodiment, n′ is 1 and n″ is 1. In another embodiment, n′ is 2 and n″ is 1. In yet another embodiment, n′ is 3 and n″ is 1. In yet a further embodiment, n′ is 4 and n″ is 1. In yet a further embodiment, n′ is 5 and n″ is 1. In yet a further embodiment, n′ is 6 and n″ is 1. In a particular embodiment, n′ is 1 and n″ is 2. In another embodiment, n′ is 2 and n″ is 2. In yet another embodiment, n′ is 3 and n″ is 2. In yet a further embodiment, n′ is 4 and n″ is 2. In yet a further embodiment, n′ is 5 and n″ is 2. In yet a further embodiment, n′ is 6 and n″ is 2. In a particular embodiment, n′ is 1 and n″ is 3. In another embodiment, n′ is 2 and n″ is 3. In yet another embodiment, n′ is 3 and n″ is 3. In yet a further embodiment, n′ is 4 and n″ is 3. In yet a further embodiment, n′ is 5 and n″ is 3. In yet a further embodiment, n′ is 6 and n″ is 3. In a particular embodiment, n′ is 1 and n″ is 4. In another embodiment, n′ is 2 and n″ is 4. In yet another embodiment, n′ is 3 and n″ is 4. In yet a further embodiment, n′ is 4 and n″ is 4. In yet a further embodiment, n′ is 5 and n″ is 4. In yet a further embodiment, n′ is 6 and n″ is 4. In a particular embodiment, n′ is 1 and n″ is 5. In another embodiment, n′ is 2 and n″ is 5. In yet another embodiment, n′ is 3 and n″ is 5. In yet a further embodiment, n′ is 4 and n″ is 5. In yet a further embodiment, n′ is 5 and n″ is 5. In yet a further embodiment, n′ is 6 and n″ is 5. In a particular embodiment, n′ is 1 and n″ is 6. In another embodiment, n′ is 2 and n″ is 6. In yet another embodiment, n′ is 3 and n″ is 6. In yet a further embodiment, n′ is 4 and n″ is 6. In yet a further embodiment, n′ is 5 and n″ is 6. In yet a further embodiment, n′ is 6 and n″ is 6.

In an embodiment, the invention provides a serotype 9N glycoconjugate comprising a serotype 9N saccharide covalently conjugated to a carrier protein (CP) through a spacer and having the general formula (VII), wherein X is NHCO(CH₂)_{n′}, where n′ is selected from 1 to 10 and wherein X′ is CH₂O(CH₂)_{n″}CH₂, where n″ is selected from 0 to 10. In an embodiment, n′ is selected from 1 to 5 and n″ is selected from 0 to 10. In an embodiment, n′ is selected from 1 to 5 and n″ is selected from 0 to 5. In an embodiment, n′ is selected from 1 to 3 and n″ is selected from 0 to 3. In an embodiment, n′ is selected from 1 to 2 and n″ is selected from 0 to 2. In a particular embodiment, n′ is 1 and n″ is 0. In another embodiment, n′ is 2 and n″ is 0. In yet another embodiment, n′ is 3 and n″ is 0. In yet a further embodiment, n′ is 4 and n″ is 0. In yet a further embodiment, n′ is 5 and n″ is 0. In yet a further embodiment, n′ is 6 and n″ is 0. In a particular embodiment, n′ is 1 and n″ is 1. In another embodiment, n′ is 2 and n″ is 1. In yet another embodiment, n′ is 3 and n″ is 1. In yet a further embodiment, n′ is 4 and n″ is 1. In yet a further embodiment, n′ is 5 and n″ is 1. In yet a further embodiment, n′ is 6 and n″ is 1. In a particular embodiment, n′ is 1 and n″ is 2. In another embodiment, n′ is 2 and n″ is 2. In yet another embodiment, n′ is 3 and n″ is 2. In yet a further embodiment, n′ is 4 and n″ is 2. In yet a further embodiment, n′ is 5 and n″ is 2. In yet a further embodiment, n′ is 6 and n″ is 2. In a particular embodiment, n′ is 1 and n″ is 3. In another embodiment, n′ is 2 and n″ is 3. In yet another embodiment, n′ is 3 and n″ is 3. In yet a further embodiment, n′ is 4 and n″ is 3. In yet a further embodiment, n′ is 5 and n″ is 3. In yet a further embodiment, n′ is 6 and n″ is 3. In a particular embodiment, n′ is 1 and n″ is 4. In another embodiment, n′ is 2 and n″ is 4. In yet another embodiment, n′ is 3 and n″ is 4. In yet a further embodiment, n′ is 4 and n″ is 4. In yet a further embodiment, n′ is 5 and n″ is 4. In yet a further embodiment, n′ is 6 and n″ is 4. In a particular embodiment, n′ is 1 and n″ is 5. In another embodiment, n′ is 2 and n″ is 5. In yet another embodiment, n′ is 3 and n″ is 5. In yet a further embodiment, n′ is 4 and n″ is 5. In yet a further embodiment, n′ is 5 and n″ is 5. In yet a further embodiment, n′ is 6 and n″ is 5. In a particular embodiment, n′ is 1 and n″ is 6. In another embodiment, n′ is 2 and n″ is 6. In yet another embodiment, n′ is 3 and n″ is 6. In yet a further embodiment, n′ is 4 and n″ is 6. In yet a further embodiment, n′ is 5 and n″ is 6. In yet a further embodiment, n′ is 6 and n″ is 6.

In an embodiment, the invention provides a serotype 9N glycoconjugate comprising a serotype 9N saccharide covalently conjugated to a carrier protein (CP) through a spacer and having the general formula (XII), wherein X is NHCO(CH₂)_{n′}, where n′ is selected from 1 to 10 and wherein X′ is CH₂O(CH₂CH₂O)_{m′}(CH₂)_{n″}CH₂, where n″ is selected from 0 to 10 and m′ is selected from 0 to 4.

In an embodiment, n′ is selected from 1 to 5, m′ is selected from 0 to 4 and n″ is selected from 0 to 10. In an embodiment, n′ is selected from 1 to 5, m′ is selected from 0 to 4 and n″ is selected from 0 to 5. In an embodiment, n′ is selected from 1 to 3, m′ is selected from 0 to 2 and n″ is selected from 0 to 3. In an embodiment, n′ is selected from 1 to 2, m′ is selected from 0 to 2 and n″ is selected from 0 to 1.

In a particular embodiment, n′ is 1, m′ is 0 and n″ is 0. In another embodiment, n′ is 1, m′ is 1 and n″ is 0. In another embodiment, n′ is 1, m′ is 2 and n″ is 0. In another embodiment, n′ is 1, m′ is 3 and n″ is 0.

In another embodiment, n′ is 2, m′ is 0 and n″ is 0. In another embodiment, n′ is 2, m′ is 1 and n″ is 0. In another embodiment, n′ is 2, m′ is 2 and n″ is 0. In another embodiment, n′ is 2, m′ is 3 and n″ is 0.

In yet another embodiment, n′ is 3, m′ is 0 and n″ is 0. In yet another embodiment, n′ is 3, m′ is 1 and n″ is 0. In yet another embodiment, n′ is 3, m′ is 2 and n″ is 0. In yet another embodiment, n′ is 3, m′ is 3 and n″ is 0.

In yet a further embodiment, n′ is 4, m′ is 0 and n″ is 0. In yet a further embodiment, n′ is 4, m′ is 1 and n″ is 0. In yet a further embodiment, n′ is 4, m′ is 2 and n″ is 0. In yet a further embodiment, n′ is 4, m′ is 3 and n″ is 0.

In yet a further embodiment, n′ is 5, m′ is 0 and n″ is 0. In yet a further embodiment, n′ is 5, m′ is 1 and n″ is 0. In yet a further embodiment, n′ is 5, m′ is 2 and n″ is 0. In yet a further embodiment, n′ is 5, m′ is 3 and n″ is 0.

In a particular embodiment, n′ is 1, m′ is 0 and n″ is 1. In a particular embodiment, n′ is 1, m′ is 1 and n″ is 1. In a particular embodiment, n′ is 1, m′ is 2 and n″ is 1. In a particular embodiment, n′ is 1, m′ is 3 and n″ is 1.

In another embodiment, n′ is 2, m′ is 0 and n″ is 1. In another embodiment, n′ is 2, m′ is 1 and n″ is 1. In another embodiment, n′ is 2, m′ is 2 and n″ is 1. In another embodiment, n′ is 2, m′ is 3 and n″ is 1.

In yet another embodiment, n′ is 3, m′ is 0 and n″ is 1. In yet another embodiment, n′ is 3, m′ is 1 and n″ is 1. In yet another embodiment, n′ is 3, m′ is 2 and n″ is 1. In yet another embodiment, n′ is 3, m′ is 3 and n″ is 1.

In yet a further embodiment, n′ is 4, m′ is 0 and n″ is 1. In yet a further embodiment, n′ is 4, m′ is 1 and n″ is 1. In yet a further embodiment, n′ is 4, m′ is 2 and n″ is 1. In yet a further embodiment, n′ is 4, m′ is 3 and n″ is 1.

In yet a further embodiment, n′ is 5, m′ is 0 and n″ is 1. In yet a further embodiment, n′ is 5, m′ is 1 and n″ is 1. In yet a further embodiment, n′ is 5, m′ is 2 and n″ is 1. In yet a further embodiment, n′ is 5, m′ is 3 and n″ is 1.

In a particular embodiment, n′ is 1, m′ is 0 and n″ is 2. In a particular embodiment, n′ is 1, m′ is 1 and n″ is 2. In a particular embodiment, n′ is 1, m′ is 2 and n″ is 2. In a particular embodiment, n′ is 1, m′ is 3 and n″ is 2.

In another embodiment, n′ is 2, m′ is 0 and n″ is 2. In another embodiment, n′ is 2, m′ is 1 and n″ is 2. In another embodiment, n′ is 2, m′ is 2 and n″ is 2. In another embodiment, n′ is 2, m′ is 3 and n″ is 2.

In yet another embodiment, n′ is 3, m′ is 0 and n″ is 2. In yet another embodiment, n′ is 3, m′ is 1 and n″ is 2. In yet another embodiment, n′ is 3, m′ is 2 and n″ is 2. In yet another embodiment, n′ is 3, m′ is 3 and n″ is 2.

In yet a further embodiment, n′ is 4, m′ is 0 and n″ is 2. In yet a further embodiment, n′ is 4, m′ is 1 and n″ is 2. In yet a further embodiment, n′ is 4, m′ is 2 and n″ is 2. In yet a further embodiment, n′ is 4, m′ is 3 and n″ is 2.

In yet a further embodiment, n′ is 5, m′ is 0 and n″ is 2. In yet a further embodiment, n′ is 5, m′ is 1 and n″ is 2. In yet a further embodiment, n′ is 5, m′ is 2 and n″ is 2. In yet a further embodiment, n′ is 5, m′ is 3 and n″ is 2.

In a particular embodiment, n′ is 1, m′ is 0 and n″ is 3. In a particular embodiment, n′ is 1, m′ is 1 and n″ is 3. In a particular embodiment, n′ is 1, m′ is 2 and n″ is 3. In a particular embodiment, n′ is 1, m′ is 3 and n″ is 3.

In another embodiment, n′ is 2, m′ is 0 and n″ is 3. In another embodiment, n′ is 2, m′ is 1 and n″ is 3. In another embodiment, n′ is 2, m′ is 2 and n″ is 3. In another embodiment, n′ is 2, m′ is 3 and n″ is 3.

In yet another embodiment, n′ is 3, m′ is 0 and n″ is 3. In yet another embodiment, n′ is 3, m′ is 1 and n″ is 3. In yet another embodiment, n′ is 3, m′ is 2 and n″ is 3. In yet another embodiment, n′ is 3, m′ is 3 and n″ is 3.

In yet a further embodiment, n′ is 4, m′ is 0 and n″ is 3. In yet a further embodiment, n′ is 4, m′ is 1 and n″ is 3. In yet a further embodiment, n′ is 4, m′ is 2 and n″ is 3. In yet a further embodiment, n′ is 4, m′ is 3 and n″ is 3.

In yet a further embodiment, n′ is 5, m′ is 0 and n″ is 3. In yet a further embodiment, n′ is 5, m′ is 1 and n″ is 3. In yet a further embodiment, n′ is 5, m′ is 2 and n″ is 3. In yet a further embodiment, n′ is 5, m′ is 3 and n″ is 3.

In a particular embodiment, n′ is 1, m′ is 0 and n″ is 4. In a particular embodiment, n′ is 1, m′ is 1 and n″ is 4. In a particular embodiment, n′ is 1, m′ is 2 and n″ is 4. In a particular embodiment, n′ is 1, m′ is 3 and n″ is 4.

In another embodiment, n′ is 2, m′ is 0 and n″ is 4. In another embodiment, n′ is 2, m′ is 1 and n″ is 4. In another embodiment, n′ is 2, m′ is 2 and n″ is 4. In another embodiment, n′ is 2, m′ is 3 and n″ is 4.

In yet another embodiment, n′ is 3, m′ is 0 and n″ is 4. In yet another embodiment, n′ is 3, m′ is 1 and n″ is 4. In yet another embodiment, n′ is 3, m′ is 2 and n″ is 4. In yet another embodiment, n′ is 3, m′ is 3 and n″ is 4.

In yet a further embodiment, n′ is 4, m′ is 0 and n″ is 4. In yet a further embodiment, n′ is 4, m′ is 1 and n″ is 4. In yet a further embodiment, n′ is 4, m′ is 2 and n″ is 4. In yet a further embodiment, n′ is 4, m′ is 3 and n″ is 4.

In yet a further embodiment, n′ is 5, m′ is 0 and n″ is 4. In yet a further embodiment, n′ is 5, m′ is 1 and n″ is 4. In yet a further embodiment, n′ is 5, m′ is 2 and n″ is 4. In yet a further embodiment, n′ is 5, m′ is 3 and n″ is 4.

In a particular embodiment, n′ is 1, m′ is 0 and n″ is 5. In a particular embodiment, n′ is 1, m′ is 1 and n″ is 5. In a particular embodiment, n′ is 1, m′ is 2 and n″ is 5. In a particular embodiment, n′ is 1, m′ is 3 and n″ is 5.

In another embodiment, n′ is 2, m′ is 0 and n″ is 5. In another embodiment, n′ is 2, m′ is 1 and n″ is 5. In another embodiment, n′ is 2, m′ is 2 and n″ is 5. In another embodiment, n′ is 2, m′ is 3 and n″ is 5.

In yet another embodiment, n′ is 3, m′ is 0 and n″ is 5. In yet another embodiment, n′ is 3, m′ is 1 and n″ is 5. In yet another embodiment, n′ is 3, m′ is 2 and n″ is 5. In yet another embodiment, n′ is 3, m′ is 3 and n″ is 5.

In yet a further embodiment, n′ is 4, m′ is 0 and n″ is 5. In yet a further embodiment, n′ is 4, m′ is 1 and n″ is 5. In yet a further embodiment, n′ is 4, m′ is 2 and n″ is 5. In yet a further embodiment, n′ is 4, m′ is 3 and n″ is 5.

In yet a further embodiment, n′ is 5, m′ is 0 and n″ is 5. In yet a further embodiment, n′ is 5, m′ is 1 and n″ is 5. In yet a further embodiment, n′ is 5, m′ is 2 and n″ is 5. In yet a further embodiment, n′ is 5, m′ is 3 and n″ is 5.

In an embodiment, the invention provides a serotype 9N glycoconjugate comprising a serotype 9N saccharide covalently conjugated to a carrier protein (CP) through a spacer and having the general formula (XII), wherein X is NHCO(CH₂CH₂O)ₘCH₂CH₂, where m is selected from 1 to 4 and wherein X′ is CH₂(CH₂)_{n″}, where n″ is selected from 0 to 10. In an embodiment, m is selected from 1 to 3 and n″ is selected from 0 to 10. In an embodiment, m is selected from 1 to 3 and n″ is selected from 0 to 5. In an embodiment, m is selected from 1 to 2 and n″ is selected from 0 to 3. In an embodiment, m is selected from 1 to 2 and n″ is selected from 0 to 2. In a particular embodiment, m is 1 and n″ is 0. In another embodiment, m is 2 and n″ is 0. In yet another embodiment, m is 3 and n″ is 0. In yet a further embodiment, m is 4 and n″ is 0. In a particular embodiment, m is 1 and n″ is 1. In another embodiment, m is 2 and n″ is 1. In yet another embodiment, m is 3 and n″ is 1. In yet a further embodiment, m is 4 and n″ is 1. In a particular embodiment, m is 1 and n″ is 2. In another embodiment, m is 2 and n″ is 2. In yet another embodiment, m is 3 and n″ is 2. In yet a further embodiment, m is 4 and n″ is 2. In a particular embodiment, m is 1 and n″ is 3. In another embodiment, m is 2 and n″ is 3. In yet another embodiment, m is 3 and n″ is 3. In yet a further embodiment, m is 4 and n″ is 3. In a particular embodiment, m is 1 and n″ is 4. In another embodiment, m is 2 and n″ is 4. In yet another embodiment, m is 3 and n″ is 4. In yet a further embodiment, m is 4 and n″ is 4. In a particular embodiment, m is 1 and n″ is 5. In another embodiment, m is 2 and n″ is 5. In yet another embodiment, m is 3 and n″ is 5. In yet a further embodiment, m is 4 and n″ is 5. In a particular embodiment, m is 1 and n″ is 6. In another embodiment, m is 2 and n″ is 6. In yet another embodiment, m is 3 and n″ is 6. In yet a further embodiment, m is 4 and n″ is 6.

In an embodiment, the invention provides a serotype 9N glycoconjugate comprising a serotype 9N saccharide covalently conjugated to a carrier protein (CP) through a spacer and having the general formula (XII), wherein X is NHCO(CH₂CH₂O)ₘCH₂CH₂, where m is selected from 1 to 4 and wherein X′ is CH₂O(CH₂)_{n″}CH₂, where n″ is selected from 0 to 10. In an embodiment, m is selected from 1 to 3 and n″ is selected from 0 to 10. In an embodiment, m is selected from 1 to 3 and n″ is selected from 0 to 5. In an embodiment, m is selected from 1 to 2 and n″ is selected from 0 to 3. In an embodiment, m is selected from 1 to 2 and n″ is selected from 0 to 2. In a particular embodiment, m is 1 and n″ is 0. In another embodiment, m is 2 and n″ is 0. In yet another embodiment, m is 3 and n″ is 0. In yet a further embodiment, m is 4 and n″ is 0. In a particular embodiment, m is 1 and n″ is 1. In another embodiment, m is 2 and n″ is 1. In yet another embodiment, m is 3 and n″ is 1. In yet a further embodiment, m is 4 and n″ is 1. In a particular embodiment, m is 1 and n″ is 2. In another embodiment, m is 2 and n″ is 2. In yet another embodiment, m is 3 and n″ is 2. In yet a further embodiment, m is 4 and n″ is 2. In a particular embodiment, m is 1 and n″ is 3. In another embodiment, m is 2 and n″ is 3. In yet another embodiment, m is 3 and n″ is 3. In yet a further embodiment, m is 4 and n″ is 3. In a particular embodiment, m is 1 and n″ is 4. In another embodiment, m is 2 and n″ is 4. In yet another embodiment, m is 3 and n″ is 4. In yet a further embodiment, m is 4 and n″ is 4. In a particular embodiment, m is 1 and n″ is 5. In another embodiment, m is 2 and n″ is 5. In yet another embodiment, m is 3 and n″ is 5. In yet a further embodiment, m is 4 and n″ is 5. In a particular embodiment, m is 1 and n″ is 6. In another embodiment, m is 2 and n″ is 6. In yet another embodiment, m is 3 and n″ is 6. In yet a further embodiment, m is 4 and n″ is 6.

In an embodiment, the invention provides a serotype 9N glycoconjugate comprising a serotype 9N saccharide covalently conjugated to a carrier protein (CP) through a spacer and having the general formula (XII), wherein X is NHCO(CH₂CH₂O)ₘCH₂CH₂, where m is selected from 1 to 4 and wherein X′ is CH₂O(CH₂CH₂O)_{m′}(CH₂)_{n″}CH₂, where n″ is selected from 0 to 10 and m′ is selected from 0 to 4.

In an embodiment, m is selected from 1 to 3, m′ is selected from 0 to 4 and n″ is selected from 0 to 10. In an embodiment, m is selected from 1 to 2, m′ is selected from 0 to 4 and n″ is selected from 0 to 5. In an embodiment, m is selected from 1 to 2, m′ is selected from 0 to 2 and n″ is selected from 0 to 3. In an embodiment, m is selected from 1 to 2, m′ is selected from 0 to 2 and n″ is selected from 0 to 1.

In a particular embodiment, m is 1, m′ is 0 and n″ is 0. In another embodiment, m is 1, m′ is 1 and n″ is 0. In another embodiment, m is 1, m′ is 2 and n″ is 0. In another embodiment, m is 1, m′ is 3 and n″ is 0.

In another embodiment, m is 2, m′ is 0 and n″ is 0. In another embodiment, m is 2, m′ is 1 and n″ is 0. In another embodiment, m is 2, m′ is 2 and n″ is 0. In another embodiment, m is 2, m′ is 3 and n″ is 0.

In yet another embodiment, m is 3, m′ is 0 and n″ is 0. In yet another embodiment, m is 3, m′ is 1 and n″ is 0. In yet another embodiment, m is 3, m′ is 2 and n″ is 0. In yet another embodiment, m is 3, m′ is 3 and n″ is 0.

In yet a further embodiment, m is 4, m′ is 0 and n″ is 0. In yet a further embodiment, m is 4, m′ is 1 and n″ is 0. In yet a further embodiment, m is 4, m′ is 2 and n″ is 0. In yet a further embodiment, m is 4, m′ is 3 and n″ is 0.

In a particular embodiment, m is 1, m′ is 0 and n″ is 1. In a particular embodiment, m is 1, m′ is 1 and n″ is 1. In a particular embodiment, m is 1, m′ is 2 and n″ is 1. In a particular embodiment, m is 1, m′ is 3 and n″ is 1.

In another embodiment, m is 2, m′ is 0 and n″ is 1. In another embodiment, m is 2, m′ is 1 and n″ is 1. In another embodiment, m is 2, m′ is 2 and n″ is 1. In another embodiment, m is 2, m′ is 3 and n″ is 1.

In yet another embodiment, m is 3, m′ is 0 and n″ is 1. In yet another embodiment, m is 3, m′ is 1 and n″ is 1. In yet another embodiment, m is 3, m′ is 2 and n″ is 1. In yet another embodiment, m is 3, m′ is 3 and n″ is 1.

In yet a further embodiment, m is 4, m′ is 0 and n″ is 1. In yet a further embodiment, m is 4, m′ is 1 and n″ is 1. In yet a further embodiment, m is 4, m′ is 2 and n″ is 1. In yet a further embodiment, m is 4, m′ is 3 and n″ is 1.

In a particular embodiment, m is 1, m′ is 0 and n″ is 2. In a particular embodiment, m is 1, m′ is 1 and n″ is 2. In a particular embodiment, m is 1, m′ is 2 and n″ is 2. In a particular embodiment, m is 1, m′ is 3 and n″ is 2.

In another embodiment, m is 2, m′ is 0 and n″ is 2. In another embodiment, m is 2, m′ is 1 and n″ is 2. In another embodiment, m is 2, m′ is 2 and n″ is 2. In another embodiment, m is 2, m′ is 3 and n″ is 2.

In yet another embodiment, m is 3, m′ is 0 and n″ is 2. In yet another embodiment, m is 3, m′ is 1 and n″ is 2. In yet another embodiment, m is 3, m′ is 2 and n″ is 2. In yet another embodiment, m is 3, m′ is 3 and n″ is 2.

In yet a further embodiment, m is 4, m′ is 0 and n″ is 2. In yet a further embodiment, m is 4, m′ is 1 and n″ is 2. In yet a further embodiment, m is 4, m′ is 2 and n″ is 2. In yet a further embodiment, m is 4, m′ is 3 and n″ is 2.

In a particular embodiment, m is 1, m′ is 0 and n″ is 3. In a particular embodiment, m is 1, m′ is 1 and n″ is 3. In a particular embodiment, m is 1, m′ is 2 and n″ is 3. In a particular embodiment, m is 1, m′ is 3 and n″ is 3.

In another embodiment, m is 2, m′ is 0 and n″ is 3. In another embodiment, m is 2, m′ is 1 and n″ is 3. In another embodiment, m is 2, m′ is 2 and n″ is 3. In another embodiment, m is 2, m′ is 3 and n″ is 3.

In yet another embodiment, m is 3, m′ is 0 and n″ is 3. In yet another embodiment, m is 3, m′ is 1 and n″ is 3. In yet another embodiment, m is 3, m′ is 2 and n″ is 3. In yet another embodiment, m is 3, m′ is 3 and n″ is 3.

In yet a further embodiment, m is 4, m′ is 0 and n″ is 3. In yet a further embodiment, m is 4, m′ is 1 and n″ is 3. In yet a further embodiment, m is 4, m′ is 2 and n″ is 3. In yet a further embodiment, m is 4, m′ is 3 and n″ is 3.

In a particular embodiment, m is 1, m′ is 0 and n″ is 4. In a particular embodiment, m is 1, m′ is 1 and n″ is 4. In a particular embodiment, m is 1, m′ is 2 and n″ is 4. In a particular embodiment, m is 1, m′ is 3 and n″ is 4.

In another embodiment, m is 2, m′ is 0 and n″ is 4. In another embodiment, m is 2, m′ is 1 and n″ is 4. In another embodiment, m is 2, m′ is 2 and n″ is 4. In another embodiment, m is 2, m′ is 3 and n″ is 4.

In yet another embodiment, m is 3, m′ is 0 and n″ is 4. In yet another embodiment, m is 3, m′ is 1 and n″ is 4. In yet another embodiment, m is 3, m′ is 2 and n″ is 4. In yet another embodiment, m is 3, m′ is 3 and n″ is 4.

In yet a further embodiment, m is 4, m′ is 0 and n″ is 4. In yet a further embodiment, m is 4, m′ is 1 and n″ is 4. In yet a further embodiment, m is 4, m′ is 2 and n″ is 4. In yet a further embodiment, m is 4, m′ is 3 and n″ is 4.

In a particular embodiment, m is 1, m′ is 0 and n″ is 5. In a particular embodiment, m is 1, m′ is 1 and n″ is 5. In a particular embodiment, m is 1, m′ is 2 and n″ is 5. In a particular embodiment, m is 1, m′ is 3 and n″ is 5.

In another embodiment, m is 2, m′ is 0 and n″ is 5. In another embodiment, m is 2, m′ is 1 and n″ is 5. In another embodiment, m is 2, m′ is 2 and n″ is 5. In another embodiment, m is 2, m′ is 3 and n″ is 5.

In yet another embodiment, m is 3, m′ is 0 and n″ is 5. In yet another embodiment, m is 3, m′ is 1 and n″ is 5. In yet another embodiment, m is 3, m′ is 2 and n″ is 5. In yet another embodiment, m is 3, m′ is 3 and n″ is 5.

In yet a further embodiment, m is 4, m′ is 0 and n″ is 5. In yet a further embodiment, m is 4, m′ is 1 and n″ is 5. In yet a further embodiment, m is 4, m′ is 2 and n″ is 5. In yet a further embodiment, m is 4, m′ is 3 and n″ is 5.

In an embodiment, the invention provides a serotype 9N glycoconjugate comprising a serotype 9N saccharide covalently conjugated to a carrier protein (CP) through a spacer and having the general formula (XII), wherein X is OCH₂(CH₂)_{n′}, where n′ is selected from 1 to 10 and wherein X′ is CH₂(CH₂)_{n″}, where n″ is selected from 0 to 10.

In an embodiment, n′ is selected from 1 to 5 and n″ is selected from 0 to 10. In an embodiment, n′ is selected from 1 to 5 and n″ is selected from 0 to 5. In an embodiment, n′ is selected from 1 to 3 and n″ is selected from 0 to 3. In an embodiment, n′ is selected from 1 to 2 and n″ is selected from 0 to 2. In a particular embodiment, n′ is 1 and n″ is 0. In another embodiment, n′ is 2 and n″ is 0. In yet another embodiment, n′ is 3 and n″ is 0. In yet a further embodiment, n′ is 4 and n″ is 0. In yet a further embodiment, n′ is 5 and n″ is 0. In yet a further embodiment, n′ is 6 and n″ is 0. In a particular embodiment, n′ is 1 and n″ is 1. In another embodiment, n′ is 2 and n″ is 1. In yet another embodiment, n′ is 3 and n″ is 1. In yet a further embodiment, n′ is 4 and n″ is 1. In yet a further embodiment, n′ is 5 and n″ is 1. In yet a further embodiment, n′ is 6 and n″ is 1. In a particular embodiment, n′ is 1 and n″ is 2. In another embodiment, n′ is 2 and n″ is 2. In yet another embodiment, n′ is 3 and n″ is 2. In yet a further embodiment, n′ is 4 and n″ is 2. In yet a further embodiment, n′ is 5 and n″ is 2. In yet a further embodiment, n′ is 6 and n″ is 2. In a particular embodiment, n′ is 1 and n″ is 3. In another embodiment, n′ is 2 and n″ is 3. In yet another embodiment, n′ is 3 and n″ is 3. In yet a further embodiment, n′ is 4 and n″ is 3. In yet a further embodiment, n′ is 5 and n″ is 3. In yet a further embodiment, n′ is 6 and n″ is 3. In a particular embodiment, n′ is 1 and n″ is 4. In another embodiment, n′ is 2 and n″ is 4. In yet another embodiment, n′ is 3 and n″ is 4. In yet a further embodiment, n′ is 4 and n″ is 4. In yet a further embodiment, n′ is 5 and n″ is 4. In yet a further embodiment, n′ is 6 and n″ is 4. In a particular embodiment, n′ is 1 and n″ is 5. In another embodiment, n′ is 2 and n″ is 5. In yet another embodiment, n′ is 3 and n″ is 5. In yet a further embodiment, n′ is 4 and n″ is 5. In yet a further embodiment, n′ is 5 and n″ is 5. In yet a further embodiment, n′ is 6 and n″ is 5. In a particular embodiment, n′ is 1 and n″ is 6. In another embodiment, n′ is 2 and n″ is 6. In yet another embodiment, n′ is 3 and n″ is 6. In yet a further embodiment, n′ is 4 and n″ is 6. In yet a further embodiment, n′ is 5 and n″ is 6. In yet a further embodiment, n′ is 6 and n″ is 6.

In an embodiment, the invention provides a serotype 9N glycoconjugate comprising a serotype 9N saccharide covalently conjugated to a carrier protein (CP) through a spacer and having the general formula (XII), wherein X is OCH₂(CH₂)_{n′}, where n′ is selected from 1 to 10 and wherein X′ is CH₂O(CH₂)_{n″}CH₂, where n″ is selected from 0 to 10.

In an embodiment, n′ is selected from 1 to 5 and n″ is selected from 0 to 10. In an embodiment, n′ is selected from 1 to 5 and n″ is selected from 0 to 5. In an embodiment, n′ is selected from 1 to 3 and n″ is selected from 0 to 3. In an embodiment, n′ is selected from 1 to 2 and n″ is selected from 0 to 2. In a particular embodiment, n′ is 1 and n″ is 0. In another embodiment, n′ is 2 and n″ is 0. In yet another embodiment, n′ is 3 and n″ is 0. In yet a further embodiment, n′ is 4 and n″ is 0. In yet a further embodiment, n′ is 5 and n″ is 0. In yet a further embodiment, n′ is 6 and n″ is 0. In a particular embodiment, n′ is 1 and n″ is 1. In another embodiment, n′ is 2 and n″ is 1. In yet another embodiment, n′ is 3 and n″ is 1. In yet a further embodiment, n′ is 4 and n″ is 1. In yet a further embodiment, n′ is 5 and n″ is 1. In yet a further embodiment, n′ is 6 and n″ is 1. In a particular embodiment, n′ is 1 and n″ is 2. In another embodiment, n′ is 2 and n″ is 2. In yet another embodiment, n′ is 3 and n″ is 2. In yet a further embodiment, n′ is 4 and n″ is 2. In yet a further embodiment, n′ is 5 and n″ is 2. In yet a further embodiment, n′ is 6 and n″ is 2. In a particular embodiment, n′ is 1 and n″ is 3. In another embodiment, n′ is 2 and n″ is 3. In yet another embodiment, n′ is 3 and n″ is 3. In yet a further embodiment, n′ is 4 and n″ is 3. In yet a further embodiment, n′ is 5 and n″ is 3. In yet a further embodiment, n′ is 6 and n″ is 3. In a particular embodiment, n′ is 1 and n″ is 4. In another embodiment, n′ is 2 and n″ is 4. In yet another embodiment, n′ is 3 and n″ is 4. In yet a further embodiment, n′ is 4 and n″ is 4. In yet a further embodiment, n′ is 5 and n″ is 4. In yet a further embodiment, n′ is 6 and n″ is 4. In a particular embodiment, n′ is 1 and n″ is 5. In another embodiment, n′ is 2 and n″ is 5. In yet another embodiment, n′ is 3 and n″ is 5. In yet a further embodiment, n′ is 4 and n″ is 5. In yet a further embodiment, n′ is 5 and n″ is 5. In yet a further embodiment, n′ is 6 and n″ is 5. In a particular embodiment, n′ is 1 and n″ is 6. In another embodiment, n′ is 2 and n″ is 6. In yet another embodiment, n′ is 3 and n″ is 6. In yet a further embodiment, n′ is 4 and n″ is 6. In yet a further embodiment, n′ is 5 and n″ is 6. In yet a further embodiment, n′ is 6 and n″ is 6.

In an embodiment, the invention provides a serotype 9N glycoconjugate comprising a serotype 9N saccharide covalently conjugated to a carrier protein (CP) through a spacer and having the general formula (XII), wherein X is OCH₂(CH₂)_{n′}, where n′ is selected from 1 to 10 and wherein X′ is CH₂O(CH₂CH₂O)_{m′}(CH₂)_{n″}CH₂, where n″ is selected from 0 to 10 and m′ is selected from 0 to 4.

In an embodiment, n′ is selected from 1 to 5, m′ is selected from 0 to 4 and n″ is selected from 0 to 10. In an embodiment, n′ is selected from 1 to 5, m′ is selected from 0 to 4 and n″ is selected from 0 to 5. In an embodiment, n′ is selected from 1 to 3, m′ is selected from 0 to 2 and n″ is selected from 0 to 3. In an embodiment, n′ is selected from 1 to 2, m′ is selected from 0 to 2 and n″ is selected from 0 to 1.

In a particular embodiment, n′ is 1, m′ is 0 and n″ is 0. In another embodiment, n′ is 1, m′ is 1 and n″ is 0. In another embodiment, n′ is 1, m′ is 2 and n″ is 0. In another embodiment, n′ is 1, m′ is 3 and n″ is 0.

In another embodiment, n′ is 2, m′ is 0 and n″ is 0. In another embodiment, n′ is 2, m′ is 1 and n″ is 0. In another embodiment, n′ is 2, m′ is 2 and n″ is 0. In another embodiment, n′ is 2, m′ is 3 and n″ is 0.

In yet another embodiment, n′ is 3, m′ is 0 and n″ is 0. In yet another embodiment, n′ is 3, m′ is 1 and n″ is 0. In yet another embodiment, n′ is 3, m′ is 2 and n″ is 0. In yet another embodiment, n′ is 3, m′ is 3 and n″ is 0.

In yet a further embodiment, n′ is 4, m′ is 0 and n″ is 0. In yet a further embodiment, n′ is 4, m′ is 1 and n″ is 0. In yet a further embodiment, n′ is 4, m′ is 2 and n″ is 0. In yet a further embodiment, n′ is 4, m′ is 3 and n″ is 0.

In yet a further embodiment, n′ is 5, m′ is 0 and n″ is 0. In yet a further embodiment, n′ is 5, m′ is 1 and n″ is 0. In yet a further embodiment, n′ is 5, m′ is 2 and n″ is 0. In yet a further embodiment, n′ is 5, m′ is 3 and n″ is 0.

In a particular embodiment, n′ is 1, m′ is 0 and n″ is 1. In a particular embodiment, n′ is 1, m′ is 1 and n″ is 1. In a particular embodiment, n′ is 1, m′ is 2 and n″ is 1. In a particular embodiment, n′ is 1, m′ is 3 and n″ is 1.

In another embodiment, n′ is 2, m′ is 0 and n″ is 1. In another embodiment, n′ is 2, m′ is 1 and n″ is 1. In another embodiment, n′ is 2, m′ is 2 and n″ is 1. In another embodiment, n′ is 2, m′ is 3 and n″ is 1.

In yet another embodiment, n′ is 3, m′ is 0 and n″ is 1. In yet another embodiment, n′ is 3, m′ is 1 and n″ is 1. In yet another embodiment, n′ is 3, m′ is 2 and n″ is 1. In yet another embodiment, n′ is 3, m′ is 3 and n″ is 1.

In yet a further embodiment, n′ is 4, m′ is 0 and n″ is 1. In yet a further embodiment, n′ is 4, m′ is 1 and n″ is 1. In yet a further embodiment, n′ is 4, m′ is 2 and n″ is 1. In yet a further embodiment, n′ is 4, m′ is 3 and n″ is 1.

In yet a further embodiment, n′ is 5, m′ is 0 and n″ is 1. In yet a further embodiment, n′ is 5, m′ is 1 and n″ is 1. In yet a further embodiment, n′ is 5, m′ is 2 and n″ is 1. In yet a further embodiment, n′ is 5, m′ is 3 and n″ is 1.

In a particular embodiment, n′ is 1, m′ is 0 and n″ is 2. In a particular embodiment, n′ is 1, m′ is 1 and n″ is 2. In a particular embodiment, n′ is 1, m′ is 2 and n″ is 2. In a particular embodiment, n′ is 1, m′ is 3 and n″ is 2.

In another embodiment, n′ is 2, m′ is 0 and n″ is 2. In another embodiment, n′ is 2, m′ is 1 and n″ is 2. In another embodiment, n′ is 2, m′ is 2 and n″ is 2. In another embodiment, n′ is 2, m′ is 3 and n″ is 2.

In yet another embodiment, n′ is 3, m′ is 0 and n″ is 2. In yet another embodiment, n′ is 3, m′ is 1 and n″ is 2. In yet another embodiment, n′ is 3, m′ is 2 and n″ is 2. In yet another embodiment, n′ is 3, m′ is 3 and n″ is 2.

In yet a further embodiment, n′ is 4, m′ is 0 and n″ is 2. In yet a further embodiment, n′ is 4, m′ is 1 and n″ is 2. In yet a further embodiment, n′ is 4, m′ is 2 and n″ is 2. In yet a further embodiment, n′ is 4, m′ is 3 and n″ is 2.

In yet a further embodiment, n′ is 5, m′ is 0 and n″ is 2. In yet a further embodiment, n′ is 5, m′ is 1 and n″ is 2. In yet a further embodiment, n′ is 5, m′ is 2 and n″ is 2. In yet a further embodiment, n′ is 5, m′ is 3 and n″ is 2.

In a particular embodiment, n′ is 1, m′ is 0 and n″ is 3. In a particular embodiment, n′ is 1, m′ is 1 and n″ is 3. In a particular embodiment, n′ is 1, m′ is 2 and n″ is 3. In a particular embodiment, n′ is 1, m′ is 3 and n″ is 3.

In another embodiment, n′ is 2, m′ is 0 and n″ is 3. In another embodiment, n′ is 2, m′ is 1 and n″ is 3. In another embodiment, n′ is 2, m′ is 2 and n″ is 3. In another embodiment, n′ is 2, m′ is 3 and n″ is 3.

In yet another embodiment, n′ is 3, m′ is 0 and n″ is 3. In yet another embodiment, n′ is 3, m′ is 1 and n″ is 3. In yet another embodiment, n′ is 3, m′ is 2 and n″ is 3. In yet another embodiment, n′ is 3, m′ is 3 and n″ is 3.

In yet a further embodiment, n′ is 4, m′ is 0 and n″ is 3. In yet a further embodiment, n′ is 4, m′ is 1 and n″ is 3. In yet a further embodiment, n′ is 4, m′ is 2 and n″ is 3. In yet a further embodiment, n′ is 4, m′ is 3 and n″ is 3.

In yet a further embodiment, n′ is 5, m′ is 0 and n″ is 3. In yet a further embodiment, n′ is 5, m′ is 1 and n″ is 3. In yet a further embodiment, n′ is 5, m′ is 2 and n″ is 3. In yet a further embodiment, n′ is 5, m′ is 3 and n″ is 3.

In a particular embodiment, n′ is 1, m′ is 0 and n″ is 4. In a particular embodiment, n′ is 1, m′ is 1 and n″ is 4. In a particular embodiment, n′ is 1, m′ is 2 and n″ is 4. In a particular embodiment, n′ is 1, m′ is 3 and n″ is 4.

In another embodiment, n′ is 2, m′ is 0 and n″ is 4. In another embodiment, n′ is 2, m′ is 1 and n″ is 4. In another embodiment, n′ is 2, m′ is 2 and n″ is 4. In another embodiment, n′ is 2, m′ is 3 and n″ is 4.

In yet another embodiment, n′ is 3, m′ is 0 and n″ is 4. In yet another embodiment, n′ is 3, m′ is 1 and n″ is 4. In yet another embodiment, n′ is 3, m′ is 2 and n″ is 4. In yet another embodiment, n′ is 3, m′ is 3 and n″ is 4.

In yet a further embodiment, n′ is 4, m′ is 0 and n″ is 4. In yet a further embodiment, n′ is 4, m′ is 1 and n″ is 4. In yet a further embodiment, n′ is 4, m′ is 2 and n″ is 4. In yet a further embodiment, n′ is 4, m′ is 3 and n″ is 4.

In yet a further embodiment, n′ is 5, m′ is 0 and n″ is 4. In yet a further embodiment, n′ is 5, m′ is 1 and n″ is 4. In yet a further embodiment, n′ is 5, m′ is 2 and n″ is 4. In yet a further embodiment, n′ is 5, m′ is 3 and n″ is 4.

In a particular embodiment, n′ is 1, m′ is 0 and n″ is 5. In a particular embodiment, n′ is 1, m′ is 1 and n″ is 5. In a particular embodiment, n′ is 1, m′ is 2 and n″ is 5. In a particular embodiment, n′ is 1, m′ is 3 and n″ is 5.

In another embodiment, n′ is 2, m′ is 0 and n″ is 5. In another embodiment, n′ is 2, m′ is 1 and n″ is 5. In another embodiment, n′ is 2, m′ is 2 and n″ is 5. In another embodiment, n′ is 2, m′ is 3 and n″ is 5.

In yet another embodiment, n′ is 3, m′ is 0 and n″ is 5. In yet another embodiment, n′ is 3, m′ is 1 and n″ is 5. In yet another embodiment, n′ is 3, m′ is 2 and n″ is 5. In yet another embodiment, n′ is 3, m′ is 3 and n″ is 5.

In yet a further embodiment, n′ is 4, m′ is 0 and n″ is 5. In yet a further embodiment, n′ is 4, m′ is 1 and n″ is 5. In yet a further embodiment, n′ is 4, m′ is 2 and n″ is 5. In yet a further embodiment, n′ is 4, m′ is 3 and n″ is 5.

In yet a further embodiment, n′ is 5, m′ is 0 and n″ is 5. In yet a further embodiment, n′ is 5, m′ is 1 and n″ is 5. In yet a further embodiment, n′ is 5, m′ is 2 and n″ is 5. In yet a further embodiment, n′ is 5, m′ is 3 and n″ is 5.

In an embodiment, the invention provides a serotype 9N glycoconjugate comprising a serotype 9N saccharide covalently conjugated to a carrier protein (CP) through a spacer and having the general formula (XII), wherein X is O(CH₂CH₂O)ₘCH₂CH₂, where m is selected from 1 to 4 and wherein X′ is CH₂(CH₂)_{n″}, where n″ is selected from 0 to 10.

In an embodiment, m is selected from 1 to 3 and n″ is selected from 0 to 10. In an embodiment, m is selected from 1 to 3 and n″ is selected from 0 to 5. In an embodiment, m is selected from 1 to 2 and n″ is selected from 0 to 3. In an embodiment, m is selected from 1 to 2 and n″ is selected from 0 to 2. In a particular embodiment, m is 1 and n″ is 0. In another embodiment, m is 2 and n″ is 0. In yet another embodiment, m is 3 and n″ is 0. In yet a further embodiment, m is 4 and n″ is 0. In a particular embodiment, m is 1 and n″ is 1. In another embodiment, m is 2 and n″ is 1. In yet another embodiment, m is 3 and n″ is 1. In yet a further embodiment, m is 4 and n″ is 1. In a particular embodiment, m is 1 and n″ is 2. In another embodiment, m is 2 and n″ is 2. In yet another embodiment, m is 3 and n″ is 2. In yet a further embodiment, m is 4 and n″ is 2. In a particular embodiment, m is 1 and n″ is 3. In another embodiment, m is 2 and n″ is 3. In yet another embodiment, m is 3 and n″ is 3. In yet a further embodiment, m is 4 and n″ is 3. In a particular embodiment, m is 1 and n″ is 4. In another embodiment, m is 2 and n″ is 4. In yet another embodiment, m is 3 and n″ is 4. In yet a further embodiment, m is 4 and n″ is 4. In a particular embodiment, m is 1 and n″ is 5. In another embodiment, m is 2 and n″ is 5. In yet another embodiment, m is 3 and n″ is 5. In yet a further embodiment, m is 4 and n″ is 5. In a particular embodiment, m is 1 and n″ is 6. In another embodiment, m is 2 and n″ is 6. In yet another embodiment, m is 3 and n″ is 6. In yet a further embodiment, m is 4 and n″ is 6.

In an embodiment, the invention provides a serotype 9N glycoconjugate comprising a serotype 9N saccharide covalently conjugated to a carrier protein (CP) through a spacer and having the general formula (XII), wherein X is O(CH₂CH₂O)ₘCH₂CH₂, where m is selected from 1 to 4 and wherein X′ is CH₂O(CH₂)_{n″}CH₂, where n″ is selected from 0 to 10.

In an embodiment, m is selected from 1 to 3 and n″ is selected from 0 to 10. In an embodiment, m is selected from 1 to 3 and n″ is selected from 0 to 5. In an embodiment, m is selected from 1 to 2 and n″ is selected from 0 to 3. In an embodiment, m is selected from 1 to 2 and n″ is selected from 0 to 2. In a particular embodiment, m is 1 and n″ is 0. In another embodiment, m is 2 and n″ is 0. In yet another embodiment, m is 3 and n″ is 0. In yet a further embodiment, m is 4 and n″ is 0. In a particular embodiment, m is 1 and n″ is 1. In another embodiment, m is 2 and n″ is 1. In yet another embodiment, m is 3 and n″ is 1. In yet a further embodiment, m is 4 and n″ is 1. In a particular embodiment, m is 1 and n″ is 2. In another embodiment, m is 2 and n″ is 2. In yet another embodiment, m is 3 and n″ is 2. In yet a further embodiment, m is 4 and n″ is 2. In a particular embodiment, m is 1 and n″ is 3. In another embodiment, m is 2 and n″ is 3. In yet another embodiment, m is 3 and n″ is 3. In yet a further embodiment, m is 4 and n″ is 3. In a particular embodiment, m is 1 and n″ is 4. In another embodiment, m is 2 and n″ is 4. In yet another embodiment, m is 3 and n″ is 4. In yet a further embodiment, m is 4 and n″ is 4. In a particular embodiment, m is 1 and n″ is 5. In another embodiment, m is 2 and n″ is 5. In yet another embodiment, m is 3 and n″ is 5. In yet a further embodiment, m is 4 and n″ is 5. In a particular embodiment, m is 1 and n″ is 6. In another embodiment, m is 2 and n″ is 6. In yet another embodiment, m is 3 and n″ is 6. In yet a further embodiment, m is 4 and n″ is 6.

In an embodiment, the invention provides a serotype 9N glycoconjugate comprising a serotype 9N saccharide covalently conjugated to a carrier protein (CP) through a spacer and having the general formula (XII), wherein X is O(CH₂CH₂O)ₘCH₂CH₂, where m is selected from 1 to 4 and wherein X′ is CH₂O(CH₂CH₂O)_{m′}(CH₂)_{n″}CH₂, where n″ is selected from 0 to 10 and m′ is selected from 0 to 4.

In an embodiment, m is selected from 1 to 3, m′ is selected from 0 to 4 and n″ is selected from 0 to 10. In an embodiment, m is selected from 1 to 2, m′ is selected from 0 to 4 and n″ is selected from 0 to 5. In an embodiment, m is selected from 1 to 2, m′ is selected from 0 to 2 and n″ is selected from 0 to 3. In an embodiment, m is selected from 1 to 2, m′ is selected from 0 to 2 and n″ is selected from 0 to 1.

In a particular embodiment, m is 1, m′ is 0 and n″ is 0. In another embodiment, m is 1, m′ is 1 and n″ is 0. In another embodiment, m is 1, m′ is 2 and n″ is 0. In another embodiment, m is 1, m′ is 3 and n″ is 0.

In another embodiment, m is 2, m′ is 0 and n″ is 0. In another embodiment, m is 2, m′ is 1 and n″ is 0. In another embodiment, m is 2, m′ is 2 and n″ is 0. In another embodiment, m is 2, m′ is 3 and n″ is 0.

In yet another embodiment, m is 3, m′ is 0 and n″ is 0. In yet another embodiment, m is 3, m′ is 1 and n″ is 0. In yet another embodiment, m is 3, m′ is 2 and n″ is 0. In yet another embodiment, m is 3, m′ is 3 and n″ is 0.

In yet a further embodiment, m is 4, m′ is 0 and n″ is 0. In yet a further embodiment, m is 4, m′ is 1 and n″ is 0. In yet a further embodiment, m is 4, m′ is 2 and n″ is 0. In yet a further embodiment, m is 4, m′ is 3 and n″ is 0.

In a particular embodiment, m is 1, m′ is 0 and n″ is 1. In a particular embodiment, m is 1, m′ is 1 and n″ is 1. In a particular embodiment, m is 1, m′ is 2 and n″ is 1. In a particular embodiment, m is 1, m′ is 3 and n″ is 1.

In another embodiment, m is 2, m′ is 0 and n″ is 1. In another embodiment, m is 2, m′ is 1 and n″ is 1. In another embodiment, m is 2, m′ is 2 and n″ is 1. In another embodiment, m is 2, m′ is 3 and n″ is 1.

In yet another embodiment, m is 3, m′ is 0 and n″ is 1. In yet another embodiment, m is 3, m′ is 1 and n″ is 1. In yet another embodiment, m is 3, m′ is 2 and n″ is 1. In yet another embodiment, m is 3, m′ is 3 and n″ is 1.

In yet a further embodiment, m is 4, m′ is 0 and n″ is 1. In yet a further embodiment, m is 4, m′ is 1 and n″ is 1. In yet a further embodiment, m is 4, m′ is 2 and n″ is 1. In yet a further embodiment, m is 4, m′ is 3 and n″ is 1.

In a particular embodiment, m is 1, m′ is 0 and n″ is 2. In a particular embodiment, m is 1, m′ is 1 and n″ is 2. In a particular embodiment, m is 1, m′ is 2 and n″ is 2. In a particular embodiment, m is 1, m′ is 3 and n″ is 2.

In another embodiment, m is 2, m′ is 0 and n″ is 2. In another embodiment, m is 2, m′ is 1 and n″ is 2. In another embodiment, m is 2, m′ is 2 and n″ is 2. In another embodiment, m is 2, m′ is 3 and n″ is 2.

In yet another embodiment, m is 3, m′ is 0 and n″ is 2. In yet another embodiment, m is 3, m′ is 1 and n″ is 2. In yet another embodiment, m is 3, m′ is 2 and n″ is 2. In yet another embodiment, m is 3, m′ is 3 and n″ is 2.

In yet a further embodiment, m is 4, m′ is 0 and n″ is 2. In yet a further embodiment, m is 4, m′ is 1 and n″ is 2. In yet a further embodiment, m is 4, m′ is 2 and n″ is 2. In yet a further embodiment, m is 4, m′ is 3 and n″ is 2.

In a particular embodiment, m is 1, m′ is 0 and n″ is 3. In a particular embodiment, m is 1, m′ is 1 and n″ is 3. In a particular embodiment, m is 1, m′ is 2 and n″ is 3. In a particular embodiment, m is 1, m′ is 3 and n″ is 3.

In another embodiment, m is 2, m′ is 0 and n″ is 3. In another embodiment, m is 2, m′ is 1 and n″ is 3. In another embodiment, m is 2, m′ is 2 and n″ is 3. In another embodiment, m is 2, m′ is 3 and n″ is 3.

In yet another embodiment, m is 3, m′ is 0 and n″ is 3. In yet another embodiment, m is 3, m′ is 1 and n″ is 3. In yet another embodiment, m is 3, m′ is 2 and n″ is 3. In yet another embodiment, m is 3, m′ is 3 and n″ is 3.

In yet a further embodiment, m is 4, m′ is 0 and n″ is 3. In yet a further embodiment, m is 4, m′ is 1 and n″ is 3. In yet a further embodiment, m is 4, m′ is 2 and n″ is 3. In yet a further embodiment, m is 4, m′ is 3 and n″ is 3.

In a particular embodiment, m is 1, m′ is 0 and n″ is 4. In a particular embodiment, m is 1, m′ is 1 and n″ is 4. In a particular embodiment, m is 1, m′ is 2 and n″ is 4. In a particular embodiment, m is 1, m′ is 3 and n″ is 4.

In another embodiment, m is 2, m′ is 0 and n″ is 4. In another embodiment, m is 2, m′ is 1 and n″ is 4. In another embodiment, m is 2, m′ is 2 and n″ is 4. In another embodiment, m is 2, m′ is 3 and n″ is 4.

In yet another embodiment, m is 3, m′ is 0 and n″ is 4. In yet another embodiment, m is 3, m′ is 1 and n″ is 4. In yet another embodiment, m is 3, m′ is 2 and n″ is 4. In yet another embodiment, m is 3, m′ is 3 and n″ is 4.

In yet a further embodiment, m is 4, m′ is 0 and n″ is 4. In yet a further embodiment, m is 4, m′ is 1 and n″ is 4. In yet a further embodiment, m is 4, m′ is 2 and n″ is 4. In yet a further embodiment, m is 4, m′ is 3 and n″ is 4.

In a particular embodiment, m is 1, m′ is 0 and n″ is 5. In a particular embodiment, m is 1, m′ is 1 and n″ is 5. In a particular embodiment, m is 1, m′ is 2 and n″ is 5. In a particular embodiment, m is 1, m′ is 3 and n″ is 5.

In another embodiment, m is 2, m′ is 0 and n″ is 5. In another embodiment, m is 2, m′ is 1 and n″ is 5. In another embodiment, m is 2, m′ is 2 and n″ is 5. In another embodiment, m is 2, m′ is 3 and n″ is 5.

In yet another embodiment, m is 3, m′ is 0 and n″ is 5. In yet another embodiment, m is 3, m′ is 1 and n″ is 5. In yet another embodiment, m is 3, m′ is 2 and n″ is 5. In yet another embodiment, m is 3, m′ is 3 and n″ is 5.

In yet a further embodiment, m is 4, m′ is 0 and n″ is 5. In yet a further embodiment, m is 4, m′ is 1 and n″ is 5. In yet a further embodiment, m is 4, m′ is 2 and n″ is 5. In yet a further embodiment, m is 4, m′ is 3 and n″ is 5.

In an embodiment of the present invention, the serotype 9N glycoconjugate of the present invention are prepared using the alternative click chemistry of the present section. The invention also relates to a method of making serotype 9N glycoconjugate, as disclosed herein above.

In an embodiment, click chemistry may comprise three steps, (a) reacting an isolated serotype 9N saccharide with a carbonic acid derivative and an alkyne linker in an aprotic solvent to produce an activated alkynyl saccharide (activation of the saccharide), (b) reacting a carrier protein with an agent bearing an N-Hydroxysuccinimide (NHS) moiety and an azido group where the NHS moiety reacts with the amino groups to form an amide linkage thereby obtaining an azido functionalized carrier protein (activation of the carrier protein), (c) reacting the activated alkynyl saccharide of step (a) with the activated azido-carrier protein of step (b) by Cu⁺¹ mediated azide-alkyne cycloaddition reaction to form a glycoconjugate.

Following step (a) the saccharide is said to be activated and is referred to herein as “activated saccharide” or “activated alkynyl saccharide”.

Following step (b) the carrier is said to be activated and is referred to as “activated carrier” or “activated azido-carrier”.

As mentioned above, before the activation (a), sizing of the serotype 9N saccharide to a target molecular weight (MW) range may be performed. Therefore, in an embodiment, the isolated serotype 9N saccharide is sized before activation with a carbonic acid derivative and an alkyne linker. In an embodiment, the isolated serotype 9N saccharide is sized to any of the target molecular weight (MW) range defined above. In an embodiment, the isolated serotype 9N saccharide is not sized before activation with a carbonic acid derivative and an alkyne linker.

In an embodiment, said carbonic acid derivative is selected from the group consisting of 1,1′-carbonyldiimidazole (CDI), 1,1′-carbonyl-di-(1,2,4-triazole) (CDT), disuccinimidyl carbonate (DSC) and N-hydroxysuccinimidyl chloroformate.

In an embodiment, said carbonic acid derivative is 1,1′-carbonyldiimidazole (CDI). In another embodiment, said carbonic acid derivative is 1,1′-Carbonyl-di-(1,2,4-triazole) (CDT). In another embodiment, said carbonic acid derivative is disuccinimidyl carbonate (DSC). In yet a further embodiment, said carbonic acid derivative is N-hydroxysuccinimidyl chloroformate.

In an embodiment, said carbonic acid derivative is 1,1′-carbonyldiimidazole (CDI) or 1,1′-Carbonyl-di-(1,2,4-triazole) (CDT). Preferably, said carbonic acid derivative is 1,1′-carbonyldiimidazole (CDI).

In an embodiment, said alkyne linker is a compound of formula (XIV),

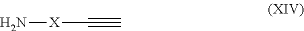

wherein X is selected from the group consisting of CH₂, CH₂(CH₂)ₙ, (CH₂CH₂O)ₘCH₂CH₂, NHCO(CH₂)ₙ, NHCO(CH₂CH₂O)ₘCH₂CH₂, OCH₂(CH₂)ₙ and O(CH₂CH₂O)ₘCH₂CH₂; where n is selected from 1 to 10 and m is selected from 1 to 4.

In an embodiment, said alkyne linker is a compound of formula (XIV), wherein X is CH₂.

In an embodiment, said alkyne linker is a compound of formula (XIV), wherein X is CH₂(CH₂)ₙ, and n is selected from 1 to 10. In an embodiment, n is selected from 1 to 5. In an embodiment, n is selected from 1 to 4. In an embodiment, n is selected from 1 to 3. In an embodiment, n is selected from 1 to 2. In a particular embodiment, n is 1. In another embodiment, n is 2. In yet another embodiment, n is 3. In yet a further embodiment, n is 4. In yet a further embodiment, n is 5. In yet a further embodiment, n is 6. In yet a further embodiment, n is 7. In yet a further embodiment, n is 8. In yet a further embodiment, n is 9. In yet a further embodiment, n is 10.

In an embodiment, said alkyne linker is a compound of formula (XIV), wherein X is (CH₂CH₂O)ₘCH₂CH₂, wherein m is selected from 1 to 4. In an embodiment, m is selected from 1 to 3. In an embodiment, m is selected from 1 to 2. In a particular embodiment, m is 1. In another embodiment, m is 2. In yet another embodiment, m is 3. In yet a further embodiment, m is 4.

In an embodiment, said alkyne linker is a compound of formula (XIV), wherein X is NHCO(CH₂)ₙ, and n is selected from 1 to 10. In an embodiment, n is selected from 1 to 5. In an embodiment, n is selected from 1 to 4. In an embodiment, n is selected from 1 to 3. In an embodiment, n is selected from 1 to 2. In a particular embodiment, n is 1. In another embodiment, n is 2. In yet another embodiment, n is 3. In yet a further embodiment, n is 4. In yet a further embodiment, n is 5. In yet a further embodiment, n is 6. In yet a further embodiment, n is 7. In yet a further embodiment, n is 8. In yet a further embodiment, n is 9. In yet a further embodiment, n is 10.

In an embodiment, said alkyne linker is a compound of formula (XIV), wherein X is NHCO(CH₂CH₂O)ₘCH₂CH₂, where m is selected from 1 to 4. In an embodiment, m is selected from 1 to 3. In an embodiment, m is selected from 1 to 2. In a particular embodiment, m is 1. In another embodiment, m is 2. In yet another embodiment, m is 3. In yet a further embodiment, m is 4.

In an embodiment, said alkyne linker is a compound of formula (XIV), wherein X is OCH₂(CH₂)ₙ, and n is selected from 1 to 10. In an embodiment, n is selected from 1 to 5. In an embodiment, n is selected from 1 to 4. In an embodiment, n is selected from 1 to 3. In an embodiment, n is selected from 1 to 2. In a particular embodiment, n is 1. In another embodiment, n is 2. In yet another embodiment, n is 3. In yet a further embodiment, n is 4. In yet a further embodiment, n is 5. In yet a further embodiment, n is 6. In yet a further embodiment, n is 7. In yet a further embodiment, n is 8. In yet a further embodiment, n is 9. In yet a further embodiment, n is 10.

In an embodiment, said alkyne linker is a compound of formula (XIV), wherein X is O(CH₂CH₂O)ₘCH₂CH₂, where m is selected from 1 to 4. In an embodiment, m is selected from 1 to 3. In an embodiment, m is selected from 1 to 2. In a particular embodiment, m is 1. In another embodiment, m is 2. In yet another embodiment, m is 3. In yet a further embodiment, m is 4.

In an embodiment, said alkyne linker is a compound of formula (XV), (XV)

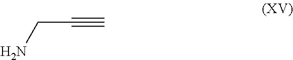

Hence in a preferred embodiment, said alkyne linker is propargylamine.

In an embodiment, said agent bearing an N-Hydroxysuccinimide (NHS) moiety and an azido group is a compound of formula (XVI),

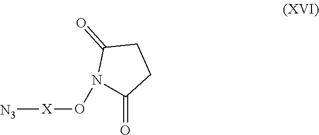

where X is selected from the group consisting of (CH₂)ₙCH₂C═O and (CH₂CH₂O)ₘCH₂CH₂═O where n is selected from 0 to 10 and m is selected from 0 to 4.

In an embodiment, said agent bearing an N-Hydroxysuccinimide (NHS) moiety and an azido group is a compound of formula (XVI), wherein X is (CH₂)_{n″}CH₂C═O, where n is selected from 0 to 10. In an embodiment, n is selected from 0 to 5. In an embodiment, n is selected from 0 to 4. In an embodiment, n is selected from 0 to 3. In an embodiment, n is selected from 0 to 2. In a particular embodiment, n is 0. In a particular embodiment, n is 1. In another embodiment, n is 2. In yet another embodiment, n is 3. In yet a further embodiment, n is 4. In yet a further embodiment, n is 5. In yet a further embodiment, n is 6. In yet a further embodiment, n is 7. In yet a further embodiment, n is 8. In yet a further embodiment, n is 9. In yet a further embodiment, n is 10.

In an embodiment, said agent bearing an N-Hydroxysuccinimide (NHS) moiety and an azido group is a compound of formula (XVI), wherein X is (CH₂CH₂O)ₘCH₂CH₂═O, where m is selected from 0 to 4. In an embodiment, m is selected from 0 to 3. In an embodiment, m is selected from 0 to 2. In an embodiment, m is selected from 0 to 1. In a particular embodiment, m is 0. In a particular embodiment, m is 1. In another embodiment, m is 2. In yet another embodiment, m is 3. In yet a further embodiment, m is 4.

In an embodiment, said agent bearing an N-Hydroxysuccinimide (NHS) moiety and an azido group is a compound of formula (XVII):

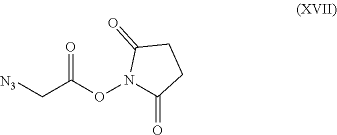 [SMILES: [N-]=[N+]=NCC(=O)ON1C(=O)CCC1=O]

Hence in an embodiment, said agent bearing an N-Hydroxysuccinimide (NHS) moiety and an azido group is (2,5-Dioxopyrrolidin-1-yl) 2-azidoacetate.

In an embodiment, step a) comprises reacting the saccharide with a carbonic acid derivative followed by reacting the carbonic acid derivative-activated saccharide with an alkyne linker in an aprotic solvent to produce an activated alkynyl saccharide.

In one embodiment step a) comprises reacting the saccharide with an amount of carbonic acid derivative that is between 0.01-10 molar equivalent to the amount of saccharide present in the reaction mixture.

In one embodiment step a) comprises reacting the saccharide with an amount of carbonic acid derivative that is between 0.1-10 molar equivalent to the amount of saccharide present in the reaction mixture.

In one embodiment step a) comprises reacting the saccharide with an amount of carbonic acid derivative that is between 0.5-5 molar equivalent to the amount of saccharide present in the reaction mixture.

In one embodiment step a) comprises reacting the saccharide with an amount of carbonic acid derivative that is between 1-5 molar equivalent to the amount of saccharide present in the reaction mixture.

In one embodiment step a) comprises reacting the saccharide with an amount of carbonic acid derivative that is between 2-5 molar equivalent to the amount of saccharide present in the reaction mixture.

In one embodiment step a) comprises reacting the saccharide with an amount of carbonic acid derivative that is between 5-10 molar equivalent to the amount of saccharide present in the reaction mixture.

In one embodiment step a) comprises reacting the saccharide with an amount of carbonic acid derivative that is between 0.1-5 molar equivalent to the amount of saccharide present in the reaction mixture.

In one embodiment step a) comprises reacting the saccharide with an amount of carbonic acid derivative that is between 0.5-2 molar equivalent to the amount of saccharide present in the reaction mixture.

In one embodiment step a) comprises reacting the saccharide with an amount of carbonic acid derivative of about 0.01 molar equivalent to the amount of saccharide present in the reaction mixture.

In one embodiment step a) comprises reacting the saccharide with an amount of carbonic acid derivative of about 0.1 molar equivalent to the amount of saccharide present in the reaction mixture.

In one embodiment step a) comprises reacting the saccharide with an amount of carbonic acid derivative of about 0.2 molar equivalent to the amount of saccharide present in the reaction mixture.

In one embodiment step a) comprises reacting the saccharide with an amount of carbonic acid derivative of about 0.5 molar equivalent to the amount of saccharide present in the reaction mixture.

In one embodiment step a) comprises reacting the saccharide with an amount of carbonic acid derivative of about 1 molar equivalent to the amount of saccharide present in the reaction mixture.

In one embodiment step a) comprises reacting the saccharide with an amount of carbonic acid derivative of about 2 molar equivalents to the amount of saccharide present in the reaction mixture.

In one embodiment step a) comprises reacting the saccharide with an amount of carbonic acid derivative of about 5 molar equivalents to the amount of saccharide present in the reaction mixture.

In one embodiment step a) comprises reacting the saccharide with an amount of carbonic acid derivative of about 10 molar equivalents to the amount of saccharide present in the reaction mixture.

In an embodiment, at step a) the isolated saccharide is reacted with a carbonic acid derivative in an aprotic solvent.

In one embodiment the isolated saccharide is reacted with a carbonic acid derivative in a solution comprising dimethylsulphoxide (DMSO) or dimethylformamide (DMF). In one embodiment the isolated saccharide is reacted with a carbonic acid derivative in a solution comprising dimethylformamide (DMF). In one embodiment the isolated saccharide is reacted with a carbonic acid derivative in a solution comprising dimethylsulphoxide (DMSO).

In a preferred embodiment the isolated saccharide is reacted with a carbonic acid derivative in a solution comprising dimethylsulphoxide (DMSO).

In one embodiment the isolated saccharide is reacted with a carbonic acid derivative in a solution consisting essentially of dimethylsulphoxide (DMSO) or dimethylformamide (DMF). In one embodiment the isolated saccharide is reacted with a carbonic acid derivative in a solution consisting essentially of dimethylformamide (DMF). In one embodiment the isolated saccharide is reacted with a carbonic acid derivative in a solution consisting essentially of dimethylsulphoxide (DMSO).

In an embodiment, the isolated saccharide is reacted with a carbonic acid derivative in a solution consisting essentially of dimethylacetamide. In an embodiment, the isolated saccharide is reacted with a carbonic acid derivative in a solution consisting essentially of N-methyl-2-pyrrolidone. In an embodiment, the isolated saccharide is reacted with a carbonic acid derivative in a solution consisting essentially of hexamethylphosphoramide (HMPA).

In a preferred embodiment the isolated saccharide is reacted with a carbonic acid derivative in a solution consisting essentially of dimethylsulphoxide (DMSO).

In one embodiment the isolated saccharide is reacted with a carbonic acid derivative in dimethylsulphoxide (DMSO) or dimethylformamide (DMF). In one embodiment the isolated saccharide is reacted with a carbonic acid derivative in dimethylformamide (DMF). In one embodiment the isolated saccharide is reacted with a carbonic acid derivative in dimethylsulphoxide (DMSO).

In an embodiment, the isolated saccharide is reacted with a carbonic acid derivative in dimethylacetamide. In an embodiment, the isolated saccharide is reacted with a carbonic acid derivative in N-methyl-2-pyrrolidone. In an embodiment, the isolated saccharide is reacted with a carbonic acid derivative in hexamethylphosphoramide (HMPA).

In a preferred embodiment the isolated saccharide is reacted with CDI in dimethylsulphoxide (DMSO). In an embodiment the isolated saccharide is reacted with CDI in anhydrous DMSO.

It has been surprisingly found that reacting the isolated saccharide with CDI in an environment with a moisture level of about 0.1% to 1% (v/v) allows to avoid side reactions.

Therefore, in one embodiment the isolated saccharide is reacted with CDI in an aprotic solvent comprising 0.1% to 1% (v/v) water. In one embodiment the isolated saccharide is reacted with CDI in an aprotic solvent comprising 0.1% to 0.8% (v/v) water. In one embodiment the isolated saccharide is reacted with CDI in an aprotic solvent comprising 0.1% to 0.5% (v/v) water. In one embodiment the isolated saccharide is reacted with CDI in an aprotic solvent comprising 0.1% to 0.4% (v/v) water. In one embodiment the isolated saccharide is reacted with CDI in an aprotic solvent comprising 0.1% to 0.3% (v/v) water. In one embodiment the isolated saccharide is reacted with CDI in an aprotic solvent comprising 0.1% to 0.2% (v/v) water.

In one embodiment the isolated saccharide is reacted with CDI in an aprotic solvent comprising 0.2% to 1% (v/v) water. In one embodiment the isolated saccharide is reacted with CDI in an aprotic solvent comprising 0.2% to 0.8% (v/v) water. In one embodiment the isolated saccharide is reacted with CDI in an aprotic solvent comprising 0.2% to 0.5% (v/v) water. In one embodiment the isolated saccharide is reacted with CDI in an aprotic solvent comprising 0.2% to 0.4% (v/v) water. In one embodiment the isolated saccharide is reacted with CDI in an aprotic solvent comprising 0.2% to 0.3% (v/v) water.

In one embodiment the isolated saccharide is reacted with CDI in an aprotic solvent comprising 0.3% to 0.8% (v/v) water. In one embodiment the isolated saccharide is reacted with CDI in an aprotic solvent comprising 0.3% to 0.5% (v/v) water. In one embodiment the isolated saccharide is reacted with CDI in an aprotic solvent comprising 0.3% to 0.4% (v/v) water.

Preferably, in one embodiment the isolated saccharide is reacted with CDI in an aprotic solvent comprising 0.1% to 0.5% (v/v) water.

In one embodiment the isolated saccharide is reacted with CDI in an aprotic solvent comprising about 0.1% (v/v) water. In one embodiment the isolated saccharide is reacted with CDI in an aprotic solvent comprising about 0.2% (v/v) water. In one embodiment the isolated saccharide is reacted with CDI in an aprotic solvent comprising about 0.3% (v/v) water. In one embodiment the isolated saccharide is reacted with CDI in an aprotic solvent comprising about 0.4% (v/v) water. In one embodiment the isolated saccharide is reacted with CDI in an aprotic solvent comprising about 0.5% (v/v) water. In one embodiment the isolated saccharide is reacted with CDI in an aprotic solvent comprising about 0.6% (v/v) water. In one embodiment the isolated saccharide is reacted with CDI in an aprotic solvent comprising about 0.7% (v/v) water. In one embodiment the isolated saccharide is reacted with CDI in an aprotic solvent comprising about 0.8% (v/v) water. In one embodiment the isolated saccharide is reacted with CDI in an aprotic solvent comprising about 0.9% (v/v) water.

In one embodiment the isolated saccharide is reacted with CDI in DMSO comprising 0.1% to 1% (v/v) water. In one embodiment the isolated saccharide is reacted with CDI in DMSO comprising 0.1% to 0.8% (v/v) water. In one embodiment the isolated saccharide is reacted with CDI in DMSO comprising 0.1% to 0.5% (v/v) water. In one embodiment the isolated saccharide is reacted with CDI in DMSO comprising 0.1% to 0.4% (v/v) water. In one embodiment the isolated saccharide is reacted with CDI in DMSO comprising 0.1% to 0.3% (v/v) water. In one embodiment the isolated saccharide is reacted with CDI in DMSO comprising 0.1% to 0.2% (v/v) water.

In one embodiment the isolated saccharide is reacted with CDI in DMSO comprising 0.2% to 1% (v/v) water. In one embodiment the isolated saccharide is reacted with CDI in DMSO comprising 0.2% to 0.8% (v/v) water. In one embodiment the isolated saccharide is reacted with CDI in DMSO comprising 0.2% to 0.5% (v/v) water. In one embodiment the isolated saccharide is reacted with CDI in DMSO comprising 0.2% to 0.4% (v/v) water. In one embodiment the isolated saccharide is reacted with CDI in DMSO comprising 0.2% to 0.3% (v/v) water.

In one embodiment the isolated saccharide is reacted with CDI in DMSO comprising 0.3% to 0.8% (v/v) water. In one embodiment the isolated saccharide is reacted with CDI in DMSO comprising 0.3% to 0.5% (v/v) water. In one embodiment the isolated saccharide is reacted with CDI in DMSO comprising 0.3% to 0.4% (v/v) water.

Preferably, in one embodiment the isolated saccharide is reacted with CDI in DMSO comprising 0.1% to 0.5% (v/v) water.

In one embodiment the isolated saccharide is reacted with CDI in DMSO comprising about 0.1% (v/v) water. In one embodiment the isolated saccharide is reacted with CDI in DMSO comprising about 0.2% (v/v) water. In one embodiment the isolated saccharide is reacted with CDI in DMSO comprising about 0.3% (v/v) water. In one embodiment the isolated saccharide is reacted with CDI in DMSO comprising about 0.4% (v/v) water. In one embodiment the isolated saccharide is reacted with CDI in DMSO comprising about 0.5% (v/v) water. In one embodiment the isolated saccharide is reacted with CDI in DMSO comprising about 0.6% (v/v) water. In one embodiment the isolated saccharide is reacted with CDI in DMSO comprising about 0.7% (v/v) water. In one embodiment the isolated saccharide is reacted with CDI in DMSO comprising about 0.8% (v/v) water. In one embodiment the isolated saccharide is reacted with CDI in DMSO comprising about 0.9% (v/v) water.

In one embodiment the free carbonic acid derivative is then quenched by the addition of water before the addition of the alkyne linker. Water can inactivate free CDI.

Therefore, in an embodiment, carbonic acid derivative activation is followed by the addition of water. In an embodiment, water is added to bring the total water content in the mixture to between about 1% to about 10% (v/v). In an embodiment, water is added to bring the total water content in the mixture to between about 1% to about 5% (v/v). In an embodiment, water is added to bring the total water content in the mixture to about 1% (v/v). In an embodiment, water is added to bring the total water content in the mixture to about 2% (v/v). In an embodiment, water is added to bring the total water content in the mixture to about 5% (v/v).

Once the saccharide has been reacted with carbonic acid derivative and following an eventual quenching of carbonic acid derivative with water, the carbonic acid derivative-activated saccharide is reacted with an alkyne linker.

In one embodiment step a) further comprises reacting the carbonic acid derivative-activated saccharide with an amount of alkyne linker that is between 0.01-10 molar equivalent to the amount of polysaccharide Repeat Unit of the activated saccharide (molar equivalent of RU).

In one embodiment step a) further comprises reacting the carbonic acid derivative-activated saccharide with an amount of alkyne linker that is between 0.1-5 molar equivalent to the amount of polysaccharide repeat unit of the activated saccharide.

In one embodiment step a) further comprises reacting the carbonic acid derivative-activated saccharide with an amount of alkyne linker that is between 0.1-5 molar equivalent to the amount of polysaccharide repeat unit of the activated saccharide.

In one embodiment step a) further comprises reacting the carbonic acid derivative-activated saccharide with an amount of alkyne linker that is between 0.5-2 molar equivalent to the amount of polysaccharide repeat unit of the activated saccharide.

In one embodiment step a) further comprises reacting the carbonic acid derivative-activated saccharide with an amount of alkyne linker that is between 1-5 molar equivalent to the amount of polysaccharide repeat unit of the activated saccharide.

In the above embodiments, said carbonic acid derivative is preferably CDI. In another embodiment, said carbonic acid derivative is CDT.

In one embodiment the degree of activation of the activated saccharide following step a) is between 0.5 to 50%. The degree of activation of the alkynyl saccharide being defined as the percentage of Repeating Unit linked to an alkyne linker.

In one embodiment the degree of activation of the activated saccharide following step a) is between 1 to 30%. In another embodiment the degree of activation of the activated saccharide following step a) is between 2 to 25%. In another embodiment the degree of activation of the activated saccharide following step a) is between 3 to 20%.

In another embodiment the degree of activation of the activated saccharide following step a) is between 3 to 15%. In another embodiment the degree of activation of the activated saccharide following step a) is between 4 to 15%. In an embodiment the degree of activation of the activated saccharide following step a) is between 1 to 6%.

In an embodiment the degree of activation of the activated saccharide following step a) is between 3 to 6%. In an embodiment the degree of activation of the activated saccharide following step a) is between 10 to 15%.

In an embodiment the degree of activation of the activated saccharide following step a) is about 1%. In an embodiment the degree of activation of the activated saccharide following step a) is about 2%. In an embodiment the degree of activation of the activated saccharide following step a) is about 3%. In an embodiment the degree of activation of the activated saccharide following step a) is about 4%. In an embodiment the degree of activation of the activated saccharide following step a) is about 5%. In an embodiment the degree of activation of the activated saccharide following step a) is about 6%. In an embodiment the degree of activation of the activated saccharide following step a) is about 7%. In an embodiment the degree of activation of the activated saccharide following step a) is about 8%. In an embodiment the degree of activation of the activated saccharide following step a) is about 9%. In an embodiment the degree of activation of the activated saccharide following step a) is about 10%. In an embodiment the degree of activation of the activated saccharide following step a) is about 11%. In an embodiment the degree of activation of the activated saccharide following step a) is about 12%. In an embodiment the degree of activation of the activated saccharide following step a) is about 13%.

In an embodiment the degree of activation of the activated saccharide following step a) is about 14%. In an embodiment the degree of activation of the activated saccharide following step a) is about 15%. In an embodiment the degree of activation of the activated saccharide following step a) is about 16%. In an embodiment the degree of activation of the activated saccharide following step a) is about 17%. In an embodiment the degree of activation of the activated saccharide following step a) is about 18%. In an embodiment the degree of activation of the activated saccharide following step a) is about 19%. In an embodiment the degree of activation of the activated saccharide following step a) is about 20%.

In one embodiment step b) comprises reacting the carrier protein with an amount of agent bearing an N-Hydroxysuccinimide (NHS) moiety and an azido group that is 0.1-10 molar equivalents to the lysines on the carrier protein.

In one embodiment step b) comprises reacting the carrier protein with an amount of agent bearing an N-Hydroxysuccinimide (NHS) moiety and an azido group that is 0.5-10 molar equivalents to the lysines on the carrier protein.

In one embodiment step b) comprises reacting the carrier protein with an amount of agent bearing an N-Hydroxysuccinimide (NHS) moiety and an azido group that is 1-5 molar equivalents to the lysines on the carrier protein.

In one embodiment step b) comprises reacting the carrier protein with an amount of agent bearing an N-Hydroxysuccinimide (NHS) moiety and an azido group that is 2-5 molar equivalents to the lysines on the carrier protein.

In one embodiment step b) comprises reacting the carrier protein with an amount of agent bearing an N-Hydroxysuccinimide (NHS) moiety and an azido group that is about 10 molar equivalents to the lysines on the carrier protein.

In one embodiment step b) comprises reacting the carrier protein with an amount of agent bearing an N-Hydroxysuccinimide (NHS) moiety and an azido group that is about 7.5 molar equivalents to the lysines on the carrier protein.

In one embodiment step b) comprises reacting the carrier protein with an amount of agent bearing an N-Hydroxysuccinimide (NHS) moiety and an azido group that is about 5 molar equivalents to the lysines on the carrier protein.

In one embodiment step b) comprises reacting the carrier protein with an amount of agent bearing an N-Hydroxysuccinimide (NHS) moiety and an azido group that is about 2 molar equivalents to the lysines on the carrier protein.

In one embodiment step b) comprises reacting the carrier protein with an amount of agent bearing an N-Hydroxysuccinimide (NHS) moiety and an azido group that is about 1 molar equivalent to the lysines on the carrier protein.

In one embodiment step b) comprises reacting the carrier protein with an amount of agent bearing an N-Hydroxysuccinimide (NHS) moiety and an azido group that is about 0.5 molar equivalents to the lysines on the carrier protein.

In one embodiment step b) comprises reacting the carrier protein with an amount of agent bearing an N-Hydroxysuccinimide (NHS) moiety and an azido group that is about 0.1 molar equivalents to the lysines on the carrier protein.

In one embodiment the degree of activation of the activated carrier following step b) is between 1 and 50. The degree of activation of the activated carrier being defined as the number of lysine residues in the carrier protein that become linked to the agent bearing an N-Hydroxysuccinimide (NHS) moiety and an azido group.

In an embodiment, the carrier protein is CRM₁₉₇, which contains 39 lysine residues. In said embodiment the degree of activation of the activated carrier following step b) may be between 1 to 30. In another embodiment the degree of activation of the activated carrier (CRM₁₉₇) following step b) is between 5 to 20. In another embodiment the degree of activation of the activated carrier (CRM₁₉₇) following step b) is between 9 to 18. In another embodiment the degree of activation of the activated carrier (CRM₁₉₇) following step b) is between 8 to 11. In another embodiment the degree of activation of the activated carrier (CRM₁₉₇) following step b) is between 15 to 20. In another embodiment the degree of activation of the activated carrier (CRM₁₉₇) following step b) is about 5. In another embodiment the degree of activation of the activated carrier (CRM₁₉₇) following step b) is about 6. In another embodiment the degree of activation of the activated carrier (CRM₁₉₇) following step b) is about 7. In another embodiment the degree of activation of the activated carrier (CRM₁₉₇) following step b) is about 8. In another embodiment the degree of activation of the activated carrier (CRM₁₉₇) following step b) is about 9. In another embodiment the degree of activation of the activated carrier (CRM₁₉₇) following step b) is about 10. In another embodiment the degree of activation of the activated carrier (CRM₁₉₇) following step b) is about 11. In another embodiment the degree of activation of the activated carrier (CRM₁₉₇) following step b) is about 12. In another embodiment the degree of activation of the activated carrier (CRM₁₉₇) following step b) is about 13. In another embodiment the degree of activation of the activated carrier (CRM₁₉₇) following step b) is about 14. In another embodiment the degree of activation of the activated carrier (CRM₁₉₇) following step b) is about 15. In another embodiment the degree of activation of the activated carrier (CRM₁₉₇) following step b) is about 16. In another embodiment the degree of activation of the activated carrier (CRM₁₉₇) following step b) is about 17. In another embodiment the degree of activation of the activated carrier (CRM₁₉₇) following step b) is about 18. In another embodiment the degree of activation of the activated carrier (CRM₁₉₇) following step b) is about 19. In another embodiment the degree of activation of the activated carrier (CRM₁₉₇) following step b) is about 20. In another embodiment the degree of activation of the activated carrier (CRM₁₉₇) following step b) is about 21. In another embodiment the degree of activation of the activated carrier (CRM₁₉₇) following step b) is about 22. In another embodiment the degree of activation of the activated carrier (CRM₁₉₇) following step b) is about 23. In another embodiment the degree of activation of the activated carrier (CRM₁₉₇) following step b) is about 24. In another embodiment the degree of activation of the activated carrier (CRM₁₉₇) following step b) is about 25.

In an embodiment, the carrier protein is SCP or a fragment thereof. In said embodiment the degree of activation of the activated carrier following step b) may be between 1 to 50. In another embodiment the degree of activation of the activated carrier (SCP) following step b) is between 5 to 50. In another embodiment the degree of activation of the activated carrier (SCP) following step b) is between 7 to 45. In another embodiment the degree of activation of the activated carrier (SCP) following step b) is between 5 to 15. In another embodiment the degree of activation of the activated carrier (SCP) following step b) is between 20 to 30. In another embodiment the degree of activation of the activated carrier (SCP) following step b) is between 30 to 50. In another embodiment the degree of activation of the activated carrier (SCP) following step b) is between 30 to 40. In another embodiment the degree of activation of the activated carrier (SCP) following step b) is between 10 to 40. In another embodiment the degree of activation of the activated carrier (SCP) following step b) is about 5. In another embodiment the degree of activation of the activated carrier (SCP) following step b) is about 7. In another embodiment the degree of activation of the activated carrier (SCP) following step b) is about 10.

In another embodiment the degree of activation of the activated carrier (SCP) following step b) is about 13. In another embodiment the degree of activation of the activated carrier (SCP) following step b) is about 15. In another embodiment the degree of activation of the activated carrier (SCP) following step b) is about 20. In another embodiment the degree of activation of the activated carrier (SCP) following step b) is about 26. In another embodiment the degree of activation of the activated carrier (SCP) following step b) is about 30. In another embodiment the degree of activation of the activated carrier (SCP) following step b) is about 35. In another embodiment the degree of activation of the activated carrier (SCP) following step b) is about 37. In another embodiment the degree of activation of the activated carrier (SCP) following step b) is about 40.

In another embodiment the degree of activation of the activated carrier (SCP) following step b) is about 45. In another embodiment the degree of activation of the activated carrier (SCP) following step b) is about 50.

In an embodiment, the carrier protein is TT or a fragment thereof. In said embodiment the degree of activation of the activated carrier following step b) may be between 1 to 30. In another embodiment the degree of activation of the activated carrier (TT) following step b) is between 5 to 25. In another embodiment the degree of activation of the activated carrier (TT) following step b) is between 7 to 25. In another embodiment the degree of activation of the activated carrier (TT) following step b) is between 10 to 20. In another embodiment the degree of activation of the activated carrier (TT) following step b) is about 5. In another embodiment the degree of activation of the activated carrier (TT) following step b) is about 7. In another embodiment the degree of activation of the activated carrier (TT) following step b) is about 10. In another embodiment the degree of activation of the activated carrier (TT) following step b) is about 12. In another embodiment the degree of activation of the activated carrier (TT) following step b) is about 15. In another embodiment the degree of activation of the activated carrier (TT) following step b) is about 20. In another embodiment the degree of activation of the activated carrier (TT) following step b) is about 25. In another embodiment the degree of activation of the activated carrier (TT) following step b) is about 30.

In an embodiment, the conjugation reaction c) is carried out in aqueous buffer. In an embodiment, the conjugation reaction c) is carried out in aqueous buffer in the presence of copper (I) as catalyst. In an embodiment, the conjugation reaction c) is carried out in aqueous buffer in the presence an oxidant and of copper (I) as catalyst. In a preferred embodiment, the conjugation reaction c) is carried out in aqueous buffer in the presence of copper (I) as catalyst and ascorbate as oxidant. In an embodiment, THPTA (tris(3-hydroxypropyltriazolylmethyl)amine) and aminoguanidine may be further added to protect the protein from side reactions. Therefore, in a preferred embodiment, the conjugation reaction c) is carried out in aqueous buffer in the presence of copper (I) as catalyst and ascorbate as oxidant, wherein the reaction mixture further comprises THPTA (tris(3-hydroxypropyltriazolylmethyl)amine) and aminoguanidine.

In an embodiment the initial input ratio (weight by weight) of activated alkynyl saccharide to activated azido-carrier at step c) is between 0.1 and 3. In an embodiment the initial input ratio (weight by weight) of activated alkynyl saccharide to activated azido-carrier at step c) is between 0.5 and 2. In an embodiment the initial input ratio (weight by weight) of activated alkynyl saccharide to activated azido-carrier at step c) is between 0.6 and 1.5. In a preferred embodiment the initial input ratio (weight by weight) of activated alkynyl saccharide to activated azido-carrier at step c) is between 0.8 and 1. In an embodiment the initial input ratio (weight by weight) of activated alkynyl saccharide to activated azido-carrier at step c) is about 0.5. In an embodiment the initial input ratio (weight by weight) of activated alkynyl saccharide to activated azido-carrier at step c) is about 0.6. In an embodiment the initial input ratio (weight by weight) of activated alkynyl saccharide to activated azido-carrier at step c) is about 0.7. In an embodiment the initial input ratio (weight by weight) of activated alkynyl saccharide to activated azido-carrier at step c) is about 0.8. In an embodiment the initial input ratio (weight by weight) of activated alkynyl saccharide to activated azido-carrier at step c) is about 0.9. In an embodiment the initial input ratio (weight by weight) of activated alkynyl saccharide to activated azido-carrier at step c) is about 1. In an embodiment the initial input ratio (weight by weight) of activated alkynyl saccharide to activated azido-carrier at step c) is about 1.1. In an embodiment the initial input ratio (weight by weight) of activated alkynyl saccharide to activated azido-carrier at step c) is about 1.2. In an embodiment the initial input ratio (weight by weight) of activated alkynyl saccharide to activated azido-carrier at step c) is about 1.3. In an embodiment the initial input ratio (weight by weight) of activated alkynyl saccharide to activated azido-carrier at step c) is about 1.4. In an embodiment the initial input ratio (weight by weight) of activated alkynyl saccharide to activated azido-carrier at step c) is about 1.5. In an embodiment the initial input ratio (weight by weight) of activated alkynyl saccharide to activated azido-carrier at step c) is about 1.6. In an embodiment the initial input ratio (weight by weight) of activated alkynyl saccharide to activated azido-carrier at step c) is about 1.7. In an embodiment the initial input ratio (weight by weight) of activated alkynyl saccharide to activated azido-carrier at step c) is about 1.8. In an embodiment the initial input ratio (weight by weight) of activated alkynyl saccharide to activated azido-carrier at step c) is about 1.9. In an embodiment the initial input ratio (weight by weight) of activated alkynyl saccharide to activated azido-carrier at step c) is about 2.

Following the click conjugation reaction, unreacted alkyne groups may remain present in the conjugates, these may be capped using a suitable alkyne group capping agent. In one embodiment this alkyne group capping agent is an agent bearing an azido group.

In an embodiment, said alkyne group capping agent is a compound of formula (XVIII),

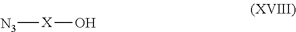

wherein X is (CH₂)ₙ wherein n is selected from 1 to 15.

In one embodiment this alkyne group capping agent is 3-azido-1-propanol.

Therefore, in an embodiment, following step (c) the process further comprises a step of capping the unreacted alkyne groups remained in the conjugates with an alkyne group capping agent.

In an embodiment the capping of the unreacted alkyne groups is performed with an amount of capping agent that is between 0.05 to 20 molar equivalents to the amount of polysaccharide repeat unit of the activated saccharide.

In an embodiment the capping of the unreacted alkyne groups is performed with an amount of capping agent that is between 0.1 to 15 molar equivalents to the amount of polysaccharide repeat unit of the activated saccharide.

In an embodiment the capping of the unreacted alkyne groups is performed with an amount of capping agent that is between 0.5 to 10 molar equivalents to the amount of polysaccharide repeat unit of the activated saccharide.

In an embodiment the capping of the unreacted alkyne groups is performed with an amount of capping agent that is between 0.5 to 5 molar equivalents to the amount of polysaccharide repeat unit of the activated saccharide.

In an embodiment the capping of the unreacted alkyne groups is performed with an amount of capping agent that is between 0.5 to 2 molar equivalents to the amount of polysaccharide repeat unit of the activated saccharide.

In an embodiment the capping of the unreacted alkyne groups is performed with an amount of capping agent that is between 0.5 to 1 molar equivalents to the amount of polysaccharide repeat unit of the activated saccharide.

In an embodiment the capping of the unreacted alkyne groups is performed with an amount of capping agent that is between 1 to 2 molar equivalents to the amount of polysaccharide repeat unit of the activated saccharide.

In an embodiment the capping of the unreacted alkyne groups is performed with an amount of capping agent that is between 1.5 to 2.5 molar equivalents to the amount of polysaccharide repeat unit of the activated saccharide.

In an embodiment the capping of the unreacted alkyne groups is performed with an amount of capping agent that about 0.5 molar equivalent to the amount of polysaccharide repeat unit of the activated saccharide.

In an embodiment the capping of the unreacted alkyne groups is performed with an amount of capping agent that about 1 molar equivalent to the amount of polysaccharide repeat unit of the activated saccharide.

In an embodiment the capping of the unreacted alkyne groups is performed with an amount of capping agent that about 1.5 molar equivalent to the amount of polysaccharide repeat unit of the activated saccharide.

In an embodiment the capping of the unreacted alkyne groups is performed with an amount of capping agent that about 2 molar equivalents to the amount of polysaccharide repeat unit of the activated saccharide.

In an embodiment the capping of the unreacted alkyne groups is performed with an amount of capping agent that about 2.5 molar equivalents to the amount of polysaccharide repeat unit of the activated saccharide.

In an embodiment the capping of the unreacted alkyne groups is performed with an amount of capping agent that about 5 molar equivalents to the amount of polysaccharide repeat unit of the activated saccharide.

Following the click conjugation reaction, there may remain unreacted azido groups in the conjugates, these may be capped using a suitable azido group capping agent. Therefore, in an embodiment, following step c), unreacted azido groups in the conjugates, are capped using a suitable azido group capping agent. In one embodiment this azido group capping agent is an agent bearing an alkyne group. In one embodiment this azido group capping agent is an agent bearing a terminal alkyne. In one embodiment this azido group capping agent is an agent bearing a cycloalkyne.

In an embodiment, said azido group capping agent is a compound of formula (XIX),

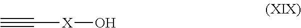

wherein X is (CH₂)ₙ wherein n is selected from 1 to 15.

In one embodiment this azido group capping agent is propargyl alcohol.

Therefore, in an embodiment, following step (c) the process further comprises a step of capping the unreacted azido groups remained in the conjugates with an azido group capping agent.

In an embodiment the capping of the unreacted azido groups is performed with an amount of capping agent that is between 0.05 to 20 molar equivalents to the amount of polysaccharide repeat unit of the activated saccharide.

In an embodiment the capping of the unreacted azido groups is performed with an amount of capping agent that is between 0.5 to 10 molar equivalents to the amount of polysaccharide repeat unit of the activated saccharide.

In an embodiment the capping of the unreacted azido groups is performed with an amount of capping agent that is between 0.5 to 5 molar equivalents to the amount of polysaccharide repeat unit of the activated saccharide.

In an embodiment the capping of the unreacted azido groups is performed with an amount of capping agent that is between 0.5 to 2 molar equivalents to the amount of polysaccharide repeat unit of the activated saccharide.

In an embodiment the capping of the unreacted azido groups is performed with an amount of capping agent that is between 0.5 to 1 molar equivalents to the amount of polysaccharide repeat unit of the activated saccharide.

In an embodiment the capping of the unreacted azido groups is performed with an amount of capping agent that is between 1 to 2 molar equivalents to the amount of polysaccharide repeat unit of the activated saccharide.

In an embodiment the capping of the unreacted azido groups is performed with an amount of capping agent that is between 0.75 to 1.5 molar equivalents to the amount of polysaccharide repeat unit of the activated saccharide.

In an embodiment the capping of the unreacted azido groups is performed with an amount of capping agent that is about 1 molar equivalent to the amount of polysaccharide repeat unit of the activated saccharide.

In an embodiment the capping of the unreacted azido groups is performed with an amount of capping agent that is about 1.5 molar equivalent to the amount of polysaccharide repeat unit of the activated saccharide.

In an embodiment the capping of the unreacted azido groups is performed with an amount of capping agent that is about 0.5 molar equivalent to the amount of polysaccharide repeat unit of the activated saccharide.

In an embodiment the capping of the unreacted azido groups is performed with an amount of capping agent that is about 2 molar equivalents to the amount of polysaccharide repeat unit of the activated saccharide.

Following conjugation to the carrier protein, the glycoconjugate can be purified (enriched with respect to the amount of saccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration. Therefore, in one embodiment the process for producing the glycoconjugate of the present invention comprises the step of purifying the glycoconjugate after it is produced.

In an aspect, the invention provides a serotype 9N glycoconjugate produced according to any of the methods disclosed herein.

In an embodiment, the serotype 9N glycoconjugate of the present invention is prepared by click chemistry and comprises a serotype 9N capsular polysaccharide wherein the weight average molecular weight (Mw) of said polysaccharide before conjugation is between 300 kDa and 800 kDa.

In an embodiment, the serotype 9N glycoconjugate of the present invention is prepared by click chemistry and comprises a serotype 9N capsular polysaccharide wherein the weight average molecular weight (Mw) of said polysaccharide before conjugation is between 400 kDa and 750 kDa.

In an embodiment, the serotype 9N glycoconjugate of the present invention is prepared by click chemistry and comprises a serotype 9N capsular polysaccharide wherein the weight average molecular weight (Mw) of said polysaccharide before conjugation is between 500 kDa and 700 kDa.

In an embodiment, the serotype 9N glycoconjugate of the present invention is prepared by click chemistry and has a weight average molecular weight (Mw) of between 1,000 kDa and 5,000 kDa.

In an embodiment, the serotype 9N glycoconjugate of the present invention is prepared by click chemistry and has a weight average molecular weight (Mw) of between 2,000 kDa and 4,000 kDa.

In an embodiment, the serotype 9N glycoconjugate of the present invention is prepared by click chemistry and the saccharide to carrier protein ratio (w/w) is between 0.5 and 1.5.

In an embodiment, the serotype 9N glycoconjugate of the present invention is prepared by click chemistry and the ratio of serotype 9N capsular polysaccharide to carrier protein in the conjugate is between 0.8 and 1.2.

In an embodiment, the serotype 9N glycoconjugate of the present invention is prepared by click chemistry and the degree of activation of the activated saccharide is between 5.0 to 35%.

In an embodiment, the serotype 9N glycoconjugate of the present invention is prepared by click chemistry and the degree of activation of the activated saccharide is between 9.0 to 32%. The degree of activation of the saccharide being defined as the percentage of Repeating Unit linked to an azido linker.

In an embodiment, the serotype 9N glycoconjugate of the present invention is prepared by click chemistry and comprises a serotype 9N capsular polysaccharide wherein the weight average molecular weight (Mw) of said polysaccharide before conjugation is between 400 kDa and 750 kDa and the serotype 9N glycoconjugate has a weight average molecular weight (Mw) of between 1,000 kDa and 5,000 kDa.

In an embodiment, the serotype 9N glycoconjugate of the present invention is prepared by click chemistry and comprises a serotype 9N capsular polysaccharide wherein the weight average molecular weight (Mw) of said polysaccharide before conjugation is between 500 kDa and 700 kDa and the serotype 9N glycoconjugate has a weight average molecular weight (Mw) of between 2,000 kDa and 4,000 kDa.

In an embodiment, the serotype 9N glycoconjugate of the present invention is prepared by click chemistry and comprises a serotype 9N capsular polysaccharide wherein the weight average molecular weight (Mw) of said polysaccharide before conjugation is between 400 kDa and 750 kDa and the serotype 9N glycoconjugate has a weight average molecular weight (Mw) of between 1,000 kDa and 5,000 kDa and the saccharide to carrier protein ratio (w/w) is between 0.5 and 1.5.

In an embodiment, the serotype 9N glycoconjugate of the present invention is prepared by click chemistry and comprises a serotype 9N capsular polysaccharide wherein the weight average molecular weight (Mw) of said polysaccharide before conjugation is between 500 kDa and 700 kDa and the serotype 9N glycoconjugate has a weight average molecular weight (Mw) of between 2,000 kDa and 4,000 kDa and the saccharide to carrier protein ratio (w/w) is between 0.8 and 1.2.

In an embodiment, the serotype 9N glycoconjugate of the present invention is prepared by click chemistry and comprises a serotype 9N capsular polysaccharide wherein the weight average molecular weight (Mw) of said polysaccharide before conjugation is between 400 kDa and 750 kDa and the serotype 9N glycoconjugate has a weight average molecular weight (Mw) of between 1,000 kDa and 5,000 kDa, the saccharide to carrier protein ratio (w/w) is between 0.5 and 1.5 and the degree of activation of the activated saccharide is between 5.0 to 35%.

In an embodiment, the serotype 9N glycoconjugate of the present invention is prepared by click chemistry and comprises a serotype 9N capsular polysaccharide wherein the weight average molecular weight (Mw) of said polysaccharide before conjugation is between 500 kDa and 700 kDa and the serotype 9N glycoconjugate has a weight average molecular weight (Mw) of between 2,000 kDa and 4,000 kDa, the saccharide to carrier protein ratio (w/w) is between 0.8 and 1.2 and the degree of activation of the activated saccharide is between 5.0 to 35%.

In an embodiment, the serotype 9N glycoconjugate of the present invention is prepared by click chemistry and comprises a serotype 9N capsular polysaccharide wherein the weight average molecular weight (Mw) of said polysaccharide before conjugation is between 400 kDa and 750 kDa and the serotype 9N glycoconjugate has a weight average molecular weight (Mw) of between 1,000 kDa and 5,000 kDa, the saccharide to carrier protein ratio (w/w) is between 0.5 and 1.5 and the degree of activation of the activated saccharide is between 9.0 to 32%.

In an embodiment, the serotype 9N glycoconjugate of the present invention is prepared by click chemistry and comprises a serotype 9N capsular polysaccharide wherein the weight average molecular weight (Mw) of said polysaccharide before conjugation is between 500 kDa and 700 kDa and the serotype 9N glycoconjugate has a weight average molecular weight (Mw) of between 2,000 kDa and 4,000 kDa, the saccharide to carrier protein ratio (w/w) is between 0.8 and 1.2 and the degree of activation of the activated saccharide is between 9.0 to 32%.

2.3 Carrier Protein of the Streptococcus pneumoniae Serotype 9N Glycoconjugates of the Invention

A component of the glycoconjugate is a carrier protein to which the saccharide is conjugated. The terms “protein carrier” or “carrier protein” or “carrier” may be used interchangeably herein. Carrier proteins should be amenable to conjugation procedures.

In a preferred embodiment, the carrier protein of the Streptococcus pneumoniae serotype 9N glycoconjugate of the invention is selected in the group consisting of: DT (Diphtheria toxoid), TT (tetanus toxoid) or fragment C of TT, CRM₁₉₇ (a nontoxic but antigenically identical variant of diphtheria toxin), other DT mutants (such as CRM₁₇₆, CRM₂₂₈, CRM₄₅ (Uchida et al. (1973) J. Biol. Chem. 218:3838-3844), CRM₉, CRM₁₀₂, CRM₁₀₃ or CRM₁₀₇; and other mutations described by Nicholls and Youle in Genetically Engineered Toxins, Ed: Frankel, Maecel Dekker Inc. (1992); deletion or mutation of Glu-148 to Asp, Gln or Ser and/or Ala 158 to Gly and other mutations disclosed in U.S. Pat. Nos. 4,709,017 and 4,950,740; mutation of at least one or more residues Lys 516, Lys 526, Phe 530 and/or Lys 534 and other mutations disclosed in U.S. Pat. Nos. 5,917,017 and 6,455,673; or fragment disclosed in U.S. Pat. No. 5,843,711, pneumococcal pneumolysin (ply) (Kuo et al. (1995) Infect Immun 63:2706-2713) including ply detoxified in some fashion, for example dPLY-GMBS (WO 2004/081515, WO 2006/032499) or dPLY-formol, PhtX, including PhtA, PhtB, PhtD, PhtE (sequences of PhtA, PhtB, PhtD or PhtE are disclosed in WO 00/37105 and WO 00/39299) and fusions of Pht proteins, for example PhtDE fusions, PhtBE fusions, Pht A-E (WO 01/98334, WO 03/054007, WO 2009/000826), OMPC (meningococcal outer membrane protein), which is usually extracted from Neisseria meningitidis serogroup B (EP0372501), PorB (from N. meningitidis), PD (Haemophilus influenzae protein D; see, e.g., EP0594610 B), or immunologically functional equivalents thereof, synthetic peptides (EP0378881, EP0427347), heat shock proteins (WO 93/17712, WO 94/03208), pertussis proteins (WO 98/58668, EP0471177), cytokines, lymphokines, growth factors or hormones (WO 91/01146), artificial proteins comprising multiple human CD4+ T cell epitopes from various pathogen derived antigens (Falugi et al. (2001) Eur J Immunol 31:3816-3824) such as N19 protein (Baraldoi et al. (2004) Infect Immun 72:4884-4887) pneumococcal surface protein PspA (WO 02/091998), iron uptake proteins (WO 01/72337), toxin A or B of Clostridium difficile (WO 00/61761), transferrin binding proteins, pneumococcal adhesion protein (PsaA), recombinant Pseudomonas aeruginosa exotoxin A (in particular non-toxic mutants thereof (such as exotoxin A bearing a substitution at glutamic acid 553 (Douglas et al. (1987) J. Bacteriol. 169(11):4967-4971)). Other proteins, such as ovalbumin, keyhole limpet hemocyanin (KLH), bovine serum albumin (BSA) or purified protein derivative of tuberculin (PPD) also can be used as carrier proteins. Other suitable carrier proteins include inactivated bacterial toxins such as cholera toxoid (e.g., as described in WO 2004/083251), Escherichia coli LT, E. coli ST, and exotoxin A from P. aeruginosa. Another suitable carrier protein is a C5a peptidase from Streptococcus (SCP). Another suitable carrier protein is rhizavidin [aa 45-179J-GGGGSSS-SP1500- AAA-SP0785](CP1) (WO2020056202). Another suitable carrier protein is Rhavi-linker-PdT(G294P)-linker-SP0435 [aa 62-185] fusion protein (SPP2), see WO2023039223. WO2020/056202 and WO2023/039223 are incorporated by reference. SPP2 is described in particular at sections [0245] to [250] of WO2023/039223.

In an embodiment, the carrier protein of the serotype 9N glycoconjugate of the present invention is rhizavidin [aa 45-179J-GGGGSSS-SP1500-AAA-SP0785](CP1).

In another embodiment, the carrier protein of the serotype 9N glycoconjugate of the present invention is Rhavi-linker-PdT(G294P)-linker-SP0435 [aa 62-185] fusion protein (SPP2).

In an embodiment, said SPP2 has the amino acid sequence as set forth at SEQ ID NO: 19 of WO2023/039223.

In a preferred embodiment, the carrier protein of the serotype 9N capsular polysaccharide glycoconjugate of the invention is selected from the group consisting of TT, DT, DT mutants (such as CRM₁₉₇), and a C5a peptidase from Streptococcus (SCP).

In a preferred embodiment, the carrier protein of the serotype 9N glycoconjugate of the present invention is TT, DT, DT mutants (such as CRM₁₉₇) or a C5a peptidase from Streptococcus (SCP).

In an embodiment, the carrier protein of the serotype 9N glycoconjugate of the present invention is DT (Diphtheria toxoid). In another embodiment, the carrier protein of the serotype 9N glycoconjugate of the present invention is TT (tetanus toxoid).

In another embodiment, the carrier protein of the serotype 9N glycoconjugate of the present invention is PD (H. influenzae protein D; see, e.g., EP0594610 B).

In a preferred embodiment, the carrier protein of the serotype 9N glycoconjugate of the present invention is CRM₁₉₇ or a C5a peptidase from Streptococcus (SCP).

In a preferred embodiment, the carrier protein of the serotype 9N glycoconjugate of the present invention is CRM₁₉₇ protein. The CRM₁₉₇ protein is a nontoxic form of diphtheria toxin but is immunologically indistinguishable from the diphtheria toxin. CRM₁₉₇ is produced by Corynebacterium diphtheriae infected by the nontoxigenic phage ρ197^{tox-} created by nitrosoguanidine mutagenesis of the toxigenic corynephage beta (Uchida et al. (1971) Nature New Biology 233:8-11). The CRM₁₉₇ protein has the same molecular weight as the diphtheria toxin but differs therefrom by a single base change (guanine to adenine) in the structural gene. This single base change causes an amino acid substitution (glutamic acid for glycine) in the mature protein and eliminates the toxic properties of diphtheria toxin. The CRM₁₉₇ protein is a safe and effective T-cell dependent carrier for saccharides. Further details about CRM₁₉₇ and production thereof can be found, e.g., in U.S. Pat. No. 5,614,382.

In an embodiment, the carrier protein of the serotype 9N glycoconjugate of the present invention is CRM₁₉₇ protein or the A chain of CRM₁₉₇ (see CN103495161). In an embodiment the carrier protein of the serotype 9N glycoconjugate of the present invention is the A chain of CRM₁₉₇ obtained via expression by genetically recombinant E. coli (see CN103495161).

In very preferred embodiments, the carrier protein of the serotype 9N glycoconjugate of the present invention is SCP (Streptococcal C5a Peptidase).

Two important species of β-hemolytic streptococci, Streptococcus pyogenes (group A Streptococcus, GAS) and Streptococcus agalactiae (group B Streptococcus, GBS), which cause a variety of serious human infections that range from mild cases of pharyngitis and impetigo to serious invasive diseases such as necrotizing fasciitis (GAS) and neonatal sepsis (GBS) have developed a way to defeat this immune response. All human isolates of β-hemolytic streptococci, including GAS and GBS, produce a highly conserved cell-wall protein SCP (Streptococcal C5a Peptidase) that specifically inactivates C5a. The scp genes from GAS and GBS encode a polypeptide containing between 1,134 and 1,181 amino acids (Brown et al., PNAS, 2005, vol. 102, no. 51 pages 18391-18396). The first 31 residues are the export signal presequence and are removed upon passing through the cytoplasmic membrane. The next 68 residues serve as a pro-sequence and must be removed to produce active SCP. The next 10 residues can be removed without loss of protease activity. At the other end, starting with Lys-1034, are four consecutive 17-residue motifs followed by a cell sorting and cell-wall attachment signal. This combined signal is composed of a 20-residue hydrophilic sequence containing an LPTTND sequence, a 17-residue hydrophobic sequence, and a short basic carboxyl terminus.

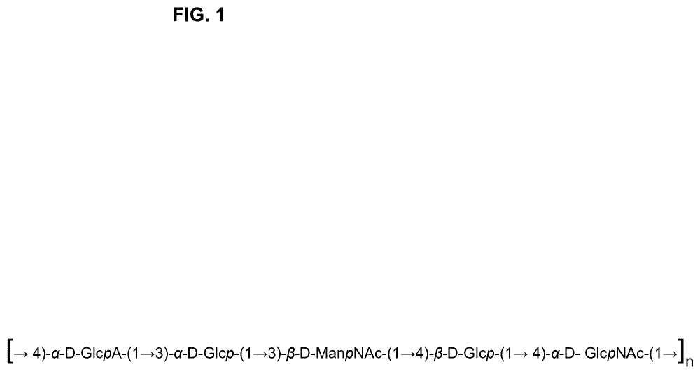

SCP can be divided in domains (see FIG. 1B of Brown et al., PNAS, 2005, vol. 102, no. 51 pages 18391-18396). These domains are the Pre/Pro domain (which comprises the export signal presequence (commonly the first 31 residues) and the pro-sequence (commonly the next 68 residues)), the protease domain (which is splitted in two part (protease part 1 commonly residues 89-333/334 and protease domain part 2 and commonly residues 467/468-583/584), the protease-associated domain (PA domain) (commonly residues 333/334-467/468), three fibronectin type III (Fn) domains (Fn1, commonly residues 583/584-712/713; Fn2, commonly residues 712/713-928/929/930; commonly Fn3, residues 929/930-1029/1030/1031) and a cell wall anchor domain (commonly residues 1029/1030/1031 to the C-terminus).

In an embodiment, the carrier protein of the serotype 9N glycoconjugate of the invention is an SCP from GBS (SCPB). An example of SCPB is provided at SEQ. ID. NO: 3 of WO97/26008. See also SEQ ID NO: 3 of WO00/34487.

In another preferred embodiments, the carrier protein of the serotype 9N glycoconjugate of the invention is an SCP from GAS (SCPA). Examples of SCPA can be found at SEQ. ID. No. 1 and SEQ. ID. No. 2 of WO97/26008. See also SEQ ID NO: 1, 2 and 23 of WO00/34487.

In a preferred embodiment, the carrier protein of the serotype 9N glycoconjugate of the invention is an enzymatically inactive SCP.

In other preferred embodiments, the carrier protein of the serotype 9N glycoconjugate of the invention is an enzymatically inactive SCP from GBS (SCPB).

In another preferred embodiments, the carrier protein of the serotype 9N glycoconjugate of the invention is an enzymatically inactive SCP from GAS (SCPA).

In an embodiment, the carrier protein of the serotype 9N glycoconjugate of the invention is a fragment of an SCP. In an embodiment, the carrier protein of the serotype 9N glycoconjugate of the invention is a fragment of an SCPA. Preferably, the carrier protein of the serotype 9N glycoconjugate of the invention is a fragment of an SCPB.

In an embodiment, the carrier protein of the serotype 9N glycoconjugate of the invention is a fragment of an SCP which comprises the protease domain, the protease-associated domain (PA domain) and the three fibronectin type III (Fn) domains but does not comprise the export signal presequence, the pro-sequence and the cell wall anchor domain.

In an embodiment, the carrier protein of the serotype 9N glycoconjugate of the invention is a fragment of an SCP which comprises the protease domain, the protease-associated domain (PA domain) and the three fibronectin type III (Fn) domains but does not comprise the export signal presequence, the pro-sequence and the cell wall anchor domain.

In an embodiment, the carrier protein of the serotype 9N glycoconjugate of the invention is an enzymatically inactive fragment of an SCP which comprises the protease domain, the protease-associated domain (PA domain) and two of the three fibronectin type III (Fn) domains but does not comprise the export signal presequence, the pro-sequence and the cell wall anchor domain.

In an embodiment, the carrier protein of the serotype 9N glycoconjugate of the invention is an enzymatically inactive fragment of an SCP. In an embodiment, said enzymatically inactive fragment of SCP comprises the protease domain, the protease-associated domain (PA domain) and the three fibronectin type III (Fn) domains but does not comprise the export signal presequence, the pro-sequence and the cell wall anchor domain.

In an embodiment, the carrier protein of the serotype 9N glycoconjugate of the invention is an enzymatically inactive fragment of an SCPA. In an embodiment, said enzymatically inactive fragment of an SCPA comprises the protease domain, the protease-associated domain (PA domain) and the three fibronectin type III (Fn) domains but does not comprise the export signal presequence, the pro-sequence and the cell wall anchor domain.

In a preferred embodiment, the carrier protein of the serotype 9N glycoconjugate of the invention is an enzymatically inactive fragment of SCPB. Preferably, said enzymatically inactive fragment of SCPB comprises the protease domain, the protease-associated domain (PA domain) and the three fibronectin type III (Fn) domains but does not comprise the export signal presequence, the pro-sequence and the cell wall anchor domain.

In an embodiment, the enzymatic activity of SCP is inactivated by replacing at least one amino acid of the wild type sequence. In an embodiment, said replacement is selected from the group consisting of D130A, H193A, N295A and S512A. The numbers indicate the amino acid residue position in the peptidase according to the numbering of SEQ ID NO: 1 of WO00/34487. Therefore, in an embodiment, the carrier protein of the serotype 9N glycoconjugate of the invention is an enzymatically inactive SCP where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the serotype 9N glycoconjugate of the invention is an enzymatically inactive SCPA where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the serotype 9N glycoconjugate of the invention is an enzymatically inactive SCPB where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the serotype 9N glycoconjugate of the invention is an enzymatically inactive fragment of an SCP where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A. In an embodiment, the carrier protein of the serotype 9N glycoconjugate of the invention is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the serotype 9N glycoconjugate of the invention is an enzymatically inactive fragment of SCPA which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the serotype 9N glycoconjugate of the invention is an enzymatically inactive fragment of SCPB which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the enzymatic activity of SCP is inactivated by replacing at least two amino acids of the wild type sequence. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. In an embodiment, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

Therefore, in an embodiment, the carrier protein of the serotype 9N glycoconjugate of the invention is an enzymatically inactive SCP where said inactivation is accomplished by replacing at least two amino acids of the wild type sequence. Preferably, said replacement of at least two amino acids is in the protease domain. In an embodiment, said replacement of at least two amino acid is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the serotype 9N glycoconjugate of the invention is an enzymatically inactive SCPA where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the serotype 9N glycoconjugate of the invention is an enzymatically inactive SCPB where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the serotype 9N glycoconjugate of the invention is an enzymatically inactive fragment of an SCP where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the serotype 9N glycoconjugate of the invention is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the serotype 9N glycoconjugate of the invention is an enzymatically inactive fragment of SCPA which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least one amino acids is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the serotype 9N glycoconjugate of the invention is an enzymatically inactive fragment of SCPB which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acids is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the enzymatic activity of SCP is inactivated by replacing at least three amino acids of the wild type sequence. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

Therefore, in an embodiment, the carrier protein of the serotype 9N glycoconjugate of the invention is an enzymatically inactive SCP where said inactivation is accomplished by replacing at least three amino acids of the wild type sequence. Preferably, said replacement of at least three amino acids is in the protease domain. In an embodiment, said replacement of at least three amino acid is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the serotype 9N glycoconjugate of the invention is an enzymatically inactive SCPA where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the serotype 9N glycoconjugate of the invention is an enzymatically inactive SCPB where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the serotype 9N glycoconjugate of the invention is an enzymatically inactive fragment of an SCP where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the serotype 9N glycoconjugate of the invention is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the serotype 9N glycoconjugate of the invention is an enzymatically inactive fragment of SCPA which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acids is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the serotype 9N glycoconjugate of the invention is an enzymatically inactive fragment of SCPB which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acids is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the enzymatic activity of SCP is inactivated by replacing at least four amino acids of the wild type sequence. In an embodiment, said at least four amino acids replacements are D130A, H193A, N295A and S512A.

Therefore, in an embodiment, the carrier protein of the serotype 9N glycoconjugate of the invention is an enzymatically inactive SCP where said inactivation is accomplished by replacing at least four amino acids of the wild type sequence. Preferably, said replacement of at least four amino acids is in the protease domain. In an embodiment, said replacement of at least four amino acid is in part 1 of the protease domain. In an embodiment, said replacement of at least four amino acid is in part 2 of the protease domain. In an embodiment, said at least four amino acids replacements are D130A, H193A, N295A and S512A

In an embodiment, the carrier protein of the serotype 9N glycoconjugate of the invention is an enzymatically inactive SCPA where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acid is in part 2 of the protease domain. In an embodiment, said at least four amino acids replacements are D130A, H193A, N295A and S512A

In an embodiment, the carrier protein of the serotype 9N glycoconjugate of the invention is an enzymatically inactive SCPB where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acid is in part 2 of the protease domain. In an embodiment, said at least four amino acids replacements are D130A, H193A, N295A and S512A

In an embodiment, the carrier protein of the serotype 9N glycoconjugate of the invention is an enzymatically inactive fragment of an SCP where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acid is in part 2 of the protease domain. In an embodiment, said at least four amino acids replacements are D130A, H193A, N295A and S512A

In an embodiment, the carrier protein of the serotype 9N glycoconjugate of the invention is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acid is in part 2 of the protease domain. In an embodiment, said at least four amino acids replacements are D130A, H193A, N295A and S512A

In an embodiment, the carrier protein of the serotype 9N glycoconjugate of the invention is an enzymatically inactive fragment of SCPA which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least one amino acids is in part 2 of the protease domain. In an embodiment, said at least four amino acids replacements are D130A, H193A, N295A and S512A

In an embodiment, the carrier protein of the serotype 9N glycoconjugate of the invention is an enzymatically inactive fragment of SCPB which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acids is in part 2 of the protease domain. In an embodiment, said at least four amino acids replacements are D130A, H193A, N295A and S512A

In a particular embodiment, the carrier protein of the serotype 9N glycoconjugate of the invention is an enzymatically inactive fragment of SCP which consists of SEQ ID NO: 41.

SEQ ID NO: 41:
MAKTADTPATSKATIRDLNDPSQVKTLQEKAGKGAGTVVAVIAAGFDKNH
EAWRLTDKAKARYQSKEDLEKAKKEHGITYGEWVNDKVAYYHDYSKDGKT
AVDQEHGTHVSGILSGNAPSETKEPYRLEGAMPEAQLLLMRVEIVNGLAD
YARNYAQAIRDAINLGAKVINMSFGNAALAYANLPDETKKAFDYAKSKGV
SIVTSAGNDSSFGGKTRLPLADHPDYGVVGTPAAADSTLTVASYSPDKQL
TETVTVKTADQQDKEMPVLSTNRFEPNKAYDYAYANRGTKEDDFKDVKGK
IALIERGDIDFKDKIAKAKKAGAVGVLIYDNQDKGFPIELPNVDQMPAAF
ISRKDGLLLKDNPQKTITFNATPKVLPTASGTKLSRFSSWGLTADGNIKP
DIAAPGQDILSSVANNKYAKLSGTAMSAPLVAGIMGLLQEQYETQYPDMT
PSERLDLAKKVLMSSATALYDEDEKAYFSPRQQGAGAVDAKKASAATMYV
TDKDNTSSKVHLNNVSDKFEVTVTVHNKSDKPQELYYQATVQTDKVDGKH
FALAPKALYETSWQKITIPANSSKQVTVPIDASRFSKDLLAQMKNGYFLE
GFVRFKQDPKKEELMSIPYIGFRGDFGNLSALEKPIYDSKDGSSYYHEAN
SDAKDQLDGDGLQFYALKNNFTALTTESNPWTIIKAVKEGVENIEDIESS
EITETIFAGTFAKQDDDSHYYIHRHANGKPYAAISPNGDGNRDYVQFQGT
FLRNAKNLVAEVLDKEGNVVWTSEVTEQVVKNYNNDLASTLGSTRFEKTR
WDGKDKDGKWVANGTYTYRVRYTPISSGAKEQHTDFDVIVDNTTPEVATS
ATFSTEDRRLTLASKPKTSQPVYRERIAYTYMDEDLPTTEYISPNEDGTF
TLPEEAETMEGATVPLKMSDFTYWVEDMAGNITYTPVTKLLEGHSNKPEQ

SEQ ID NO: 41 is 950 amino acids long.

In a particular embodiment, the carrier protein of the serotype 9N glycoconjugate of the invention is an enzymatically inactive fragment of SCP which consists of SEQ ID NO: 42.

SEQ ID NO: 42:
AKTADTPATSKATIRDLNDPSQVKTLQEKAGKGAGTVVAVIAAGFDKNH
EAWRLTDKAKARYQSKEDLEKAKKEHGITYGEWVNDKVAYYHDYSKDGKT
AVDQEHGTHVSGILSGNAPSETKEPYRLEGAMPEAQLLLMRVEIVNGLAD
YARNYAQAIRDAINLGAKVINMSFGNAALAYANLPDETKKAFDYAKSKGV
SIVTSAGNDSSFGGKTRLPLADHPDYGWVGTPAAADSTLTVASYSPDKQL
TETVTVKTADQQDKEMPVLSTNRFEPNKAYDYAYANRGTKEDDFKDVKGK
IALIERGDIDFKDKIAKAKKAGAVGVLIYDNQDKGFPIELPNVDQMPAAF
ISRKDGLLLKDNPQKTITFNATPKVLPTASGTKLSRFSSWGLTADGNIKP
DIAAPGQDILSSVANNKYAKLSGTAMSAPLVAGIMGLLQEQYETQYPDMT
PSERLDLAKKVLMSSATALYDEDEKAYFSPRQQGAGAVDAKKASAATMYV
TDKDNTSSKVHLNNVSDKFEVTVTVHNKSDKPQELYYQATVQTDKVDGKH
FALAPKALYETSWQKITIPANSSKQVTVPIDASRFSKDLLAQMKNGYFLE
GFVRFKQDPKKEELMSIPYIGFRGDFGNLSALEKPIYDSKDGSSYYHEAN
SDAKDQLDGDGLQFYALKNNFTALTTESNPWTIIKAVKEGVENIEDIESS
EITETIFAGTFAKQDDDSHYYIHRHANGKPYAAISPNGDGNRDYVQFQGT
FLRNAKNLVAEVLDKEGNVVWTSEVTEQVVKNYNNDLASTLGSTRFEKTR
WDGKDKDGKWVANGTYTYRVRYTPISSGAKEQHTDFDVIVDNTTPEVATS
ATFSTEDRRLTLASKPKTSQPVYRERIAYTYMDEDLPTTEYISPNEDGTF
TLPEEAETMEGATVPLKMSDFTYWVEDMAGNITYTPVTKLLEGHSNKPEQ

SEQ ID NO: 42 is 949 amino acids long.

In a particular embodiment, the carrier protein of the serotype 9N glycoconjugate of the invention is an enzymatically inactive fragment of SCP consisting of a polypeptide having at least 90% identity with SEQ ID NO: 41.

In a particular embodiment, the carrier protein of the serotype 9N glycoconjugate of the invention is an enzymatically inactive fragment of SCP consisting of a polypeptide having at least 95% identity with SEQ ID NO: 41.

In a particular embodiment, the carrier protein of the serotype 9N glycoconjugate of the invention is an enzymatically inactive fragment of SCP consisting of a polypeptide having at least 99% identity with SEQ ID NO: 41.

In a particular embodiment, the carrier protein of the serotype 9N glycoconjugate of the invention is an enzymatically inactive fragment of SCP consisting of a polypeptide having at least 99.5% identity with SEQ ID NO: 41.

In a particular embodiment, the carrier protein of the serotype 9N glycoconjugate of the invention is an enzymatically inactive fragment of SCP consisting of a polypeptide having at least 99.8% identity with SEQ ID NO: 41.

In a particular embodiment, the carrier protein of the serotype 9N glycoconjugate of the invention is an enzymatically inactive fragment of SCP consisting of a polypeptide having at least 99.85% identity with SEQ ID NO: 41.

In a particular embodiment, the carrier protein of the serotype 9N glycoconjugate of the invention is an enzymatically inactive fragment of SCP consisting of a polypeptide having at least 90% identity with SEQ ID NO: 42.

In a particular embodiment, the carrier protein of the serotype 9N glycoconjugate of the invention is an enzymatically inactive fragment of SCP consisting of a polypeptide having at least 95% identity with SEQ ID NO: 42.

In a particular embodiment, the carrier protein of the serotype 9N glycoconjugate of the invention is an enzymatically inactive fragment of SCP consisting of a polypeptide having at least 99% identity with SEQ ID NO: 42.

In a particular embodiment, the carrier protein of the serotype 9N glycoconjugate of the invention is an enzymatically inactive fragment of SCP consisting of a polypeptide having at least 99.5% identity with SEQ ID NO: 42.

In a particular embodiment, the carrier protein of the serotype 9N glycoconjugate of the invention is an enzymatically inactive fragment of SCP consisting of a polypeptide having at least 99.8% identity with SEQ ID NO: 42.

In a particular embodiment, the carrier protein of the serotype 9N glycoconjugate of the invention is an enzymatically inactive fragment of SCP consisting of a polypeptide having at least 99.85% identity with SEQ ID NO: 42.

### 3 Immunogenic Compositions

### 3.1 Combinations of Glycoconjugates of the Invention

In an embodiment the invention relates to an immunogenic composition comprising a Streptococcus pneumoniae serotype 9N glycoconjugate of the invention.

In an embodiment the invention relates to an immunogenic composition comprising a Streptococcus pneumoniae serotype 9N glycoconjugate of the invention and comprising from 1 to 35 different glycoconjugates.

In an embodiment the invention relates to an immunogenic composition comprising a Streptococcus pneumoniae serotype 9N glycoconjugate of the invention and comprising from 1 to 35 glycoconjugates from different serotypes of S. pneumoniae (1 to 35 pneumococcal conjugates). In one embodiment the invention relates to an immunogenic composition comprising glycoconjugates from 7, 8, 9, 10, 11, 12, 13, 14, 15, 16, 17, 18, 19, 20, 21, 22, 23, 24, 25, 26, 27, 28, 29, 30, 31, 32, 33, 34 or 35 different serotypes of S. pneumoniae.

In one embodiment the immunogenic composition comprises glycoconjugates from 21 to 35 different serotypes of S. pneumoniae. In an embodiment the immunogenic composition is a 21-valent pneumococcal conjugate composition. In an embodiment the immunogenic composition is a 22-valent pneumococcal conjugate composition. In an embodiment the immunogenic composition is a 23-valent pneumococcal conjugate composition. In an embodiment the immunogenic composition is a 24-valent pneumococcal conjugate composition. In an embodiment the immunogenic composition is a 25-valent pneumococcal conjugate composition. In an embodiment the immunogenic composition is a 26-valent pneumococcal conjugate composition. In an embodiment the immunogenic composition is a 27-valent pneumococcal conjugate composition. In an embodiment the immunogenic composition is a 28-valent pneumococcal conjugate composition. In an embodiment the immunogenic composition is a 29-valent pneumococcal conjugate composition. In an embodiment the immunogenic composition is a 30-valent pneumococcal conjugate composition. In an embodiment the immunogenic composition is a 31-valent pneumococcal conjugate composition. In an embodiment the immunogenic composition is a 32-valent pneumococcal conjugate composition. In an embodiment the immunogenic composition is a 33-valent pneumococcal conjugate composition. In an embodiment the immunogenic composition is a 34-valent pneumococcal conjugate composition. In an embodiment the immunogenic composition is a 35-valent pneumococcal conjugate composition.

In a preferred embodiment the immunogenic composition is a 35-valent pneumococcal conjugate composition.

In an embodiment the invention relates to an immunogenic composition comprising a Streptococcus pneumoniae serotype 9N glycoconjugate of the invention and further comprising glycoconjugates from S. pneumoniae serotypes 4, 6B, 9V, 14, 18C, 19F and 23F. In an embodiment the immunogenic composition is an 8-valent pneumococcal conjugate compositions.

In an embodiment the invention relates to an immunogenic composition comprising a Streptococcus pneumoniae serotype 9N glycoconjugate of the invention and further comprising glycoconjugates from S. pneumoniae serotypes 1, 4, 5, 6A, 6B, 7F, 9V, 14, 18C, 19F and 23F. In an embodiment the immunogenic composition is a 12-valent pneumococcal conjugate compositions.

In an embodiment the invention relates to an immunogenic composition comprising a Streptococcus pneumoniae serotype 9N glycoconjugate of the invention and further comprising glycoconjugates from S. pneumoniae serotypes 1, 4, 5, 6A, 6B, 7F, 9V, 14, 18C, 19A, 19F and 23F. In an embodiment the immunogenic composition is a 13-valent pneumococcal conjugate compositions.

In an embodiment the invention relates to an immunogenic composition comprising a Streptococcus pneumoniae serotype 9N glycoconjugate of the invention and further comprising glycoconjugates from S. pneumoniae serotypes 1, 3, 4, 5, 6A, 6B, 7F, 9V, 14, 18C, 19A, 19F and 23F. In an embodiment the immunogenic composition is a 14-valent pneumococcal conjugate compositions.

In an embodiment the invention relates to an immunogenic composition comprising a Streptococcus pneumoniae serotype 9N glycoconjugate of the invention and further comprising glycoconjugates from S. pneumoniae serotypes 1, 3, 4, 5, 6A, 6B, 7F, 9V, 14, 18C, 19A, 19F, 22F, 23F and 33F. In an embodiment the immunogenic composition is a 16-valent pneumococcal conjugate compositions.

In an embodiment the invention relates to an immunogenic composition comprising a Streptococcus pneumoniae serotype 9N glycoconjugate of the invention and further comprising glycoconjugates from S. pneumoniae serotypes 1, 2, 3, 4, 5, 6A, 6B, 7F, 9V, 14, 18C, 19A, 19F, 22F, 23F and 33F. In an embodiment the immunogenic composition is a 17-valent pneumococcal conjugate compositions.

In an embodiment the invention relates to an immunogenic composition comprising a Streptococcus pneumoniae serotype 9N glycoconjugate of the invention and further comprising glycoconjugates from S. pneumoniae serotypes 1, 3, 4, 5, 6A, 6B, 8, 7F, 9V, 10A, 11A, 12F, 14, 15B, 18C, 19A, 19F, 22F, 23F and 33F. In an embodiment the immunogenic composition is a 21-valent pneumococcal conjugate compositions.

In an embodiment the invention relates to an immunogenic composition comprising a Streptococcus pneumoniae serotype 9N glycoconjugate of the invention and further comprising glycoconjugates from S. pneumoniae serotypes 1, 2, 3, 4, 5, 6A, 6B, 8, 7F, 9V, 10A, 11A, 12F, 14, 15B, 18C, 19A, 19F, 22F, 23F and 33F. In an embodiment the immunogenic composition is a 22-valent pneumococcal conjugate compositions.

In an embodiment the invention relates to an immunogenic composition comprising a Streptococcus pneumoniae serotype 9N glycoconjugate of the invention and further comprising glycoconjugates from S. pneumoniae serotypes 1, 2, 3, 4, 5, 6A, 6B, 8, 7F, 9V, 10A, 11A, 12F, 14, 15B, 18C, 19A, 19F, 20B, 22F, 23F and 33F. In an embodiment the immunogenic composition is a 23-valent pneumococcal conjugate compositions.

In an embodiment the invention relates to an immunogenic composition comprising a Streptococcus pneumoniae serotype 9N glycoconjugate of the invention and further comprising glycoconjugates from S. pneumoniae serotypes 1, 2, 3, 4, 5, 6A, 6B, 7F, 8, 9V, 10A, 11A, 12F, 14, 15B, 17F, 18C, 19A, 19F, 22F, 23F and 33F. In an embodiment the immunogenic composition is a 23-valent pneumococcal conjugate compositions.

In an embodiment the invention relates to an immunogenic composition comprising a Streptococcus pneumoniae serotype 9N glycoconjugate of the invention and further comprising glycoconjugates from S. pneumoniae serotypes 1, 2, 3, 4, 5, 6B, 8, 7F, 9V, 10A, 11A, 12F, 14, 15B, 17F, 18C, 19A, 19F, 20, 22F, 23F and 33F. In an embodiment the immunogenic composition is a 23-valent pneumococcal conjugate compositions.

In an embodiment the invention relates to an immunogenic composition comprising a Streptococcus pneumoniae serotype 9N glycoconjugate of the invention and further comprising glycoconjugates from S. pneumoniae serotypes 1, 2, 3, 4, 5, 6A, 6B, 8, 7F, 9V, 10A, 11A, 12F, 14, 15B, 17F, 18C, 19A, 19F, 20, 22F, 23F and 33F. In an embodiment the immunogenic composition is a 24-valent pneumococcal conjugate compositions.

In an embodiment the invention relates to an immunogenic composition comprising a Streptococcus pneumoniae serotype 9N glycoconjugate of the invention and further comprising glycoconjugates from S. pneumoniae serotypes 1, 2, 3, 4, 5, 6A, 6B, 8, 7F, 9V, 10A, 11A, 12F, 14, 15B, 17F, 18C, 19A, 19F, 20B, 22F, 23F and 33F. In an embodiment the immunogenic composition is a 24-valent pneumococcal conjugate compositions.

In an embodiment the invention relates to an immunogenic composition comprising a Streptococcus pneumoniae serotype 9N glycoconjugate of the invention and further comprising glycoconjugates from S. pneumoniae serotypes 3, 6A, 7F, 8, 10A, 11A, 12F, 15A, 15C, 16F, 17F, 19A, 20, 22F, 23A, 23B, 24F, 31, 33F and 35B. In an embodiment the immunogenic composition is a 21-valent pneumococcal conjugate compositions.

In an embodiment the invention relates to an immunogenic composition comprising a Streptococcus pneumoniae serotype 9N glycoconjugate of the invention and further comprising glycoconjugates from S. pneumoniae serotypes 3, 6A, 7F, 8, 10A, 11A, 12F, 15A, 15B, 16F, 17F, 19A, 20, 22F, 23A, 23B, 24F, 31, 33F and 35B. In an embodiment the immunogenic composition is a 21-valent pneumococcal conjugate compositions.

In an embodiment the invention relates to an immunogenic composition comprising a Streptococcus pneumoniae serotype 9N glycoconjugate of the invention and further comprising glycoconjugates from S. pneumoniae serotypes 3, 6A, 7F, 8, 10A, 11A, 12F, 15A, 15C, 16F, 17F, 19A, 20B, 22F, 23A, 23B, 24F, 31, 33F and 35B. In an embodiment the immunogenic composition is a 21-valent pneumococcal conjugate compositions.

In an embodiment the invention relates to an immunogenic composition comprising a Streptococcus pneumoniae serotype 9N glycoconjugate of the invention and further comprising glycoconjugates from S. pneumoniae serotypes 3, 6A, 7F, 8, 10A, 11A, 12F, 15A, 15B, 16F, 17F, 19A, 20, 22F, 23A, 23B, 24F, 31, 33F and 35B. In an embodiment the immunogenic composition is a 21-valent pneumococcal conjugate compositions.

In an embodiment the invention relates to an immunogenic composition comprising a Streptococcus pneumoniae serotype 9N glycoconjugate of the invention and further comprising glycoconjugates from S. pneumoniae serotypes 1, 3, 4, 5, 6A, 6B, 8, 7F, 9V, 10A, 11A, 12F, 14, 15A, 15B, 18C, 19A, 19F, 22F, 23A, 23B, 23F, 24F, 33F and 35B. In an embodiment the immunogenic composition is a 26-valent pneumococcal conjugate compositions.

In an embodiment the invention relates to an immunogenic composition comprising a Streptococcus pneumoniae serotype 9N glycoconjugate of the invention and further comprising glycoconjugates from S. pneumoniae serotypes 1, 3, 4, 5, 6A, 6B, 8, 7F, 9V, 10A, 11A, 12F, 14, 15A, 15B, 18C, 19A, 19F, 22F, 23A, 23B, 23F, 24F, 33F and 35B and additionally comprising at least one glycoconjugate from S. pneumoniae serotypes 7C, 17F, 20B, 21, 27, 31, 34, 35F and 38. In an embodiment the immunogenic composition is a 27-valent pneumococcal conjugate composition.

In an embodiment the invention relates to an immunogenic composition comprising a Streptococcus pneumoniae serotype 9N glycoconjugate of the invention and further comprising glycoconjugates from S. pneumoniae serotypes 1, 3, 4, 5, 6A, 6B, 8, 7F, 9V, 10A, 11A, 12F, 14, 15A, 15B, 18C, 19A, 19F, 22F, 23A, 23B, 23F, 24F, 33F and 35B and additionally comprising at least two glycoconjugates from S. pneumoniae serotypes 7C, 17F, 20B, 21, 27, 31, 34, 35F and 38. In an embodiment the immunogenic composition is a 28-valent pneumococcal conjugate composition.

In an embodiment the invention relates to an immunogenic composition comprising a Streptococcus pneumoniae serotype 9N glycoconjugate of the invention and further comprising glycoconjugates from S. pneumoniae serotypes 1, 3, 4, 5, 6A, 6B, 8, 7F, 9V, 10A, 11A, 12F, 14, 15A, 15B, 18C, 19A, 19F, 22F, 23A, 23B, 23F, 24F, 33F and 35B and additionally comprising at least three glycoconjugates from S. pneumoniae serotypes 7C, 17F, 20B, 21, 27, 31, 34, 35F and 38. In an embodiment the immunogenic composition is a 29-valent pneumococcal conjugate composition.

In an embodiment the invention relates to an immunogenic composition comprising a Streptococcus pneumoniae serotype 9N glycoconjugate of the invention and further comprising glycoconjugates from S. pneumoniae serotypes 1, 3, 4, 5, 6A, 6B, 8, 7F, 9V, 10A, 11A, 12F, 14, 15A, 15B, 18C, 19A, 19F, 22F, 23A, 23B, 23F, 24F, 33F and 35B and additionally comprising at least four glycoconjugates from S. pneumoniae serotypes 7C, 17F, 20B, 21, 27, 31, 34, 35F and 38. In an embodiment the immunogenic composition is a 30-valent pneumococcal conjugate composition.

In an embodiment the invention relates to an immunogenic composition comprising a Streptococcus pneumoniae serotype 9N glycoconjugate of the invention and further comprising glycoconjugates from S. pneumoniae serotypes 1, 3, 4, 5, 6A, 6B, 8, 7F, 9V, 10A, 11A, 12F, 14, 15A, 15B, 18C, 19A, 19F, 22F, 23A, 23B, 23F, 24F, 33F and 35B and additionally comprising at least five glycoconjugates from S. pneumoniae serotypes 7C, 17F, 20B, 21, 27, 31, 34, 35F and 38. In an embodiment the immunogenic composition is a 31-valent pneumococcal conjugate composition.

In an embodiment the invention relates to an immunogenic composition comprising a Streptococcus pneumoniae serotype 9N glycoconjugate of the invention and further comprising glycoconjugates from S. pneumoniae serotypes 1, 3, 4, 5, 6A, 6B, 8, 7F, 9V, 10A, 11A, 12F, 14, 15A, 15B, 18C, 19A, 19F, 22F, 23A, 23B, 23F, 24F, 33F and 35B and additionally comprising at least six glycoconjugates from S. pneumoniae serotypes 7C, 17F, 20B, 21, 27, 31, 34, 35F and 38. In an embodiment the immunogenic composition is a 32-valent pneumococcal conjugate composition.

In an embodiment the invention relates to an immunogenic composition comprising a Streptococcus pneumoniae serotype 9N glycoconjugate of the invention and further comprising glycoconjugates from S. pneumoniae serotypes 1, 3, 4, 5, 6A, 6B, 8, 7F, 9V, 10A, 11A, 12F, 14, 15A, 15B, 18C, 19A, 19F, 22F, 23A, 23B, 23F, 24F, 33F and 35B and additionally comprising at least seven glycoconjugates from S. pneumoniae serotypes 7C, 17F, 20B, 21, 27, 31, 34, 35F and 38. In an embodiment the immunogenic composition is a 33-valent pneumococcal conjugate composition.

In an embodiment the invention relates to an immunogenic composition comprising a Streptococcus pneumoniae serotype 9N glycoconjugate of the invention and further comprising glycoconjugates from S. pneumoniae serotypes 1, 3, 4, 5, 6A, 6B, 8, 7F, 9V, 10A, 11A, 12F, 14, 15A, 15B, 18C, 19A, 19F, 22F, 23A, 23B, 23F, 24F, 33F and 35B and additionally comprising at least eight glycoconjugates from S. pneumoniae serotypes 7C, 17F, 20B, 21, 27, 31, 34, 35F and 38. In an embodiment the immunogenic composition is a 34-valent pneumococcal conjugate composition.

In an embodiment the invention relates to an immunogenic composition comprising a Streptococcus pneumoniae serotype 9N glycoconjugate of the invention and further comprising glycoconjugates from S. pneumoniae serotypes 1, 3, 4, 5, 6A, 6B, 8, 7F, 9V, 10A, 11A, 12F, 14, 15A, 15B, 18C, 19A, 19F, 22F, 23A, 23B, 23F, 24F, 33F and 35B and additionally comprising at least eight glycoconjugates from S. pneumoniae serotypes 7C, 17F, 20B, 21, 27, 31, 34, 35F and 38. In an embodiment the immunogenic composition is a 35-valent pneumococcal conjugate composition.

In a preferred embodiment the invention relates to an immunogenic composition comprising a Streptococcus pneumoniae serotype 9N glycoconjugate of the invention and further comprising glycoconjugates from S. pneumoniae serotypes 1, 3, 4, 5, 6A, 6B, 8, 7C, 7F, 9V, 10A, 11A, 12F, 14, 15A, 15B, 17F, 18C, 19A, 19F, 20B, 21, 22F, 23A, 23B, 23F, 24F, 27, 31, 33F, 34, 35B, 35F and 38. In an embodiment the immunogenic composition is a 35-valent pneumococcal conjugate composition.

In an embodiment the invention relates to an immunogenic composition comprising a Streptococcus pneumoniae serotype 9N glycoconjugate of the invention and further comprising glycoconjugates from S. pneumoniae serotypes 1, 2, 3, 4, 5, 6A, 6B, 8, 7C, 7F, 9V, 10A, 11A, 12F, 14, 15A, 15B, 17F, 18C, 19A, 19F, 20, 22F, 23A, 23B, 23F, 27, 31, 33F and 35B. In an embodiment the immunogenic composition is a 31-valent pneumococcal conjugate composition.

In an embodiment the invention relates to an immunogenic composition comprising a Streptococcus pneumoniae serotype 9N glycoconjugate of the invention and further comprising glycoconjugates from S. pneumoniae serotypes 1, 2, 3, 4, 5, 6A, 6B, 8, 7C, 7F, 9V, 10A, 11A, 12F, 14, 15A, 15B, 17F, 18C, 19A, 19F, 20A, 22F, 23A, 23B, 23F, 27, 31, 33F and 35B. In an embodiment the immunogenic composition is a 31-valent pneumococcal conjugate composition.

In an embodiment the invention relates to an immunogenic composition comprising a Streptococcus pneumoniae serotype 9N glycoconjugate of the invention and further comprising glycoconjugates from S. pneumoniae serotypes 1, 2, 3, 4, 5, 6A, 6B, 8, 7C, 7F, 9V, 10A, 11A, 12F, 14, 15A, 15B, 17F, 18C, 19A, 19F, 20B, 22F, 23A, 23B, 23F, 27, 31, 33F and 35B. In an embodiment the immunogenic composition is a 31-valent pneumococcal conjugate composition.

In an embodiment the invention relates to an immunogenic composition comprising a Streptococcus pneumoniae serotype 9N glycoconjugate of the invention and further comprising glycoconjugates from S. pneumoniae serotypes 1, 2, 3, 4, 5, 6A, 6B, 6C, 8, 7C, 7F, 9V, 10A, 11A, 12F, 14, 15A, 15B, 16F, 17F, 18C, 19A, 19F, 20, 22F, 23A, 23B, 23F, 24F, 31, 33F, 35B and 38.

In an embodiment the immunogenic composition is a 34-valent pneumococcal conjugate composition.

In an embodiment the invention relates to an immunogenic composition comprising a Streptococcus pneumoniae serotype 9N glycoconjugate of the invention and further comprising glycoconjugates from S. pneumoniae serotypes 1, 2, 3, 4, 5, 6A, 6B, 6C, 8, 7C, 7F, 9V, 10A, 11A, 12F, 14, 15A, 15B, 16F, 17F, 18C, 19A, 19F, 20B, 22F, 23A, 23B, 23F, 24F, 31, 33F, 35B and 38. In an embodiment the immunogenic composition is a 34-valent pneumococcal conjugate composition.

In an embodiment the invention relates to an immunogenic composition comprising a Streptococcus pneumoniae serotype 9N glycoconjugate of the invention and further comprising glycoconjugates from S. pneumoniae serotypes 1, 2, 3, 4, 5, 6A, 6B, 6C, 8, 7C, 7F, 9V, 10A, 11A, 12F, 14, 15A, 15B, 16F, 17F, 18C, 19A, 19F, 20A, 22F, 23A, 23B, 23F, 24F, 31, 33F, 35B and 38. In an embodiment the immunogenic composition is a 34-valent pneumococcal conjugate composition.

In a preferred embodiment, the saccharides are each individually conjugated to different molecules of the protein carrier (each molecule of protein carrier only having one type of saccharide conjugated to it). In said embodiment, the capsular saccharides are said to be individually conjugated to the carrier protein. Preferably, all the glycoconjugates of the above immunogenic compositions are individually conjugated to the carrier protein.

In an embodiment of any of the above immunogenic compositions, the glycoconjugate from S. pneumoniae serotype 9N is conjugated to CRM₁₉₇. In an embodiment of any of the above immunogenic compositions, the glycoconjugate from S. pneumoniae serotype 9N is conjugated to SCP.

In an embodiment of any of the above immunogenic compositions, the glycoconjugate from S. pneumoniae serotype 22F is conjugated to CRM₁₉₇. In an embodiment of any of the above immunogenic compositions, the glycoconjugate from S. pneumoniae serotype 33F is conjugated to CRM₁₉₇. In an embodiment of any of the above immunogenic compositions, the glycoconjugate from S. pneumoniae serotype 15B is conjugated to CRM₁₉₇. In an embodiment of any of the above immunogenic compositions, the glycoconjugate from S. pneumoniae serotype 12F is conjugated to CRM₁₉₇. In an embodiment of any of the above immunogenic compositions, the glycoconjugate from S. pneumoniae serotype 10A is conjugated to CRM197. In an embodiment of any of the above immunogenic compositions, the glycoconjugate from S. pneumoniae serotype 11A is conjugated to CRM₁₉₇. In an embodiment of any of the above immunogenic compositions, the glycoconjugate from S. pneumoniae serotype 8 is conjugated to CRM₁₉₇. In an embodiment of any of the above immunogenic compositions, the glycoconjugates from S. pneumoniae serotypes 4, 6B, 9V, 14, 18C, 19F and 23F are conjugated to CRM₁₉₇. In an embodiment of any of the above immunogenic compositions, the glycoconjugates from S. pneumoniae serotypes 1, 5 and 7F are conjugated to CRM₁₉₇. In an embodiment of any of the above immunogenic compositions, the glycoconjugates from S. pneumoniae serotypes 6A and 19A are conjugated to CRM₁₉₇.

In an embodiment of any of the above immunogenic compositions, the glycoconjugates of any of the above immunogenic compositions are all individually conjugated to CRM₁₉₇.

In an embodiment of any of the above immunogenic compositions, the glycoconjugate from S. pneumoniae serotype 9N is conjugated to SCP and the other glycoconjugate(s) is/are all individually conjugated to CRM₁₉₇.

In an embodiment of any of the above immunogenic compositions, the glycoconjugate from S. pneumoniae serotype 9N is conjugated to SCP, at least one other glycoconjugate is conjugated to TT and the other glycoconjugate(s) is/are all individually conjugated to CRM₁₉₇.

In an embodiment of any of the above immunogenic compositions, the glycoconjugate from S. pneumoniae serotype 9N is conjugated to SCP, one other glycoconjugate is conjugated to TT and the other glycoconjugate(s) is/are all individually conjugated to CRM₁₉₇.

In an embodiment of any of the above immunogenic compositions, the glycoconjugate from S. pneumoniae serotype 9N is conjugated to SCP, at least two other glycoconjugates are conjugated to TT and the other glycoconjugate(s) is/are all individually conjugated to CRM₁₉₇.

In an embodiment of any of the above immunogenic compositions, the glycoconjugate from S. pneumoniae serotype 9N is conjugated to SCP, two other glycoconjugates are conjugated to TT and the other glycoconjugate(s) is/are all individually conjugated to CRM₁₉₇.

In an embodiment of any of the above immunogenic compositions, the glycoconjugate from S. pneumoniae serotype 9N is conjugated to SCP, at least three other glycoconjugates are conjugated to TT and the other glycoconjugate(s) is/are all individually conjugated to CRM₁₉₇.

In an embodiment of any of the above immunogenic compositions, the glycoconjugate from S. pneumoniae serotype 9N is conjugated to SCP, three other glycoconjugates are conjugated to TT and the other glycoconjugate(s) is/are all individually conjugated to CRM₁₉₇.

In an embodiment of any of the above immunogenic compositions, the glycoconjugate from S. pneumoniae serotype 9N is conjugated to SCP, at least four other glycoconjugates are conjugated to TT and the other glycoconjugate(s) is/are all individually conjugated to CRM₁₉₇.

In an embodiment of any of the above immunogenic compositions, the glycoconjugate from S. pneumoniae serotype 9N is conjugated to SCP, four other glycoconjugates are conjugated to TT and the other glycoconjugate(s) is/are all individually conjugated to CRM₁₉₇.

In an embodiment of any of the above immunogenic compositions, the glycoconjugate from S. pneumoniae serotype 9N is conjugated to SCP, at least five other glycoconjugates are conjugated to TT and the other glycoconjugate(s) is/are all individually conjugated to CRM₁₉₇.

In an embodiment of any of the above immunogenic compositions, the glycoconjugate from S. pneumoniae serotype 9N is conjugated to SCP, five other glycoconjugates are conjugated to TT and the other glycoconjugate(s) is/are all individually conjugated to CRM₁₉₇.

In an embodiment of any of the above immunogenic compositions, the glycoconjugate from S. pneumoniae serotype 9N is conjugated to SCP, at least one other glycoconjugate is conjugated to SCP and the other glycoconjugate(s) is/are all individually conjugated to CRM₁₉₇.

In an embodiment of any of the above immunogenic compositions, the glycoconjugate from S. pneumoniae serotype 9N is conjugated to SCP, one other glycoconjugate is conjugated to SCP and the other glycoconjugate(s) is/are all individually conjugated to CRM₁₉₇.

In an embodiment of any of the above immunogenic compositions, the glycoconjugate from S. pneumoniae serotype 9N is conjugated to SCP, at least two other glycoconjugates are conjugated to SCP and the other glycoconjugate(s) is/are all individually conjugated to CRM₁₉₇.

In an embodiment of any of the above immunogenic compositions, the glycoconjugate from S. pneumoniae serotype 9N is conjugated to SCP, two other glycoconjugates are conjugated to SCP and the other glycoconjugate(s) is/are all individually conjugated to CRM₁₉₇.

In an embodiment of any of the above immunogenic compositions, the glycoconjugate from S. pneumoniae serotype 9N is conjugated to SCP, at least three other glycoconjugates are conjugated to SCP and the other glycoconjugate(s) is/are all individually conjugated to CRM₁₉₇.

In an embodiment of any of the above immunogenic compositions, the glycoconjugate from S. pneumoniae serotype 9N is conjugated to SCP, three other glycoconjugates are conjugated to SCP and the other glycoconjugate(s) is/are all individually conjugated to CRM₁₉₇.

In an embodiment of any of the above immunogenic compositions, the glycoconjugate from S. pneumoniae serotype 9N is conjugated to SCP, at least four other glycoconjugates are conjugated to SCP and the other glycoconjugate(s) is/are all individually conjugated to CRM₁₉₇.

In an embodiment of any of the above immunogenic compositions, the glycoconjugate from S. pneumoniae serotype 9N is conjugated to SCP, four other glycoconjugates are conjugated to SCP and the other glycoconjugate(s) is/are all individually conjugated to CRM₁₉₇.

In an embodiment of any of the above immunogenic compositions, the glycoconjugate from S. pneumoniae serotype 9N is conjugated to SCP, at least five other glycoconjugates are conjugated to SCP and the other glycoconjugate(s) is/are all individually conjugated to CRM₁₉₇.

In an embodiment of any of the above immunogenic compositions, the glycoconjugate from S. pneumoniae serotype 9N is conjugated to SCP, five other glycoconjugates are conjugated to SCP and the other glycoconjugate(s) is/are all individually conjugated to CRM₁₉₇.

Compositions of the invention may include a small amount of free carrier. When a given carrier protein is present in both free and conjugated form in a composition of the invention, the unconjugated form is preferably no more than 5% of the total amount of the carrier protein in the composition as a whole, and more preferably present at less than 2% by weight.

### 3.2 Dosage of the Immunogenic Compositions of the Invention

The amount of glycoconjugate(s) in each dose is selected as an amount which induces an immunoprotective response without significant, adverse side effects in typical vaccinees. Such amount will vary depending upon which specific immunogen is employed and how it is presented.

The amount of a particular glycoconjugate in an immunogenic composition can be calculated based on total polysaccharide for that conjugate (conjugated and non-conjugated). For example, a glycoconjugate with 20% free polysaccharide will have about 80 μg of conjugated polysaccharide and about 20 μg of nonconjugated polysaccharide in a 100 μg polysaccharide dose. The amount of glycoconjugate can vary depending upon the pneumococcal serotype. The saccharide concentration can be determined by the uronic acid assay.

The “immunogenic amount” of the different polysaccharide components in the immunogenic composition, may diverge and each may comprise about 0.5 μg, about 0.75 μg, about 1 μg, about 2 μg, about 3 μg, about 4 μg, about 5 μg, about 6 μg, about 7 μg, about 8 μg, about 9 μg, about 10 μg, about 15 μg, about 20 μg, about 30 μg, about 40 μg, about 50 μg, about 60 μg, about 70 μg, about 80 μg, about 90 μg, or about 100 μg of any particular polysaccharide antigen.

Generally, each dose will comprise 0.1 μg to 100 μg of serotype 9N polysaccharide. In an embodiment each dose will comprise 0.1 μg to 100 μg of serotype 9N polysaccharide. In a preferred embodiment each dose will comprise 0.5 μg to 20 μg. In a preferred embodiment each dose will comprise 1.0 μg to 10 μg. In an even preferred embodiment, each dose will comprise 2.0 μg to 5.0 μg of serotype 9N polysaccharide. Any whole number integer within any of the above ranges is contemplated as an embodiment of the disclosure.

In an embodiment, each dose will comprise about 0.5 μg of serotype 9N polysaccharide.

In an embodiment, each dose will comprise about 0.55 μg of serotype 9N polysaccharide. In an embodiment, each dose will comprise about 0.75 μg of serotype 9N polysaccharide. In an embodiment, each dose will comprise about 1.0 μg of serotype 9N polysaccharide.

In an embodiment, each dose will comprise about 1.1 μg of serotype 9N polysaccharide. In an embodiment, each dose will comprise about 1.5 μg of serotype 9N polysaccharide. In an embodiment, each dose will comprise about 2.0 μg of serotype 9N polysaccharide. In an embodiment, each dose will comprise about 2.2 μg of serotype 9N polysaccharide. In an embodiment, each dose will comprise about 2.5 μg of serotype 9N polysaccharide. In an embodiment, each dose will comprise about 3.0 μg of serotype 9N polysaccharide. In an embodiment, each dose will comprise about 3.5 μg of serotype 9N polysaccharide. In an embodiment, each dose will comprise about 4.0 μg of serotype 9N polysaccharide. In an embodiment, each dose will comprise about 4.4 μg of serotype 9N polysaccharide. In an embodiment, each dose will comprise about 5.0 μg of serotype 9N polysaccharide. In an embodiment, each dose will comprise about 5.5 μg of serotype 9N polysaccharide. In an embodiment, each dose will comprise about 6.0 μg of serotype 9N polysaccharide.

Generally, each dose will comprise 0.1 μg to 100 μg of polysaccharide for a given serotype. In an embodiment each dose will comprise 0.1 μg to 100 μg of polysaccharide for a given serotype. In a preferred embodiment each dose will comprise 0.5 μg to 20 μg. In a preferred embodiment each dose will comprise 1.0 μg to 10 μg. In an even preferred embodiment, each dose will comprise 2.0 μg to 5.0 μg of polysaccharide for a given serotype. Any whole number integer within any of the above ranges is contemplated as an embodiment of the disclosure.

In an embodiment, each dose will comprise about 0.5 μg of polysaccharide for each particular glycoconjugate. In an embodiment, each dose will comprise about 0.55 μg of polysaccharide for each particular glycoconjugate. In an embodiment, each dose will comprise about 0.75 μg of polysaccharide for each particular glycoconjugate. In an embodiment, each dose will comprise about 1.0 μg of polysaccharide for each particular glycoconjugate. In an embodiment, each dose will comprise about 1.1 μg of polysaccharide for each particular glycoconjugate. In an embodiment, each dose will comprise about 1.5 μg of polysaccharide for each particular glycoconjugate. In an embodiment, each dose will comprise about 2.0 μg of polysaccharide for each particular glycoconjugate. In an embodiment, each dose will comprise about 2.2 μg of polysaccharide for each particular glycoconjugate. In an embodiment, each dose will comprise about 2.5 μg of polysaccharide for each particular glycoconjugate. In an embodiment, each dose will comprise about 3.0 μg of polysaccharide for each particular glycoconjugate. In an embodiment, each dose will comprise about 3.5 μg of polysaccharide for each particular glycoconjugate. In an embodiment, each dose will comprise about 4.0 μg of polysaccharide for each particular glycoconjugate. In an embodiment, each dose will comprise about 4.4 μg of polysaccharide for each particular glycoconjugate. In an embodiment, each dose will comprise about 5.0 μg of polysaccharide for each particular glycoconjugate. In an embodiment, each dose will comprise about 5.5 μg of polysaccharide for each particular glycoconjugate. In an embodiment, each dose will comprise about 6.0 μg of polysaccharide for each particular glycoconjugate.

### 3.3 Carrier Amount

Generally, each dose will comprise 10 μg to 150 μg of carrier protein (total amount), particularly 15 μg to 100 μg of carrier protein, more particularly 25 μg to 75 μg of carrier protein, and even more particularly 40 μg to 60 μg of carrier protein. In an embodiment, said carrier protein is CRM₁₉₇. In an embodiment, said carrier protein is SCP.

In an embodiment, said carrier protein is CRM₁₉₇.

In an embodiment, said carrier protein is SCP.

In an embodiment, said carrier protein is CRM₁₉₇ and SCP.

### 3.4 Further Antigens

Immunogenic compositions of the invention comprise conjugated S. pneumoniae saccharide antigen(s) (glycoconjugate(s)). They may also further include antigen(s) from other pathogen(s), particularly from bacteria and/or viruses. Preferred further antigens are selected from: a diphtheria toxoid (D), a tetanus toxoid (T), a pertussis antigen (P), which is typically acellular (Pa), a hepatitis B virus (HBV) surface antigen (HBsAg), a hepatitis A virus (HAV) antigen, a conjugated Haemophilus influenzae type b capsular saccharide (Hib), inactivated poliovirus vaccine (IPV). In an embodiment, the immunogenic compositions of the invention comprise D-T-Pa. In an embodiment, the immunogenic compositions of the invention comprise D-T-Pa-Hib, D-T-Pa-IPV or D-T-Pa-HBsAg. In an embodiment, the immunogenic compositions of the invention comprise D-T-Pa-HBsAg-IPV or D-T-Pa-HBsAg-Hib. In an embodiment, the immunogenic compositions of the invention comprise D-T-Pa-HBsAg—IPV-Hib.

Pertussis antigens: Bordetella pertussis causes whooping cough. Pertussis antigens in vaccines are either cellular (whole cell, in the form of inactivated B. pertussis cells) or acellular. Preparation of cellular pertussis antigens is well documented (e.g., it may be obtained by heat inactivation of phase I culture of B. pertussis). Preferably, however, the invention uses acellular antigens. Where acellular antigens are used, it is preferred to use one, two or (preferably) three of the following antigens: (1) detoxified pertussis toxin (pertussis toxoid, or PT); (2) filamentous hemagglutinin (FHA); (3) pertactin (also known as the 69 kilodalton outer membrane protein). FHA and pertactin may be treated with formaldehyde prior to use according to the invention. PT is preferably detoxified by treatment with formaldehyde and/or glutaraldehyde. Acellular pertussis antigens are preferably adsorbed onto one or more aluminum salt adjuvants. As an alternative, they may be added in an unadsorbed state. Where pertactin is added then it is preferably already adsorbed onto an aluminum hydroxide adjuvant. PT and FHA may be adsorbed onto an aluminum hydroxide adjuvant or an aluminum phosphate. Adsorption of all of PT, FHA and pertactin to aluminum hydroxide is most preferred.

Inactivated poliovirus vaccine: Poliovirus causes poliomyelitis. Rather than use oral poliovirus vaccine, preferred embodiments of the invention use IPV. Prior to administration to patients, polioviruses must be inactivated, and this can be achieved by treatment with formaldehyde.

Poliomyelitis can be caused by one of three types of poliovirus. The three types are similar and cause identical symptoms, but they are antigenically different and infection by one type does not protect against infection by others. It is therefore preferred to use three poliovirus antigens in the invention: poliovirus Type 1 (e.g., Mahoney strain), poliovirus Type 2 (e.g., MEF-1 strain), and poliovirus Type 3 (e.g., Saukett strain). The viruses are preferably grown, purified and inactivated individually, and are then combined to give a bulk trivalent mixture for use with the invention.

Diphtheria toxoid: Corynebacterium diphtheriae causes diphtheria. Diphtheria toxin can be treated (e.g., using formalin or formaldehyde) to remove toxicity while retaining the ability to induce specific anti-toxin antibodies after injection. These diphtheria toxoids are used in diphtheria vaccines. Preferred diphtheria toxoids are those prepared by formaldehyde treatment. The diphtheria toxoid can be obtained by growing C. diphtheriae in growth medium, followed by formaldehyde treatment, ultrafiltration and precipitation. The toxoided material may then be treated by a process comprising sterile filtration and/or dialysis. The diphtheria toxoid is preferably adsorbed onto an aluminum hydroxide adjuvant.

Tetanus toxoid: Clostridium tetani causes tetanus. Tetanus toxin can be treated to give a protective toxoid. The toxoids are used in tetanus vaccines. Preferred tetanus toxoids are those prepared by formaldehyde treatment. The tetanus toxoid can be obtained by growing C. tetani in growth medium, followed by formaldehyde treatment, ultrafiltration and precipitation. The material may then be treated by a process comprising sterile filtration and/or dialysis.

Hepatitis A virus antigens: Hepatitis A virus (HAV) is one of the known agents which causes viral hepatitis. A preferred HAV component is based on inactivated virus, and inactivation can be achieved by formalin treatment.

Hepatitis B virus (HBV) is one of the known agents which causes viral hepatitis. The major component of the capsid is a protein known as HBV surface antigen or, more commonly, HBsAg, which is typically a 226-amino acid polypeptide with a molecular weight of ˜24 kDa. All existing hepatitis B vaccines contain HBsAg, and when this antigen is administered to a normal vaccinee it stimulates the production of anti-HBsAg antibodies which protect against HBV infection.

For vaccine manufacture, HBsAg has been made in two ways: purification of the antigen in particulate form from the plasma of chronic hepatitis B carriers or expression of the protein by recombinant DNA methods (e.g., recombinant expression in yeast cells). Unlike native HBsAg (i.e., as in the plasma-purified product), yeast-expressed HBsAg is generally non-glycosylated, and this is the most preferred form of HBsAg for use with the invention.

Conjugated Haemophilus influenzae type b antigens: Haemophilus influenzae type b (Hib) causes bacterial meningitis. Hib vaccines are typically based on the capsular saccharide antigen, the preparation of which is well documented. The Hib saccharide can be conjugated to a carrier protein in order to enhance its immunogenicity, especially in children. Typical carrier proteins are tetanus toxoid, diphtheria toxoid, CRM₁₉₇, H. influenzae protein D, and an outer membrane protein complex from serogroup B meningococcus. The saccharide moiety of the conjugate may comprise full-length polyribosylribitol phosphate (PRP) as prepared from Hib bacteria, and/or fragments of full-length PRP. Hib conjugates may or may not be adsorbed to an aluminum salt adjuvant.

In an embodiment the immunogenic compositions of the invention further include a conjugated N. meningitidis serogroup Y capsular saccharide (MenY), and/or a conjugated N. meningitidis serogroup C capsular saccharide (MenC).

In an embodiment the immunogenic compositions of the invention further include a conjugated N. meningitidis serogroup A capsular saccharide (MenA), a conjugated N. meningitidis serogroup W135 capsular saccharide (MenW135), a conjugated N. meningitidis serogroup Y capsular saccharide (MenY), and/or a conjugated N. meningitidis serogroup C capsular saccharide (MenC).

In an embodiment the immunogenic compositions of the invention further include a conjugated N. meningitidis serogroup W135 capsular saccharide (MenW135), a conjugated N. meningitidis serogroup Y capsular saccharide (MenY), and/or a conjugated N. meningitidis serogroup C capsular saccharide (MenC).

### 3.5 Adjuvant(s)

In some embodiments, the immunogenic compositions disclosed herein may further comprise at least one, two or three adjuvants. In some embodiments, the immunogenic compositions disclosed herein may further comprise at least one adjuvant. In some embodiments, the immunogenic compositions disclosed herein may further comprise one adjuvant. In some embodiments, the immunogenic compositions disclosed herein may further comprise two adjuvants. The term “adjuvant” refers to a compound or mixture that enhances the immune response to an antigen. Antigens may act primarily as a delivery system, primarily as an immune modulator or have strong features of both. Suitable adjuvants include those suitable for use in mammals, including humans.

Examples of known suitable delivery-system type adjuvants that can be used in humans include, but are not limited to, alum (e.g., aluminum phosphate, aluminum sulfate or aluminum hydroxide), calcium phosphate, liposomes, oil-in-water emulsions such as MF59 (4.3% w/v squalene, 0.5% w/v polysorbate 80 (Tween 80), 0.5% w/v sorbitan trioleate (Span 85)), water-in-oil emulsions such as Montanide, and poly(D,L-lactide-co-glycolide) (PLG) microparticles or nanoparticles.

In an embodiment, the immunogenic compositions disclosed herein comprise aluminum salts (alum) as adjuvant (e.g., aluminum phosphate, aluminum sulfate or aluminum hydroxide). In a preferred embodiment, the immunogenic compositions disclosed herein comprise aluminum phosphate or aluminum hydroxide as adjuvant. In a preferred embodiment, the immunogenic compositions disclosed herein comprise aluminum phosphate as adjuvant.

Further exemplary adjuvants to enhance effectiveness of the immunogenic compositions as disclosed herein include, but are not limited to: (1) oil-in-water emulsion formulations (with or without other specific immunostimulating agents such as muramyl peptides (see below) or bacterial cell wall components), such as for example (a) SAF, containing 10% Squalene, 0.4% Tween 80, 5% pluronic-blocked polymer L121, and thr-MDP either microfluidized into a submicron emulsion or vortexed to generate a larger particle size emulsion, and (b) RIBI™ adjuvant system (RAS), (Ribi Immunochem, Hamilton, MT) containing 2% Squalene, 0.2% Tween 80, and one or more bacterial cell wall components such as monophosphorylipid A (MPL), trehalose dimycolate (TDM), and cell wall skeleton (CWS), preferably MPL+CWS (DETOX™); (2) saponin adjuvants, such as QS21, STIMULON™ (Cambridge Bioscience, Worcester, MA), ABISCO® (Isconova, Sweden), or ISCOMATRIX® (Commonwealth Serum Laboratories, Australia), may be used or particles generated therefrom such as ISCOMs (immunostimulating complexes), which ISCOMS may be devoid of additional detergent (e.g., WO 00/07621); (3) Complete Freund's Adjuvant (CFA) and Incomplete Freund's Adjuvant (IFA); (4) cytokines, such as interleukins (e.g., IL-1, IL-2, IL-4, IL-5, IL-6, IL-7, IL-12 (e.g., WO 99/44636)), interferons (e.g., gamma interferon), macrophage colony stimulating factor (M-CSF), tumor necrosis factor (TNF), etc.; (5) monophosphoryl lipid A (MPL) or 3-O-deacylated MPL (3dMPL) (see, e.g., GB-2220221, EP0689454), optionally in the substantial absence of alum when used with pneumococcal saccharides (see, e.g., WO 00/56358); (6) combinations of 3dMPL with, for example, QS21 and/or oil-in-water emulsions (see, e.g., EP0835318, EP0735898, EP0761231); (7) a polyoxyethylene ether or a polyoxyethylene ester (see, e.g., WO 99/52549); (8) a polyoxyethylene sorbitan ester surfactant in combination with an octoxynol (e.g., WO 01/21207) or a polyoxyethylene alkyl ether or ester surfactant in combination with at least one additional non-ionic surfactant such as an octoxynol (e.g., WO 01/21152); (9) a saponin and an immunostimulatory oligonucleotide (e.g., a CpG oligonucleotide) (e.g., WO 00/62800); (10) an immunostimulant and a particle of metal salt (see, e.g., WO 00/23105); (11) a saponin and an oil-in-water emulsion (e.g., WO 99/11241); (12) a saponin (e.g., QS21)+3dMPL+IM2 (optionally+a sterol) (e.g., WO 98/57659); (13) other substances that act as immunostimulating agents to enhance the efficacy of the composition. Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipalmitoyl-sn-glycero-3-hydroxyphosphoryloxy)-ethylamine MTP-PE), etc.

In an embodiment of the present invention, the immunogenic compositions as disclosed herein comprise a CpG Oligonucleotide as adjuvant. A CpG oligonucleotide as used herein refers to an immunostimulatory CpG oligodeoxynucleotide (CpG ODN), and accordingly these terms are used interchangeably unless otherwise indicated. Immunostimulatory CpG oligodeoxynucleotides contain one or more immunostimulatory CpG motifs that are unmethylated cytosine-guanine dinucleotides, optionally within certain preferred base contexts. The methylation status of the CpG immunostimulatory motif generally refers to the cytosine residue in the dinucleotide. An immunostimulatory oligonucleotide containing at least one unmethylated CpG dinucleotide is an oligonucleotide which contains a 5′ unmethylated cytosine linked by a phosphate bond to a 3′ guanine, and which activates the immune system through binding to Toll-like receptor 9 (TLR-9). In another embodiment the immunostimulatory oligonucleotide may contain one or more methylated CpG dinucleotides, which will activate the immune system through TLR9 but not as strongly as if the CpG motif(s) was/were unmethylated. CpG immunostimulatory oligonucleotides may comprise one or more palindromes that in turn may encompass the CpG dinucleotide. CpG oligonucleotides have been described in a number of issued patents, published patent applications, and other publications, including U.S. Pat. Nos. 6,194,388; 6,207,646; 6,214,806; 6,218,371; 6,239,116; and 6,339,068.

In an embodiment of the present invention, the immunogenic compositions as disclosed herein comprise any of the CpG Oligonucleotide described at page 3, line 22, to page 12, line 36, of WO 2010/125480.

Different classes of CpG immunostimulatory oligonucleotides have been identified. These are referred to as A, B, C and P class, and are described in greater detail at page 3, line 22, to page 12, line 36, of WO 2010/125480. Methods of the invention embrace the use of these different classes of CpG immunostimulatory oligonucleotides.

In an embodiment of the present invention, the immunogenic compositions as disclosed herein comprise an A class CpG oligonucleotide. Preferably, the “A class” CpG oligonucleotide of the invention has the following nucleic acid sequence: 5′ GGGGACGACGTCGTGGGGGGG 3′ (SEQ ID NO: 1). Some non-limiting examples of A-Class oligonucleotides include: 5′ G*G*G_G_A_C_G_A_C_G_T_C_G_T_G_G*G*G*G*G*G 3′ (SEQ ID NO: 2); wherein “*” refers to a phosphorothioate bond and “_” refers to a phosphodiester bond.

In an embodiment of the present invention, the immunogenic compositions as disclosed herein comprise a B class CpG Oligonucleotide. In one embodiment, the CpG oligonucleotide for use in 5 the present invention is a B class CpG oligonucleotide represented by at least the formula: 5′ X₁X₂CGX₃X₄ 3′, wherein X1, X2, X3, and X4 are nucleotides. In one embodiment, X₂ is adenine, guanine, or thymine. In another embodiment, X₃ is cytosine, adenine, or thymine.

The B class CpG oligonucleotide sequences of the invention are those broadly described above as well as disclosed in WO 96/02555, WO 98/18810 and U.S. Pat. Nos. 6,194,388; 6,207,646; 6,214,806; 6,218,371; 6,239,116 and 6,339,068. Exemplary sequences include but are not limited to those disclosed in these latter applications and patents.

In an embodiment, the “B class” CpG oligonucleotide of the invention has the following nucleic acid sequence:

(SEQ ID NO: 3)
5′ TCGTCGTTTTTCGGTGCTTTT 3′,
or
(SEQ ID NO: 4)
5′ TCGTCGTTTTTCGGTCGTTTT 3′,
or
(SEQ ID NO: 5)
5′ TCGTCGTTTTGTCGTTTTGTCGTT 3′,
or
(SEQ ID NO: 6)
5′ TCGTCGTTTCGTCGTTTTGTCGTT 3′,
or
(SEQ ID NO: 7)
5′ TCGTCGTTTTGTCGTTTTTTTCGA 3′.

In any of these sequences, all of the linkages may be all phosphorothioate bonds. In another embodiment, in any of these sequences, one or more of the linkages may be phosphodiester, preferably between the “C” and the “G” of the CpG motif making a semi-soft CpG oligonucleotide. In any of these sequences, an ethyl-uridine or a halogen may substitute for the 5′ T; examples of halogen substitutions include but are not limited to bromo-uridine or iodo-uridine substitutions.

Some non-limiting examples of B-Class oligonucleotides include:

(SEQ ID NO: 8)
5′ T*C*G*T*C*G*T*T*T*T*T*C*G*G*T*G*C*T*T*T*T 3′,
or
(SEQ ID NO: 9)
5′ T*C*G*T*C*G*T*T*T*T*T*C*G*G*T*C*G*T*T*T*T 3′,
or
(SEQ ID NO: 10)
5′ T*C*G*T*C*G*T*T*T*T*G*T*C*G*T*T*T*T*G*T*C*G*T*
T 3′,
or
(SEQ ID NO: 11)
5′ T*C*G*T*C*G*T*T*T*C*G*T*C*G*T*T*T*T*G*T*C*G*T*
T 3 ,
or
(SEQ ID NO: 12)
5′ T*C*G*T*C*G*T*T*T*T*G*T*C*G*T*T*T*T*T*T*T*C*G*
A 3′.

wherein “*” refers to a phosphorothioate bond.

In an embodiment of the present invention, the immunogenic compositions as disclosed herein comprise a C class CpG Oligonucleotide. In an embodiment, the “C class” CpG oligonucleotides of the invention have the following nucleic acid sequence:

(SEQ ID NO: 13)
5′ TCGCGTCGTTCGGCGCGCGCCG 3′,
or
(SEQ ID NO: 14)
5′ TCGTCGACGTTCGGCGCGCGCCG 3′,
or
(SEQ ID NO: 15)
5′ TCGGACGTTCGGCGCGCGCCG 3′,
or
(SEQ ID NO: 16)
5′ TCGGACGTTCGGCGCGCCG 3′,
or
(SEQ ID NO: 17)
5′ TCGCGTCGTTCGGCGCGCCG 3′,
or
(SEQ ID NO: 18)
5′ TCGACGTTCGGCGCGCGCCG 3′,
or
(SEQ ID NO: 19)
5′ TCGACGTTCGGCGCGCCG 3′,
or
(SEQ ID NO: 20)
5′ TCGCGTCGTTCGGCGCCG 3′,
or
(SEQ ID NO: 21)
5′ TCGCGACGTTCGGCGCGCGCCG 3′,
or
(SEQ ID NO: 22)
5′ TCGTCGTTTTCGGCGCGCGCCG 3′,
or
(SEQ ID NO: 23)
5′ TCGTCGTTTTCGGCGGCCGCCG 3′,
or
(SEQ ID NO: 24)
5′ TCGTCGTTTTACGGCGCCGTGCCG 3′,
or
(SEQ ID NO: 25)
5′ TCGTCGTTTTCGGCGCGCGCCGT 3′.

In any of these sequences, all of the linkages may be all phosphorothioate bonds. In another embodiment, in any of these sequences, one or more of the linkages may be phosphodiester, preferably between the “C” and the “G” of the CpG motif making a semi-soft CpG oligonucleotide. Some non-limiting examples of C-Class oligonucleotides include:

(SEQ ID NO: 26)
5′ T*C_G*C_G*T*C_G*T*T*C_G*G*C*G*C_G*C*G*C*C*G 3′,
or
(SEQ ID NO: 27)
5′ T*C_G*T*C_G*A*C_G*T*T*C_G*G*C*G*C_G*C*G*C*C*G
3′,
or
(SEQ ID NO: 28)
5′ T*C_G*G*A*C_G*T*T*C_G*G*C*G*C_G*C*G*C*C*G 3′,
or
(SEQ ID NO: 29)
5′ T*C_G*G*A*C_G*T*T*C_G*G*C*G*C*G*C*C*G 3′,
or
(SEQ ID NO: 30)
5′ T*C_G*C_G*T*C_G*T*T*C_G*G*C*G*C*G*C*C*G 3′,
or
(SEQ ID NO: 31)
5′ T*C_G*A*C_G*T*T*C_G*G*C*G*C_G*C*G*C*C*G 3′,
or
(SEQ ID NO: 32)
5′ T*C_G*A*C_G*T*T*C_G*G*C*G*C*G*C*C*G 3′,
or
(SEQ ID NO: 33)
5′ T*C_G*C_G*T*C_G*T*T*C_G*G*C*G*C*C*G 3′,
or
(SEQ ID NO: 34)
5′ T*C_G*C_G*A*C_G*T*T*C_G*G*C*G*C_G*C*G*C*C*G 3′,
or
(SEQ ID NO: 35)
5′ T*C*G*T*C*G*T*T*T*T*C*G*G*C*G*C*G*C*G*C*C*G 3′,
or
(SEQ ID NO: 36)
5′ T*C*G*T*C*G*T*T*T*T*C*G*G*C*G*G*C*C*G*C*C*G 3′,
or
(SEQ ID NO: 37)
5′ T*C*G*T*C_G*T*T*T*T*A*C_G*G*C*G*C*C_G*T*G*C*C*
G 3′,
or
(SEQ ID NO: 38)
5′ T*C_G*T*C*G*T*T*T*T*C*G*G*C*G*C*G*C*G*C*C*G*T
3′

wherein “*” refers to a phosphorothioate bond and “_” refers to a phosphodiester bond.

In any of these sequences, an ethyl-uridine or a halogen may substitute for the 5′ T; examples of halogen substitutions include but are not limited to bromo-uridine or iodo-uridine substitutions.

In an embodiment of the present invention, the immunogenic compositions as disclosed herein comprise a P class CpG Oligonucleotide. In an embodiment, the CpG oligonucleotide for use in the present invention is a P class CpG oligonucleotide containing a 5′ TLR activation domain and at least two palindromic regions, one palindromic region being a 5′ palindromic region of at least 6 nucleotides in length and connected to a 3′ palindromic region of at least 8 nucleotides in length either directly or through a spacer, wherein the oligonucleotide includes at least one YpR dinucleotide. In an embodiment, said oligonucleotide is not T*C_G*T*C_G*A*C_G*T*T*C_G*G*C*G*C_G*C*G*C*C*G (SEQ ID NO: 27). In one embodiment the P class CpG oligonucleotide includes at least one unmethylated CpG dinucleotide. In another embodiment the TLR activation domain is TCG, TTCG, TTTCG, TYpR, TTYpR, TTTYpR, UCG, UUCG, UUUCG, TTT, or TTTT. In yet another embodiment the TLR activation domain is within the 5′ palindromic region. In another embodiment the TLR activation domain is immediately 5′ to the 5′ palindromic region.

In an embodiment, the “P class” CpG oligonucleotides of the invention have the following nucleic acid sequence: 5′ TCGTCGACGATCGGCGCGCGCCG 3′ (SEQ ID NO: 39).

In said sequences, all of the linkages may be all phosphorothioate bonds. In another embodiment, one or more of the linkages may be phosphodiester, preferably between the “C” and the “G” of the CpG motif making a semi-soft CpG oligonucleotide. In any of these sequences, an ethyl-uridine or a halogen may substitute for the 5′ T; examples of halogen substitutions include but are not limited to bromo-uridine or iodo-uridine substitutions.

A non-limiting example of P-Class oligonucleotides include:

(SEQ ID NO: 40)
5′ T*C_G*T*C_G*A*C_G*A*T*C_G*G*C*G*C_G*C*G*C*C*G 3′

wherein “*” refers to a phosphorothioate bond and “_” refers to a phosphodiester bond.

In one embodiment the oligonucleotide includes at least one phosphorothioate linkage. In another embodiment all internucleotide linkages of the oligonucleotide are phosphorothioate linkages. In another embodiment the oligonucleotide includes at least one phosphodiester-like linkage. In another embodiment the phosphodiester-like linkage is a phosphodiester linkage. In another embodiment a lipophilic group is conjugated to the oligonucleotide. In one embodiment the lipophilic group is cholesterol.

In an embodiment, all the internucleotide linkages of the CpG oligonucleotides disclosed herein are phosphodiester bonds (“soft” oligonucleotides, as described in WO 2007/026190). In another embodiment, CpG oligonucleotides of the invention are rendered resistant to degradation (e.g., are stabilized). A “stabilized oligonucleotide” refers to an oligonucleotide that is relatively resistant to in vivo degradation (e.g., via an exo- or endo-nuclease). Nucleic acid stabilization can be accomplished via backbone modifications. Oligonucleotides having phosphorothioate linkages provide maximal activity and protect the oligonucleotide from degradation by intracellular exo- and endo-nucleases.

The immunostimulatory oligonucleotides may have a chimeric backbone, which have combinations of phosphodiester and phosphorothioate linkages. For purposes of the instant invention, a chimeric backbone refers to a partially stabilized backbone, wherein at least one internucleotide linkage is phosphodiester or phosphodiester-like, and wherein at least one other internucleotide linkage is a stabilized internucleotide linkage, wherein the at least one phosphodiester or phosphodiester-like linkage and the at least one stabilized linkage are different. When the phosphodiester linkage is preferentially located within the CpG motif such molecules are called “semi-soft” as described in WO 2007/026190.

Other modified oligonucleotides include combinations of phosphodiester, phosphorothioate, methylphosphonate, methylphosphorothioate, phosphorodithioate, and/or p-ethoxy linkages.

Mixed backbone modified ODN may be synthesized as described in WO 2007/026190.

The size of the CpG oligonucleotide (i.e., the number of nucleotide residues along the length of the oligonucleotide) also may contribute to the stimulatory activity of the oligonucleotide. For facilitating uptake into cells, CpG oligonucleotide of the invention preferably have a minimum length of 6 nucleotide residues. Oligonucleotides of any size greater than 6 nucleotides (even many kb long) are capable of inducing an immune response if sufficient immunostimulatory motifs are present, because larger oligonucleotides are degraded inside cells. In certain embodiments, the CpG oligonucleotides are 6 to 100 nucleotides long, preferentially 8 to 30 nucleotides long. In important embodiments, nucleic acids and oligonucleotides of the invention are not plasmids or expression vectors.

In an embodiment, the CpG oligonucleotide disclosed herein comprise substitutions or modifications, such as in the bases and/or sugars as described at paragraphs 134 to 147 of WO 2007/026190.

In an embodiment, the CpG oligonucleotide of the present invention is chemically modified. Examples of chemical modifications are known to the skilled person and are described, for example in Uhlmann et al. (1990) Chem. Rev. 90:543; S. Agrawal, Ed., Humana Press, Totowa, USA 1993; Crooke et al. (1996) Annu. Rev. Pharmacol. Toxicol. 36:107-129; and Hunziker et al. (1995) Mod. Synth. Methods 7:331-417. An oligonucleotide according to the invention may have one or more modifications, wherein each modification is located at a particular phosphodiester internucleoside bridge and/or at a particular β-D-ribose unit and/or at a particular natural nucleoside base position in comparison to an oligonucleotide of the same sequence which is composed of natural DNA or RNA.

In some embodiments of the invention, CpG-containing nucleic acids might be simply mixed with immunogenic carriers according to methods known to those skilled in the art (see, e.g., WO 03/024480).

In a particular embodiment of the present invention, any of the immunogenic compositions disclosed herein comprise from 2 μg to 100 mg of CpG oligonucleotide. In a particular embodiment of the present invention, the immunogenic composition of the invention comprises 0.1 mg to 50 mg of CpG oligonucleotide, preferably from 0.2 mg to 10 mg CpG oligonucleotide, more preferably from 0.3 mg to 5 mg CpG oligonucleotide. In a particular embodiment of the present invention, the immunogenic composition of the invention comprises from 0.3 mg to 5 mg CpG oligonucleotide. Even preferably, the immunogenic composition of the invention may comprise from 0.5 to 2 mg CpG oligonucleotide. Most preferably, the immunogenic composition of the invention may comprise from 0.75 to 1.5 mg CpG oligonucleotide. In a preferred embodiment, any of the immunogenic composition disclosed herein may comprise about 1 mg CpG oligonucleotide.

### 4 Formulation

The immunogenic compositions of the invention may be formulated in liquid form (i.e., solutions or suspensions) or in a lyophilized form. In an embodiment, the immunogenic composition of the invention is formulated in a liquid form. In an embodiment, the immunogenic composition of the invention is formulated in a lyophilized form. Liquid formulations may advantageously be administered directly from their packaged form and are thus ideal for injection without the need for reconstitution in aqueous medium as otherwise required for lyophilized compositions of the invention.

Formulation of the immunogenic composition of the present disclosure can be accomplished using art-recognized methods. For instance, the individual polysaccharides and/or conjugates can be formulated with a physiologically acceptable vehicle to prepare the composition. Examples of such vehicles include, but are not limited to, water, buffered saline, polyols (e.g., glycerol, propylene glycol, liquid polyethylene glycol) and dextrose solutions.

The present disclosure provides an immunogenic composition comprising any of combination of glycoconjugates disclosed herein and a pharmaceutically acceptable excipient, carrier, or diluent. In an embodiment, the immunogenic composition of the disclosure is in liquid form, preferably in aqueous liquid form.

Immunogenic compositions of the disclosure may comprise one or more of a buffer, a salt, a divalent cation, a non-ionic detergent, a cryoprotectant such as a sugar, and an anti-oxidant such as a free radical scavenger or chelating agent, or any multiple combinations thereof.

In an embodiment, the immunogenic compositions of the disclosure comprise a buffer. In an embodiment, said buffer has a pKa of about 3.5 to about 7.5. In some embodiments, the buffer is phosphate, succinate, histidine or citrate. In some embodiments, the buffer is succinate. In some embodiments, the buffer is histidine. In certain embodiments, the buffer is succinate at a final concentration of 1 mM to 10 mM. In one particular embodiment, the final concentration of the succinate buffer is about 5 mM.

In an embodiment, the immunogenic compositions of the disclosure comprise a salt. In some embodiments, the salt is selected from the groups consisting of magnesium chloride, potassium chloride, sodium chloride and a combination thereof. In one particular embodiment, the salt is sodium chloride. In one particular embodiment, the immunogenic compositions of the invention comprise sodium chloride at 150 mM.

In an embodiment, the immunogenic compositions of the disclosure comprise a surfactant. In an embodiment, the surfactant is selected from the group consisting of polysorbate 20 (TWEEN™20), polysorbate 40 (TWEEN™40), polysorbate 60 (TWEEN™60), polysorbate 65 (TWEEN™65), polysorbate 80 (TWEEN™80), polysorbate 85 (TWEEN™85), TRITON™ N-101, TRITON™ X-100, oxtoxynol 40, nonoxynol-9, triethanolamine, triethanolamine polypeptide oleate, polyoxyethylene-660 hydroxystearate (PEG-15, Solutol H 15), polyoxyethylene-35-ricinoleate (CREMOPHOR® EL), soy lecithin and a poloxamer. In one particular embodiment, the surfactant is polysorbate 80. In some said embodiment, the final concentration of polysorbate 80 in the formulation is at least 0.0001% to 10% polysorbate 80 weight to weight (w/w). In some said embodiments, the final concentration of polysorbate 80 in the formulation is at least 0.001% to 1% polysorbate 80 weight to weight (w/w). In some said embodiments, the final concentration of polysorbate 80 in the formulation is at least 0.01% to 1% polysorbate 80 weight to weight (w/w). In other embodiments, the final concentration of polysorbate 80 in the formulation is 0.01%, 0.02%, 0.03%, 0.04%, 0.05%, 0.06%, 0.07%, 0.08%, 0.09% or 0.1% polysorbate 80 (w/w). In another embodiment, the final concentration of the polysorbate 80 in the formulation is 0.02% polysorbate 80 (w/w). In another embodiment, the final concentration of the polysorbate 80 in the formulation is 0.01% polysorbate 80 (w/w). In another embodiment, the final concentration of the polysorbate 80 in the formulation is 0.03% polysorbate 80 (w/w). In another embodiment, the final concentration of the polysorbate 80 in the formulation is 0.04% polysorbate 80 (w/w). In another embodiment, the final concentration of the polysorbate 80 in the formulation is 0.05% polysorbate 80 (w/w). In another embodiment, the final concentration of the polysorbate 80 in the formulation is 1% polysorbate 80 (w/w).

In one particular embodiment, the surfactant is polysorbate 20. In some said embodiment, the final concentration of polysorbate 20 in the formulation is at least 0.0001% to 10% polysorbate 20 weight to weight (w/w). In some said embodiments, the final concentration of polysorbate 20 in the formulation is at least 0.001% to 1% polysorbate 20 weight to weight (w/w). In some said embodiments, the final concentration of polysorbate 20 in the formulation is at least 0.01% to 1% polysorbate 20 weight to weight (w/w). In other embodiments, the final concentration of polysorbate 20 in the formulation is 0.01%, 0.02%, 0.03%, 0.04%, 0.05%, 0.06%, 0.07%, 0.08%, 0.09% or 0.1% polysorbate 20 (w/w). In another embodiment, the final concentration of the polysorbate 20 in the formulation is 0.02% polysorbate 20 (w/w). In another embodiment, the final concentration of the polysorbate 20 in the formulation is 0.01% polysorbate 20 (w/w). In another embodiment, the final concentration of the polysorbate 20 in the formulation is 0.03% polysorbate 20 (w/w). In another embodiment, the final concentration of the polysorbate 20 in the formulation is 0.04% polysorbate 80 (w/w). In another embodiment, the final concentration of the polysorbate 20 in the formulation is 0.05% polysorbate 20 (w/w). In another embodiment, the final concentration of the polysorbate 20 in the formulation is 1% polysorbate 20 (w/w).

In one particular embodiment, the surfactant is polysorbate 40. In some said embodiment, the final concentration of polysorbate 40 in the formulation is at least 0.0001% to 10% polysorbate 40 weight to weight (w/w). In some said embodiments, the final concentration of polysorbate 40 in the formulation is at least 0.001% to 1% polysorbate 40 weight to weight (w/w). In some said embodiments, the final concentration of polysorbate 40 in the formulation is at least 0.01% to 1% polysorbate 40 weight to weight (w/w). In other embodiments, the final concentration of polysorbate 40 in the formulation is 0.01%, 0.02%, 0.03%, 0.04%, 0.05%, 0.06%, 0.07%, 0.08%, 0.09% or 0.1% polysorbate 40 (w/w). In another embodiment, the final concentration of the polysorbate 40 in the formulation is 1% polysorbate 40 (w/w).

In one particular embodiment, the surfactant is polysorbate 60. In some said embodiment, the final concentration of polysorbate 60 in the formulation is at least 0.0001% to 10% polysorbate 60 weight to weight (w/w). In some said embodiments, the final concentration of polysorbate 60 in the formulation is at least 0.001% to 1% polysorbate 60 weight to weight (w/w). In some said embodiments, the final concentration of polysorbate 60 in the formulation is at least 0.01% to 1% polysorbate 60 weight to weight (w/w). In other embodiments, the final concentration of polysorbate 60 in the formulation is 0.01%, 0.02%, 0.03%, 0.04%, 0.05%, 0.06%, 0.07%, 0.08%, 0.09% or 0.1% polysorbate 60 (w/w). In another embodiment, the final concentration of the polysorbate 60 in the formulation is 1% polysorbate 60 (w/w).

In one particular embodiment, the surfactant is polysorbate 65. In some said embodiment, the final concentration of polysorbate 65 in the formulation is at least 0.0001% to 10% polysorbate 65 weight to weight (w/w). In some said embodiments, the final concentration of polysorbate 65 in the formulation is at least 0.001% to 1% polysorbate 65 weight to weight (w/w). In some said embodiments, the final concentration of polysorbate 65 in the formulation is at least 0.01% to 1% polysorbate 65 weight to weight (w/w). In other embodiments, the final concentration of polysorbate 65 in the formulation is 0.01%, 0.02%, 0.03%, 0.04%, 0.05%, 0.06%, 0.07%, 0.08%, 0.09% or 0.1% polysorbate 65 (w/w). In another embodiment, the final concentration of the polysorbate 65 in the formulation is 1% polysorbate 65 (w/w).

In one particular embodiment, the surfactant is polysorbate 85. In some said embodiment, the final concentration of polysorbate 85 in the formulation is at least 0.0001% to 10% polysorbate 85 weight to weight (w/w). In some said embodiments, the final concentration of polysorbate 85 in the formulation is at least 0.001% to 1% polysorbate 85 weight to weight (w/w). In some said embodiments, the final concentration of polysorbate 85 in the formulation is at least 0.01% to 1% polysorbate 85 weight to weight (w/w). In other embodiments, the final concentration of polysorbate 85 in the formulation is 0.01%, 0.02%, 0.03%, 0.04%, 0.05%, 0.06%, 0.07%, 0.08%, 0.09% or 0.1% polysorbate 85 (w/w). In another embodiment, the final concentration of the polysorbate 85 in the formulation is 1% polysorbate 85 (w/w).

In certain embodiments, the immunogenic composition of the disclosure has a pH of 5.5 to 7.5, more preferably a pH of 5.6 to 7.0, even more preferably a pH of 5.8 to 6.0.

In one embodiment, the present disclosure provides a container filled with any of the immunogenic compositions disclosed herein. In one embodiment, the container is selected from the group consisting of a vial, a syringe, a flask, a fermentor, a bioreactor, a bag, a jar, an ampoule, a cartridge and a disposable pen. In certain embodiments, the container is siliconized.

In an embodiment, the container of the present disclosure is made of glass, metals (e.g., steel, stainless steel, aluminum, etc.) and/or polymers (e.g., thermoplastics, elastomers, thermoplastic-elastomers). In an embodiment, the container of the present disclosure is made of glass.

In one embodiment, the present disclosure provides a syringe filled with any of the immunogenic compositions disclosed herein. In certain embodiments, the syringe is siliconized and/or is made of glass.

A typical dose of the immunogenic composition of the invention for injection has a volume of 0.1 mL to 2 mL. In an embodiment, the immunogenic composition of the invention for injection has a volume of 0.2 mL to 1 mL, even more preferably a volume of about 0.5 mL.

### 5 Uses of the Glycoconjugate and Immunogenic Compositions of the Invention

The glycoconjugates disclosed herein may be use as antigens. For example, they may be part of a vaccine.

Therefore, in an embodiment, the immunogenic compositions of the invention are for use as a medicament.

In an embodiment, the immunogenic compositions of the invention are for use as a vaccine.

Therefore, in an embodiment, the immunogenic compositions described herein are for use in generating an immune response in a subject. In one aspect, the subject is a mammal, such as a human, cat, sheep, pig, horse, bovine or dog. In one aspect, the subject is a human.

The immunogenic compositions described herein may be used in therapeutic or prophylactic methods for preventing, treating or ameliorating a bacterial infection, disease or condition in a subject. In particular, immunogenic compositions described herein may be used to prevent, treat or ameliorate a S. pneumoniae serotype 9N infection, disease or condition in a subject.

Thus, in one aspect, the disclosure provides a method of preventing, treating or ameliorating an infection, disease or condition associated with S. pneumoniae serotype 9N in a subject, comprising administering to the subject an immunologically effective amount of an immunogenic composition of the disclosure.

In some such embodiments, the infection, disease or condition is selected from the group consisting of pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection and brain abscess.

In an embodiment, the disclosure provides a method of inducing an immune response to S. pneumoniae serotype 9N in a subject comprising administering to the subject an immunologically effective amount of an immunogenic composition of the invention. In one aspect, the subject is a mammal, such as a human, cat, sheep, pig, horse, bovine or dog. In one aspect, the subject is a human.

In an embodiment, the immunogenic compositions disclosed herein are for use as a vaccine. In such embodiments the immunogenic compositions described herein may be used to prevent S. pneumoniae serotype 9N infection in a subject. Thus, in one aspect, the invention provides a method of preventing an infection by S. pneumoniae serotype 9N in a subject comprising administering to the subject an immunologically effective amount of an immunogenic composition of the disclosure. In some such embodiments, the infection is selected from the group consisting of pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection and brain abscess. In one aspect, the subject is a mammal, such as a human, cat, sheep, pig, horse, bovine or dog. In one aspect, the subject is a human.

The immunogenic composition of the present disclosure can be used to protect or treat a human susceptible to a S. pneumoniae serotype 9N infection, by means of administering the immunogenic composition via a systemic or mucosal route. In an embodiment, the immunogenic composition of the invention is administered by intramuscular, intraperitoneal, intradermal or subcutaneous routes. In an embodiment, the immunogenic composition of the invention is administered by intramuscular, intraperitoneal, intradermal or subcutaneous injection. In an embodiment, the immunogenic composition of the invention is administered by intramuscular or subcutaneous injection. In an embodiment, the immunogenic composition of the invention is administered by intramuscular injection. In an embodiment, the immunogenic composition of the invention is administered by subcutaneous injection.

6 Subject to be Treated with the Immunogenic Compositions of the Invention

As disclosed herein, the immunogenic compositions described herein may be used in various therapeutic or prophylactic methods for preventing, treating or ameliorating a bacterial infection, disease or condition in a subject.

In a preferred embodiment, said subject is a human. In a most preferred embodiment, said subject is a newborn (i.e., under three months of age), an infant (i.e., from 3 months to one year of age) or a toddler (i.e., from one year to four years of age).

In an embodiment, the immunogenic compositions disclosed herein are for use as a vaccine.

In such embodiment, the subject to be vaccinated may be less than 1 year of age. For example, the subject to be vaccinated can be about 1, about 2, about 3, about 4, about 5, about 6, about 7, about 8, about 9, about 10, about 11 or about 12 months of age. In an embodiment, the subject to be vaccinated is about 2, about 4 or about 6 months of age. In another embodiment, the subject to be vaccinated is less than 2 years of age. For example, the subject to be vaccinated can be about 12 to about 15 months of age. In some cases, as little as one dose of the immunogenic composition according to the invention is needed, but under some circumstances, a second, third or fourth dose may be given (see section 8 below).

In an embodiment of the present invention, the subject to be vaccinated is a human adult 50 years of age or older, more preferably a human adult 55 years of age or older. In an embodiment, the subject to be vaccinated is a human adult 65 years of age or older, 70 years of age or older, 75 years of age or older or 80 years of age or older.

In an embodiment the subject to be vaccinated is an immunocompromised individual, in particular a human. An immunocompromised individual is generally defined as a person who exhibits an attenuated or reduced ability to mount a normal humoral or cellular defense to challenge by infectious agents.

In an embodiment of the present invention, the immunocompromised subject to be vaccinated suffers from a disease or condition that impairs the immune system and results in an antibody response that is insufficient to protect against or treat pneumococcal disease.

In an embodiment, said disease is a primary immunodeficiency disorder. Preferably, said primary immunodeficiency disorder is selected from the group consisting of: combined T- and B-cell immunodeficiencies, antibody deficiencies, well-defined syndromes, immune dysregulation diseases, phagocyte disorders, innate immunity deficiencies, autoinflammatory disorders, and complement deficiencies. In an embodiment, said primary immunodeficiency disorder is selected from the one disclosed on page 24, line 11, to page 25, line 19, of WO 2010/125480.

In a particular embodiment of the present invention, the immunocompromised subject to be vaccinated suffers from a disease selected from the groups consisting of: HIV-infection, acquired immunodeficiency syndrome (AIDS), cancer, chronic heart or lung disorders, congestive heart failure, diabetes mellitus, chronic liver disease, alcoholism, cirrhosis, spinal fluid leaks, cardiomyopathy, chronic bronchitis, emphysema, chronic obstructive pulmonary disease (COPD), spleen dysfunction (such as sickle cell disease), lack of spleen function (asplenia), blood malignancy, leukemia, multiple myeloma, Hodgkin's disease, lymphoma, kidney failure, nephrotic syndrome and asthma.

In an embodiment of the present invention, the immunocompromised subject to be vaccinated suffers from malnutrition.

In a particular embodiment of the present invention, the immunocompromised subject to be vaccinated is taking a drug or treatment that lowers the body's resistance to infection. In an embodiment, said drug is selected from the one disclosed on page 26, line 33, to page 26, line 4, of WO 2010/125480.

In a particular embodiment of the present invention, the immunocompromised subject to be vaccinated is a smoker.

In a particular embodiment of the present invention, the immunocompromised subject to be vaccinated has a white blood cell count (leukocyte count) below 5×10⁹ cells per liter, or below 4×10⁹ cells per liter, or below 3×10⁹ cells per liter, or below 2×10⁹ cells per liter, or below 1×10⁹ cells per liter, or below 0.5×10⁹ cells per liter, or below 0.3×10⁹ cells per liter, or below 0.1×10⁹ cells per liter.

White blood cell count (leukocyte count): The number of white blood cells (WBC) in the blood. The WBC is usually measured as part of the CBC (complete blood count). White blood cells are the infection-fighting cells in the blood and are distinct from the red (oxygen-carrying) blood cells known as erythrocytes. There are different types of white blood cells, including neutrophils (polymorphonuclear leukocytes; PMN), band cells (slightly immature neutrophils), T-type lymphocytes (T-cells), B-type lymphocytes (B-cells), monocytes, eosinophils, and basophils. All the types of white blood cells are reflected in the white blood cell count. The normal range for the white blood cell count is usually between 4,300 and 10,800 cells per cubic millimeter of blood. This can also be referred to as the leukocyte count and can be expressed in international units as 4.3-10.8×10⁹ cells per liter.

In a particular embodiment of the present invention, the immunocompromised subject to be vaccinated suffers from neutropenia. In a particular embodiment of the present invention, the immunocompromised subject to be vaccinated has a neutrophil count below 2×10⁹ cells per liter, or below 1×10⁹ cells per liter, or below 0.5×10⁹ cells per liter, or below 0.1×10⁹ cells per liter, or below 0.05×10⁹ cells per liter.

A low white blood cell count or “neutropenia” is a condition characterized by abnormally low levels of neutrophils in the circulating blood. Neutrophils are a specific kind of white blood cell that help to prevent and fight infections. The most common reason that cancer patients experience neutropenia is as a side effect of chemotherapy. Chemotherapy-induced neutropenia increases a patient's risk of infection and disrupts cancer treatment.

In a particular embodiment of the present invention, the immunocompromised subject to be vaccinated has a CD4+ cell count below 500/mm³, or CD4+ cell count below 300/mm³, or CD4+ cell count below 200/mm³, CD4+ cell count below 100/mm³, CD4+ cell count below 75/mm³, or CD4+ cell count below 50/mm³.

CD4 cell tests are normally reported as the number of cells in mm³. Normal CD4 counts are between 500 and 1,600, and CD8 counts are between 375 and 1,100. CD4 counts drop dramatically in people with HIV.

In an embodiment of the invention, any of the immunocompromised subjects disclosed herein is a human male or a human female.

### 7 Regimen

In some cases, as little as one dose of the immunogenic composition according to the invention is needed, but under some circumstances, such as conditions of greater immune deficiency, a second, third or fourth dose may be given. Following an initial vaccination, subjects can receive one or several booster immunizations adequately spaced.

In an embodiment, the schedule of vaccination of the immunogenic composition according to the invention is a single dose. In a particular embodiment, said single dose schedule is for healthy persons being at least 2 years of age.

In an embodiment, the schedule of vaccination of the immunogenic composition according to the invention is a multiple dose schedule. In a particular embodiment, said multiple dose schedule consists of a series of 2 doses separated by an interval of about 1 month to about 2 months. In a particular embodiment, said multiple dose schedule consists of a series of 2 doses separated by an interval of about 1 month, or a series of 2 doses separated by an interval of about 2 months.

In another embodiment, said multiple dose schedule consists of a series of 3 doses separated by an interval of about 1 month to about 2 months. In another embodiment, said multiple dose schedule consists of a series of 3 doses separated by an interval of about 1 month, or a series of 3 doses separated by an interval of about 2 months.

In another embodiment, said multiple dose schedule consists of a series of 3 doses separated by an interval of about 1 month to about 2 months followed by a fourth dose about 10 months to about 13 months after the first dose. In another embodiment, said multiple dose schedule consists of a series of 3 doses separated by an interval of about 1 month followed by a fourth dose about 10 months to about 13 months after the first dose, or a series of 3 doses separated by an interval of about 2 months followed by a fourth dose about 10 months to about 13 months after the first dose.

In an embodiment, the multiple dose schedule consists of at least one dose (e.g., 1, 2 or 3 doses) in the first year of age followed by at least one toddler dose.

In an embodiment, the multiple dose schedule consists of a series of 2 or 3 doses separated by an interval of about 1 month to about 2 months (for example 28-56 days between doses), starting at 2 months of age, and followed by a toddler dose at 12-18 months of age. In an embodiment, said multiple dose schedule consists of a series of 3 doses separated by an interval of about 1 month to about 2 months (for example 28-56 days between doses), starting at 2 months of age, and followed by a toddler dose at 12-15 months of age. In another embodiment, said multiple dose schedule consists of a series of 2 doses separated by an interval of about 2 months, starting at 2 months of age, and followed by a toddler dose at 12-18 months of age.

In an embodiment, the multiple dose schedule consists of a 4-dose series of vaccine at 2, 4, 6, and 12-15 months of age.

In an embodiment, a prime dose is given at day 0 and one or more boosts are given at intervals that range from about 2 to about 24 weeks, preferably with a dosing interval of 4-8 weeks.

In an embodiment, a prime dose is given at day 0 and a boost is given about 3 months later.

### 8 Particular Embodiments of the Invention are Set Forth in the Following Numbered Paragraphs 1 to 350:

1. A Streptococcus pneumoniae serotype 9N glycoconjugate comprising a serotype 9N capsular polysaccharide wherein the weight average molecular weight (Mw) of said polysaccharide before conjugation is between 50 kDa and 1,000 kDa.
2. The Streptococcus pneumoniae serotype 9N glycoconjugate of paragraph 1 wherein the weight average molecular weight (Mw) of said polysaccharide before conjugation is between 150 kDa and 650 kDa.
3. The Streptococcus pneumoniae serotype 9N glycoconjugate of paragraph 1 wherein the weight average molecular weight (Mw) of said polysaccharide before conjugation is between 200 kDa and 500 kDa.
4. The Streptococcus pneumoniae serotype 9N glycoconjugate of paragraph 1 wherein the weight average molecular weight (Mw) of said polysaccharide before conjugation is between 200 kDa and 400 kDa.
5. The Streptococcus pneumoniae serotype 9N glycoconjugate of any one of paragraphs 1-4 wherein said serotype 9N glycoconjugate has a weight average molecular weight (Mw) of between 250 kDa and 20,000 kDa.
6. The Streptococcus pneumoniae serotype 9N glycoconjugate of any one of paragraphs 1-4 wherein said serotype 9N glycoconjugate has a weight average molecular weight (Mw) of between 500 kDa and 15,000 kDa.
7. The Streptococcus pneumoniae serotype 9N glycoconjugate of any one of paragraphs 1-4 wherein said serotype 9N glycoconjugate has a weight average molecular weight (Mw) of between 500 kDa and 10,000 kDa.
8. The Streptococcus pneumoniae serotype 9N glycoconjugate of any one of paragraphs 1-4 wherein said serotype 9N glycoconjugate has a weight average molecular weight (Mw) of between 500 kDa and 5,000 kDa.
9. The Streptococcus pneumoniae serotype 9N glycoconjugate of any one of paragraphs 1-4 wherein said serotype 9N glycoconjugate has a weight average molecular weight (Mw) of between 800 kDa and 8,000 kDa.
10. The Streptococcus pneumoniae serotype 9N glycoconjugate of any one of paragraphs 1-4 wherein said serotype 9N glycoconjugate has a weight average molecular weight (Mw) of between 800 kDa and 6,000 kDa.
11. The Streptococcus pneumoniae serotype 9N glycoconjugate of any one of paragraphs 1-4 wherein said serotype 9N glycoconjugate has a weight average molecular weight (Mw) of between 1000 kDa and 5,000 kDa.
12. The Streptococcus pneumoniae serotype 9N glycoconjugate of any one of paragraphs 1-4 wherein said serotype 9N glycoconjugate has a weight average molecular weight (Mw) of between 1,500 kDa and 4,500 kDa.
13. The Streptococcus pneumoniae serotype 9N glycoconjugate of paragraph 1 wherein the weight average molecular weight (Mw) of said polysaccharide before conjugation is between 200 kDa and 500 kDa and wherein said serotype 9N glycoconjugate has a weight average molecular weight (Mw) of between 1,500 kDa and 4,500 kDa.
14. The Streptococcus pneumoniae serotype 9N glycoconjugate of paragraph 1 wherein the weight average molecular weight (Mw) of said polysaccharide before conjugation is between 200 kDa and 400 kDa and wherein said serotype 9N glycoconjugate has a weight average molecular weight (Mw) of between 1,500 kDa and 4,500 kDa.
15. The Streptococcus pneumoniae serotype 9N glycoconjugate of any one of paragraphs 1-14, wherein the degree of conjugation of said serotype 9N glycoconjugate is between 2 and 15.
16. The Streptococcus pneumoniae serotype 9N glycoconjugate of any one of paragraphs 1-14, wherein the degree of conjugation of said serotype 9N glycoconjugate is between 4 and 12.
17. The Streptococcus pneumoniae serotype 9N glycoconjugate of any one of paragraphs 1-14, wherein the degree of conjugation of said serotype 9N glycoconjugate is between 4 and 7.
18. The Streptococcus pneumoniae serotype 9N glycoconjugate of paragraph 1 wherein the weight average molecular weight (Mw) of said polysaccharide before conjugation is between 200 kDa and 500 kDa, wherein the degree of conjugation of said serotype 9N glycoconjugate is between 4 and 12 and wherein said serotype 9N glycoconjugate has a weight average molecular weight (Mw) of between 1,500 kDa and 4,500 kDa.
19. The Streptococcus pneumoniae serotype 9N glycoconjugate of paragraph 1 wherein the weight average molecular weight (Mw) of said polysaccharide before conjugation is between 200 kDa and 400 kDa, wherein the degree of conjugation of said serotype 9N glycoconjugate is between 4 and 12 and wherein said serotype 9N glycoconjugate has a weight average molecular weight (Mw) of between 1,500 kDa and 4,500 kDa.
20. The Streptococcus pneumoniae serotype 9N glycoconjugate of paragraph 1 wherein the weight average molecular weight (Mw) of said polysaccharide before conjugation is between 200 kDa and 400 kDa, wherein the degree of conjugation of said serotype 9N glycoconjugate is between 4 and 7 and wherein said serotype 9N glycoconjugate has a weight average molecular weight (Mw) of between 1,500 kDa and 4,500 kDa.
21. The Streptococcus pneumoniae serotype 9N glycoconjugate of any one of paragraphs 1-20, wherein the ratio of serotype 9N polysaccharide to carrier protein in the glycoconjugate (w/w) is between 0.5 and 3.0.
22. The Streptococcus pneumoniae serotype 9N glycoconjugate of any one of paragraphs 1-20, wherein the ratio of serotype 9N polysaccharide to carrier protein in the glycoconjugate (w/w) is between 0.5 and 1.5.
23. The Streptococcus pneumoniae serotype 9N glycoconjugate of any one of paragraphs 1-20, wherein the ratio of serotype 9N polysaccharide to carrier protein in the glycoconjugate (w/w) is between 0.8 and 1.2.
24. The Streptococcus pneumoniae serotype 9N glycoconjugate of paragraph 1 wherein the weight average molecular weight (Mw) of said polysaccharide before conjugation is between 200 kDa and 500 kDa, wherein the degree of conjugation of said serotype 9N glycoconjugate is between 4 and 12, wherein the ratio of serotype 9N polysaccharide to carrier protein in the glycoconjugate (w/w) is between 0.5 and 1.5 and wherein said serotype 9N glycoconjugate has a weight average molecular weight (Mw) of between 1,500 kDa and 4,500 kDa.
25. The Streptococcus pneumoniae serotype 9N glycoconjugate of paragraph 1 wherein the weight average molecular weight (Mw) of said polysaccharide before conjugation is between 200 kDa and 400 kDa, wherein the degree of conjugation of said serotype 9N glycoconjugate is between 4 and 12, wherein the ratio of serotype 9N polysaccharide to carrier protein in the glycoconjugate (w/w) is between 0.5 and 1.5 and wherein said serotype 9N glycoconjugate has a weight average molecular weight (Mw) of between 1,500 kDa and 4,500 kDa.
26. The Streptococcus pneumoniae serotype 9N glycoconjugate of paragraph 1 wherein the weight average molecular weight (Mw) of said polysaccharide before conjugation is between 200 kDa and 400 kDa, wherein the degree of conjugation of said serotype 9N glycoconjugate is between 4 and 7, wherein the ratio of serotype 9N polysaccharide to carrier protein in the glycoconjugate (w/w) is between 0.5 and 1.5 and wherein said serotype 9N glycoconjugate has a weight average molecular weight (Mw) of between 1,500 kDa and 4,500 kDa.
27. The Streptococcus pneumoniae serotype 9N glycoconjugate of paragraph 1 wherein the weight average molecular weight (Mw) of said polysaccharide before conjugation is between 200 kDa and 400 kDa, wherein the degree of conjugation of said serotype 9N glycoconjugate is between 4 and 12, wherein the ratio of serotype 9N polysaccharide to carrier protein in the glycoconjugate (w/w) is between 0.8 and 1.2 and wherein said serotype 9N glycoconjugate has a weight average molecular weight (Mw) of between 1,500 kDa and 4,500 kDa.
28. The Streptococcus pneumoniae serotype 9N glycoconjugate of any one of paragraphs 1-27, wherein said serotype 9N glycoconjugate comprises at least one covalent linkage between the carrier protein and the polysaccharide for every 4 saccharide repeat units of the polysaccharide.
29. The Streptococcus pneumoniae serotype 9N glycoconjugate of any one of paragraphs 1-27, wherein said serotype 9N glycoconjugate comprises at least one covalent linkage between the carrier protein and the polysaccharide for every 10 saccharide repeat units of the polysaccharide.
30. The Streptococcus pneumoniae serotype 9N glycoconjugate of any one of paragraphs 1-27, wherein the covalent linkage between the carrier protein and the polysaccharide occurs at least once in every 15 saccharide repeat units of the polysaccharide.
31. The Streptococcus pneumoniae serotype 9N glycoconjugate of any one of paragraphs 1-27, wherein the covalent linkage between the carrier protein and the polysaccharide occurs at least once in every 25 saccharide repeat units of the polysaccharide.
32. The Streptococcus pneumoniae serotype 9N glycoconjugate of any one of paragraphs 1-27, wherein the covalent linkage between the carrier protein and the polysaccharide occurs at least once in every 50 saccharide repeat units of the polysaccharide.
33. The Streptococcus pneumoniae serotype 9N glycoconjugate of any one of paragraphs 1-27, wherein the covalent linkage between the carrier protein and the polysaccharide occurs at least once in every 100 saccharide repeat units of the polysaccharide.
34. The Streptococcus pneumoniae serotype 9N glycoconjugate of any one of paragraphs 1-27, wherein said serotype 9N glycoconjugate comprises at least one covalent linkage between the carrier protein and the polysaccharide for every 5 to 30 saccharide repeat units of the polysaccharide.
35. The Streptococcus pneumoniae serotype 9N glycoconjugate of any one of paragraphs 1-27, wherein said serotype 9N glycoconjugate comprises at least one covalent linkage between the carrier protein and the polysaccharide for every 10 to 20 saccharide repeat units of the polysaccharide.
36. The Streptococcus pneumoniae serotype 9N glycoconjugate of paragraph 1 wherein the weight average molecular weight (Mw) of said polysaccharide before conjugation is between 200 kDa and 500 kDa, wherein the degree of conjugation of said serotype 9N glycoconjugate is between 4 and 12, wherein the ratio of serotype 9N polysaccharide to carrier protein in the glycoconjugate (w/w) is between 0.5 and 1.5, wherein said serotype 9N glycoconjugate comprises at least one covalent linkage between the carrier protein and the polysaccharide for every 5 to 30 saccharide repeat units of the polysaccharide and wherein said serotype 9N glycoconjugate has a weight average molecular weight (Mw) of between 1,500 kDa and 4,500 kDa.
37. The Streptococcus pneumoniae serotype 9N glycoconjugate of paragraph 1 wherein the weight average molecular weight (Mw) of said polysaccharide before conjugation is between 200 kDa and 400 kDa, wherein the degree of conjugation of said serotype 9N glycoconjugate is between 4 and 12, wherein the ratio of serotype 9N polysaccharide to carrier protein in the glycoconjugate (w/w) is between 0.5 and 1.5, wherein said serotype 9N glycoconjugate comprises at least one covalent linkage between the carrier protein and the polysaccharide for every 5 to 30 saccharide repeat units of the polysaccharide and wherein said serotype 9N glycoconjugate has a weight average molecular weight (Mw) of between 1,500 kDa and 4,500 kDa.
38. The Streptococcus pneumoniae serotype 9N glycoconjugate of paragraph 1 wherein the weight average molecular weight (Mw) of said polysaccharide before conjugation is between 200 kDa and 400 kDa, wherein the degree of conjugation of said serotype 9N glycoconjugate is between 4 and 7, wherein the ratio of serotype 9N polysaccharide to carrier protein in the glycoconjugate (w/w) is between 0.5 and 1.5, wherein said serotype 9N glycoconjugate comprises at least one covalent linkage between the carrier protein and the polysaccharide for every 5 to 30 saccharide repeat units of the polysaccharide and wherein said serotype 9N glycoconjugate has a weight average molecular weight (Mw) of between 1,500 kDa and 4,500 kDa.
39. The Streptococcus pneumoniae serotype 9N glycoconjugate of paragraph 1 wherein the weight average molecular weight (Mw) of said polysaccharide before conjugation is between 200 kDa and 400 kDa, wherein the degree of conjugation of said serotype 9N glycoconjugate is between 4 and 12, wherein the ratio of serotype 9N polysaccharide to carrier protein in the glycoconjugate (w/w) is between 0.8 and 1.2, wherein said serotype 9N glycoconjugate comprises at least one covalent linkage between the carrier protein and the polysaccharide for every 5 to 30 saccharide repeat units of the polysaccharide and wherein said serotype 9N glycoconjugate has a weight average molecular weight (Mw) of between 1,500 kDa and 4,500 kDa.
40. The Streptococcus pneumoniae serotype 9N glycoconjugate of any one of paragraphs 1-39, wherein said serotype 9N glycoconjugate comprises less than about 50% of free serotype 9N polysaccharide compared to the total amount of serotype 9N polysaccharide.
41. The Streptococcus pneumoniae serotype 9N glycoconjugate of any one of paragraphs 1-39, wherein said serotype 9N glycoconjugate comprises less than about 40% of free serotype 9N polysaccharide compared to the total amount of serotype 9N polysaccharide.
42. The Streptococcus pneumoniae serotype 9N glycoconjugate of any one of paragraphs 1-39, wherein said serotype 9N glycoconjugate comprises less than about 25% of free serotype 9N polysaccharide compared to the total amount of serotype 9N polysaccharide.
43. The Streptococcus pneumoniae serotype 9N glycoconjugate of any one of paragraphs 1-39, wherein said serotype 9N glycoconjugate comprises less than about 20% of free serotype 9N polysaccharide compared to the total amount of serotype 9N polysaccharide.
44. The Streptococcus pneumoniae serotype 9N glycoconjugate of any one of paragraphs 1-39, wherein said serotype 9N glycoconjugate comprises less than about 15% of free serotype 9N polysaccharide compared to the total amount of serotype 9N polysaccharide.
45. The Streptococcus pneumoniae serotype 9N glycoconjugate of paragraph 1 wherein the weight average molecular weight (Mw) of said polysaccharide before conjugation is between 200 kDa and 500 kDa, wherein the degree of conjugation of said serotype 9N glycoconjugate is between 4 and 12, wherein the ratio of serotype 9N polysaccharide to carrier protein in the glycoconjugate (w/w) is between 0.5 and 1.5, wherein said serotype 9N glycoconjugate comprises at least one covalent linkage between the carrier protein and the polysaccharide for every 5 to 30 saccharide repeat units of the polysaccharide, wherein said serotype 9N glycoconjugate comprises less than about 20% of free serotype 9N polysaccharide compared to the total amount of serotype 9N polysaccharide and wherein said serotype 9N glycoconjugate has a weight average molecular weight (Mw) of between 1,500 kDa and 4,500 kDa.
46. The Streptococcus pneumoniae serotype 9N glycoconjugate of paragraph 1 wherein the weight average molecular weight (Mw) of said polysaccharide before conjugation is between 200 kDa and 400 kDa, wherein the degree of conjugation of said serotype 9N glycoconjugate is between 4 and 12, wherein the ratio of serotype 9N polysaccharide to carrier protein in the glycoconjugate (w/w) is between 0.5 and 1.5, wherein said serotype 9N glycoconjugate comprises at least one covalent linkage between the carrier protein and the polysaccharide for every 5 to 30 saccharide repeat units of the polysaccharide, wherein said serotype 9N glycoconjugate comprises less than about 15% of free serotype 9N polysaccharide compared to the total amount of serotype 9N polysaccharide and wherein said serotype 9N glycoconjugate has a weight average molecular weight (Mw) of between 1,500 kDa and 4,500 kDa.
47. The Streptococcus pneumoniae serotype 9N glycoconjugate of paragraph 1 wherein the weight average molecular weight (Mw) of said polysaccharide before conjugation is between 200 kDa and 400 kDa, wherein the degree of conjugation of said serotype 9N glycoconjugate is between 4 and 7, wherein the ratio of serotype 9N polysaccharide to carrier protein in the glycoconjugate (w/w) is between 0.5 and 1.5, wherein said serotype 9N glycoconjugate comprises at least one covalent linkage between the carrier protein and the polysaccharide for every 5 to 30 saccharide repeat units of the polysaccharide, wherein said serotype 9N glycoconjugate comprises less than about 15% of free serotype 9N polysaccharide compared to the total amount of serotype 9N polysaccharide and wherein said serotype 9N glycoconjugate has a weight average molecular weight (Mw) of between 1,500 kDa and 4,500 kDa.
48. The Streptococcus pneumoniae serotype 9N glycoconjugate of paragraph 1 wherein the weight average molecular weight (Mw) of said polysaccharide before conjugation is between 200 kDa and 400 kDa, wherein the degree of conjugation of said serotype 9N glycoconjugate is between 4 and 12, wherein the ratio of serotype 9N polysaccharide to carrier protein in the glycoconjugate (w/w) is between 0.8 and 1.2, wherein said serotype 9N glycoconjugate comprises at least one covalent linkage between the carrier protein and the polysaccharide for every 5 to 30 saccharide repeat units of the polysaccharide, wherein said serotype 9N glycoconjugate comprises less than about 15% of free serotype 9N polysaccharide compared to the total amount of serotype 9N polysaccharide and wherein said serotype 9N glycoconjugate has a weight average molecular weight (Mw) of between 1,500 kDa and 4,500 kDa.
49. The Streptococcus pneumoniae serotype 9N glycoconjugate of any one of paragraphs 1-48, wherein at least 30% of said serotype 9N glycoconjugate has a K_{d} below or equal to 0.3 in a CL-4B column.
50. The Streptococcus pneumoniae serotype 9N glycoconjugate of any one of paragraphs 1-48, wherein at least 40% of said serotype 9N glycoconjugate has a K_{d} below or equal to 0.3 in a CL-4B column/
51. The Streptococcus pneumoniae serotype 9N glycoconjugate of any one of paragraphs 1-48, wherein at least 45%, 50%, 55%, 60%, 65%, 70%, 75%, 80%, or 85% of said serotype 9N glycoconjugate has a K_{d} below or equal to 0.3 in a CL-4B column.
52. The Streptococcus pneumoniae serotype 9N glycoconjugate of any one of paragraphs 1-48, wherein at least 60% of said serotype 9N glycoconjugate has a K_{d} below or equal to 0.3 in a CL-4B column.
53. The Streptococcus pneumoniae serotype 9N glycoconjugate of any one of paragraphs 1-48, wherein between 50% and 80% of said serotype 9N glycoconjugate has a K_{d} below or equal to 0.3 in a CL-4B column.
54. The Streptococcus pneumoniae serotype 9N glycoconjugate of any one of paragraphs 1-48, wherein between 65% and 80% of said serotype 9N glycoconjugate has a K_{d} below or equal to 0.3 in a CL-4B column.
55. The Streptococcus pneumoniae serotype 9N glycoconjugate of paragraph 1 wherein the weight average molecular weight (Mw) of said polysaccharide before conjugation is between 200 kDa and 500 kDa, wherein the degree of conjugation of said serotype 9N glycoconjugate is between 4 and 12, wherein the ratio of serotype 9N polysaccharide to carrier protein in the glycoconjugate (w/w) is between 0.5 and 1.5, wherein said serotype 9N glycoconjugate comprises at least one covalent linkage between the carrier protein and the polysaccharide for every 5 to 30 saccharide repeat units of the polysaccharide, wherein said serotype 9N glycoconjugate comprises less than about 20% of free serotype 9N polysaccharide compared to the total amount of serotype 9N polysaccharide, wherein between 50% and 80% of said serotype 9N glycoconjugate has a K_{d} below or equal to 0.3 in a CL-4B column and wherein said serotype 9N glycoconjugate has a weight average molecular weight (Mw) of between 1,500 kDa and 4,500 kDa.
56. The Streptococcus pneumoniae serotype 9N glycoconjugate of paragraph 1 wherein the weight average molecular weight (Mw) of said polysaccharide before conjugation is between 200 kDa and 400 kDa, wherein the degree of conjugation of said serotype 9N glycoconjugate is between 4 and 12, wherein the ratio of serotype 9N polysaccharide to carrier protein in the glycoconjugate (w/w) is between 0.5 and 1.5, wherein said serotype 9N glycoconjugate comprises at least one covalent linkage between the carrier protein and the polysaccharide for every 5 to 30 saccharide repeat units of the polysaccharide, wherein said serotype 9N glycoconjugate comprises less than about 15% of free serotype 9N polysaccharide compared to the total amount of serotype 9N polysaccharide, wherein between 65% and 80% of said serotype 9N glycoconjugate has a K_{d} below or equal to 0.3 in a CL-4B column and wherein said serotype 9N glycoconjugate has a weight average molecular weight (Mw) of between 1,500 kDa and 4,500 kDa.
57. The Streptococcus pneumoniae serotype 9N glycoconjugate of paragraph 1 wherein the weight average molecular weight (Mw) of said polysaccharide before conjugation is between 200 kDa and 400 kDa, wherein the degree of conjugation of said serotype 9N glycoconjugate is between 4 and 7, wherein the ratio of serotype 9N polysaccharide to carrier protein in the glycoconjugate (w/w) is between 0.5 and 1.5, wherein said serotype 9N glycoconjugate comprises at least one covalent linkage between the carrier protein and the polysaccharide for every 5 to 30 saccharide repeat units of the polysaccharide, wherein said serotype 9N glycoconjugate comprises less than about 15% of free serotype 9N polysaccharide compared to the total amount of serotype 9N polysaccharide, wherein between 65% and 80% of said serotype 9N glycoconjugate has a K_{d} below or equal to 0.3 in a CL-4B column and wherein said serotype 9N glycoconjugate has a weight average molecular weight (Mw) of between 1,500 kDa and 4,500 kDa.
58. The Streptococcus pneumoniae serotype 9N glycoconjugate of paragraph 1 wherein the weight average molecular weight (Mw) of said polysaccharide before conjugation is between 200 kDa and 400 kDa, wherein the degree of conjugation of said serotype 9N glycoconjugate is between 4 and 12, wherein the ratio of serotype 9N polysaccharide to carrier protein in the glycoconjugate (w/w) is between 0.8 and 1.2, wherein said serotype 9N glycoconjugate comprises at least one covalent linkage between the carrier protein and the polysaccharide for every 5 to 30 saccharide repeat units of the polysaccharide, wherein said serotype 9N glycoconjugate comprises less than about 15% of free serotype 9N polysaccharide compared to the total amount of serotype 9N polysaccharide, wherein between 65% and 80% of said serotype 9N glycoconjugate has a K_{d} below or equal to 0.3 in a CL-4B column and wherein said serotype 9N glycoconjugate has a weight average molecular weight (Mw) of between 1,500 kDa and 4,500 kDa.
59. The Streptococcus pneumoniae serotype 9N glycoconjugate of any one of paragraphs 1-58, wherein said serotype 9N saccharide is coupled to the carrier protein via non-covalent bonds.
60. The Streptococcus pneumoniae serotype 9N glycoconjugate of any one of paragraphs 1-58, wherein said serotype 9N glycoconjugate is prepared by CDI chemistry.
61. The Streptococcus pneumoniae serotype 9N glycoconjugate of any one of paragraphs 1-58, wherein said serotype 9N glycoconjugate is prepared by CDT chemistry
62. The Streptococcus pneumoniae serotype 9N glycoconjugate of any one of paragraphs 1-58, wherein said serotype 9N glycoconjugate is prepared by eTEC chemistry.
63. The Streptococcus pneumoniae serotype 9N glycoconjugate of any one of paragraphs 1-58, wherein said serotype 9N glycoconjugate is prepared by direct reductive amination.
64. The Streptococcus pneumoniae serotype 9N glycoconjugate of any one of paragraphs 1-58, wherein said serotype 9N glycoconjugate is prepared by a process comprising the step of: (a) reacting said serotype 9N saccharide with an oxidizing agent; (b) compounding the activated saccharide of step (a) with a carrier protein; and (c) reacting the compounded activated saccharide and carrier protein with a reducing agent to form a glycoconjugate.
65. The Streptococcus pneumoniae serotype 9N glycoconjugate of any one of paragraphs 1-58, wherein said serotype 9N glycoconjugate is prepared by a process comprising the step of: (a) reacting said serotype 9N saccharide with an oxidizing agent; (a′) quenching the oxidation reaction by addition of a quenching agent; (b) compounding the activated saccharide of step (a′) with a carrier protein; and (c) reacting the compounded activated saccharide and carrier protein with a reducing agent to form a glycoconjugate.
66. The Streptococcus pneumoniae serotype 9N glycoconjugate of any one of paragraphs 64-65, wherein said the oxidizing agent is a mixture of a stable nitroxyl radical compound with an oxidant.
67. The Streptococcus pneumoniae serotype 9N glycoconjugate of paragraph 66, wherein said said stable nitroxyl radical compound is 2,2,6,6-Tetramethyl-1-piperidinyloxy free radical (TEMPO) and said oxidant is N-Chlorosuccinimide (NCS).
68. The Streptococcus pneumoniae serotype 9N glycoconjugate of any one of paragraphs 64-67, wherein the initial input ratio (weight by weight) of activated serotype 9N saccharide to carrier protein at step b) is between 1.5:1 and 0.5:1.
69. The Streptococcus pneumoniae serotype 9N glycoconjugate of any one of paragraphs 64-68, wherein the reduction reaction (c) is carried out in aqueous solvent.
70. The Streptococcus pneumoniae serotype 9N glycoconjugate of any one of paragraphs 64-68, wherein the reduction reaction (c) is carried out in aprotic solvent.
71. The Streptococcus pneumoniae serotype 9N glycoconjugate of any one of paragraphs 64-68, wherein the reduction reaction (c) is carried out in a solution consisting essentially of dimethylsulphoxide (DMSO).
72. The Streptococcus pneumoniae serotype 9N glycoconjugate of any one of paragraphs 1-58, wherein said serotype 9N glycoconjugate is prepared click chemistry.
73. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (IV):

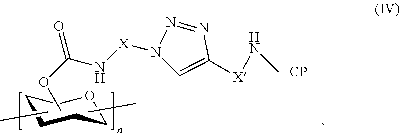

wherein X is selected from the group consisting of CH₂(CH₂)_{n′}, (CH₂CH₂O)ₘCH₂CH₂, NHCO(CH₂)_{n′}, NHCO(CH₂CH₂O)ₘCH₂CH₂, OCH₂(CH₂)_{n′} and O(CH₂CH₂O)ₘCH₂CH₂; where n′ is selected from 1 to 10 and m is selected from 1 to 4, wherein X′ is selected from the group consisting of CH₂O(CH₂)_{n″}CH₂C═O, CH₂O(CH₂CH₂O)_{m′}(CH₂)_{n″}CH₂C═O, where n″ is selected from 0 to 10 and m′ is selected from 0 to 4, wherein the structure in square backet represents a repeat unit of the serotype 9N saccharide and wherein n represents the number of repeating units.
74. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (IV), wherein X is CH₂(CH₂)_{n′}, where n′ is 2 and wherein X′ is CH₂O(CH₂)_{n″}CH₂C═O where n″ is 1.
75. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (V),

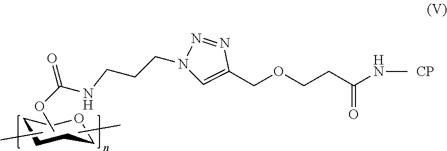

wherein the structure in square backet represents a repeat unit of the serotype 9N saccharide and wherein n represents the number of repeating units.
76. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (IV), wherein X is CH₂(CH₂)_{n′}, where n′ is selected from 1 to 10 and wherein X′ is CH₂O(CH₂)_{n″}CH₂C═O where n″ is selected from 0 to 10.
77. I The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (IV), wherein X is CH₂(CH₂)_{n′}, where n′ is selected from 1 to 10 and wherein CH₂O(CH₂CH₂O)_{m′}(CH₂)_{n″}CH₂C═O, where n″ is selected from 0 to 10 and m′ is selected from 0 to 4.
78. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (IV), wherein X is (CH₂CH₂O)ₘCH₂CH₂, where m is selected from 1 to 4 and wherein X′ is CH₂O(CH₂)_{n″}CH₂C═O, where n″ is selected from 0 to 10.
79. In an embodiment, the invention provides a serotype 9N glycoconjugate comprising a serotype 9N saccharide covalently conjugated to a carrier protein (CP) through a spacer and having the general formula (IV), wherein X is (CH₂CH₂O)ₘCH₂CH₂, where m is selected from 1 to 4 and wherein X′ is CH₂O(CH₂CH₂O)_{m′}(CH₂)_{n″}CH₂C═O, where n″ is selected from 0 to 10 and m′ is selected from 0 to 4.
80. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (IV), wherein X is NHCO(CH₂)_{n′}, where n′ is selected from 1 to 10 and wherein X′ is CH₂O(CH₂CH₂O)_{m′}(CH₂)_{n″}CH₂C═O, where n″ is selected from 0 to 10 and m′ is selected from 0 to 4.
81. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (IV), wherein X is NHCO(CH₂CH₂O)ₘCH₂CH₂, where m is selected from 1 to 4 and wherein X′ is CH₂O(CH₂)_{n″}CH₂C═O, where n″ is selected from 0 to 10. In an embodiment, m is selected from 1 to 3 and n″ is selected from 0 to 10.
82. I The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (IV), wherein X is NHCO(CH₂CH₂O)ₘCH₂CH₂, where m is selected from 1 to 4 and wherein X′ is CH₂O(CH₂CH₂O)_{m′}(CH₂)_{n″}CH₂C═O, where n″ is selected from 0 to 10 and m′ is selected from 0 to 4.
83. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (IV), wherein X is OCH₂(CH₂)_{n′}, where n′ is selected from 1 to 10 and wherein X′ is CH₂O(CH₂CH₂O)_{m′}(CH₂)_{n″}CH₂C═O, where n″ is selected from 0 to 10 and m′ is selected from 0 to 4.
84. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (IV), wherein X is O(CH₂CH₂O)ₘCH₂CH₂, where m is selected from 1 to 4 and wherein X′ is CH₂O(CH₂)_{n″}CH₂C═O, where n″ is selected from 0 to 10.
85. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (IV), wherein X is O(CH₂CH₂O)ₘCH₂CH₂, where m is selected from 1 to 4 and wherein X′ is CH₂O(CH₂CH₂O)_{m′}(CH₂)_{n″}CH₂C═O, where n″ is selected from 0 to 10 and m′ is selected from 0 to 4.
86. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (XII):

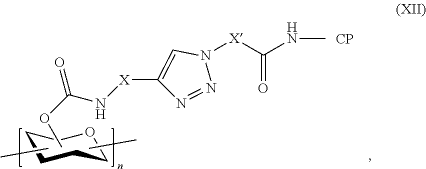

wherein X is selected from the group consisting of CH₂(CH₂)_{n′}, (CH₂CH₂O)ₘCH₂CH₂, NHCO(CH₂)_{n′}, NHCO(CH₂CH₂O)ₘCH₂CH₂, OCH₂(CH₂)_{n′} and O(CH₂CH₂O)ₘCH₂CH₂; where n′ is selected from 0 to 10 and m is selected from 1 to 4, and wherein X′ is selected from the group consisting of CH₂(CH₂)_{n″}, CH₂O(CH₂)_{n″}CH₂, CH₂O(CH₂CH₂O)_{m′}(CH₂)_{n″}CH₂, where n″ is selected from 0 to 10 and m′ is selected from 0 to 4, wherein the structure in square backet represents a repeat unit of the serotype 9N saccharide and wherein n represents the number of repeating units.
87. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (XII), wherein X is CH₂(CH₂)_{n′}, where n′ is 0 and wherein X′ is CH₂(CH₂)_{n″} where n″ is 0.
88. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (XIII),

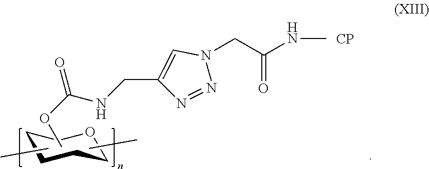

wherein the structure in square backet represents a repeat unit of the serotype 9N saccharide and wherein n represents the number of repeating units.
89. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (XII), wherein X is CH₂(CH₂)_{n′}, where n′ is selected from 0 to 10 and wherein X′ is CH₂(CH₂)_{n″} where n″ is selected from 0 to 10.
90. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (XII), wherein X is CH₂(CH₂)_{n′}, where n′ is selected from 0 to 10 and wherein X′ is CH₂O(CH₂)_{n″}CH₂ where n″ is selected from 0 to 10.
91. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (XII), wherein X is CH₂(CH₂)_{n′}, where n′ is selected from 0 to 10 and wherein X′ is CH₂O(CH₂CH₂O)_{m′}(CH₂)_{n″}CH₂, where n″ is selected from 0 to 10 and m′ is selected from 0 to 4.
92. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (XII), wherein X is (CH₂CH₂O)ₘCH₂CH₂, where m is selected from 1 to 4 and wherein X′ is CH₂(CH₂)_{n″}, where n″ is selected from 0 to 10.
93. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (XII), wherein X is (CH₂CH₂O)ₘCH₂CH₂, where m is selected from 1 to 4 and wherein X′ is CH₂O(CH₂)_{n″}CH₂, where n″ is selected from 0 to 10.
94. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (XII), wherein X is (CH₂CH₂O)ₘCH₂CH₂, where m is selected from 1 to 4 and wherein X′ is CH₂O(CH₂CH₂O)_{m′}(CH₂)_{n″}CH₂, where n″ is selected from 0 to 10 and m′ is selected from 0 to 4.
95. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (XII), wherein X is NHCO(CH₂)_{n′}, where n′ is selected from 1 to 10 and wherein X′ is CH₂(CH₂)_{n″}, where n″ is selected from 0 to 10.
96. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (VII), wherein X is NHCO(CH₂)_{n′}, where n′ is selected from 1 to 10 and wherein X′ is CH₂O(CH₂)_{n″}CH₂, where n″ is selected from 0 to 10. In an embodiment, n′ is selected from 1 to 5 and n″ is selected from 0 to 10.
97. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (XII), wherein X is NHCO(CH₂)_{n′}, where n′ is selected from 1 to 10 and wherein X′ is CH₂O(CH₂CH₂O)_{m′}(CH₂)_{n″}CH₂, where n″ is selected from 0 to 10 and m′ is selected from 0 to 4.
98. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (XII), wherein X is NHCO(CH₂CH₂O)ₘCH₂CH₂, where m is selected from 1 to 4 and wherein X′ is CH₂(CH₂)_{n″}, where n″ is selected from 0 to 10.
99. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (XII), wherein X is NHCO(CH₂CH₂O)ₘCH₂CH₂, where m is selected from 1 to 4 and wherein X′ is CH₂O(CH₂)_{n″}CH₂, where n″ is selected from 0 to 10.
100. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (XII), wherein X is NHCO(CH₂CH₂O)ₘCH₂CH₂, where m is selected from 1 to 4 and wherein X′ is CH₂O(CH₂CH₂O)_{m′}(CH₂)_{n″}CH₂, where n″ is selected from 0 to 10 and m′ is selected from 0 to 4.
101. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (XII), wherein X is OCH₂(CH₂)_{n′}, where n′ is selected from 1 to 10 and wherein X′ is CH₂(CH₂)_{n″}, where n″ is selected from 0 to 10.
102. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (XII), wherein X is OCH₂(CH₂)_{n′}, where n′ is selected from 1 to 10 and wherein X′ is CH₂O(CH₂)_{n″}CH₂, where n″ is selected from 0 to 10.
103. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (XII), wherein X is OCH₂(CH₂)_{n′}, where n′ is selected from 1 to 10 and wherein X′ is CH₂O(CH₂CH₂O)_{m′}(CH₂)_{n″}CH₂, where n″ is selected from 0 to 10 and m′ is selected from 0 to 4.
104. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (XII), wherein X is O(CH₂CH₂O)ₘCH₂CH₂, where m is selected from 1 to 4 and wherein X′ is CH₂(CH₂)_{n″}, where n″ is selected from 0 to 10.
105. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (XII), wherein X is O(CH₂CH₂O)ₘCH₂CH₂, where m is selected from 1 to 4 and wherein X′ is CH₂O(CH₂)_{n″}CH₂, where n″ is selected from 0 to 10.
106. The Streptococcus pneumoniae serotype 9N glycoconjugate of any one of paragraphs 1-58, wherein said serotype 9N glycoconjugate comprises a serotype 9N saccharide covalently conjugated to a carrier protein (CP) through a spacer and having the general formula (XII), wherein X is O(CH₂CH₂O)ₘCH₂CH₂, where m is selected from 1 to 4 and wherein X′ is CH₂O(CH₂CH₂O)_{m′}(CH₂)_{n″}CH₂, where n″ is selected from 0 to 10 and m′ is selected from 0 to 4.
107. The Streptococcus pneumoniae serotype 9N glycoconjugate of any one of paragraphs 1-106, wherein the carrier protein of said serotype 9N capsular polysaccharide glycoconjugate is TT, DT, DT mutants (such as CRM₁₉₇), or a C5a peptidase from Streptococcus (SCP).
108. The Streptococcus pneumoniae serotype 9N glycoconjugate of any one of paragraphs 1-106, wherein the carrier protein of said serotype 9N capsular polysaccharide glycoconjugate is DT (Diphtheria toxoid).
109. The Streptococcus pneumoniae serotype 9N glycoconjugate of any one of paragraphs 1-106, wherein the carrier protein of said serotype 9N capsular polysaccharide glycoconjugate is is TT (tetanus toxoid).
110. The Streptococcus pneumoniae serotype 9N glycoconjugate of any one of paragraphs 1-106, wherein the carrier protein of said serotype 9N capsular polysaccharide glycoconjugate is is PD (H. influenzae protein D).
111. The Streptococcus pneumoniae serotype 9N glycoconjugate of any one of paragraphs 1-106, wherein the carrier protein of said serotype 9N capsular polysaccharide glycoconjugate is is CRM₁₉₇ or a C5a peptidase from Streptococcus (SCP).
112. The Streptococcus pneumoniae serotype 9N glycoconjugate of any one of paragraphs 1-106, wherein the carrier protein of said serotype 9N capsular polysaccharide glycoconjugate is CRM₁₉₇.
113. The Streptococcus pneumoniae serotype 9N glycoconjugate of any one of paragraphs 1-106, wherein the carrier protein of said serotype 9N capsular polysaccharide glycoconjugate is the A chain of CRM₁₉₇.
114. The Streptococcus pneumoniae serotype 9N glycoconjugate of any one of paragraphs 1-106, wherein the carrier protein of said serotype 9N capsular polysaccharide glycoconjugate is is SCP (Streptococcal C5a Peptidase).
115. The Streptococcus pneumoniae serotype 9N glycoconjugate of any one of paragraphs 1-106, wherein the carrier protein of said serotype 9N capsular polysaccharide glycoconjugate is is an SCP from GBS (SCPB).
116. The Streptococcus pneumoniae serotype 9N glycoconjugate of any one of paragraphs 1-106, wherein the carrier protein of said serotype 9N capsular polysaccharide glycoconjugate is is an SCP from GAS (SCPA).
117. The Streptococcus pneumoniae serotype 9N glycoconjugate of any one of paragraphs 1-106, wherein the carrier protein of said serotype 9N capsular polysaccharide glycoconjugate is is an enzymatically inactive SCP.
118. The Streptococcus pneumoniae serotype 9N glycoconjugate of any one of paragraphs 1-106, wherein the carrier protein of said serotype 9N capsular polysaccharide glycoconjugate is is an enzymatically inactive SCP from GBS (SCPB).
119. The Streptococcus pneumoniae serotype 9N glycoconjugate of any one of paragraphs 1-106, wherein the carrier protein of said serotype 9N capsular polysaccharide glycoconjugate is is an enzymatically inactive SCP from GAS (SCPA).
120. The Streptococcus pneumoniae serotype 9N glycoconjugate of any one of paragraphs 1-106, wherein the carrier protein of said serotype 9N capsular polysaccharide glycoconjugate is a fragment of an SCP.
121. The Streptococcus pneumoniae serotype 9N glycoconjugate of any one of paragraphs 1-106, wherein the carrier protein of said serotype 9N capsular polysaccharide glycoconjugate is is a fragment of an SCPA.
122. The Streptococcus pneumoniae serotype 9N glycoconjugate of any one of paragraphs 1-106, wherein the carrier protein of said serotype 9N capsular polysaccharide glycoconjugate is is a fragment of an SCPB.
123. The Streptococcus pneumoniae serotype 9N glycoconjugate of any one of paragraphs 1-106, wherein the carrier protein of said serotype 9N capsular polysaccharide glycoconjugate is is a fragment of an SCP which comprises the protease domain, the protease-associated domain (PA domain) and the three fibronectin type III (Fn) domains but does not comprise the export signal presequence, the pro-sequence and the cell wall anchor domain.
124. The Streptococcus pneumoniae serotype 9N glycoconjugate of any one of paragraphs 1-106, wherein the carrier protein of said serotype 9N capsular polysaccharide glycoconjugate is is a fragment of an SCP which comprises the protease domain, the protease-associated domain (PA domain) and the three fibronectin type III (Fn) domains but does not comprise the export signal presequence, the pro-sequence and the cell wall anchor domain.
125. The Streptococcus pneumoniae serotype 9N glycoconjugate of any one of paragraphs 1-106, wherein the carrier protein of said serotype 9N capsular polysaccharide glycoconjugate is is an enzymatically inactive fragment of an SCP which comprises the protease domain, the protease-associated domain (PA domain) and two of the three fibronectin type III (Fn) domains but does not comprise the export signal presequence, the pro-sequence and the cell wall anchor domain.
126. The Streptococcus pneumoniae serotype 9N glycoconjugate of any one of paragraphs 1-106, wherein the carrier protein of said serotype 9N capsular polysaccharide glycoconjugate is is an enzymatically inactive fragment of an SCP.
127. The Streptococcus pneumoniae serotype 9N glycoconjugate of any one of paragraphs 1-106, wherein the carrier protein of said serotype 9N capsular polysaccharide glycoconjugate is an enzymatically inactive fragment of an SCP comprising the protease domain, the protease-associated domain (PA domain) and the three fibronectin type III (Fn) domains but not comprising the export signal presequence, the pro-sequence and the cell wall anchor domain.
128. The Streptococcus pneumoniae serotype 9N glycoconjugate of any one of paragraphs 1-106, wherein the carrier protein of said serotype 9N capsular polysaccharide glycoconjugate is an enzymatically inactive fragment of an SCPA.
129. The Streptococcus pneumoniae serotype 9N glycoconjugate of any one of paragraphs 1-106, wherein the carrier protein of said serotype 9N capsular polysaccharide glycoconjugate is an enzymatically inactive fragment of an SCPA comprising the protease domain, the protease-associated domain (PA domain) and the three fibronectin type III (Fn) domains but not comprising the export signal presequence, the pro-sequence and the cell wall anchor domain.
130. The Streptococcus pneumoniae serotype 9N glycoconjugate of any one of paragraphs 1-106, wherein the carrier protein of said serotype 9N capsular polysaccharide glycoconjugate is an enzymatically inactive fragment of SCPB.
131. The Streptococcus pneumoniae serotype 9N glycoconjugate of any one of paragraphs 1-106, wherein the carrier protein of said serotype 9N capsular polysaccharide glycoconjugate is an enzymatically inactive fragment of SCPB comprising the protease domain, the protease-associated domain (PA domain) and the three fibronectin type III (Fn) domains but not comprising the export signal presequence, the pro-sequence and the cell wall anchor domain.
132. The Streptococcus pneumoniae serotype 9N glycoconjugate of any one of paragraphs 1-106, wherein the carrier protein of said serotype 9N capsular polysaccharide glycoconjugate is an enzymatically inactive SCP where said inactivation is accomplished by replacing at least one amino acid of the wild type sequence.
133. The Streptococcus pneumoniae serotype 9N glycoconjugate of any one of paragraphs 1-106, wherein the carrier protein of said serotype 9N capsular polysaccharide glycoconjugate is an enzymatically inactive fragment of an SCP where said inactivation is accomplished by replacing at least one amino acid of the wild type sequence.
134. The Streptococcus pneumoniae serotype 9N glycoconjugate of any one of paragraphs 1-106, wherein the carrier protein of said serotype 9N capsular polysaccharide glycoconjugate is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least one amino acid of the wild type sequence.
135. The Streptococcus pneumoniae serotype 9N glycoconjugate of any one of paragraphs 1-106, wherein the carrier protein of said serotype 9N capsular polysaccharide glycoconjugate is an enzymatically inactive SCP where said inactivation is accomplished by replacing at least two amino acids of the wild type sequence.
136. The Streptococcus pneumoniae serotype 9N glycoconjugate of any one of paragraphs 1-106, wherein the carrier protein of said serotype 9N capsular polysaccharide glycoconjugate is is an enzymatically inactive fragment of an SCP where said inactivation is accomplished by replacing at least two amino acids of the wild type sequence.
137. The Streptococcus pneumoniae serotype 9N glycoconjugate of any one of paragraphs 1-106, wherein the carrier protein of said serotype 9N capsular polysaccharide glycoconjugate is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least two amino acids of the wild type sequence.
138. The Streptococcus pneumoniae serotype 9N glycoconjugate of any one of paragraphs 1-106, wherein the carrier protein of said serotype 9N capsular polysaccharide glycoconjugate is an enzymatically inactive SCP where said inactivation is accomplished by replacing at least three amino acids of the wild type sequence.
139. The Streptococcus pneumoniae serotype 9N glycoconjugate of any one of paragraphs 1-106, wherein the carrier protein of said serotype 9N capsular polysaccharide glycoconjugate is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least three amino acids of the wild type sequence.
140. The Streptococcus pneumoniae serotype 9N glycoconjugate of any one of paragraphs 1-106, wherein the carrier protein of said serotype 9N capsular polysaccharide glycoconjugate is an enzymatically inactive fragment of SCPA which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least three amino acids of the wild type sequence.
141. The Streptococcus pneumoniae serotype 9N glycoconjugate of any one of paragraphs 1-106, wherein the carrier protein of said serotype 9N capsular polysaccharide glycoconjugate is an enzymatically inactive fragment of SCPB which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least three amino acids of the wild type sequence.
142. The Streptococcus pneumoniae serotype 9N glycoconjugate of any one of paragraphs 1-106, wherein the carrier protein of said serotype 9N capsular polysaccharide glycoconjugate is an enzymatically inactive SCP where said inactivation is accomplished by replacing at least four amino acids of the wild type sequence.
143. The Streptococcus pneumoniae serotype 9N glycoconjugate of any one of paragraphs 1-106, wherein the carrier protein of said serotype 9N capsular polysaccharide glycoconjugate is an enzymatically inactive SCPA where said inactivation is accomplished by replacing at least four amino acids of the wild type sequence.
144. The Streptococcus pneumoniae serotype 9N glycoconjugate of any one of paragraphs 1-106, wherein the carrier protein of said serotype 9N capsular polysaccharide glycoconjugate is an enzymatically inactive SCPB where said inactivation is accomplished by replacing at least four amino acids of the wild type sequence.
145. The Streptococcus pneumoniae serotype 9N glycoconjugate of any one of paragraphs 1-106, wherein the carrier protein of said serotype 9N capsular polysaccharide glycoconjugate is an enzymatically inactive fragment of an SCP where said inactivation is accomplished by replacing at least four amino acids of the wild type sequence.
146. The Streptococcus pneumoniae serotype 9N glycoconjugate of any one of paragraphs 1-106, wherein the carrier protein of said serotype 9N capsular polysaccharide glycoconjugate is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least four amino acids of the wild type sequence.
147. The Streptococcus pneumoniae serotype 9N glycoconjugate of any one of paragraphs 1-106, wherein the carrier protein of said serotype 9N capsular polysaccharide glycoconjugate is an enzymatically inactive fragment of SCPA which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least four amino acids of the wild type sequence.
148. The Streptococcus pneumoniae serotype 9N glycoconjugate of any one of paragraphs 1-106, wherein the carrier protein of said serotype 9N capsular polysaccharide glycoconjugate is an enzymatically inactive fragment of SCPB which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least four amino acids of the wild type sequence.
149. The Streptococcus pneumoniae serotype 9N glycoconjugate of any one of paragraphs 1-106, wherein the carrier protein of said serotype 9N capsular polysaccharide glycoconjugate is an enzymatically inactive fragment of SCP which consists of SEQ ID NO: 41.
150. The Streptococcus pneumoniae serotype 9N glycoconjugate of any one of paragraphs 1-106, wherein the carrier protein of said serotype 9N capsular polysaccharide glycoconjugate is an enzymatically inactive fragment of SCP which consists of SEQ ID NO: 42.
151. The Streptococcus pneumoniae serotype 9N glycoconjugate of any one of paragraphs 1-106, wherein the carrier protein of said serotype 9N capsular polysaccharide glycoconjugate is an enzymatically inactive fragment of SCP consisting of a polypeptide having at least 90% identity with SEQ ID NO: 41.
152. The Streptococcus pneumoniae serotype 9N glycoconjugate of any one of paragraphs 1-106, wherein the carrier protein of said serotype 9N capsular polysaccharide glycoconjugate is an enzymatically inactive fragment of SCP consisting of a polypeptide having at least 95% identity with SEQ ID NO: 41.
153. The Streptococcus pneumoniae serotype 9N glycoconjugate of any one of paragraphs 1-106, wherein the carrier protein of said serotype 9N capsular polysaccharide glycoconjugate is an enzymatically inactive fragment of SCP consisting of a polypeptide having at least 99% identity with SEQ ID NO: 41.
154. The Streptococcus pneumoniae serotype 9N glycoconjugate of any one of paragraphs 1-106, wherein the carrier protein of said serotype 9N capsular polysaccharide glycoconjugate is an enzymatically inactive fragment of SCP consisting of a polypeptide having at least 99.5% identity with SEQ ID NO: 41.
155. The Streptococcus pneumoniae serotype 9N glycoconjugate of any one of paragraphs 1-106, wherein the carrier protein of said serotype 9N capsular polysaccharide glycoconjugate is an enzymatically inactive fragment of SCP consisting of a polypeptide having at least 99.8% identity with SEQ ID NO: 41.
156. The Streptococcus pneumoniae serotype 9N glycoconjugate of any one of paragraphs 1-106, wherein the carrier protein of said serotype 9N capsular polysaccharide glycoconjugate is an enzymatically inactive fragment of SCP consisting of a polypeptide having at least 99.85% identity with SEQ ID NO: 41.
157. The Streptococcus pneumoniae serotype 9N glycoconjugate of any one of paragraphs 1-106, wherein the carrier protein of said serotype 9N capsular polysaccharide glycoconjugate is an enzymatically inactive fragment of SCP consisting of a polypeptide having at least 90% identity with SEQ ID NO: 42.
158. The Streptococcus pneumoniae serotype 9N glycoconjugate of any one of paragraphs 1-106, wherein the carrier protein of said serotype 9N capsular polysaccharide glycoconjugate is an enzymatically inactive fragment of SCP consisting of a polypeptide having at least 95% identity with SEQ ID NO: 42.
159. The Streptococcus pneumoniae serotype 9N glycoconjugate of any one of paragraphs 1-106, wherein the carrier protein of said serotype 9N capsular polysaccharide glycoconjugate is an enzymatically inactive fragment of SCP consisting of a polypeptide having at least 99% identity with SEQ ID NO: 42.
160. The Streptococcus pneumoniae serotype 9N glycoconjugate of any one of paragraphs 1-106, wherein the carrier protein of said serotype 9N capsular polysaccharide glycoconjugate is an enzymatically inactive fragment of SCP consisting of a polypeptide having at least 99.5% identity with SEQ ID NO: 42.
161. The Streptococcus pneumoniae serotype 9N glycoconjugate of any one of paragraphs 1-106, wherein the carrier protein of said serotype 9N capsular polysaccharide glycoconjugate is an enzymatically inactive fragment of SCP consisting of a polypeptide having at least 99.8% identity with SEQ ID NO: 42.
162. The Streptococcus pneumoniae serotype 9N glycoconjugate of any one of paragraphs 1-106, wherein the carrier protein of said serotype 9N capsular polysaccharide glycoconjugate is an enzymatically inactive fragment of SCP consisting of a polypeptide having at least 99.85% identity with SEQ ID NO: 42.
163. An immunogenic composition comprising a Streptococcus pneumoniae serotype 9N glycoconjugate of any one of paragraphs 1-162.
164. An immunogenic composition comprising a Streptococcus pneumoniae serotype 9N glycoconjugate of any one of paragraphs 1-162 and comprising from 1 to 35 different glycoconjugates.
165. An immunogenic composition comprising a Streptococcus pneumoniae serotype 9N glycoconjugate of any one of paragraphs 1-162 and comprising from 1 to 35 glycoconjugates from different serotypes of S. pneumoniae.
166. An immunogenic composition comprising a Streptococcus pneumoniae serotype 9N glycoconjugate of any one of paragraphs 1-162 and comprising glycoconjugates from 7, 8, 9, 10, 11, 12, 13, 14, 15, 16, 17, 18, 19, 20, 21, 22, 23, 24, 25, 26, 27, 28, 29, 30, 31, 32, 33, 34 or 35 different serotypes of S. pneumoniae.
167. An immunogenic composition comprising a Streptococcus pneumoniae serotype 9N glycoconjugate of any one of paragraphs 1-162 and comprising glycoconjugates from 21 to 35 different serotypes of S. pneumoniae.
168. An immunogenic composition comprising a Streptococcus pneumoniae serotype 9N glycoconjugate of any one of paragraphs 1-162 wherein said immunogenic composition is a 21-valent pneumococcal conjugate composition.
169. An immunogenic composition comprising a Streptococcus pneumoniae serotype 9N glycoconjugate of any one of paragraphs 1-162 wherein said immunogenic composition is a 22-valent pneumococcal conjugate composition.
170. An immunogenic composition comprising a Streptococcus pneumoniae serotype 9N glycoconjugate of any one of paragraphs 1-162 wherein said immunogenic composition is a 23-valent pneumococcal conjugate composition.
171. An immunogenic composition comprising a Streptococcus pneumoniae serotype 9N glycoconjugate of any one of paragraphs 1-162 wherein said immunogenic composition is a 24-valent pneumococcal conjugate composition.
172. An immunogenic composition comprising a Streptococcus pneumoniae serotype 9N glycoconjugate of any one of paragraphs 1-162 wherein said immunogenic composition is a 25-valent pneumococcal conjugate composition.
173. An immunogenic composition comprising a Streptococcus pneumoniae serotype 9N glycoconjugate of any one of paragraphs 1-162 wherein said immunogenic composition is a 26-valent pneumococcal conjugate composition.
174. An immunogenic composition comprising a Streptococcus pneumoniae serotype 9N glycoconjugate of any one of paragraphs 1-162 wherein said immunogenic composition is a 27-valent pneumococcal conjugate composition. 175. An immunogenic composition comprising a Streptococcus pneumoniae serotype 9N glycoconjugate of any one of paragraphs 1-162 wherein said immunogenic composition is a 28-valent pneumococcal conjugate composition.
176. An immunogenic composition comprising a Streptococcus pneumoniae serotype 9N glycoconjugate of any one of paragraphs 1-162 wherein said immunogenic composition is a 29-valent pneumococcal conjugate composition.
177. An immunogenic composition comprising a Streptococcus pneumoniae serotype 9N glycoconjugate of any one of paragraphs 1-162 wherein said immunogenic composition is a 30-valent pneumococcal conjugate composition.
178. An immunogenic composition comprising a Streptococcus pneumoniae serotype 9N glycoconjugate of any one of paragraphs 1-162 wherein said immunogenic composition is a 31-valent pneumococcal conjugate composition.
179. An immunogenic composition comprising a Streptococcus pneumoniae serotype 9N glycoconjugate of any one of paragraphs 1-162 wherein said immunogenic composition is a 32-valent pneumococcal conjugate composition.
180. An immunogenic composition comprising a Streptococcus pneumoniae serotype 9N glycoconjugate of any one of paragraphs 1-162 wherein said immunogenic composition is a 33-valent pneumococcal conjugate composition.
181. An immunogenic composition comprising a Streptococcus pneumoniae serotype 9N glycoconjugate of any one of paragraphs 1-162 wherein said immunogenic composition is a 34-valent pneumococcal conjugate composition.
182. An immunogenic composition comprising a Streptococcus pneumoniae serotype 9N glycoconjugate of any one of paragraphs 1-162 wherein said immunogenic composition is a 35-valent pneumococcal conjugate composition.
183. An immunogenic composition comprising a Streptococcus pneumoniae serotype 9N glycoconjugate of any one of paragraphs 1-162 and further comprising glycoconjugates from S. pneumoniae serotypes 4, 6B, 9V, 14, 18C, 19F and 23F.
184. An immunogenic composition comprising a Streptococcus pneumoniae serotype 9N glycoconjugate of any one of paragraphs 1-162 and further comprising glycoconjugates from S. pneumoniae serotypes 1, 4, 5, 6A, 6B, 7F, 9V, 14, 18C, 19F and 23F.
185. An immunogenic composition comprising a Streptococcus pneumoniae serotype 9N glycoconjugate of any one of paragraphs 1-162 and further comprising glycoconjugates from S. pneumoniae serotypes 1, 4, 5, 6A, 6B, 7F, 9V, 14, 18C, 19A, 19F and 23F.
186. An immunogenic composition comprising a Streptococcus pneumoniae serotype 9N glycoconjugate of any one of paragraphs 1-162 and further comprising glycoconjugates from S. pneumoniae serotypes 1, 3, 4, 5, 6A, 6B, 7F, 9V, 14, 18C, 19A, 19F and 23F.
187. An immunogenic composition comprising a Streptococcus pneumoniae serotype 9N glycoconjugate of any one of paragraphs 1-162 and further comprising glycoconjugates from S. pneumoniae serotypes 1, 3, 4, 5, 6A, 6B, 7F, 9V, 14, 18C, 19A, 19F, 22F, 23F and 33F.
188. An immunogenic composition comprising a Streptococcus pneumoniae serotype 9N glycoconjugate of any one of paragraphs 1-162 and further comprising glycoconjugates from S. pneumoniae serotypes 1, 2, 3, 4, 5, 6A, 6B, 7F, 9V, 14, 18C, 19A, 19F, 22F, 23F and 33F.
189. An immunogenic composition comprising a Streptococcus pneumoniae serotype 9N glycoconjugate of any one of paragraphs 1-162 and further comprising glycoconjugates from S. pneumoniae serotypes 1, 3, 4, 5, 6A, 6B, 8, 7F, 9V, 10A, 11A, 12F, 14, 15B, 18C, 19A, 19F, 22F, 23F and 33F.
190. An immunogenic composition comprising a Streptococcus pneumoniae serotype 9N glycoconjugate of any one of paragraphs 1-162 and further comprising glycoconjugates from S. pneumoniae serotypes 1, 2, 3, 4, 5, 6A, 6B, 8, 7F, 9V, 10A, 11A, 12F, 14, 15B, 18C, 19A, 19F, 22F, 23F and 33F.
191. An immunogenic composition comprising a Streptococcus pneumoniae serotype 9N glycoconjugate of any one of paragraphs 1-162 and further comprising glycoconjugates from S. pneumoniae serotypes 1, 2, 3, 4, 5, 6A, 6B, 8, 7F, 9V, 10A, 11A, 12F, 14, 15B, 18C, 19A, 19F, 20B, 22F, 23F and 33F. In an embodiment the immunogenic composition is a 23-valent pneumococcal conjugate compositions.
192. An immunogenic composition comprising a Streptococcus pneumoniae serotype 9N glycoconjugate of any one of paragraphs 1-162 and further comprising glycoconjugates from S. pneumoniae serotypes 1, 2, 3, 4, 5, 6A, 6B, 7F, 8, 9V, 10A, 11A, 12F, 14, 15B, 17F, 18C, 19A, 19F, 22F, 23F and 33F.
193. An immunogenic composition comprising a Streptococcus pneumoniae serotype 9N glycoconjugate of any one of paragraphs 1-162 and further comprising glycoconjugates from S. pneumoniae serotypes 1, 2, 3, 4, 5, 6B, 8, 7F, 9V, 10A, 11A, 12F, 14, 15B, 17F, 18C, 19A, 19F, 20, 22F, 23F and 33F.
194. An immunogenic composition comprising a Streptococcus pneumoniae serotype 9N glycoconjugate of any one of paragraphs 1-162 and further comprising glycoconjugates from S. pneumoniae serotypes 1, 2, 3, 4, 5, 6A, 6B, 8, 7F, 9V, 10A, 11A, 12F, 14, 15B, 17F, 18C, 19A, 19F, 20, 22F, 23F and 33F.
195. An immunogenic composition comprising a Streptococcus pneumoniae serotype 9N glycoconjugate of any one of paragraphs 1-162 and further comprising glycoconjugates from S. pneumoniae serotypes 1, 2, 3, 4, 5, 6A, 6B, 8, 7F, 9V, 10A, 11A, 12F, 14, 15B, 17F, 18C, 19A, 19F, 20B, 22F, 23F and 33F.
196. An immunogenic composition comprising a Streptococcus pneumoniae serotype 9N glycoconjugate of any one of paragraphs 1-162 and further comprising glycoconjugates from S. pneumoniae serotypes 3, 6A, 7F, 8, 10A, 11A, 12F, 15A, 15C, 16F, 17F, 19A, 20, 22F, 23A, 23B, 24F, 31, 33F and 35B.
197. An immunogenic composition comprising a Streptococcus pneumoniae serotype 9N glycoconjugate of any one of paragraphs 1-162 and further comprising glycoconjugates from S. pneumoniae serotypes 3, 6A, 7F, 8, 10A, 11A, 12F, 15A, 15B, 16F, 17F, 19A, 20, 22F, 23A, 23B, 24F, 31, 33F and 35B.
198. An immunogenic composition comprising a Streptococcus pneumoniae serotype 9N glycoconjugate of any one of paragraphs 1-162 and further comprising glycoconjugates from S. pneumoniae serotypes 3, 6A, 7F, 8, 10A, 11A, 12F, 15A, 15C, 16F, 17F, 19A, 20B, 22F, 23A, 23B, 24F, 31, 33F and 35B.
199. An immunogenic composition comprising a Streptococcus pneumoniae serotype 9N glycoconjugate of any one of paragraphs 1-162 and further comprising glycoconjugates from S. pneumoniae serotypes 3, 6A, 7F, 8, 10A, 11A, 12F, 15A, 15B, 16F, 17F, 19A, 20, 22F, 23A, 23B, 24F, 31, 33F and 35B.
200. An immunogenic composition comprising a Streptococcus pneumoniae serotype 9N glycoconjugate of any one of paragraphs 1-162 and further comprising glycoconjugates from S. pneumoniae serotypes 1, 3, 4, 5, 6A, 6B, 8, 7F, 9V, 10A, 11A, 12F, 14, 15A, 15B, 18C, 19A, 19F, 22F, 23A, 23B, 23F, 24F, 33F and 35B.
201. An immunogenic composition comprising a Streptococcus pneumoniae serotype 9N glycoconjugate of any one of paragraphs 1-162 and further comprising glycoconjugates from S. pneumoniae serotypes 1, 3, 4, 5, 6A, 6B, 8, 7F, 9V, 10A, 11A, 12F, 14, 15A, 15B, 18C, 19A, 19F, 22F, 23A, 23B, 23F, 24F, 33F and 35B and additionally comprising at least one glycoconjugate from S. pneumoniae serotypes 7C, 17F, 20B, 21, 27, 31, 34, 35F and 38.
202. An immunogenic composition comprising a Streptococcus pneumoniae serotype 9N glycoconjugate of any one of paragraphs 1-162 and further comprising glycoconjugates from S. pneumoniae serotypes 1, 3, 4, 5, 6A, 6B, 8, 7F, 9V, 10A, 11A, 12F, 14, 15A, 15B, 18C, 19A, 19F, 22F, 23A, 23B, 23F, 24F, 33F and 35B and additionally comprising at least two glycoconjugates from S. pneumoniae serotypes 7C, 17F, 20B, 21, 27, 31, 34, 35F and 38.
203. An immunogenic composition comprising a Streptococcus pneumoniae serotype 9N glycoconjugate of any one of paragraphs 1-162 and further comprising glycoconjugates from S. pneumoniae serotypes 1, 3, 4, 5, 6A, 6B, 8, 7F, 9V, 10A, 11A, 12F, 14, 15A, 15B, 18C, 19A, 19F, 22F, 23A, 23B, 23F, 24F, 33F and 35B and additionally comprising at least three glycoconjugates from S. pneumoniae serotypes 7C, 17F, 20B, 21, 27, 31, 34, 35F and 38.
204. An immunogenic composition comprising a Streptococcus pneumoniae serotype 9N glycoconjugate of any one of paragraphs 1-162 and further comprising glycoconjugates from S. pneumoniae serotypes 1, 3, 4, 5, 6A, 6B, 8, 7F, 9V, 10A, 11A, 12F, 14, 15A, 15B, 18C, 19A, 19F, 22F, 23A, 23B, 23F, 24F, 33F and 35B and additionally comprising at least four glycoconjugates from S. pneumoniae serotypes 7C, 17F, 20B, 21, 27, 31, 34, 35F and 38.
205. An immunogenic composition comprising a Streptococcus pneumoniae serotype 9N glycoconjugate of any one of paragraphs 1-162 and further comprising glycoconjugates from S. pneumoniae serotypes 1, 3, 4, 5, 6A, 6B, 8, 7F, 9V, 10A, 11A, 12F, 14, 15A, 15B, 18C, 19A, 19F, 22F, 23A, 23B, 23F, 24F, 33F and 35B and additionally comprising at least five glycoconjugates from S. pneumoniae serotypes 7C, 17F, 20B, 21, 27, 31, 34, 35F and 38.
206. An immunogenic composition comprising a Streptococcus pneumoniae serotype 9N glycoconjugate of any one of paragraphs 1-162 and further comprising glycoconjugates from S. pneumoniae serotypes 1, 3, 4, 5, 6A, 6B, 8, 7F, 9V, 10A, 11A, 12F, 14, 15A, 15B, 18C, 19A, 19F, 22F, 23A, 23B, 23F, 24F, 33F and 35B and additionally comprising at least six glycoconjugates from S. pneumoniae serotypes 7C, 17F, 20B, 21, 27, 31, 34, 35F and 38.
207. An immunogenic composition comprising a Streptococcus pneumoniae serotype 9N glycoconjugate of any one of paragraphs 1-162 and further comprising glycoconjugates from S. pneumoniae serotypes 1, 3, 4, 5, 6A, 6B, 8, 7F, 9V, 10A, 11A, 12F, 14, 15A, 15B, 18C, 19A, 19F, 22F, 23A, 23B, 23F, 24F, 33F and 35B and additionally comprising at least seven glycoconjugates from S. pneumoniae serotypes 7C, 17F, 20B, 21, 27, 31, 34, 35F and 38.
208. An immunogenic composition comprising a Streptococcus pneumoniae serotype 9N glycoconjugate of any one of paragraphs 1-162 and further comprising glycoconjugates from S. pneumoniae serotypes 1, 3, 4, 5, 6A, 6B, 8, 7F, 9V, 10A, 11A, 12F, 14, 15A, 15B, 18C, 19A, 19F, 22F, 23A, 23B, 23F, 24F, 33F and 35B and additionally comprising at least eight glycoconjugates from S. pneumoniae serotypes 7C, 17F, 20B, 21, 27, 31, 34, 35F and 38.
209. An immunogenic composition comprising a Streptococcus pneumoniae serotype 9N glycoconjugate of any one of paragraphs 1-162 and further comprising glycoconjugates from S. pneumoniae serotypes 1, 3, 4, 5, 6A, 6B, 8, 7F, 9V, 10A, 11A, 12F, 14, 15A, 15B, 18C, 19A, 19F, 22F, 23A, 23B, 23F, 24F, 33F and 35B and additionally comprising at least eight glycoconjugates from S. pneumoniae serotypes 7C, 17F, 20B, 21, 27, 31, 34, 35F and 38.
210. An immunogenic composition comprising a Streptococcus pneumoniae serotype 9N glycoconjugate of any one of paragraphs 1-162 and further comprising glycoconjugates from S. pneumoniae serotypes 1, 3, 4, 5, 6A, 6B, 8, 7C, 7F, 9V, 10A, 11A, 12F, 14, 15A, 15B, 17F, 18C, 19A, 19F, 20B, 21, 22F, 23A, 23B, 23F, 24F, 27, 31, 33F, 34, 35B, 35F and 38.
211. An immunogenic composition comprising a Streptococcus pneumoniae serotype 9N glycoconjugate of any one of paragraphs 1-162 and further comprising glycoconjugates from S. pneumoniae serotypes 1, 2, 3, 4, 5, 6A, 6B, 8, 7C, 7F, 9V, 10A, 11A, 12F, 14, 15A, 15B, 17F, 18C, 19A, 19F, 20, 22F, 23A, 23B, 23F, 27, 31, 33F and 35B.
212. An immunogenic composition comprising a Streptococcus pneumoniae serotype 9N glycoconjugate of any one of paragraphs 1-162 and further comprising glycoconjugates from S. pneumoniae serotypes 1, 2, 3, 4, 5, 6A, 6B, 8, 7C, 7F, 9V, 10A, 11A, 12F, 14, 15A, 15B, 17F, 18C, 19A, 19F, 20A, 22F, 23A, 23B, 23F, 27, 31, 33F and 35B.
213. An immunogenic composition comprising a Streptococcus pneumoniae serotype 9N glycoconjugate of any one of paragraphs 1-162 and further comprising glycoconjugates from S. pneumoniae serotypes 1, 2, 3, 4, 5, 6A, 6B, 8, 7C, 7F, 9V, 10A, 11A, 12F, 14, 15A, 15B, 17F, 18C, 19A, 19F, 20B, 22F, 23A, 23B, 23F, 27, 31, 33F and 35B.
214. An immunogenic composition comprising a Streptococcus pneumoniae serotype 9N glycoconjugate of any one of paragraphs 1-162 and further comprising glycoconjugates from S. pneumoniae serotypes 1, 2, 3, 4, 5, 6A, 6B, 6C, 8, 7C, 7F, 9V, 10A, 11A, 12F, 14, 15A, 15B, 16F, 17F, 18C, 19A, 19F, 20, 22F, 23A, 23B, 23F, 24F, 31, 33F, 35B and 38.
215. An immunogenic composition comprising a Streptococcus pneumoniae serotype 9N glycoconjugate of any one of paragraphs 1-162 and further comprising glycoconjugates from S. pneumoniae serotypes 1, 2, 3, 4, 5, 6A, 6B, 6C, 8, 7C, 7F, 9V, 10A, 11A, 12F, 14, 15A, 15B, 16F, 17F, 18C, 19A, 19F, 20B, 22F, 23A, 23B, 23F, 24F, 31, 33F, 35B and 38.
216. An immunogenic composition comprising a Streptococcus pneumoniae serotype 9N glycoconjugate of any one of paragraphs 1-162 and further comprising glycoconjugates from S. pneumoniae serotypes 1, 2, 3, 4, 5, 6A, 6B, 6C, 8, 7C, 7F, 9V, 10A, 11A, 12F, 14, 15A, 15B, 16F, 17F, 18C, 19A, 19F, 20A, 22F, 23A, 23B, 23F, 24F, 31, 33F, 35B and 38.
217. The immunogenic composition of any one of paragraphs 163-216 wherein the glycoconjugate from S. pneumoniae serotype 9N is conjugated to CRM₁₉₇.
218. The immunogenic composition of any one of paragraphs 163-216 wherein the glycoconjugate from S. pneumoniae serotype 9N is conjugated to SCP.
219. The immunogenic composition of any one of paragraphs 163-218 wherein the glycoconjugate from S. pneumoniae serotype 22F if present is conjugated to CRM₁₉₇.
220. The immunogenic composition of any one of paragraphs 163-219 wherein the glycoconjugate from S. pneumoniae serotype 33F if present is conjugated to CRM₁₉₇.
221. The immunogenic composition of any one of paragraphs 163-220 wherein the glycoconjugate from S. pneumoniae serotype 15B if present is conjugated to CRM₁₉₇.
222. The immunogenic composition of any one of paragraphs 163-221 wherein the glycoconjugate from S. pneumoniae serotype 12F if present is conjugated to CRM₁₉₇.
223. The immunogenic composition of any one of paragraphs 163-222 wherein the glycoconjugate from S. pneumoniae serotype 10A if present is conjugated to CRM₁₉₇.
224. The immunogenic composition of any one of paragraphs 163-223 wherein the glycoconjugate from S. pneumoniae serotype 11A if present is conjugated to CRM₁₉₇.
225. The immunogenic composition of any one of paragraphs 163-224 wherein the glycoconjugate from S. pneumoniae serotype 8 if present is conjugated to CRM₁₉₇.
226. The immunogenic composition of any one of paragraphs 163-225 wherein the glycoconjugates from S. pneumoniae serotypes 4, 6B, 9V, 14, 18C, 19F and 23F if present are conjugated to CRM₁₉₇.
227. The immunogenic composition of any one of paragraphs 163-226 wherein the glycoconjugates from S. pneumoniae serotypes 1, 5 and 7F if present are conjugated to CRM₁₉₇.
228. The immunogenic composition of any one of paragraphs 163-227 wherein the glycoconjugate from S. pneumoniae serotypes 6A and 19A if present are conjugated to CRM₁₉₇.
229. The immunogenic composition of any one of paragraphs 163-216 wherein the glycoconjugates are all individually conjugated to CRM₁₉₇.
230. The immunogenic composition of any one of paragraphs 163-216 wherein the glycoconjugate from S. pneumoniae serotype 9N is conjugated to SCP and the other glycoconjugate(s) is/are all individually conjugated to CRM₁₉₇.
231. The immunogenic composition of any one of paragraphs 163-216 wherein the glycoconjugate from S. pneumoniae serotype 9N is conjugated to SCP, at least one other glycoconjugate is conjugated to TT and the other glycoconjugate(s) is/are all individually conjugated to CRM₁₉₇.
232. The immunogenic composition of any one of paragraphs 163-216 wherein the glycoconjugate from S. pneumoniae serotype 9N is conjugated to SCP, one other glycoconjugate is conjugated to TT and the other glycoconjugate(s) is/are all individually conjugated to CRM₁₉₇.
233. The immunogenic composition of any one of paragraphs 163-216 wherein the glycoconjugate from S. pneumoniae serotype 9N is conjugated to SCP, at least two other glycoconjugates are conjugated to TT and the other glycoconjugate(s) is/are all individually conjugated to CRM₁₉₇.
234. The immunogenic composition of any one of paragraphs 163-216 wherein the glycoconjugate from S. pneumoniae serotype 9N is conjugated to SCP, two other glycoconjugates are conjugated to TT and the other glycoconjugate(s) is/are all individually conjugated to CRM₁₉₇.
235. The immunogenic composition of any one of paragraphs 163-216 wherein the glycoconjugate from S. pneumoniae serotype 9N is conjugated to SCP, at least three other glycoconjugates are conjugated to TT and the other glycoconjugate(s) is/are all individually conjugated to CRM₁₉₇.
236. The immunogenic composition of any one of paragraphs 163-216 wherein the glycoconjugate from S. pneumoniae serotype 9N is conjugated to SCP, three other glycoconjugates are conjugated to TT and the other glycoconjugate(s) is/are all individually conjugated to CRM₁₉₇.
237. The immunogenic composition of any one of paragraphs 163-216 wherein the glycoconjugate from S. pneumoniae serotype 9N is conjugated to SCP, at least four other glycoconjugates are conjugated to TT and the other glycoconjugate(s) is/are all individually conjugated to CRM₁₉₇.
238. The immunogenic composition of any one of paragraphs 163-216 wherein the glycoconjugate from S. pneumoniae serotype 9N is conjugated to SCP, four other glycoconjugates are conjugated to TT and the other glycoconjugate(s) is/are all individually conjugated to CRM₁₉₇.
239. The immunogenic composition of any one of paragraphs 163-216 wherein the glycoconjugate from S. pneumoniae serotype 9N is conjugated to SCP, at least five other glycoconjugates are conjugated to TT and the other glycoconjugate(s) is/are all individually conjugated to CRM₁₉₇.
240. The immunogenic composition of any one of paragraphs 163-216 wherein the glycoconjugate from S. pneumoniae serotype 9N is conjugated to SCP, five other glycoconjugates are conjugated to TT and the other glycoconjugate(s) is/are all individually conjugated to CRM₁₉₇.
241. The immunogenic composition of any one of paragraphs 163-216 wherein the glycoconjugate from S. pneumoniae serotype 9N is conjugated to SCP, at least one other glycoconjugate is conjugated to SCP and the other glycoconjugate(s) is/are all individually conjugated to CRM₁₉₇.
242. The immunogenic composition of any one of paragraphs 163-216 wherein the glycoconjugate from S. pneumoniae serotype 9N is conjugated to SCP, one other glycoconjugate is conjugated to SCP and the other glycoconjugate(s) is/are all individually conjugated to CRM₁₉₇.
243. The immunogenic composition of any one of paragraphs 163-216 wherein the glycoconjugate from S. pneumoniae serotype 9N is conjugated to SCP, at least two other glycoconjugates are conjugated to SCP and the other glycoconjugate(s) is/are all individually conjugated to CRM₁₉₇.
244. The immunogenic composition of any one of paragraphs 163-216 wherein the glycoconjugate from S. pneumoniae serotype 9N is conjugated to SCP, two other glycoconjugates are conjugated to SCP and the other glycoconjugate(s) is/are all individually conjugated to CRM₁₉₇.
245. The immunogenic composition of any one of paragraphs 163-216 wherein the glycoconjugate from S. pneumoniae serotype 9N is conjugated to SCP, at least three other glycoconjugates are conjugated to SCP and the other glycoconjugate(s) is/are all individually conjugated to CRM₁₉₇.
246. The immunogenic composition of any one of paragraphs 163-216 wherein the glycoconjugate from S. pneumoniae serotype 9N is conjugated to SCP, three other glycoconjugates are conjugated to SCP and the other glycoconjugate(s) is/are all individually conjugated to CRM₁₉₇.
247. The immunogenic composition of any one of paragraphs 163-216 wherein the glycoconjugate from S. pneumoniae serotype 9N is conjugated to SCP, at least four other glycoconjugates are conjugated to SCP and the other glycoconjugate(s) is/are all individually conjugated to CRM₁₉₇.
248. The immunogenic composition of any one of paragraphs 163-216 wherein the glycoconjugate from S. pneumoniae serotype 9N is conjugated to SCP, four other glycoconjugates are conjugated to SCP and the other glycoconjugate(s) is/are all individually conjugated to CRM₁₉₇.
249. The immunogenic composition of any one of paragraphs 163-216 wherein the glycoconjugate from S. pneumoniae serotype 9N is conjugated to SCP, at least five other glycoconjugates are conjugated to SCP and the other glycoconjugate(s) is/are all individually conjugated to CRM₁₉₇.
250. The immunogenic composition of any one of paragraphs 163-216 wherein the glycoconjugate from S. pneumoniae serotype 9N is conjugated to SCP, five other glycoconjugates are conjugated to SCP and the other glycoconjugate(s) is/are all individually conjugated to CRM₁₉₇.
251. The immunogenic composition of any one of paragraphs 163-250 wherein each dose comprises between 0.1 μg and 100 μg of serotype 9N polysaccharide.
252. The immunogenic composition of any one of paragraphs 163-250 wherein each dose comprises between 0.5 μg and 20 μg of serotype 9N polysaccharide.
253. The immunogenic composition of any one of paragraphs 163-250 wherein each dose comprises between 1.0 μg and 10 μg of serotype 9N polysaccharide.
254. The immunogenic composition of any one of paragraphs 163-250 wherein each dose comprises between 2.0 μg and 5.0 μg of serotype 9N polysaccharide.
255. The immunogenic composition of any one of paragraphs 163-254 wherein each dose comprises between 10 μg and 150 μg of carrier protein (total amount).
256. The immunogenic composition of any one of paragraphs 163-254 wherein each dose comprises between 15 μg and 100 μg of carrier protein (total amount).
257. The immunogenic composition of any one of paragraphs 163-254 wherein each dose comprises between 25 μg and 75 μg of carrier protein (total amount).
258. The immunogenic composition of any one of paragraphs 163-254 wherein each dose comprises between 40 μg and 60 μg of carrier protein (total amount).
259. The immunogenic composition of any one of paragraphs 163-258 further comprising at least one, two or three adjuvants.
260. The immunogenic composition of any one of paragraphs 163-258 further comprising one adjuvant.
261. The immunogenic composition of any one of paragraphs 163-258 further comprising two adjuvants.
262. The immunogenic composition of any one of paragraphs 163-262 wherein said adjuvants is/are alum (e.g., aluminum phosphate, aluminum sulfate or aluminum hydroxide), calcium phosphate, liposomes, oil-in-water emulsions such as MF59 (4.3% w/v squalene, 0.5% w/v polysorbate 80 (Tween 80), 0.5% w/v sorbitan trioleate (Span 85)) and/or water-in-oil emulsions such as Montanide, and poly(D,L-lactide-co-glycolide) (PLG) microparticles or nanoparticles.
263. The immunogenic composition of paragraph 260 wherein said adjuvant comprises aluminum salts (alum).
264. The immunogenic composition of paragraph 260 wherein said adjuvant is aluminum phosphate or aluminum hydroxide.
265. The immunogenic composition of paragraph 260 wherein said adjuvant is aluminum phosphate.
266. The immunogenic composition of paragraph 260 wherein said adjuvant is a CpG oligonucleotide.
267. The immunogenic composition of any one of paragraphs 163-266 formulated in a liquid form.
268. The immunogenic composition of any one of paragraphs 163-266 formulated in a lyophilized form.
269. The immunogenic composition of any one of paragraphs 163-267 comprising a pharmaceutically acceptable excipient, carrier, or diluent.
270. The glycoconjugate of any one of paragraphs 1-162 for use as an antigen.
271. The glycoconjugate of any one of paragraphs 1-162 for use as a medicament.
272. The glycoconjugate of any one of paragraphs 1-162 for use as a vaccine.
273. The glycoconjugate of any one of paragraphs 1-162 for use in generating an immune response in a subject.
274. The glycoconjugate for use of paragraph 273 wherein said subject is a human.
275. The immunogenic composition of any one of paragraphs 163-269 for use as a medicament.
276. The immunogenic composition of any one of paragraphs 163-269 for use as a vaccine.
277. The immunogenic composition for use of paragraph 276 wherein said subject is a human.
278. The immunogenic composition of any one of paragraphs 163-269 for use is a method of preventing, treating or ameliorating a bacterial infection, disease or condition in a subject.
279. The immunogenic composition of any one of paragraphs 163-269 for use is a method of preventing, treating or ameliorating a S. pneumoniae serotype 9N infection, disease or condition in a subject.
280. The immunogenic composition of any one of paragraphs 163-269 for use is a method of inducing an immune response to S. pneumoniae serotype 9N in a subject.
281. The immunogenic composition of any one of paragraphs 163-269 for use is a method of preventing an infection by S. pneumoniae serotype 9N in a subject.
282. The immunogenic composition of any one of paragraphs 163-269 for use is a method of protecting a human susceptible to a S. pneumoniae serotype 9N infection.
283. A method of making a Streptococcus pneumoniae serotype 9N glycoconjugate, using click chemistry said method comprising the steps of (a) reacting an isolated serotype 9N saccharide with a carbonic acid derivative and an azido linker in an aprotic solvent to produce an activated azido saccharide (activation of the saccharide), (b) reacting a carrier protein with an agent bearing an N-Hydroxysuccinimide (NHS) moiety and an alkyne group where the NHS moiety reacts with the amino groups to form an amide linkage thereby obtaining an alkyne functionalized carrier protein (activation of the carrier protein), (c) reacting the activated azido saccharide of step (a) with the activated alkyne-carrier protein of step (b) by Cu⁺¹ mediated azide-alkyne cycloaddition reaction to form a glycoconjugate.
284. The method of paragraph 283 wherein before the activation (a), sizing of the saccharide to a target molecular weight (MW) range has been performed.
285. The method of paragraph 283 wherein the isolated serotype 9N saccharide is sized before activation with a carbonic acid derivative and an azido linker.
286. The method of paragraph 285 wherein the isolated serotype 9N capsular polysaccharide is sized to a weight average molecular weight between 5 kDa and 500 kDa.
287. The method of paragraph 285 wherein the isolated serotype 9N capsular polysaccharide is sized to a weight average molecular weight between 50 kDa and 450 kDa.
288. The method of paragraph 285 wherein the isolated serotype 9N capsular polysaccharide is sized to a weight average molecular weight between 100 kDa and 400 kDa.
289. The method of paragraph 285 wherein the isolated serotype 9N capsular polysaccharide is sized to a weight average molecular weight between about 200 kDa and about 300 kDa.
290. The method of paragraph 285 wherein the isolated serotype 9N capsular polysaccharide is sized to a weight average molecular weight between about 100 kDa and about 200 kDa.
291. The method of paragraph 283 wherein the isolated serotype 9N capsular polysaccharide is not sized before activation with a carbonic acid derivative and an azido linker.
292. The method of any one of paragraphs 283 to 292 wherein said carbonic acid derivative is selected from the group consisting of 1,1′-carbonyldiimidazole (CDI), 1,1′-carbonyl-di-(1,2,4-triazole) (CDT), N,N′-Disuccinimidyl carbonate (DSC) and N-hydroxysuccinimidyl chloroformate.
293. The method of any one of paragraphs 283 to 292 wherein said carbonic acid derivative is 1,1′-carbonyldiimidazole (CDI).
294. The method of any one of paragraphs 283 to 292 wherein said carbonic acid derivative is 1,1′-Carbonyl-di-(1,2,4-triazole) (CDT).
295. The method of any one of paragraphs 283 to 292 wherein said carbonic acid derivative is N,N′-Disuccinimidyl carbonate (DSC).
296. The method of any one of paragraphs 283 to 292 wherein said carbonic acid derivative is N-hydroxysuccinimidyl chloroformate.
297. The method of any one of paragraphs 283 to 292 wherein said azido linker is a compound of formula (VI),

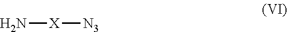

wherein X is selected from the group consisting of CH₂(CH₂)ₙ, (CH₂CH₂O)ₘCH₂CH₂, NHCO(CH₂)ₙ, NHCO(CH₂CH₂O)ₘCH₂CH₂, OCH₂(CH₂)ₙ and O(CH₂CH₂O)ₘCH₂CH₂, where n is selected from 1 to 10 and m is selected from 1 to 4.
298. The method of any one of paragraphs 283 to 292 wherein said azido linker is a compound of formula (VI), wherein X is CH₂(CH₂)ₙ, and n is selected from 1 to 10.
299. The method of any one of paragraphs 283 to 292 wherein said azido linker is a compound of formula (VI), wherein X is (CH₂CH₂O)ₘCH₂CH₂, wherein m is selected from 1 to 4.
300. The method of any one of paragraphs 283 to 292 wherein said azido linker is a compound of formula (VI), wherein X is NHCO(CH₂)ₙ, and n is selected from 1 to 10.
301. The method of any one of paragraphs 283 to 292 wherein said azido linker is a compound of formula (VI), wherein X is NHCO(CH₂CH₂O)ₘCH₂CH₂, where m is selected from 1 to 4.
302. The method of any one of paragraphs 283 to 292 wherein said azido linker is a compound of formula (VI), wherein X is OCH₂(CH₂)ₙ, and n is selected from 1 to 10.
303. The method of any one of paragraphs 283 to 292 wherein said azido linker is a compound of formula (VI), wherein X is O(CH₂CH₂O)ₘCH₂CH₂, where m is selected from 1 to 4.
304. The method of any one of paragraphs 283 to 292 wherein said azido linker is a compound of formula (VII),

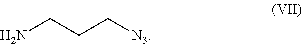

305. The method of any one of paragraphs 283 to 292 wherein said azido linker is 3-azido-propylamine.
306. The method of any one of paragraphs 283 to 305 wherein said agent bearing an N-Hydroxysuccinimide (NHS) moiety and an alkyne group is an agent bearing an N-Hydroxysuccinimide (NHS) moiety and a terminal alkyne.
307. The method of any one of paragraphs 283 to 305 wherein said agent bearing an N-Hydroxysuccinimide (NHS) moiety and an alkyne group is an agent bearing an N-Hydroxysuccinimide (NHS) moiety and a cycloalkyne.
308. The method of any one of paragraphs 283 to 305 wherein said agent bearing an N-Hydroxysuccinimide (NHS) moiety and an alkyne group is a compound of formula (VIII),

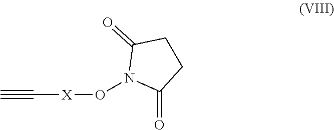

where X is selected from the group consisting of CH₂O(CH₂)ₙCH₂C═O and CH₂O(CH₂CH₂O)ₘ(CH₂)ₙCH₂C═O, where n is selected from 0 to 10 and m is selected from 0 to 4.
309. The method of any one of paragraphs 283 to 305 wherein said agent bearing an N-Hydroxysuccinimide (NHS) moiety and an alkyne group is a compound of formula (VIII), wherein X is CH₂O(CH₂)ₙCH₂C═O, where n is selected from 0 to 10.
310. The method of any one of paragraphs 283 to 305 wherein said agent bearing an N-Hydroxysuccinimide (NHS) moiety and an alkyne group is a compound of formula (VIII), wherein X is CH₂O(CH₂CH₂O)ₘ(CH₂)ₙCH₂C═O, where n is selected from 0 to 10 and m is selected from 0 to 4. The method of any one of paragraphs 283 to 305 wherein said agent bearing an N-Hydroxysuccinimide (NHS) moiety and an alkyne group is a compound of formula (IX):

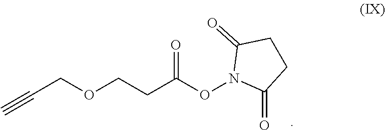 [SMILES: C#CCOCCC(=O)ON1C(=O)CCC1=O]

311. The method of any one of paragraphs 283 to 310 wherein, step a) comprises reacting the saccharide with a carbonic acid derivative followed by reacting the carbonic acid derivative-activated saccharide with an azido linker in an aprotic solvent to produce an activated azido saccharide.
312. The method of any one of paragraphs 283 to 311 wherein step a) comprises reacting the saccharide with an amount of carbonic acid derivative that is between 0.01-10 molar equivalent to the amount of saccharide present in the reaction mixture.
313. The method of any one of paragraphs 283 to 312 wherein at step a) the isolated saccharide is reacted with a carbonic acid derivative in an aprotic solvent.
314. The method of any one of paragraphs 283 to 313 wherein the isolated saccharide is reacted with CDI in an aprotic solvent comprising 0.1% to 1% (v/v) water.
315. The method of any one of paragraphs 283 to 314 wherein step a) further comprises reacting the carbonic acid derivative-activated saccharide with an amount of azido linker that is between 0.01-10 molar equivalent to the amount of polysaccharide Repeat Unit of the activated saccharide (molar equivalent of RU).
316. The method of any one of paragraphs 283 to 315 wherein the degree of activation of the activated saccharide following step a) is between 1.0 to 100%.
317. The method of any one of paragraphs 283 to 315 wherein the degree of activation of the activated saccharide following step a) is between 5 to 70%.
318. The method of any one of paragraphs 283 to 315 wherein the degree of activation of the activated saccharide following step a) is between 5 to 50%.
319. The method of any one of paragraphs 283 to 315 wherein the degree of activation of the activated saccharide following step a) is between 10 to 40%.
320. The method of any one of paragraphs 283 to 315 wherein the degree of activation of the activated saccharide following step a) is between 5 to 15%.
321. The method of any one of paragraphs 283 to 315 wherein the degree of activation of the activated saccharide following step a) is between 15 to 35%.
322. The method of any one of paragraphs 283 to 315 wherein the degree of activation of the activated saccharide following step a) is between 15 to 25%.
323. The method of any one of paragraphs 283 to 315 wherein the degree of activation of the activated saccharide following step a) is about 25%.
324. The method of any one of paragraphs 283 to 323 wherein step b) comprises reacting the carrier protein with an amount of agent bearing an N-Hydroxysuccinimide (NHS) moiety and an alkyne group that is 0.1-10 molar equivalents to the lysines on the carrier.
325. The method of any one of paragraphs 283 to 324 wherein the degree of activation of the activated carrier following step b) is between 1 and 50.
326. The method of any one of paragraphs 283 to 324 wherein the degree of activation of the activated carrier following step b) may be between 1 to 30.
327. The method of any one of paragraphs 283 to 324 wherein the degree of activation of the activated carrier following step b) is between 5 to 20.
328. The method of any one of paragraphs 283 to 324 wherein the degree of activation of the activated carrier following step b) is between 9 to 18.
329. The method of any one of paragraphs 283 to 324 wherein the degree of activation of the activated carrier following step b) is between 8 to 11.
330. The method of any one of paragraphs 283 to 324 wherein the degree of activation of the activated carrier following step b) is between 15 to 20.
331. The method of any one of paragraphs 283 to 330 wherein the conjugation reaction c) is carried out in aqueous buffer.
332. The method of any one of paragraphs 283 to 330 wherein the conjugation reaction c) is carried out in aqueous buffer in the presence of copper (I) as catalyst.
333. The method of any one of paragraphs 283 to 330 wherein the conjugation reaction c) is carried out in aqueous buffer in the presence an oxidant and of copper (I) as catalyst.
334. The method of any one of paragraphs 283 to 333 wherein the initial input ratio (weight by weight) of activated azido saccharide to activated alkyne-carrier at step c) is between 0.1 and 3.
335. The method of any one of paragraphs 283 to 334 wherein following step c), unreacted azido groups in the conjugates, are capped using a suitable azido group capping agent.
336. The method of any one of paragraphs 283 to 335 wherein following step c) unreacted alkyne groups are capped using a suitable alkyne group capping agent.
337. The method of any one of paragraphs 283 to 336 wherein said Streptococcus pneumoniae serotype 9N glycoconjugate is according to any one of paragraphs 1-57.
338. A method of making a Streptococcus pneumoniae serotype 9N glycoconjugate, using reductive amination said method comprising the steps of (1) oxidation (activation) of the serotype 9N purified saccharide, (2) reduction of the activated saccharide and the carrier protein to form a glycoconjugate.
339. The method of paragraph 338 wherein the isolated serotype 9N polysaccharide is sized before oxidation.
340. A method of making a Streptococcus pneumoniae serotype 9N glycoconjugate, comprising the step of: (a) reacting said serotype 9N saccharide with an oxidizing agent; (b) compounding the activated saccharide of step (a) with a carrier protein; and (c) reacting the compounded activated saccharide and carrier protein with a reducing agent to form a glycoconjugate.
341. The method of paragraph 340 wherein the oxidizing agent is periodate.
342. The method of paragraph 341 wherein the degree of oxidation of the activated serotype 9N saccharide is between 2 and 30.
343. The method of paragraph 341 wherein the degree of oxidation (DO) of the activated serotype 9N saccharide is between 5 and 15.
344. The method of paragraph 341 wherein the degree of oxidation (DO) of the activated serotype 9N saccharide is between 5 and 15 and wherein the initial input ratio (weight by weight) of activated serotype 9N saccharide to carrier protein at step b) is between 1.5:1 and 0.5:1.
345. The method of paragraph 341 wherein the degree of oxidation (DO) of the activated serotype 9N saccharide is between 5 and 15 and wherein the initial input ratio (weight by weight) of activated serotype 9N saccharide to carrier protein at step b) is between 1.5:1 and 0.5:1, wherein the reduction reaction (c) is carried out in an aprotic solvent.
346. The method of paragraph 341 wherein the degree of oxidation (DO) of the activated serotype 9N saccharide is between 5 and 15 and wherein the initial input ratio (weight by weight) of activated serotype 9N saccharide to carrier protein at step b) is between 1.5:1 and 0.5:1, wherein the reduction reaction (c) is carried out in a solution consisting essentially of dimethylsulphoxide (DMSO).
347. The method of paragraph 341 wherein the degree of oxidation (DO) of the activated serotype 9N saccharide is between 5 and 15 and wherein the initial input ratio (weight by weight) of activated serotype 9N saccharide to carrier protein at step b) is between 1.5:1 and 0.5:1, wherein the reduction reaction (c) is carried out in DMSO (dimethylsulfoxide) solvent.
348. The method of paragraph 341 wherein the degree of oxidation (DO) of the activated serotype 9N saccharide is between 5 and 15 and wherein the initial input ratio (weight by weight) of activated serotype 9N saccharide to carrier protein at step b) is between 1.5:1 and 0.5:1, wherein the reduction reaction (c) is carried out in DMSO (dimethylsulfoxide) solvent and wherein the reducing agent is sodium cyanoborohydride.
349. The method of any one of paragraphs 340 to 348 wherein said Streptococcus pneumoniae serotype 9N glycoconjugate is according to any one of paragraphs 1-57.
350. The method of paragraph 348 wherein said Streptococcus pneumoniae serotype 9N glycoconjugate is according to any one of paragraphs 1-57.

As used herein, the term “about” means within a statistically meaningful range of a value, such as a stated concentration range, time frame, molecular weight, temperature or pH. Such a range can be within an order of magnitude, typically within 20%, more typically within 10%, and even more typically within 5% or within 1% of a given value or range. Sometimes, such a range can be within the experimental error typical of standard methods used for the measurement and/or determination of a given value or range. The allowable variation encompassed by the term “about” will depend upon the particular system under study, and can be readily appreciated by one of ordinary skill in the art. Whenever a range is recited within this application, every number within the range is also contemplated as an embodiment of the disclosure.

The terms “comprising”, “comprise” and “comprises” herein are intended by the inventors to be optionally substitutable with the terms “consisting essentially of”, “consist essentially of”, “consists essentially of”, “consisting of’, “consist of’ and “consists of’, respectively, in every instance.

An “immunogenic amount”, an “immunologically effective amount”, a “therapeutically effective amount”, a “prophylactically effective amount”, or “dose”, each of which is used interchangeably herein, generally refers to the amount of antigen or immunogenic composition sufficient to elicit an immune response, either a cellular (T cell) or humoral (B cell or antibody) response, or both, as measured by standard assays known to one skilled in the art.

Any whole number integer within any of the ranges of the present document is contemplated as an embodiment of the disclosure.

All references or patent applications cited within this patent specification are incorporated by reference herein.

The invention is illustrated in the accompanying examples. The examples below are carried out using standard techniques, which are well known and routine to those of skill in the art, except where otherwise described in detail. The examples are illustrative, but do not limit the invention.

### EXAMPLE

### Example 1. Preparation of Serotype 9N Glycoconjugate Using Reductive Amination in Aqueous Buffer (RAC/Aq.)

1. Hydrolysis The native polysaccharide was hydrolyzed to reduce the molecular weight prior to activation. A calculated volume of 2M acetic acid was added to the polysaccharide solution to achieve a final polysaccharide concentration of 2.0±0.2 g/L and a final acetic acid concentration of 0.1-0.3M. The diluted polysaccharide solution was heated to 80±5° C. The hydrolysis reaction was maintained for certain time depending on the target polysaccharide Mw. At the end of reaction, the mixture was cooled to 23±2° C. The mixture was then purified by UF/DF using 10K MWCO membrane against water

### 2. Oxidation

Polysaccharide oxidation was carried out in 50 mM potassium phosphate buffer (pH 6.0) by addition of of 500 mM potassium phosphate buffer (pH 6.0) and WFI to give final polysaccharide concentration of 2.0 g/L. Sodium periodate was then added to the polysaccharide solution to initiate the oxidation reaction (added as a 50 mg/mL solution in WFI). The required molar equivalent of sodium periodic was selected based on the target Degree of Oxidation (DO). The target range for the DO was 8.0±4.0. The oxidation reaction time was 20±4 hours, at 23±2° C.

### 3. Purification of the Activated Polysaccharide

The activated polysaccharide was purified by tangential flow filtration against WFI. The diafiltration was performed using polyethersulfone (PES) flat sheet membranes with a molecular weight cut-off (MWCO) of 10 kDa. Once the diafiltration was complete, the activated polysaccharide was characterized, by (i) saccharide concentration by colorimetric assay; (ii) aldehyde concentration by colorimetric assay; (iii) degree of oxidation; and (iv) molecular weight by SEC-MALLS. The pH of purified saccharide was adjusted to 6.3±0.3. Protein (CRM₁₉₇) was then added to a pre-determined ratio. The mixture was then shell frozen and lyophilized to dry.

### 4. Conjugation Reaction

The lyophilized activated polysaccharide and protein (CRM197) were reconstituted in 0.1M sodium phosphate buffer. After the reconstitution was complete, the pH was adjusted to a final pH of 6.5±0.2 using 1N hydrochloric acid or 1N sodium hydroxide. To initiate the conjugation reaction, predetermined molar equivalent of sodium cyanoborohydride (1.5 MEq) was added to the reaction mixture. The conjugation proceeded for a period of 40±4 hours at 30±2° C. with continuous mixing at 100±10 rpm.

### 5. Dilution and Capping Reaction

After the conjugation reaction time was complete, the reaction solution was cooled to 23±2° C. and diluted by a factor of 0.5-1.0 times the reaction volume with 0.9% NaCl buffer, 1 molar equivalent of sodium borohydride was then added to the mixture. The capping reaction proceeded for a period of 3-6 hours at 23±2° C. with continuous mixing at 100±10 rpm.

### 6. Purification of Conjugate

The diluted conjugate solution was passed through a 5 μm filter, and diafiltration was performed using 5 mM succinate/0.9% saline (pH 6.0) as the medium. After the diafiltration was completed, the conjugate retentate was filtered through a 0.45 um/0.22 μm filter.

TABLE 1
Conjugate obtained using reductive amination in aqueous buffer

| | RAC/Aq. |
|---|---|
| | Protein: CRM |
| Degree of Oxidation (DO) | 8.3 |
| Act. Poly MW (kDa) | 278 |
| Saccharide/Protein Ratio (input) | 1.0 |

Conjugate Data

| | |
|---|---|
| Saccharide/Protein Ratio | 1.23 |
| Yield (%) | 53 |
| % Free Saccharide | <5% |
| Conjugate MW (kDa) | 1926 |

### Example 2. Preparation of Serotype 9N Glycoconjugate Using Reductive Amination in Dimethylsulfoxide (RAC/DMSO)

### 1. Hydrolysis and Oxidation

Polysaccharide hydrolysis, activation and diafiltration were performed in the same manner as described for above aqueous based conjugation. The required molar equivalent of sodium periodate was selected based on the target DO. The target range for the DO is 8.0±4.0. The oxidation reaction time is 20±4 hours, at 23±2° C.

### 2. Compounding and Lyophilization

The activated polysaccharide was compounded with sucrose to a ratio of 10-100 grams of sucrose per gram of activated polysaccharide, preferably at a ratio of 20-40 grams of sucrose per gram of activated polysaccharide. The compounded mixture was then lyophilized. Calculated amount of carrier protein (CRM₁₉₇) was shell-frozen and lyophilized separately.

### 3. Conjugating and Capping

Lyophilized activated polysaccharide was reconstituted in anhydrous dimethyl sulfoxide (DMSO), an equal amount of anhydrous DMSO was used to reconstitute the carrier protein.

Reconstituted activated polysaccharide was combined with reconstituted carrier protein in the reaction vessel, followed by mixing thoroughly to obtain a clear solution before initiating the conjugation with sodium cyanoborohydride. The final polysaccharide concentration in reaction solution was approximately 1-3 g/L. Conjugation was initiated by adding 1.0 MEq of sodium cyanoborohydride to the reaction mixture and incubating at 23±2° C. for 20-48 hrs. The conjugation reaction was terminated by adding 2 MEq of sodium borohydride (NaBH4) to cap unreacted aldehydes. This capping reaction continued at 23±2° C. for 3±1 hrs.

### 4. Purification

The conjugate solution was diluted 1:10 with chilled 5 mM succinate-0.9% saline (pH 6.0) in preparation for purification by tangential flow filtration using 100-300K MWCO membranes. The diafiltration was then performed using 5 mM succinate/0.9% saline (pH 6.0) as the medium. After the diafiltration was completed, the conjugate retentate was transferred through a 0.22 μm filter. The conjugate was diluted further with 5 mM succinate/0.9% saline (pH 6), to a target saccharide concentration of approximately 0.5 mg/mL. Alternatively, the conjugate was purified using 20 mM Histidine-0.9% saline (pH 6.5) by tangential flow filtration using 100-300K MWCO membranes. Final 0.22 μm filtration step was completed to obtain the immunogenic conjugate.

TABLE 2
Conjugate obtained using reductive amination in DMSO (RAC/DMSO, carrier protein: CRM₁₉₇)

| Conjugate # | #1 | #2 | #3 | #4 | #5 | #6 | #7 | #8 | #9 |
|---|---|---|---|---|---|---|---|---|---|
| Degree of Oxidation (DO) | 4.0 | 6.0 | 10.0 | 11.6 | 11.6 | 11.6 | 6.0 | 6.0 | 6.0 |
| Act. Poly MW (kDa) | 243 | 277 | 309 | 453 | 453 | 453 | 271 | 271 | 271 |
| Conjug. condition | 2.5 mg/ml (poly/DMSO), NaCNBH₃: 1.0 Meq | | | | | | | | |
| Saccharide/Protein | 0.8 | 0.8 | 0.8 | 0.8 | 1.0 | 1.2 | 0.8 | 1.0 | 1.2 |
| Ratio (input) | | | | | | | | | |

Conjugate Data

| | | | | | | | | | |
|---|---|---|---|---|---|---|---|---|---|
| Saccharide/Protein | 0.66 | 0.73 | 0.83 | 0.68 | 0.78 | 0.93 | 0.72 | 0.82 | 0.92 |
| Ratio (Output) | | | | | | | | | |
| Yield (%) | 57 | 70 | 64 | 62 | 69 | 70 | 72 | 67 | 59 |
| % Free Saccharide | 6 | <5 | <5 | <5 | <5 | <5 | <5 | <5 | <5 |
| Conjugate MW (kDa) | 736 | 1128 | 1575 | 2863 | 2474 | 2267 | 959 | 816 | 807 |

| Conjugate # | #10 | #11 | #12 | #13 | #14 |
|---|---|---|---|---|---|
| Degree of Oxidation (DO) | 6.0 | 6.0 | 7.0 | 7.0 | 9.2 |
| Act. Poly MW (kDa) | 170 | 277 | 253 | 329 | 244 |
| Conjug. condition | 2.5 mg/ml (poly/DMSO), NaCNBH₃: 1.0 Meq | | | | |
| Saccharide/Protein Ratio | 0.8 | 0.8 | 0.8 | 0.8 | 0.8 |
| (input) | | | | | |

Conjugate Data

| | | | | | |
|---|---|---|---|---|---|
| Saccharide/Protein Ratio | 0.56 | 0.73 | 0.69 | 0.70 | 1.12 |
| (Output) | | | | | |
| Yield (%) | 53 | 70 | 71 | 69 | 54 |
| % Free Saccharide | 10 | <5 | 6 | <5 | <5 |
| Conjugate MW (kDa) | 901 | 1128 | 970 | 1196 | 3689 |

### Example 3. Preparation of Serotype 9N Glycoconjugate Using Click Chemistry

1. Activation of Serotype 9N Capsular Polysaccharide with Azido Linker

Serotype 9N capsular polysaccharide was mixed with imidazole (3×-10×, w/w), then shell-frozen and lyophilized.

After 3 days lyophilization, the lyophilized polysaccharide was reconstituted with anhydrous DMSO (3-4 mg/mL), and CDI (0.4-0.6 MEq) was added. The reaction mixture was stirred at 23° C. for 3 hrs. WFI (2% v/v) was added to quench free CDI and then stirred further for 30 min at 23° C. To the reaction mixture 3-azido-propylamine (2-6 MEq) is added. After 20 hrs reaction at 23° C., the reaction mixture was diluted to chilled (at 5° C.) 10 mM NaH2PO4 buffer (5×, v/v). The diluted reaction mixture was then purified by UF/DF using 10K MWCO PES membrane against WFI (30×, v/v).

2. Activation of SCP to Alkyne-SCP with Alkyne NHS Ester

To the SCP solution (1000 mg) WFI 57 mL and 0.5 M sodium phosphate buffer (pH 8.3) 50 mL were added. After cooled to 8° C., 3-Propargyloxy-propanoic acid NHS ester (POPS) (20 mg/mL in DMSO) 18 mL (2.4 MEq to lysine on SCP) was added to the reaction mixture dropwise maintaining the reaction temperature at 8±3° C. After the reaction mixture was stirred for 2 hrs at 8° C., purified by UF/DF using 10 K MWCO PES membrane (Millipore Pellicon 2 Mini) against 100 mM sodium phosphate buffer in saline (pH 7.0) (30× diavolume). After UF/DF, sucrose 23 g (15% v/v) was added.

### 3. Click Conjugation: Activated Azido Poly and Alkyne-SCP is Conjugated by Cu+1 Mediated Azide-Alkyne Cycloaddition Reaction, Referred as “Click Reaction”

The mixture of 5 mM copper sulfate (CuSO₄) (1 mL) and 25 mM Tris(3-hydroxypropyltriazolylmethyl)amine (THPTA) (1 mL) were added to the mixture of Serotype 9N Capsular Polysaccharide activated with azido linker (see step 1 above) and alkyne-SCP (see step 2 above) (in 100 mM Sodium Phosphate Buffer (SPB) in saline, pH 7.0) at 23° C. and followed by the addition of 100 mM aminoguanidine (2 mL) and 100 mM sodium ascorbate (2 mL). After the reaction mixture was stirred for 2 hours at 23° C., the unreacted azido group was capped by propargyl alcohol (1 MEq) for 2 hours at 23° C. and after the first capping, subsequently the unreacted alkyne group was capped by 3-azido-1-propanol (2 MEq) for 2 hours at 23° C. Then, the reaction mixture was purified by UF/DF using 100K MWCO PES membrane against 10 mM EDTA+10 mM SPB in saline (pH 7.0) (30× diavolume) and then followed by 5 mM succinate in saline (pH 6.0) (30× diavolume).

TABLE 3
Conjugate obtained using click chemistry (carrier protein: SCP)

| Conjugate # | #15 | #16 |
|---|---|---|
| Poly MW (kDa) | 258 | 616 |
| Azido Poly DoA (%) per Poly RU | 14 | 22 |
| Azido Poly Mw (kDa) | 307 | 580 |
| Alkyne-SCP Mw (kDa) | 101 | 101 |
| Alkyne-SCP DoA (AAA), alkynes/SCP | 19 | 20 |

Conjugate Data

| | | |
|---|---|---|
| Conjugate Yield (%) | 76 | 81 |
| Saccharide/Protein Ratio (Output) | 0.83 | 0.8 |
| % Free Saccharide | <5 | <5 |
| Conjugate MW (kDa) | 1084 | 3274 |

### Example 4. Evaluation of Immunogenicity of S. pneumoniae Serotype 9N Glycoconjugates Using Different Chemistries

The opsonophagocytic activity (OPA) titers in mice vaccinated with serotype 9N conjugates with different (RAC/DMSO or Click), carriers (CRM₁₉₇ or SCP) and comprising of polysaccharide of different size were determined under standard conditions.

Sized or native serotype 9N polysaccharides conjugated to either CRM₁₉₇ or SCP, using either RAC/DMSO (see example 2) or Click (see example 3) conjugation was used to vaccinate mice in the presence of adjuvant. See attributes of the tested conjugates at Tables 2 and 3 (tested conjugates are #14, #15 and #16).

Groups of twenty-five 6-8 weeks old female Swiss Webster mice were immunized with 0.01 μg/animal or 0.1 μg/animal of test conjugates via the subcutaneous route on week 0. Due to a discrepancy in saccharide concentration, mice vaccinated with 9N native conjugate (#16) were administered 0.13 μg/animal or 0.013 μg/animal at week 0. The vaccination dose was adjusted to the correct dose for week 3.

The mice were boosted with 0.01 μg/animal or 0.1 μg/animal dose of conjugate on week 3 and then bled at week 5. Each vaccination was formulated with 100 μg/dose of AIPO₄ as an adjuvant. All preclinical immunogenicity studies were powered to detect a 4 to 5-fold difference in OPA titers using 25 mice per group. Whole blood was collected from mice two weeks after the second vaccination (Week 5, PD 2) and sera used for analyses. Serotype-specific OPAs were performed on week 5 sera samples.

Opsonophagocytic activity (OPA) assays are used to measure functional antibodies in murine sera specific for S. pneumonia serotype 9N. Test serum is set up in assay reactions that measure the ability of capsular polysaccharide specific immunoglobulin to opsonize bacteria, trigger complement deposition, thereby facilitating phagocytosis and killing of bacteria by phagocytes. The OPA titer is defined as the reciprocal dilution that results in a 50% reduction in bacterial count over control wells without test serum. The OPA titer is interpolated from the two dilutions that encompass this 50% killing cut-off. OPA procedures were based on methods described in Hu et al. (2005) Clin Diagn Lab Immunol 12 (2):287-295 with the following modifications. Test serum was serially diluted 2.5-fold and added to microtiter assay plates. Live serotype 9N target bacterial strains were added to the wells and the plates were shaken at 37° C. for 30 minutes. Differentiated HL-60 cells (phagocytes) and baby rabbit serum (3- to 4-week old, PEL-FREEZ®, 12% final concentration) were added to the wells, and the plates were shaken at 37° C. for 45 minutes. A 10 μL aliquot were transferred to the wells of MULTISCREEN® HTS HV filter plates (MILLIPORE®) containing 50 μL of water. Liquid was filtered through the plates under vacuum, and 50 μL of HYSOY® medium was added to each well and filtered through. The filter plates were then incubated at 37° C., 5% CO₂ overnight and were then fixed with 70% Ethanol (Decon Labs, Inc., King of Prussia, PA). The plates were then stained with Coomassie Blue diluted 1:1 with Destain Solution (Bio-Rad Laboratories, Inc., Hercules, CA), and destained with 35% Ethanol. Colonies were imaged and enumerated on a Cellular Technology Limited (CTL) (Shaker Heights, OH) ImmunoSpot® Analyzer. Raw colony counts were used to plot kill curves and calculate OPA titers.

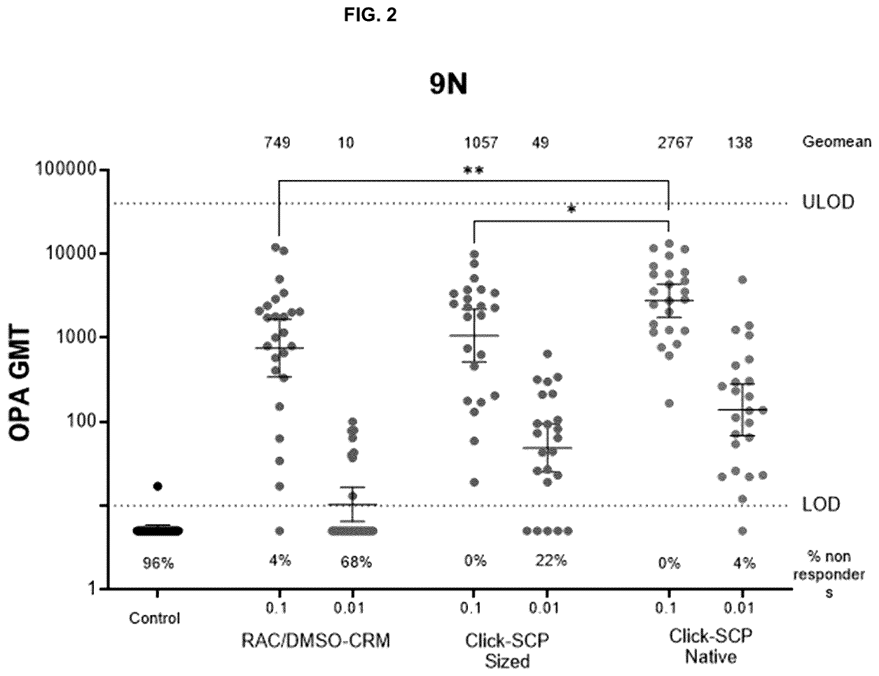

OPA titers (geometric mean titer (GMT) and % non-responders at five weeks at different doses are shown in Table 4. The results are presented in FIG. 2.

TABLE 4
st9N OPA titers following vaccination with st9N conjugates using
the either RAC/DMSO or Click chemistry. The conjugates were used
to vaccinate animals in the presence of adjuvant. Female Swiss-Webster
mice, 6-8 weeks old; Doses: 0.01 or 0.1 μg/animal + AlPO₄;
Vaccinate: 0 and 3 wk.; terminal bleed wk. 5 Readout: OPA

| | | 0.01 | 0.1 |
|---|---|---|---|
| RAC/DMSO - CRM₁₉₇ | Mean | 10 | 749 |
| | % of non-responders | 68 | 4 |
| Click-SCP (Sized) | Mean | 49 | 1057 |
| | % of non-responders | 22 | 0 |
| Click-SCP (Native) | Mean | 138 | 2767 |
| | % of non-responders | 4 | 0 |

The data of Table 4 and FIG. 2 indicate that the serotype 9N conjugates elicited dose dependent OPA titers in a murine immunogenicity model. As shown in Table 4, serotype 9N conjugates with Click chemistry induced higher OPA GMT at all doses, particularly as the polysaccharide size increase.

### Example 5. Preparation of Further Serotype 9N Glycoconjugate Using Click Chemistry

Further serotype 9N glycoconjugates have been produced using click chemistry (see Example 3). The attributes of these conjugates are shown at Table 5.

TABLE 5
Conjugate obtained using click chemistry (carrier protein: SCP)

| Conjugate # | #17 | #18 | #19 | #20 |
|---|---|---|---|---|
| Poly MW (kDa) | 683 | 531 | 576 | 576 |
| Azido Poly DoA (%) per Poly RU | 32 | 32 | 9 | 41 |
| Azido Poly Mw (kDa) | 605 | 471 | 521 | 534 |
| Alkyne-SCP Mw (kDa) | 101 | 101 | 101 | 101 |
| Alkyne-SCP DoA (AAA), alkynes/ | 19 | 19 | 19 | 19 |
| SCP | | | | |

Conjugate Data

| | | | | |
|---|---|---|---|---|
| Conjugate Yield (%) | 87 | 95 | 88 | 88 |
| Saccharide/Protein Ratio (Output) | 1.01 | 1.10 | 1.06 | 0.82 |
| % Free Saccharide | <5 | <5 | 6 | <5 |
| Conjugate MW (kDa) | 3765 | 2647 | 2065 | 1826 |

### Example 6. Evaluation of Immunogenicity of S. pneumoniae Serotype 9N Glycoconjugates Prepared Using Click Chemistry

The opsonophagocytic activity (OPA) titers in mice vaccinated with serotype 9N Click-SCP conjugates #15, #17, #18, #19 and #20 (Table 5) were determined under standard conditions. See attributes of the tested conjugates at Tables 3 and 5.

Groups of twenty-five 6-8 weeks old female Swiss Webster mice were immunized with 0.01 μg/animal or 0.1 μg/animal of test conjugates via the subcutaneous route on week 0. The mice were boosted with the same dose of conjugate on week 3 and then bled at week 5. Each vaccination was formulated with 100 μg/dose of AIPO₄ as an adjuvant. All preclinical immunogenicity studies were powered to detect a 4 to 5-fold difference in OPA titers using 25 mice per group. Whole blood was collected from mice two weeks after the second vaccination (Week 5, PD 2) and sera used for analyses. Serotype-specific OPAs were performed on week 5 sera samples.

Opsonophagocytic activity (OPA) assays are used to measure functional antibodies in murine sera specific for S. pneumonia serotype 9N (see Example 4).

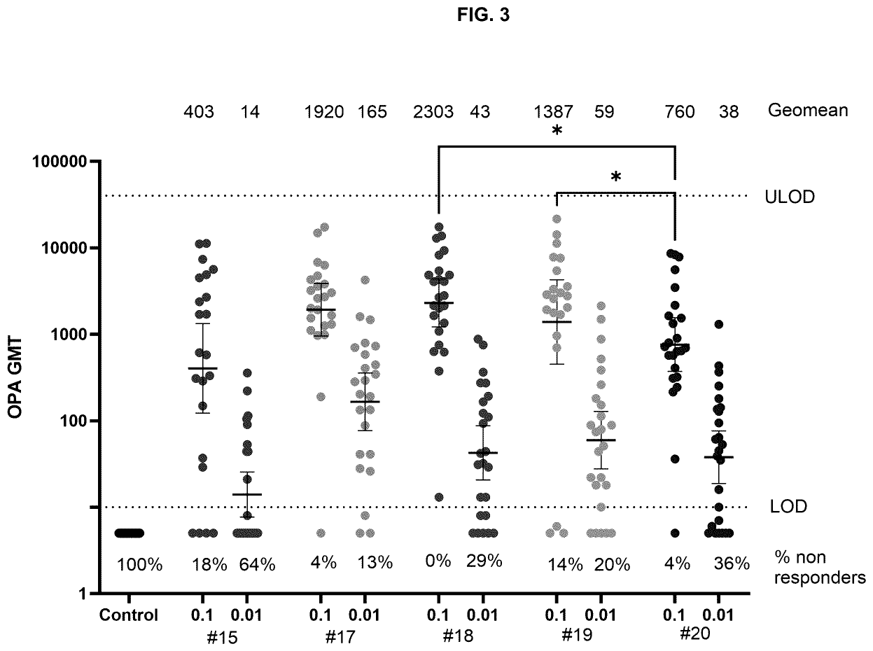

OPA titers (geometric mean titer (GMT) and % non-responders at five weeks at different doses are shown in Table 5. The results are presented in FIG. 3.

TABLE 6
st9N OPA titers following vaccination with st9N conjugates
using Click chemistry. The conjugates were used to vaccinate
animals in the presence of adjuvant. Female Swiss-Webster mice, 6-8
weeks old; Doses: 0.01 or 0.1 μg/animal + AlPO₄; Vaccinate:
0 and 3 wk.; terminal bleed wk. 5 Readout: OPA

| Conjugate | | 0.01 | 0.1 |
|---|---|---|---|
| #15 | Mean | 14 | 403 |
| | % of non-responders | 64 | 18 |
| #17 | Mean | 165 | 1920 |
| | % of non-responders | 13 | 4 |
| #18 | Mean | 43 | 2303 |
| | % of non-responders | 29 | 0 |
| #19 | Mean | 59 | 1387 |
| | % of non-responders | 20 | 14 |
| #20 | Mean | 38 | 760 |
| | % of non-responders | 36 | 4 |

As shown in Table 6, serotype 9N conjugates with Click chemistry induced high OPA GMT in mice, particularly as the polysaccharide size increase and the degree of activation of the activated saccharide is between 9 to 32%.

All publications and patent applications mentioned in the specification are indicative of the level of those skilled in the art to which this invention pertains. All publications and patent applications are hereby incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

1.-4. (canceled)

5. A Streptococcus pneumoniae serotype 9N glycoconjugate comprising a serotype 9N saccharide covalently conjugated to a carrier protein (CP) through a spacer and having the general formula (I): 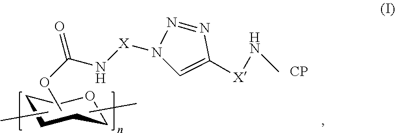
wherein X is selected from the group consisting of CH₂(CH₂)_{n′}, (CH₂CH₂O)ₘCH₂CH₂, NHCO(CH₂)_{n′}, NHCO(CH₂CH₂O)ₘCH₂CH₂, OCH₂(CH₂)_{n′} and O(CH₂CH₂O)ₘCH₂CH₂, where n′ is 1 to 10 and m is 1 to 4; wherein X′ is CH₂O(CH₂)_{n″}CH₂C═O or CH₂O(CH₂CH₂O)_{m′}(CH₂)_{n″}CH₂C═O, where n″ is 0 to 10 and m′ is 0 to 4; wherein the structure in square brackets represents a repeat unit of the serotype 9N saccharide and n represents the number of repeating units.

6. The Streptococcus pneumoniae serotype 9N glycoconjugate of claim 44, wherein said serotype 9N glycoconjugate comprises a serotype 9N saccharide covalently conjugated to a carrier protein (CP) through a spacer and having the general formula (VI), 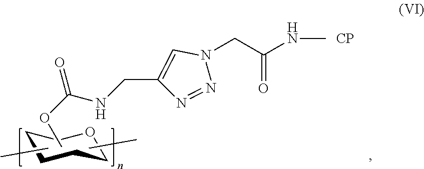
wherein the structure in square brackets represents a repeat unit of the serotype 9N saccharide and n represents the number of repeating units.

7. The Streptococcus pneumoniae serotype 9N glycoconjugate of claim 5, wherein the weight average molecular weight (Mw) of said polysaccharide before conjugation is between 50 kDa and 1,000 kDa, or 200 kDa and 500 kDa, or 300 kDa and 800 kDa, or 400 kDa and 750 kDa, or 500 kDa and 700 kDa.

8.-9. (canceled)

10. The Streptococcus pneumoniae serotype 9N glycoconjugate of claim 5, having a weight average molecular weight (Mw) of between 1,500 kDa and 4,500 kDa, or 1,000 kDa and 5,000 kDa, or 2000 kDa and 4,000 kDa.

11.-12. (canceled)

13. The Streptococcus pneumoniae serotype 9N glycoconjugate of claim 5, wherein the ratio of serotype 9N capsular polysaccharide to carrier protein in the conjugate is between 0.5 and 1.5, or 0.8 and 1.2.

14. The Streptococcus pneumoniae serotype 9N glycoconjugate of claim 5, wherein the degree of activation of the activated saccharide is between 5.0 to 35%, or 9.0 to 32%.

15.-23. (canceled)

24. The Streptococcus pneumoniae serotype 9N glycoconjugate of claim 5, wherein the carrier protein of said serotype 9N capsular polysaccharide glycoconjugate is Cross-Reactive Material 197 (CRM₁₉₇), Streptococcal C5a Peptidase (SCP), diphtheria toxoid (DT), tetanus toxoid (TT), or H. influenzae protein D (PD).

25.-34. (canceled)

35. The Streptococcus pneumoniae serotype 9N glycoconjugate of claim 5 wherein the degree of conjugation of said serotype 9N glycoconjugate is between 4 and 12.

36. The Streptococcus pneumoniae serotype 9N glycoconjugate of claim 44, wherein the weight average molecular weight (Mw) of said polysaccharide before conjugation is between 50 kDa and 1,000 kDa, or 200 kDa and 500 kDa, or 300 kDa and 800 kDa, or 400 kDa and 750 kDa, or 500 kDa and 700 kDa.

37. The Streptococcus pneumoniae serotype 9N glycoconjugate of claim 44, having a weight average molecular weight (Mw) of between 1,500 kDa and 4,500 kDa, or 1,000 kDa and 5,000 kDa, or 2000 kDa and 4,000 kDa.

38. The Streptococcus pneumoniae serotype 9N glycoconjugate of claim 44, wherein the ratio of serotype 9N capsular polysaccharide to carrier protein in the conjugate is between 0.5 and 1.5, or 0.8 and 1.2.

39. The Streptococcus pneumoniae serotype 9N glycoconjugate of claim 44, wherein the degree of activation of the activated saccharide is between 5.0 to 35%, or 9.0 to 32%.

40. The Streptococcus pneumoniae serotype 9N glycoconjugate of claim 44 wherein the degree of conjugation of said serotype 9N glycoconjugate is between 4 and 12.

41. The Streptococcus pneumoniae serotype 9N glycoconjugate of claim 44, wherein the carrier protein of said serotype 9N capsular polysaccharide glycoconjugate is Cross-Reactive Material 197 (CRM₁₉₇), Streptococcal C5a Peptidase (SCP), diphtheria toxoid (DT), tetanus toxoid (TT), or H. influenzae protein D (PD).

42. A method of making the Streptococcus pneumoniae serotype 9N glycoconjugate of claim 5 comprising reacting, via a Cu⁺¹ mediated azide-alkyne cycloaddition reaction, a carrier protein activated with an agent bearing an N-hydroxysuccinimide (NHS) moiety and an alkyne group with a serotype 9N saccharide activated with an azido linker, wherein said agent bearing an NHS moiety and alkyne group is a compound of formula (III): 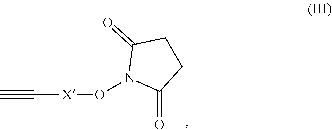
wherein X′ is CH₂O(CH₂)_{n″}CH₂C═O or CH₂O(CH₂CH₂O)_{m′}(CH₂)_{n″}CH₂C═O, where n″ is 0 to 10 and m′ is 0 to 4; and wherein said azido linker is a compound of formula (IV): 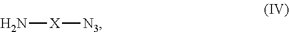
wherein X is selected from the group consisting of CH₂(CH₂)_{n′}, (CH₂CH₂O)ₘCH₂CH₂, NHCO(CH₂)_{n′}, NHCO(CH₂CH₂O)ₘCH₂CH₂, OCH₂(CH₂)_{n′} and O(CH₂CH₂O)ₘCH₂CH₂, where n′ is 1 to 10 and m is 1 to 4.

43. The method of claim 42, wherein the carrier protein of said serotype 9N capsular polysaccharide glycoconjugate is Cross-Reactive Material 197 (CRM₁₉), Streptococcal C5a Peptidase (SCP), diphtheria toxoid (DT), tetanus toxoid (TT), or H. influenzae protein D (PD).

44. A Streptococcus pneumoniae serotype 9N glycoconjugate comprising a serotype 9N saccharide covalently conjugated to a carrier protein (CP) through a spacer and having the general formula (V), 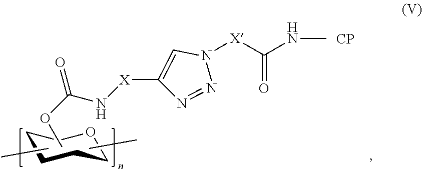
wherein X is selected from the group consisting of CH₂(CH₂)_{n′}, (CH₂CH₂O)ₘCH₂CH₂, NHCO(CH₂)_{n′}, NHCO(CH₂CH₂O)ₘCH₂CH₂, OCH₂(CH₂)_{n′} and O(CH₂CH₂O)ₘCH₂CH₂, where n′ is 0 to 10 and m is 1 to 4; wherein X′ is selected from the group consisting of CH₂(CH₂)_{n″}, CH₂O(CH₂)_{n″}CH₂ and CH₂O(CH₂CH₂O)_{m′}(CH₂)_{n″}CH₂, where n″ is 0 to 10 and m′ is 0 to 4; and wherein the structure in square brackets represents a repeat unit of the serotype 9N saccharide and where n represents the number of repeating units.

45. A method of making the Streptococcus pneumoniae serotype 9N glycoconjugate of claim 44 comprising reacting, via a Cu⁺¹ mediated azide-alkyne cycloaddition reaction, a carrier protein activated with an azido linker with a serotype 9N saccharide activated with an agent bearing an N-hydroxysuccinimide (NHS) moiety and an alkyne group, wherein said alkyne linker comprises X and wherein said agent bearing an N-hydroxysuccinimide (NHS) moiety and an azido group comprises X′.

46. The method of claim 45, wherein the carrier protein of said serotype 9N capsular polysaccharide glycoconjugate is Cross-Reactive Material 197 (CRM₁₉₇), Streptococcal C5a Peptidase (SCP), diphtheria toxoid (DT), tetanus toxoid (TT), or H. influenzae protein D (PD).

47. The Streptococcus pneumoniae serotype 9N glycoconjugate of claim 5, wherein said serotype 9N glycoconjugate comprises a serotype 9N saccharide covalently conjugated to a carrier protein (CP) through a spacer and having the general formula (II), 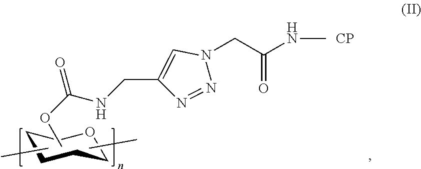
wherein the structure in square brackets represents a repeat unit of the serotype 9N saccharide and n represents the number of repeating units.
